(19) United States
(12) Patent Application Publication
(10) Pub. No.: US 2022/0127267 A1
(43) Pub. Date: Apr. 28, 2022

(54) THIENO[3,2-B] PYRROLE[3,2-D]PYRIDAZINONE DERIVATIVES AND THEIR USE AS PKM2 DERIVATIVES FOR THE TREATMENT OF CANCER, OBESITY AND DIABETES RELATED DISORDERS

(71) Applicant: Agios Pharmaceuticals, Inc., Cambridge, MA (US)

(72) Inventors: Tao Liu, Wellesley, MA (US); Zhihua Sui, Cambridge, MA (US); Jingjing Ji, Shanghai (CN)

(21) Appl. No.: 17/429,073

(22) PCT Filed: Feb. 12, 2020
(86) PCT No.: PCT/US2020/017965

§ 371 Date: Aug. 6, 2021

Related U.S. Application Data
(60) Provisional application No. 62/805,040, filed on Feb. 13, 2019.

(51) Int. Cl. C07D 471/14; C07D 471/04; C07D 513/14; C07D 498/14; C07D 495/04; C07D 495/14

(52) U.S. Cl. CPC C07D 471/14; C07D 471/04; C07D 495/14; C07D 498/14; C07D 495/04; C07D 513/14

(57) ABSTRACT

Described herein are compounds that regulate pyruvate kinase activity, pharmaceutical compositions and methods of use thereof. These compounds are represented by Formula (I) wherein R₂, L₁-L₂, U₁-U₇, m, ring A, and Q are as defined herein.

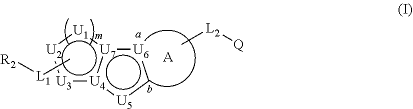

### RELATED APPLICATIONS

This application claims the benefit of U.S. Provisional Application No. 62/805,040, filed Feb. 13, 2019, the entire contents of which are incorporated herein by reference.

### BACKGROUND

Pyruvate kinase (PK) is a metabolic enzyme that converts phosphoenolpyruvate to pyruvate during glycolysis. Four PK isoforms exist in mammals: the L and R isoforms (from the PKLR gene) are expressed in liver and red blood cells respectively, and the PKM gene encodes two splice variants, the M1 isoform that is expressed in most adult tissues, and the M2 isoform that is expressed during embryonic development and in some adult tissues including the kidney and hematopoietic stem cells. Many tumor cells also express PKM2. Modulation (e.g. inhibition or activation) of PKM2 may be effective in the treatment of a number of disorders, e.g., cancer, obesity, diabetic diseases (e.g. diabetic nephropathy (DN)), coronary artery disease (CAD), Bloom Syndrome (BS), autoimmune conditions, and proliferation-dependent diseases (e.g., benign prostatic hyperplasia (BPH)).

### SUMMARY

Described herein are compounds of and encompassed within Formula (I), the compounds of Tables 1-3 (collectively referred to herein as the “Disclosed Compounds”), that activate PKR and/or regulate PKM2, wild type and/or mutant enzymes (such as those described herein), and pharmaceutically acceptable salts thereof.

In one embodiment, provided herein a compound represented by the following structural formula:

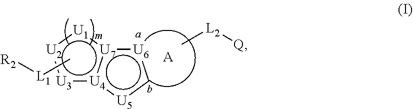

or a pharmaceutically acceptable salt thereof. The definition of each variable is provided below.

In one specific embodiment, the compound or pharmaceutically acceptable salt thereof is selected from any of the compounds of Tables 1-3.

In another embodiment, provided herein is a pharmaceutical composition comprising a Disclosed Compound or a pharmaceutically acceptable salt thereof and a pharmaceutically acceptable carrier or diluent.

The present disclosure further provides a method of treating anemia in a subject comprising administering to the subject an effective amount of (1) a Disclosed Compound or a pharmaceutically acceptable salt thereof; (2) a pharmaceutically acceptable composition comprising a Disclosed Compound or a pharmaceutically acceptable salt thereof, and a pharmaceutically acceptable carrier. In certain embodiments, the anemia is a dyserythropoietic anemia such as congenital dyserythropoietic anemia type I, II, III, or IV.

The present disclosure further provides a method for treating sickle cell disease in a subject comprising administering to a subject an effective amount of (1) a Disclosed Compound or a pharmaceutically acceptable salt thereof; (2) a pharmaceutical composition comprising a Disclosed Compound or a pharmaceutically acceptable salt thereof, and a pharmaceutically acceptable carrier.

The present disclosure further provides a method for treating hemolytic anemia (e.g., chronic hemolytic anemia caused by phosphoglycerate kinase deficiency, Blood Cells Mol Dis, 2011; 46(3):206) in a subject comprising administering to the subject an effective amount of (1) a Disclosed Compound or a pharmaceutically acceptable salt thereof; (2) a pharmaceutical composition comprising a Disclosed Compound or a pharmaceutically acceptable salt thereof, and a pharmaceutically acceptable carrier.

In certain embodiments, the hemolytic anemia is hereditary and/or congenital hemolytic anemia, acquired hemolytic anemia, chronic hemolytic anemia caused by phosphoglycerate kinase deficiency, anemia of chronic diseases, non-spherocytic hemolytic anemia, or hereditary spherocytosis. In certain embodiments, the hemolytic anemia is hereditary and/or congenital hemolytic anemia, acquired hemolytic anemia, or anemia as part of a multi-system disease. In certain embodiments, the hemolytic anemia is congenital anemia. In certain embodiments, the hemolytic anemia is hereditary (e.g. non-spherocytic hemolytic anemia or hereditary spherocytosis).

The present disclosure further provides a method for treating thalassemia (e.g., beta-thalassemia), hereditary spherocytosis, hereditary elliptocytosis, abetalipoproteinemia (or Bassen-Kornzweig syndrome), paroxysmal nocturnal hemoglobinuria, acquired hemolytic anemia (e.g., congenital anemias (e.g., enzymopathies)), sickle cell disease, or anemia of chronic diseases in a subject comprising administering to the subject an effective amount of (1) a Disclosed Compound or a pharmaceutically acceptable salt thereof; (2) a pharmaceutical composition comprising a Disclosed Compound or a pharmaceutically acceptable salt thereof, and a pharmaceutically acceptable carrier. In one embodiment, the acquired hemolytic anemia comprises congenital anemias. In certain embodiments, the provided method is to treat thalassemia. In certain embodiments, the thalassemia is beta-thalassemia.

The present disclosure further provides a method for treating pyruvate kinase deficiency (PKD) in a subject, the method comprising administering to the subject an effective amount of (1) a Disclosed Compound or a pharmaceutically acceptable salt thereof; (2) a pharmaceutical composition comprising a Disclosed Compound or a pharmaceutically acceptable salt thereof, and a pharmaceutically acceptable carrier. In certain embodiments, the PKD is a deficiency of PKR. In certain embodiments, the deficiency of PKR is associated with a pyruvate kinase R mutation.

Compounds and pharmaceutical compositions described herein are activators of PKR having lower activities compared to the wild type, thus are useful for methods of the present disclosure. In certain embodiments, the PKR is a wild type. In certain embodiments, the PKR is a mutant. Such mutations in PKR can affect enzyme activity (catalytic efficiency), regulatory properties (modulation by fructose bisphosphate (FBP)/ATP), and/or thermostability of the enzyme. Examples of such mutations are described in Valentini et al, JBC 2002. Some examples of the mutants that are activated by the Disclosed Compounds include G332S, G364D, T384M, R479H, R479K, R486W, R532W, K410E, R510Q, and R490W. Without being bound by theory, in certain embodiments, the Disclosed Compounds affect the activities of PKR mutants by activating FBP non-responsive PKR mutants, restoring thermostability to mutants with decreased stability, or restoring catalytic efficiency to impaired mutants. The activating activity of the present compounds against PKR mutants may be tested following a method described in the Examples. In certain embodiments, the Disclosed Compounds are also activators of wild type PKR.

In an embodiment, the disclosure provides a method for activating PKR in red blood cells in a subject in need thereof comprising administering to the subject an effective amount of (1) a Disclosed Compound or a pharmaceutically acceptable salt thereof; (2) a pharmaceutical composition comprising a Disclosed Compound or a pharmaceutically acceptable salt thereof, and a pharmaceutically acceptable carrier. In certain embodiments, the PKR is a wild type. In certain embodiments, the PKR is a mutant.

In an embodiment, the mutant PKR is selected from G332S, G364D, T384M, K410E, R479H, R479K, R486W, R532W, R510Q, and R490W. In certain embodiments, the mutant PKR is selected from A468V, A495V, I90N, T408I, and Q421K, and R498H. In certain embodiments, the mutant PKR is R532W, K410E, or R510Q.

The present disclosure further provides a method of modulating pyruvate kinase M2 (PKM2) activity (that regulate PKM2, wild type and/or mutant enzymes, such as those described herein) in a subject in need thereof, comprising administering to the subject an effective amount of (1) a Disclosed Compound or a pharmaceutically acceptable salt thereof; (2) a pharmaceutical composition comprising a Disclosed Compound or a pharmaceutically acceptable salt thereof, and a pharmaceutically acceptable carrier.

In another embodiment, provided is a method of modulating (e.g., increasing or decreasing) the level of PKM2 activity in a subject in need thereof comprising administering an effective amount of a Disclosed Compound to the subject. In some embodiments, a compound or a composition described herein is used to maintain PKM2 in its active conformation or activate pyruvate kinase activity in proliferating cells as means to divert glucose metabolites into catabolic rather than anabolic processes in the patient. In certain embodiments, the provided method increases the level of (i.e. activating) PKM2 activity in the subject. In certain embodiments, the provided method decreases the level of PKM2 activity in the subject.

In another embodiment, provided is a method of modulating (e.g., increasing or decreasing) the level of plasma glucose in a subject in need thereof comprising administering an effective amount of a Disclosed Compound to the subject. In certain embodiments, the provided method increases the level of plasma glucose in the subject. In certain embodiments, the provided method decreases the level of plasma glucose in the subject.

In another embodiment, provided is a method of inhibiting cell proliferation in a subjectin need thereof comprising administering an effective amount of a Disclosed Compound to the subject. E.g., this method can inhibit growth of a transformed cell, e.g., a cancer cell, or generally inhibiting growth in a PKM2-dependent cell that undergoes aerobic glycolysis.

In another embodiment, provided is a method of treating a subject suffering from or susceptible to a disease or disorder associated with the function of PKM2 (i.e., a disease associated with the aberrant activity of PKM2) comprising administering an effective amount of a Disclosed Compound to the subject.

In certain embodiments, the disease is a neoplastic disorder. In certain embodiments, the disease is cancer, obesity, a diabetic disease (e.g. diabetic nephropathy (DN)), atherosclerosis, restenosis, coronary artery disease (CAD), Bloom Syndrome (BS), benign prostatic hyperplasia (BPH), or an autoimmune disease. In certain embodiments, the disease is cancer. In certain embodiments, the disease is a diabetic disease. In certain embodiments, the diabetic disease is diabetic nephropathy (DN). In certain embodiments, the disease is coronary artery disease (CAD).

In certain embodiment, the methods described above further comprise identifying or selecting a subject who would benefit from modulation (e.g., activation) of PKM2 (and/or plasma glucose). For example, the patient can be identified on the basis of the level of PKM2 activity in a cell of the patient for treatment of cancer associated with PKM2 function. In another embodiment, the selected patient is a subject suffering from or susceptible to a disorder or disease identified herein, e.g., a disorder characterized by unwanted cell growth or proliferation.

In one embodiment, provided is use of a Disclosed Compound or a pharmaceutically acceptable salt thereof or a pharmaceutical composition comprising the same in any of the methods of the invention described above. In one embodiment, provided is a Disclosed Compound or a pharmaceutically acceptable salt thereof or a pharmaceutical composition comprising the same for use in any of the method of the invention described above. In another embodiment, provided is use of a Disclosed Compound or a pharmaceutically acceptable salt thereof or a pharmaceutical composition comprising the same for the manufacture of a medicament for any of the method of the invention described.

### DETAILED DESCRIPTION OF THE INVENTION

The details of construction and the arrangement of components set forth in the following description or illustrated in the drawings are not meant to be limiting. Embodiments can be practiced or carried out in various ways. The phraseology and terminology used herein is for purpose of description and shouldn't be regarded as limiting.

### Definitions

Disclosed Compounds can comprise one or more asymmetric centers, and thus can exist in various stereoisomeric forms, e.g., enantiomers and/or diastereomers. For example, the Disclosed Compounds can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H. Tables of Resolving Agents and Optical Resolutions p. 268 (E. L. Eliel, Ed., Univ. of Notre Dame Press, Notre Dame, Ind. 1972).

The Disclosed Compounds may exist in various tautomeric forms. The term “tautomers” or “tautomeric” refers to a compound that is a mixture of two or more structurally distinct compounds that are in rapid equilibrium at room temperature. Exemplary tautomerizations include keto-to-enol, amide-to-imide, lactam-to-lactim, enamine-to-imine, and enamine-to-(a different enamine) tautomerizations. The present teachings encompass compounds in the form of tautomers, which includes forms not depicted structurally. All such isomeric forms of such compounds are expressly included. If a tautomer of a compound is aromatic, this compound is aromatic. If a tautomer of a compound is a heteroaryl, this compound is heteroaryl. For example, the compound pyridine-2-ol may exist in both amide and imide tautomeric forms shown here:

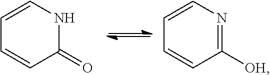 [SMILES: O=c1cccc[nH]1 ; Oc1ccccn1]

and is considered to be aromatic.

It is to be understood that when a compound herein is represented by a structural formula or designated by a chemical name herein, all other tautomeric forms which may exist for the compound are encompassed by the structural formula.

The term “alkyl” refers to a radical of a straight-chain or branched saturated hydrocarbon group having from 1 to 10 carbon atoms (“C₁-C₁₀ alkyl”). Examples of C₁-C₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl).

The term “halo” or “halogen” refers to fluorine, chlorine, bromine, or iodine.

The term “haloalkyl” refers to a substituted alkyl group, wherein one or more of the hydrogen atoms are independently replaced by a halo group, e.g., fluoro, bromo, chloro, or iodo and includes alkyl moieties in which all hydrogens have been replaced by halo (e.g., perfluoroalkyl). In some embodiments, the haloalkyl moiety has 1 to 6 carbon atoms (“C₁-C₆ haloalkyl”).

The term “hydroxyalkyl” refers to a substituted alkyl group, wherein one or more of the hydrogen atoms are independently replaced by a hydroxyl group. In some embodiments, the hydroxyalkyl moiety has 1 to 6 carbon atoms (“C₁-C₆ hydroxyalkyl”).

The term “alkoxy” or “alkoxyl” refers to an —O-alkyl radical, e.g., with between 1 and 6 carbon atoms.

The term “alkenyl” refers to branched or straight-chain monovalent hydrocarbon radical containing at least one double bond. Alkenyl may be mono or polyunsaturated, and may exist in the E or Z configuration. Unless otherwise specified, an alkenyl group typically has 2-6 carbon atoms, i.e., (C₂-C₆)alkenyl. For example, “(C₂-C₄)alkenyl” means a radical having from 2-4 carbon atoms in a linear or branched arrangement.

The term “alkynyl” refers to branched or straight-chain monovalent hydrocarbon radical containing at least one triple bond. Unless otherwise specified, an alkynyl group typically has 2-6 carbon atoms, i.e., (C₂-C₆)alkynyl. For example, “(C₂-C₄)alkynyl” means a radical having from 2-4 carbon atoms in a linear or branched arrangement.

The term “carbocyclyl” or “carbocyclic” refers to an aromatic or a non-aromatic monocyclic, bicyclic, or tricyclic or polycyclic hydrocarbon ring system having from 3 to 14 ring carbon atoms (“C₃-C₁₄ carbocyclyl”) and zero heteroatoms in the non-aromatic ring system. Carbocyclyl groups include fully saturated ring systems (e.g., cycloalkyls), partially saturated ring systems, and fully unsaturated systems (e.g., aromatics). In some embodiments, a carbocyclyl group has 3 to 10 ring carbon atoms (“C₃-C₁₀ carbocyclyl”).

The term “cycloalkyl” refers to completely saturated monocyclic or bicyclic (e.g., fused) hydrocarbon groups of 3-12 carbon atoms. In some embodiments, “cycloalkyl” is a monocyclic cycloalkyl. Examples of monocyclic cycloalkyls include cyclopropyl, cyclobutyl, cyclopentyl, cyclohexyl, cycloheptyl and cyclooctyl. In some embodiments, “cycloalkyl” is a fused bicyclic cycloalkyl. Examples of fused bicyclic cycloalkyls include bicycloheptane, bicyclooctane, octahydropentalene, octahydroindene, decahydronaphthalene.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more double bonds. Heterocyclyl polycyclic ring systems can include one or more heteroatoms in one or more rings. “Heterocyclyl” also includes (1) ring systems wherein the heterocyclyl ring, as defined above, is fused with one or more carbocyclyl groups; or (2) ring systems wherein the heterocyclyl ring, as defined above, is fused with one or more aryl or heteroaryl groups. In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”).

Exemplary heterocyclyl groups include aziridinyl, oxiranyl, thiiranyl, azetidinyl, oxetanyl, thietanyl, tetrahydrofuranyl, dihydrofuranyl, tetrahydrothiophenyl, dihydrothiophenyl, pyrrolidinyl, dihydropyrrolyl, pyrrolyl-2,5-dine, dioxolanyl, oxathiolanyl, dithiolanyl, triazolinyl, oxadiazolinyl, thiadiazolinyl, piperidinyl, tetrahydropyranyl, dihydropyridinyl, thianyl, piperazinyl, morpholinyl, dithianyl, dioxanyl, triazinanyl, azepanyl, oxepanyl, thiepanyl, azocanyl, oxecanyl, thiocanyl, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) carbocyclic aromatic ring system having 6-14 ring carbon atoms and zero heteroatoms provided in the aromatic ring system (“C₆-C₁₄ aryl”), including phenyl, naphthyl or anthracyl.

The term “heteroaryl” refers to a radical of a 5-14 membered monocyclic or polycyclic (e.g., bicyclic, tricyclic) aromatic ring system having ring carbon atoms and 1-4 ring heteroatoms provided in the aromatic ring system, wherein each heteroatom is independently selected from nitrogen, oxygen, and sulfur (“5-14 membered heteroaryl”). In some embodiments the heteroaryl can be a 5- or 6-membered monocyclic heteroaryl containing 1-4 heteroatoms. In some embodiments the heteroaryl can be an 8-12 membered bicyclic heteroaryl having 1-6 heteroatoms (“8-12 membered bicyclic heteroaryl”). In some embodiments the heteroaryl can be an 11-14 membered tricyclic heteroaryl ring system having 1-9 heteroatoms.

Exemplary monocyclic 5- or 6-membered heteroaryl groups include pyrrolyl, furanyl, thiophenyl, imidazolyl, pyrazolyl, oxazolyl, isoxazolyl, thiazolyl, isothiazolyl, triazolyl, oxadiazolyl, thiadiazolyl, tetrazolyl, pyridinyl, pyridazinyl, pyrimidinyl, pyrazinyl, triazinyl, and tetrazinyl.

Exemplary 8-12 membered bicyclic heteroaryl groups include benzimidazolyl, benzofuryl, benzoisoxazolyl, benzoisothiazolyl, benzothiadiazolyl, benzothiazolyl, benzothienyl, benzotriazolyl, benzoxadiazolyl, benzoxazolyl, imidazo[1,2-a]pyridyl, indazolyl, indolizinyl, indolyl, isoquinolinyl, oxazolopyridinyl, purinyl, pyridopyrimidinyl, pyrrolo[2,3]pyrimidinyl, pyrrolopyrazolyl, pyrroloimidazolyl, quinazolinyl, quinolinyl, thiazolopyridinyl, napthyridyl.

In heteroaryl groups that contain one or more nitrogen atoms, the point of attachment can be a carbon or nitrogen atom, as valency permits. Heteroaryl polycyclic ring systems can include one or more heteroatoms in one or both rings.

The term “saturated” refers to a moiety that does not contain a double or triple bond, i.e., the moiety only contains single bonds.

The term “optionally substituted” refers to being substituted or unsubstituted. In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent (e.g. C₁-C₆ alkyl, halogen, nitro, azide, cyano, hydroxyl, C₁-C₆ haloalkyl, C₁-C₆ hydroxyalkyl, C₁₋₆ haloalkoxy, C₃₋₆ cycloalkyl, C₆-C₁₀ aryl, monocyclic or bicyclic heteroaryl, and monocyclic or bicyclic heterocyclyl), at one or more substitutable positions of the group, and when more than one position in any given structure is substituted, the substituent is either the same or different at each position. The term “substituted” is contemplated to include substitution with all permissible substituents of organic compounds, and includes any of the substituents described herein that result in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valences of the heteroatoms and results in the formation of a stable moiety. The invention is not intended to be limited in any manner by the exemplary substituents described herein.

A “substitutable ring carbon atom” refers to a carbon atom on an aryl/heteroaryl/carbocyclyl/heterocyclyl ring with at least one hydrogen present on the carbon atom that is replaced with a permissible substituent as defined above. A “substitutable ring nitrogen atom” refers to a nitrogen atom on a heteroaryl or heterocyclyl ring with at least one hydrogen present on the nitrogen atom that is replaced with a permissible substituent.

Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that result in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valences of the heteroatoms and results in the formation of a stable moiety. The invention is not intended to be limited in any manner by the exemplary substituents described herein.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art, for example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, ammonium, quaternary ammonium, and amine cations formed using counterions such as halide, hydroxide, carboxylate, sulfate, phosphate, nitrate, lower alkyl sulfonate, and aryl sulfonate.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomologus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. In certain embodiments, the subject is a patient. The term “patient” refers to a human subject in need of treatment of a disease. In certain embodiments, the term “patient” is a human adult over 18 years old in need of treatment of a disease. In certain embodiments, the term “patient” is a human child no more than 18 years old in need of treatment of a disease. In certain embodiments, the patient is not under regular transfusion (e.g. having had no more than 4 transfusion episodes in the 12-month period). In certain embodiments, the patient is under regular transfusion (e.g. having had at least 4 transfusion episodes in the 12-month period). In certain embodiments, the subject has undergone splenectomy. In certain embodiments, the subject has undergone splenectomy and is under regular transfusion. In certain embodiments, the subject has undergone splenectomy and is not under regular transfusion.

The term “administer,” “administering,” or “administration” refers to implanting, absorbing, ingesting, injecting, inhaling, or otherwise introducing a Disclosed Compound, or a composition thereof, in or on a subject.

The terms “treatment,” “treat,” and “treating” refer to reversing, alleviating, or inhibiting the progress of a disease described herein. In some embodiments, treatment may be administered after one or more signs or symptoms of the disease have developed or have been observed (i.e., therapeutic treatment). In other embodiments, treatment may be administered in the absence of signs or symptoms of the disease. For example, treatment may be administered to a susceptible subject prior to the onset of symptoms (i.e., prophylactic treatment) (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a Disclosed Compound refers to an amount sufficient to elicit the desired biological response. An effective amount of a Disclosed Compound may vary depending on such factors as the desired biological endpoint, the pharmacokinetics of the compound, the condition being treated, the mode of administration, and the age and health of the subject. In certain embodiments, an effective amount is an amount sufficient for eliciting measurable activation of wild-type or mutant PKR. In certain embodiments, an effective amount is an amount sufficient for regulating 2,3-diphosphoglycerate and/or ATP levels in blood in need thereof or for treating pyruvate kinase deficiency (PKD), hemolytic anemia (e.g., chronic hemolytic anemia, hereditary non-spherocytic anemia), sickle cell disease, thalassemia (e.g., beta-thalassemia), hereditary spherocytosis, hereditary elliptocytosis, abetalipoproteinemia (or Bassen-Kornzweig syndrome), paroxysmal nocturnal hemoglobinuria, acquired hemolytic anemia (e.g., congenital anemias (e.g., enzymopathies)), anemia of chronic diseases or treating diseases or conditions that are associated with increased 2,3-diphosphoglycerate levels (e.g., liver diseases). In certain embodiments, an effective amount is an amount sufficient for eliciting measurable activation of wild-type or mutant PKR and for regulating 2,3-diphosphoglycerate levels in blood in need thereof or for treating pyruvate kinase deficiency (PKD), hemolytic anemia (e.g., chronic hemolytic anemia, hereditary non-spherocytic anemia), sickle cell disease, thalassemia (e.g., beta-thalassemia), hereditary spherocytosis, hereditary elliptocytosis, abetalipoproteinemia (or Bassen-Kornzweig syndrome), paroxysmal nocturnal hemoglobinuria, acquired hemolytic anemia (e.g., congenital anemias (e.g., enzymopathies)), anemia of chronic diseases or treating diseases or conditions that are associated with increased 2,3-diphosphoglycerate levels (e.g., liver diseases). In certain aspects, the effective amount is the amount required to reduce the patient's transfusion burden. In one aspect, the effective amount is between 0.01-100 mg/kg body weight/day of the provided compound, such as e.g., 0.1-100 mg/kg body weight/day. In certain embodiments, the effective amount is to reduce the patient's transfusion burden.

As used herein, reduction in transfusion burden means at least 20% reduction in the number of RBC units transfused within at least 5 weeks of treatment. In certain embodiments, the reduction in transfusion burden is ≥33% reduction in the number of RBC units transfused within at least 5 weeks of treatment. In certain embodiments, reduction of transfusion burden is observed in at least 10 weeks (e.g., at least 20 weeks or at least 24 weeks) of treatment.

As used herein, sickle cell disease (SCD), Hemoglobin SS disease, and sickle cell anemia are used interchangeably. Sickle cell disease (SCD) describes a group of inherited red blood cell disorders. In certain embodiments, subjects with SCD have abnormal hemoglobin, called hemoglobin S or sickle hemoglobin, in their red blood cells. In certain embodiments, people having SCD have at least one abnormal genes causing the body to make hemoglobin S. In certain embodiments, people having SCD have two hemoglobin S genes, Hemoglobin SS.

Thalassemia is an inherited blood disorder in which the body makes an abnormal form of hemoglobin. In certain embodiments, the abnormal form of hemoglobin results in deficiency of either alpha or beta globin. In certain embodiments, the disorder results in large numbers of red blood cells being destroyed, leading to anemia. In certain embodiments, the thalassemia is alpha thalassemia. In certain embodiments, the thalassemia is beta thalassemia.

The term “activator” as used herein also means an agent that (measurably) increases the activity of a pyruvate kinase (e.g., PKM2) or causes pyruvate kinase (e.g., PKM2) activity to increase to a level that is greater than PKM2's basal levels of activity. For example, the activator may mimic the effect caused by a natural ligand (e.g., FBP). The activator effect caused by a compound provided herein may be to the same, or to a greater, or to a lesser extent than the activating effect caused by a natural ligand, but the same type of effect is caused. A compound provided herein can be evaluated to determine if it is an activator by measuring either directly or indirectly the activity of the pyruvate kinase when subjected to said compound. The activity of a compound provided herein can be measured, for example, against a control substance. In some instances, the activity measured of the test compound is for activation of PKM2. The activity of PKM2 can be measured, for example, by monitoring the concentration of a product such as ATP or levels of a cofactor such as NADH used in a coupled enzyme assay system (see WO2011/002817).

The term “activator” as used herein also means an agent that (measurably) increases the activity of wild type pyruvate kinase R (wt PKR) or causes wild type pyruvate kinase R (wt PKR) activity to increase to a level that is greater than wt PKR's basal levels of activity or an agent that (measurably) increases the activity of a mutant pyruvate kinase R (mPKR) or causes mutant pyruvate kinase R (mPKR) activity to increase to a level that is greater than that mutant PKR's basal levels of activity, for examples, to a level that is 20%, 40%, 50%, 60%, 70%, 80%, 90% or 100% of the activity of wild type PKR.

The term “inhibitor” as used herein means an agent that (measurably) slows, stops, decreases, or inactivates the enzymatic activity of a pyruvate kinase (e.g., PKM2) to decrease to a level that is less than the pyruvate kinases (e.g. PKM2's) basal levels or activity.

The term “packed red blood cells” or PRBCs as used herein refer to red blood cells made from a unit of whole blood by centrifugation and removal of most of the plasma. In certain embodiments, a PRBC unit has a hematocrit of at least about 95%. In certain embodiments, a PRBC unit has a hematocrit of at least about 90%, 80%, 70%, 60%, 50%, 40%, 30%, 20%, or 10%.

The term “ex vivo” referring to a method as used herein means that the method takes place outside an organism. For example, a cell (e.g., red blood cells), a tissue or blood (containing at least red blood cells, plasma and hemoglobin) may be extracted from the organism to be contacted with one or more compounds provided herein or a pharmaceutically acceptable salt thereof or a pharmaceutical composition thereof, optionally under artificially controlled conditions (e.g., temperature).

The term “in vitro” referring to a method as used herein means that the method takes place outside an organism and is contained within an artificial environment. For example, a cell (e.g., red blood cells), a tissue or blood (containing at least red blood cells, plasma and hemoglobin) may be extracted from the organism to be contacted with one or more compounds provided herein or a pharmaceutically acceptable salt thereof or a pharmaceutical composition thereof, in a contained, artificial environment (e.g., a culture system), such as in a test tube, in a culture, in flask, in a microtiter plate, on a Petri dish, and the like.

### Compounds

Described herein are compounds and pharmaceutical compositions that activate wild type PKR and/or mutant PKRs such as those described herein. In one embodiment, provided is a compound of and encompassed within Formula (I), or a pharmaceutically acceptable salt thereof, or a pharmaceutical composition comprising a compound of and encompassed within Formula (I), or a pharmaceutically acceptable salt thereof.

Also described herein are compounds and pharmaceutical compositions that modulate PKM2. In one embodiment, the compounds and compositions described herein modulate PKM2 by binding in an allosteric binding pocket. In one embodiment, the compounds and compositions described herein inhibit PKM2. In one embodiment, the compounds and compositions described herein activate PKM2. In one embodiment, the Disclosed Compound is a compound of and encompassed within Formula (I), or a pharmaceutically acceptable salt thereof, or a pharmaceutical composition comprising a compound of and encompassed within Formula (I), or a pharmaceutically acceptable salt thereof.

In a first embodiment, the invention provides a compound represented by structural formula (I):

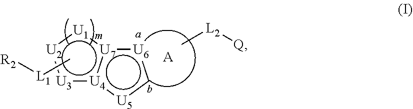

or a pharmaceutically acceptable salt thereof, wherein:
U₁, U₂, and U₃ are each independently N, O, S, C, or CR₁, as valency permits;

U₄, U₆, and U₇ are each independently N or C, as valency permits;

U₅ is N, NR₃, or CR₄, as valency permits;

m is 1 or 2;

Ring A is phenyl,

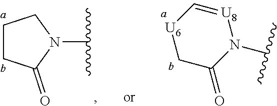

U₈ is N or CR₁;

each instance of R₁ is independently hydrogen or C₁-C₆ alkyl; L₁ is —S—, —S—CH₂—, —CH₂—S—, —S(═O)₂—, —S(═O)—, —S(═O)₂O—, —OS(═O)₂—, —S(═O)O—, —OS(═O)—, —S(═O)CH₂—, —CH₂S(═O)—, —S(═O)₂CH₂—, —CH₂S(═O)₂—, —S(═O)₂NR₅—, —NR₅S(═O)₂—, —S(═O)NR₅—, —NR₅S(═O)—, —NR₅S(═O)₂O—, —OS(═O)₂NR₅—, —NR₅S(═O)O—, —OS(═O)NR₅—, —S(═O)(═NR₅)—, —C(═O)—, —C(═O)O—, —OC(═O)—, —C(═O)NR₅—, —N(R₅)C(═O)—, —NR₅C(═O)O—, —OC(═O)NR₅—, —NR₅C(═O)NR₅—, —NR₅—, —C(═S)NR₅—, —N(R₅)C(═S)—, or —(CRⱼRₖ)_{q}—;

R₂ is C₁-C₆ alkyl, C₃-C₁₂ cycloalkyl, 3- to 8-membered heterocyclyl, 6- to 14-membered aryl, or 5- to 14-membered heteroaryl, wherein the alkyl is optionally substituted with 0 to 3 groups each independently selected from halogen, OH, CN, and NR₅R₅, and wherein each cycloalkyl, heterocyclyl, aryl, and heteroaryl is optionally substituted at each substitutable ring carbon atom with R^{p} and optionally substituted at each substitutable ring nitrogen atom by R^{nc}; or

-L₁-R₂ is —H, —CN, —CH₃, —OH, Br, C₁-C₆ haloalkyl, C₂-C₆ alkenyl, C₁-C₆ alkyl, C₃-C₁₂ cycloalkyl, 3- to 8-membered heterocyclyl, 6- to 14-membered aryl, or 5- to 14-membered heteroaryl; wherein each alkyl and alkenyl is optionally substituted with 0 to 3 groups each independently selected from halogen, OH, CN, and NR₅R₅, and wherein each cycloalkyl, heterocyclyl, aryl, and heteroaryl is optionally substituted at each substitutable ring carbon atom with R^{p} and optionally substituted at each substitutable ring nitrogen atom by R^{nc};
each instance of R^{p} is independently hydrogen, halogen, —CN, —NO₂, —N₃, C₁-C₆ alkyl, C₂-C₆ alkenyl, C₂-C₆ alkynyl, —OR^{c3}, —SR^{c3}, —N(R^{c3})₂, —C(═O)N(R^{c3})₂, —N(R^{c3})C(═O)R^{c3}, —C(═O)R^{c3}, —C(═O)OR^{c3}, —OC(═O)R^{c3}, —S(═O)R^{c3}, —S(═O)₂R^{c3}, —S(═O)OR^{c3}, —OS(═O)R^{c3}, —S(═O)₂OR^{c3}, —OS(═O)₂R^{c3}, —S(═O)N(R^{c3})₂, —S(═O)₂N(R^{c3})₂, —N(R^{c3})S(═O)R^{c3}, —N(R^{c3})S(═O)₂R^{c3}, —N(R^{c3})C(═O)OR^{c3}, —OC(═O)N(R^{c3})₂, —N(R^{c3})C(═O)N(R^{c3})₂, —N(R^{c3})S(═O)N(R^{c3})₂, —N(R^{c3})S(═O)₂N(R^{c3})₂, —N(R^{c3})S(═O)OR^{c3}, —N(R^{c3})S(═O)₂OR^{c3}, —OS(═O)N(R^{c3})₂, —OS(═O)₂N(R^{c3})₂; or alternatively
two instances of R^{p} attached to the adjacent ring carbon atoms, can be taken together with the carbon atoms to which they are attached to form 3- to 8-membered cycloalkyl, 5- to 6-membered saturated or partially saturated monocyclic heterocyclyl, or 5- to 6-membered monocyclic heteroaryl; wherein:
   each instance of R^{c3} is independently hydrogen or C₁-C₆ alkyl;

L₂ is —S—, —S—CH₂—, —CH₂—S—, —S(═O)₂—, —S(═O)—, —S(═O)₂O—, —OS(═O)₂—, —S(═O)O—, —OS(═O)—, —S(═O)CH₂—, —CH₂S(═O)—, —S(═O)₂CH₂—, —CH₂S(═O)₂—, —S(═O)₂NR₅—, —NR₅S(═O)₂—, —S(═O)NR₅—, —NR₅S(═O)—, —NR₅S(═O)₂O—, —OS(═O)₂NR₅—, —NR₅S(═O)O—, —OS(═O)NR₅—, —S(═O)(═NR₅)—, —C(═O)—, —C(═O)O—, —OC(═O)—, —C(═O)NR₅—, —N(R₅)C(═O)—, —NR₅C(═O)O—, —OC(═O)NR₅—, —NR₅C(═O)NR₅—, —NR₅—, —C(═S)NR₅—, —N(R₅)C(═S)—, or —(CRₐR_{b})ᵣ—;
each instance of R^{a} and R^{b} are independently hydrogen, halogen, CN, OH, NO₂, N₃, or C₁-C₆ alkyl; wherein the C₁-C₆ alkyl represented by R^{a} or R^{b} are each optionally substituted with 0 to 3 groups each independently selected from halogen, OH, CN, and NR₅R₅;
each instance of R^{j} and R^{k} are independently hydrogen, halogen, CN, OH, NO₂, N₃, or C₁-C₆ alkyl; wherein the C₁-C₆ alkyl represented by R^{a} or R^{b} are each optionally substituted with 0 to 3 groups each independently selected from halogen, OH, CN, and NR₅R₅;
q is 1 or 2;
r is 1 or 2;

Q is C₃-C₁₂ cycloalkyl, 3- to 8-membered heterocyclyl, 6- to 14-membered aryl, or 5- to 14-membered heteroaryl, each of which is optionally substituted at each substitutable ring carbon atom with R^{a} and optionally substituted at each substitutable ring nitrogen atom by R^{na}; or

-L₂-Q is —H, —CN, —CH₃, —OH, Br, C₁-C₆ haloalkyl, C₂-C₆ alkenyl, C₁-C₆ alkyl, C₃-C₁₂ cycloalkyl, 3- to 8-membered heterocyclyl, 6- to 14-membered aryl, or 5- to 14-membered heteroaryl; wherein each alkyl and alkenyl is optionally substituted with 0 to 3 groups each independently selected from halogen, OH, CN, and NR₅R₅, and wherein each cycloalkyl, heterocyclyl, aryl, and heteroaryl is optionally substituted at each substitutable ring carbon atom with Rⁿ and optionally substituted at each substitutable ring nitrogen atom by R^{na};
each instance of Rⁿ is independently hydrogen, halogen, —CN, —NO₂, —N₃, C₁-C₆ alkyl, C₂-C₆ alkenyl, C₂-C₆ alkynyl, —OR^{c4}, —SR^{c4}, —N(R^{c4})₂, —C(═O)N(R^{c4})₂, —N(R^{c4})C(═O)R^{c4}, —C(═O)R^{c4}, —C(═O)OR^{c4}, —OC(═O)R^{c4}, —S(═O)R^{c4}, —S(═O)₂R^{c4}, —S(═O)OR^{c4}, —OS(═O)R^{c4}, —S(═O)₂OR^{c4}, —OS(═O)₂R^{c4}, —S(═O)N(R^{c4})₂, —S(═O)₂N(R^{c4})₂, —N(R^{c4})S(═O)R^{c4}, —N(R^{c4})S(═O)₂R^{c4}, —N(R^{c4})C(═O)OR^{c4}, —OC(═O)N(R^{c4})₂, —N(R^{c4})C(═O)N(R^{c4})₂, —N(R^{c4})S(═O)N(R^{c4})₂, —N(R^{c4})S(═O)₂N(R^{c4})₂, —N(R^{c4})S(═O)OR^{c4}, —N(R^{c4})S(═O)₂OR^{c4}, —OS(═O)N(R^{c4})₂, or —OS(═O)₂N(R^{c4})₂; or alternatively
two instances of Rⁿ attached to the adjacent ring carbon atoms, can be taken together with the carbon atoms to which they are attached to form an optionally substituted 3- to 8-membered cycloalkyl, 5- to 6-membered saturated or partially saturated monocyclic heterocyclyl, or 5- to 6-membered monocyclic heteroaryl; wherein:
   each instance of R^{c4} is independently hydrogen or C₁-C₆ alkyl;

R₃ is hydrogen or C₁-C₆ alkyl;

R₄ is hydrogen, C₁-C₆ alkyl, C₁-C₆ haloalkyl, C₂-C₆ alkynyl, halogen, CN, —C(═O)NR₅R₅, or C≡C(CH₂)_{w}OH, wherein w is 1, 2, 3, 4, 5, or 6, and wherein each alkyl, haloalkyl, and alkynyl is independently optionally substituted with 1-3 instances of C₁-C₄ alkyl or halogen;

each instance of R^{na} and R^{nc} is independently hydrogen, C₁-C₆ alkyl, or C₁-C₆ haloalkyl; and each instance of R₅ is independently hydrogen or C₁-C₆ alkyl;

provided that

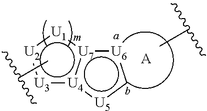

is other than

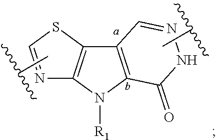

and provided that when

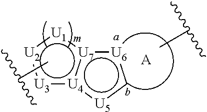

is

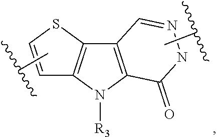

L₂ is —(CRₐR_{b})ᵣ— and Q is phenyl optionally substituted with Rⁿ and R^{na}, then L₁ is —(CRⱼRₖ)_{q}— and R₂ is cycloalkyl, heterocyclyl, aryl, or heteroaryl optionally substituted with R^{p} and R^{nc}.

In one aspect of the first embodiment, L₁ is —S—, —S—CH₂—, —CH₂—S—, —S(═O)₂—, —S(═O)—, —S(═O)₂O—, —OS(═O)₂—, —S(═O)O—, —OS(═O)—, —S(═O)CH₂—, —CH₂S(═O)—, —S(═O)₂CH₂—, —CH₂S(═O)₂—, —S(═O)₂NR₅—, —NR₅S(═O)₂—, —S(═O)NR₅—, —NR₅S(═O)—, —NR₅S(═O)₂O—, —OS(═O)₂NR₅—, —NR₅S(═O)O—, —OS(═O)NR₅—, —S(═O)(═NR₅)—, —C(═O)—, —C(═O)O—, —OC(═O)—, —C(═O)NR₅—, —N(R₅)C(═O)—, —NR₅C(═O)O—, —OC(═O)NR₅—, —NR₅C(═O)NR₅—, —NR₅—, —C(═S)NR₅—, —N(R₅)C(═S)—, or —(CRⱼRₖ)_{q}—;

R₂ is C₁-C₆ alkyl, C₃-C₁₂ cycloalkyl, 3- to 8-membered heterocyclyl, 6- to 14-membered aryl, or 5- to 14-membered heteroaryl, wherein the alkyl is optionally substituted with 0 to 3 groups each independently selected from halogen, OH, CN, and NR₅R₅, and wherein each cycloalkyl, heterocyclyl, aryl, and heteroaryl is optionally substituted at each substitutable ring carbon atom with R^{p} and optionally substituted at each substitutable ring nitrogen atom by R^{nc}; or

-L₁-R₂ is —H, —CN, —CH₃, —OH, Br, C₁-C₆ haloalkyl, or C₂-C₆ alkenyl wherein the alkenyl is optionally substituted with 0 to 3 groups each independently selected from halogen, OH, CN, and NR₅R₅;

Q is C₃-C₁₂ cycloalkyl, 3- to 8-membered heterocyclyl, 6- to 14-membered aryl, or 5- to 14-membered heteroaryl, each of which is optionally substituted at each substitutable ring carbon atom with Rⁿ and optionally substituted at each substitutable ring nitrogen atom by R^{na}; and

R₄ is hydrogen, C₁-C₆ alkyl, C₁-C₆ haloalkyl, halogen, CN, —C(═O)NR₅R₅, or C≡C(CH₂)_{w}OH, wherein w is 1, 2, 3, 4, 5, or 6.

In a second embodiment, the invention provides a compound according to structural formula (I), or a pharmaceutically acceptable salt thereof, wherein:

each instance of R^{p} is independently hydrogen, halogen, CN, OH, NO₂, N₃, C₁-C₆ alkyl, C₁-C₆ alkoxy, —C(═O)NR₅R₅, or NR₅R₅; or alternatively two instances of R^{p} attached to the adjacent ring carbon atoms, can be taken together with the carbon atoms to which they are attached to form 3- to 8-membered cycloalkyl, 5- to 6-membered saturated or partially saturated monocyclic heterocyclyl, or 5- to 6-membered monocyclic heteroaryl;

each instance of Rⁿ is independently hydrogen, halogen, CN, OH, NO₂, N₃, C₁-C₆ alkyl, C₁-C₆ alkoxy, —C(═O)NR₅R₅, or NR₅R₅; or alternatively two instances of Rⁿ attached to the adjacent ring carbon atoms, can be taken together with the carbon atoms to which they are attached to form 3- to 8-membered cycloalkyl, 5- to 6-membered saturated or partially saturated monocyclic heterocyclyl, or 5- to 6-membered monocyclic heteroaryl; and

the remaining variables are as defined in the first embodiment.

In a third embodiment, the invention provides a compound according to structural formula (I), or a pharmaceutically acceptable salt thereof, wherein:

L₁ is —S(═O)₂—, —S(═O)—, —C(═O)—, —C(═O)O—, —OC(═O)—, —C(═O)NR₅—, —N(R₅)C(═O)—, —NR₅—, or —(CRⱼRₖ)_{q}—; and

R₂ is C₁-C₆ alkyl, phenyl or 5- to 14-membered heteroaryl, wherein each phenyl and heteroaryl is optionally substituted at each substitutable ring carbon atom with R^{p} and optionally substituted at each substitutable ring nitrogen atom by R^{nc}; or

-L₁-R₂ is —H, —CN, —CH₃, —OH, Br, C₁-C₂ haloalkyl, —CH═CH₂, or C₁-C₆ hydroxyalkyl; and
each instance of R^{p} is independently hydrogen, halogen, CN, OH, NO₂, N₃, C₁-C₆ alkyl, C₁-C₆ alkoxy, —C(═O)NR₅R₅, or NR₅R₅; or alternatively
two instances of R^{p} attached to the adjacent ring carbon atoms, can be taken together with the carbon atoms to which they are attached to form 5- to 6-membered monocyclic heteroaryl;

L₂ is —S(═O)₂—, —S(═O)—, —C(═O)—, —C(═O)O—, —OC(═O)—, —C(═O)NR₅—, —N(R₅)C(═O)—, —NR₅—, or —(CRₐR_{b})ᵣ—;
each instance of R^{a} and R^{b} are independently hydrogen, halogen, CN, OH, NO₂, N₃, or C₁-C₆ alkyl; wherein the C₁-C₆ alkyl represented by R^{a} or R^{b} are each optionally substituted with 0 to 3 groups each independently selected from halogen, OH, CN, and NR₅R₅;
each instance of R^{j} and R^{k} are independently hydrogen, halogen, CN, OH, NO₂, N₃, or C₁-C₆ alkyl; wherein the C₁-C₆ alkyl represented by R^{a} or R^{b} are each optionally substituted with 0 to 3 groups each independently selected from halogen, OH, CN, and NR₅R₅;
q is 1 or 2;
r is 1 or 2;

Q is phenyl or 5- to 14-membered heteroaryl, each of which is optionally substituted at each substitutable ring carbon atom with Rⁿ and optionally substituted at each substitutable ring nitrogen atom by R^{na};
each instance of Rⁿ is independently hydrogen, halogen, CN, OH, NO₂, N₃, C₁-C₆ alkyl, C₁-C₆ alkoxy, —C(═O)NR₅R₅, or NR₅R₅; or alternatively
two instances of Rⁿ attached to the adjacent ring carbon atoms, can be taken together with the carbon atoms to which they are attached to form 5- to 6-membered monocyclic heteroaryl;

provided that

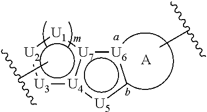

is other than

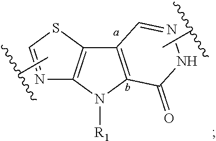

and provided that when

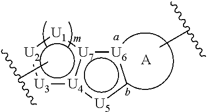

is

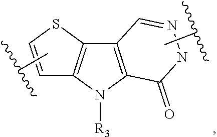

L₂ is —(CRₐR_{b})ᵣ— and Q is phenyl optionally substituted with Rⁿ and R^{na}, then L₁ is —(CRⱼRₖ)_{q}— and R₂ is phenyl or heteroaryl optionally substituted with R^{p} and R^{nc}; and

the remaining variables are as defined in the first embodiment.

In a fourth embodiment, the invention provides a compound or a pharmaceutically acceptable salt thereof, represented by a structural formula selected from:

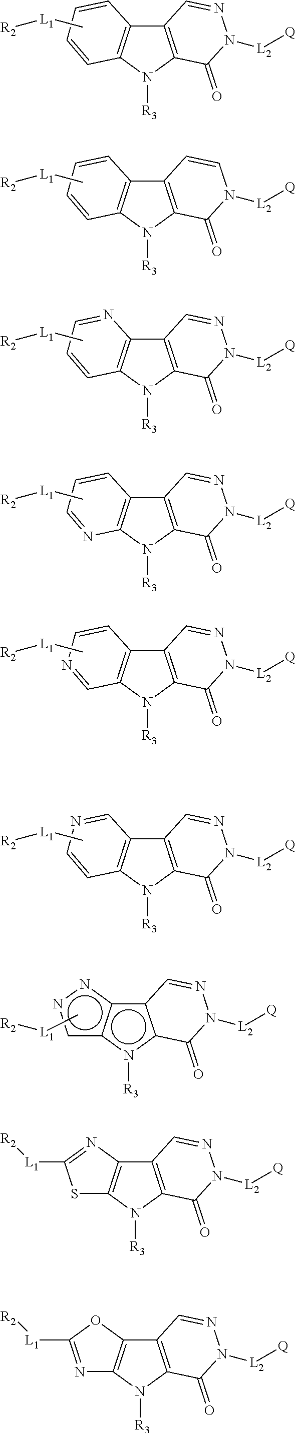

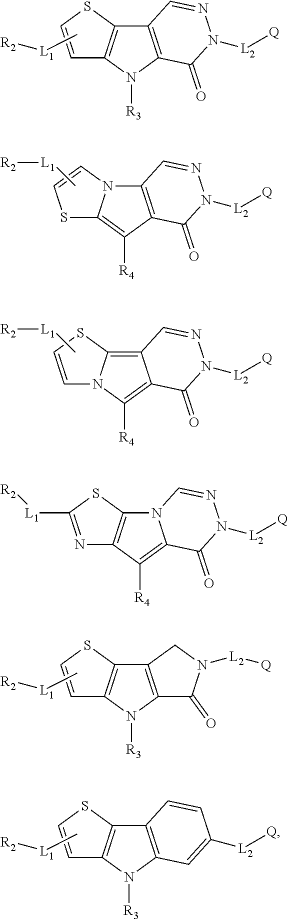

wherein the remaining variables are as defined the first, second, or third embodiment.

In a fifth embodiment, the invention provides a compound or a pharmaceutically acceptable salt thereof, represented by a structural formula selected from:

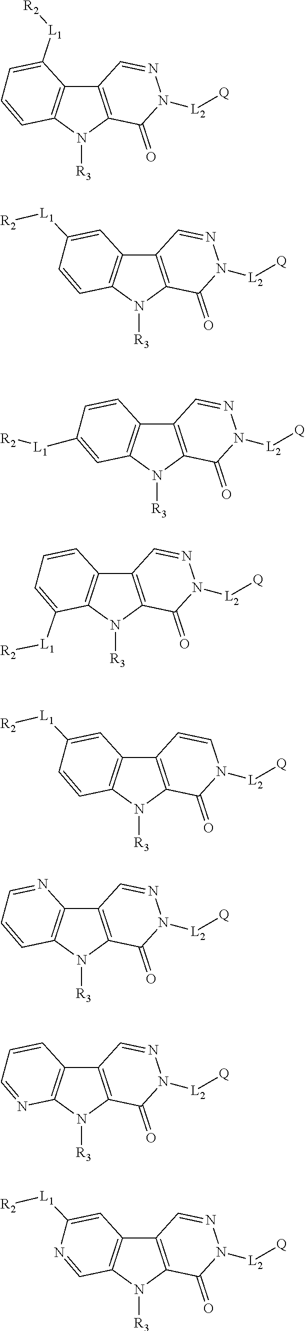

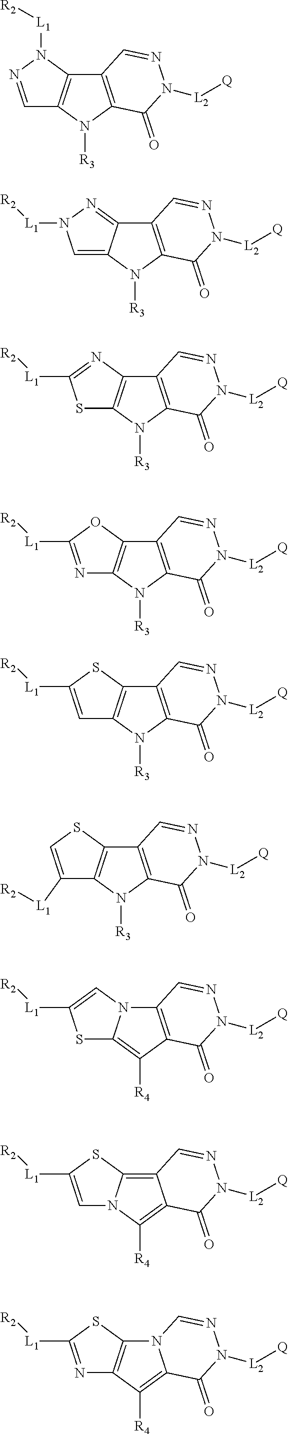

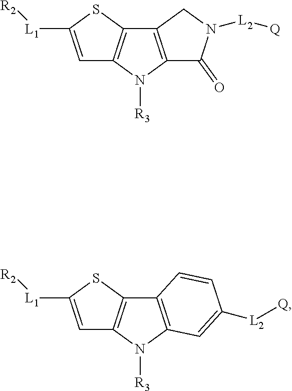

wherein the remaining variables are as defined in the first, second, or third embodiment.

In a sixth embodiment, the invention provides a compound according to structural formula (I), or a structural formula recited in the fourth or fifth embodiment, or a pharmaceutically acceptable salt thereof, wherein R₃ is C₁-C₂ alkyl; R₄ is C₁-C₂ alkyl, C₁-C₂ haloalkyl, halogen, CN, —C(═O)NR₅R₅, or C≡C(CH₂)_{w}OH, wherein w is 1 or 2; and the remaining variables are as defined in the first, second, or third embodiment.

In a seventh embodiment, the invention provides a compound according to structural formula (I), or a structural formula recited in the fourth or fifth embodiment, or a pharmaceutically acceptable salt thereof, wherein R₃ is CH₃; and R₄ is CH₃, CF₃, Br, CN, C(═O)NH₂, or C≡CCH₂OH; and the remaining variables are as defined in the first, second, or third embodiment.

In an eighth embodiment, the invention provides a compound according to structural formula (I), or a structural formula recited in the fourth or fifth embodiment, or a pharmaceutically acceptable salt thereof, wherein R₁ is H or CH₃; each instance of R₅ is H or CH₃; and the remaining variables are as defined in the first, second, third, sixth, or seventh embodiment.

In a ninth embodiment, the invention provides a compound according to structural formula (I), or a structural formula recited in the fourth or fifth embodiment, or a pharmaceutically acceptable salt thereof, wherein:

L₁ is —S(═O)₂—, —S(═O)—, —C(═O)O—*, —C(═O)NRs-*, —NR₅—, or —(CRⱼRₖ)_{q}—, wherein “*” designates the connection to R₂;

L₂ is —(CRₐR_{b})ᵣ—;

wherein R^{a}, R^{b}, R^{j} and R^{k} are each independently hydrogen or halogen; and

the remaining variables are as defined in the first, second, third, sixth, seventh, or eighth embodiment.

In a tenth embodiment, the invention provides a compound according to structural formula (I), or a structural formula recited in the fourth or fifth embodiment, or a pharmaceutically acceptable salt thereof, wherein L₁ is —S(═O)₂—, —S(═O)—, —C(═O)O—*, —C(═O)NH—*, —NH—, —CH₂—, or —CF₂—, wherein “*” designates the connection to R₂; and the remaining variables are as defined in the first, second, third, sixth, seventh, eighth, or ninth embodiment.

In an eleventh embodiment, the invention provides a compound according to structural formula (I), or a structural formula recited in the fourth or fifth embodiment, or a pharmaceutically acceptable salt thereof, wherein L₂ is —CH₂—; and the remaining variables are as defined in the first, second, third, sixth, seventh, eighth, ninth, or tenth embodiment.

In a twelfth embodiment, the invention provides a compound according to structural formula (I), or a structural formula recited in the fourth or fifth embodiment, or a pharmaceutically acceptable salt thereof, wherein:

each instance of R^{na} is independently hydrogen, C₁-C₂ alkyl, or C₁-C₂ haloalkyl;

each instance of Rⁿ is independently hydrogen, CN, OH, C₁-C₄ alkyl, C₁-C₄ alkoxy, —C(═O)NR₅R₅, or NR₅R₅, or two Rⁿ attached to the adjacent carbon atoms of the phenyl ring of Q, can be taken together with the carbon atoms to which they are attached to form 5- to 6-membered monocyclic heteroaryl; and

the remaining variables are as defined in the first, second, third, sixth, seventh, eighth, ninth, tenth, or eleventh embodiment.

In a thirteenth embodiment, the invention provides a compound according to structural formula (I), or a structural formula recited in the fourth or fifth embodiment, or a pharmaceutically acceptable salt thereof, wherein Q is selected from one of the following structural formulae:

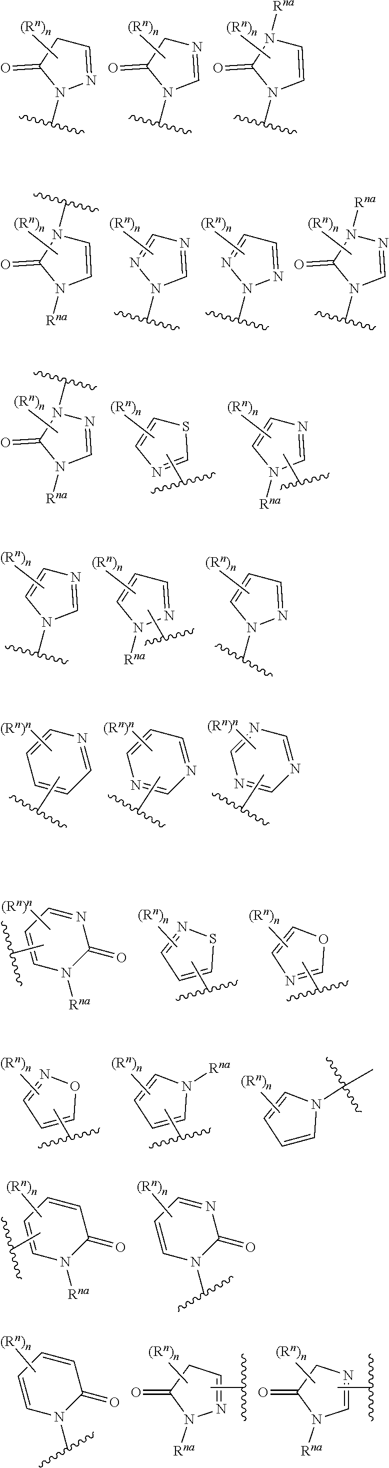

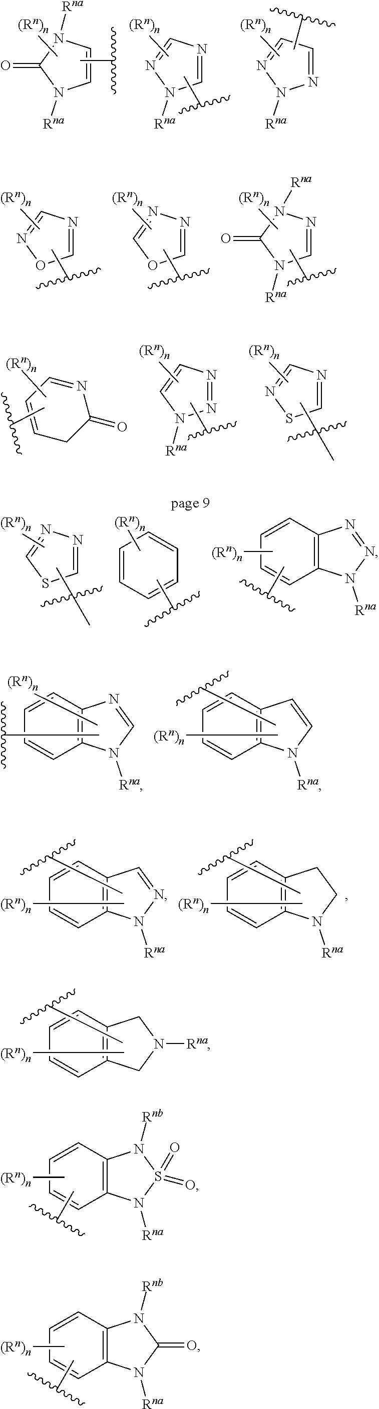

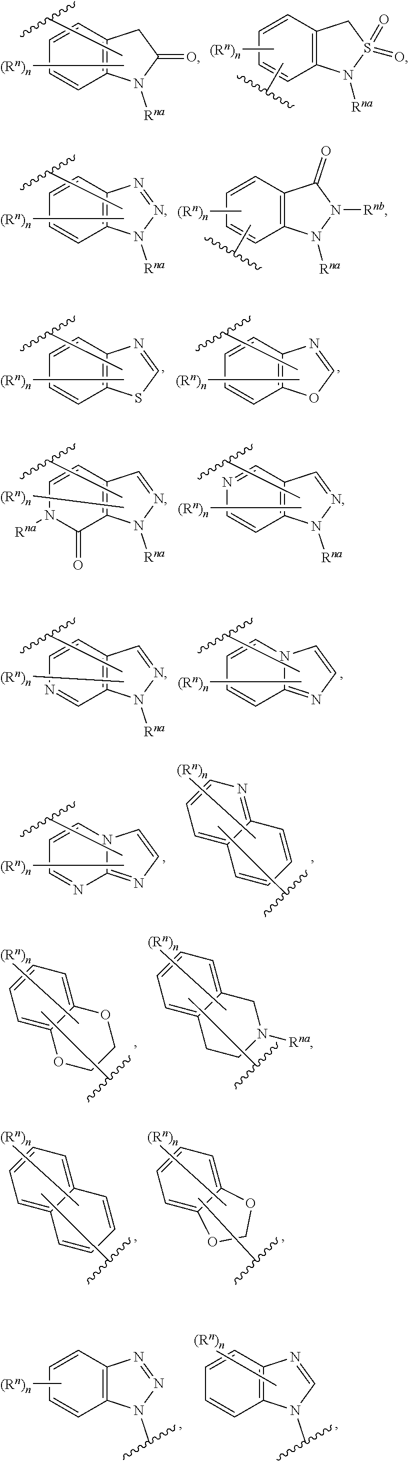

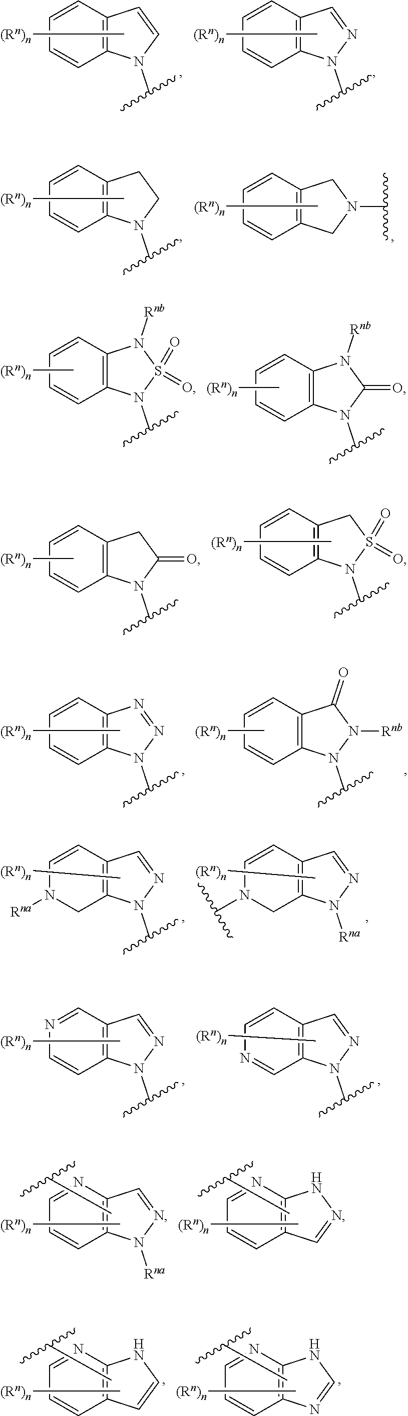

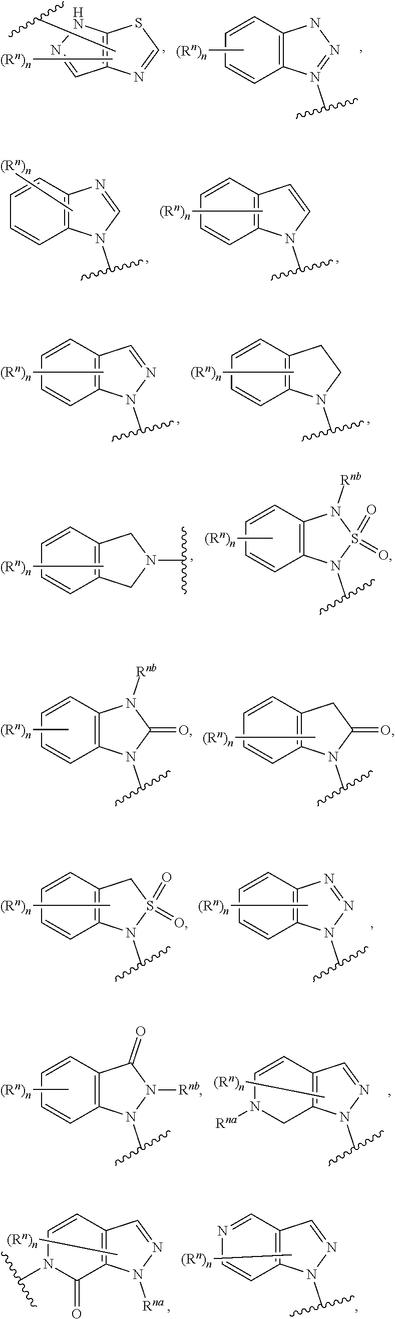

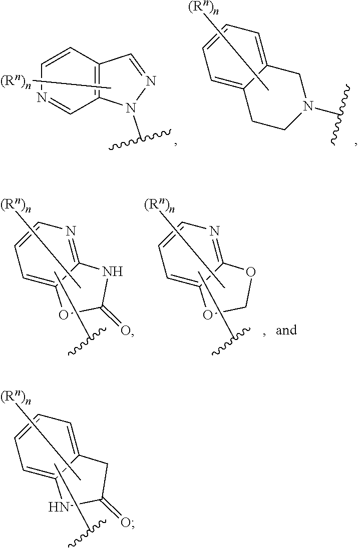

wherein n is 0, 1, or 2, as valency permits; R^{nb} is hydrogen, C₁-C₆ alkyl, or C₁-C₆ haloalkyl; and the remaining variables are as defined in the first, second, third, sixth, seventh, eighth, ninth, tenth, eleventh, or twelfth embodiment.

In a fourteenth embodiment, the invention provides a compound according to structural formula (I), or a structural formula recited in the fourth or fifth embodiment, or a pharmaceutically acceptable salt thereof, wherein Q is selected from one of the following structural formulae:

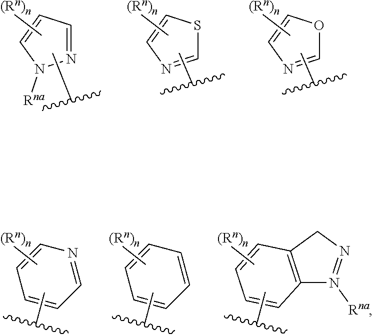

Wherein n is 0 or 1; and the remaining variables are as defined in the first, second, third, sixth, seventh, eighth, ninth, tenth, eleventh, or twelfth embodiment.

In a fifteenth embodiment, the invention provides a compound according to structural formula (I), or a structural formula recited in the fourth or fifth embodiment, or a pharmaceutically acceptable salt thereof, wherein R^{na} is hydrogen or CH₃; Rⁿ is H, CH₃, CN, OCH₃, NH₂, or C(═O)NH₂; n is 0 or 1; and the remaining variables are as defined in the first, second, third, sixth, seventh, eighth, ninth, tenth, eleventh, twelfth, thirteenth, or fourteenth embodiment.

In a sixteenth embodiment, the invention provides a compound according to structural formula (I), or a structural formula recited in the fourth or fifth embodiment, or a pharmaceutically acceptable salt thereof, wherein:

each instance of R^{nc} is independently hydrogen, C₁-C₂ alkyl, or C₁-C₂ haloalkyl;

each instance of R^{p} is independently hydrogen, CN, OH, C₁-C₄ alkyl, C₁-C₄ alkoxy, —C(═O)NR₅R₅, or NR₅R₅, or two R^{p} attached to the adjacent carbon atoms of the phenyl ring of Q, can be taken together with the carbon atoms to which they are attached to form 5- to 6-membered monocyclic heteroaryl; and

the remaining variables are as defined in the first, second, third, sixth, seventh, eighth, ninth, tenth, eleventh, twelfth, thirteenth, fourteenth, or fifteenth embodiment.

In a seventeenth embodiment, the invention provides a compound according to structural formula (I), or a structural formula recited in the fourth or fifth embodiment, or a pharmaceutically acceptable salt thereof, wherein R₂ is selected from one of the following structural formulae:

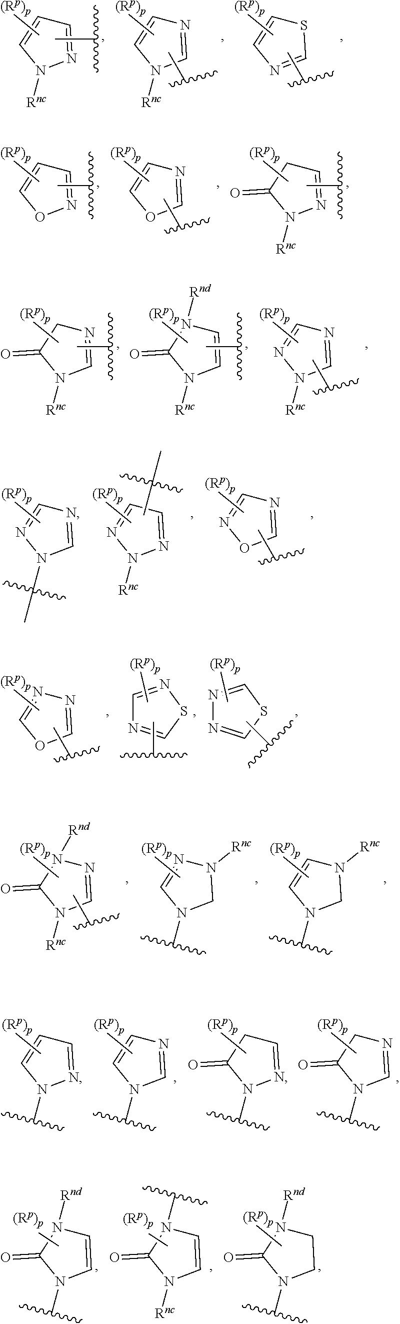

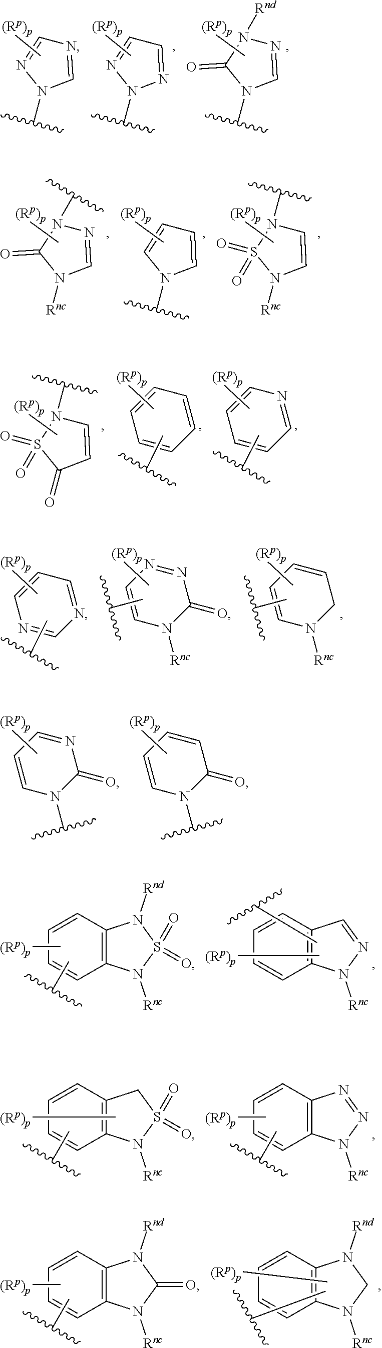

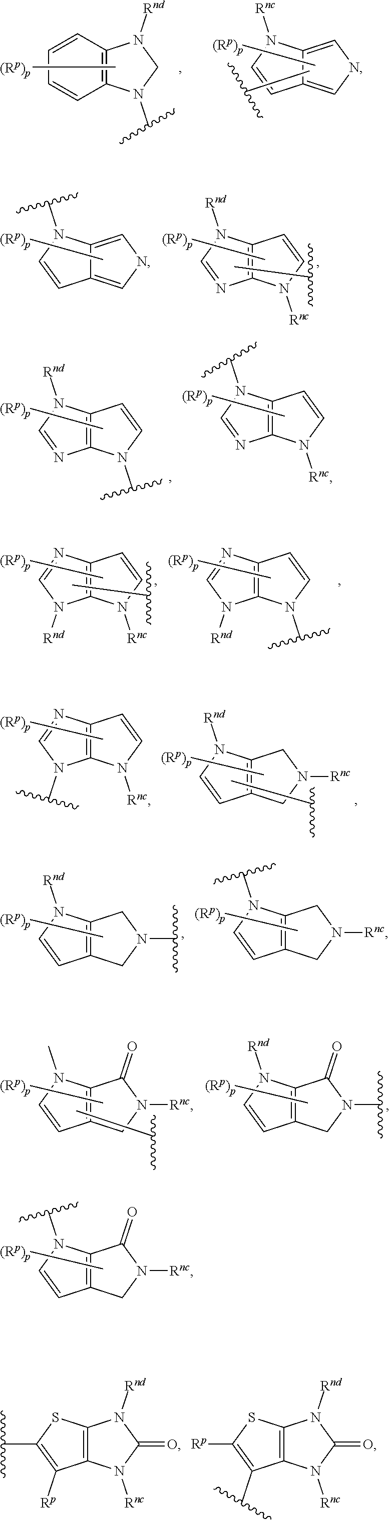

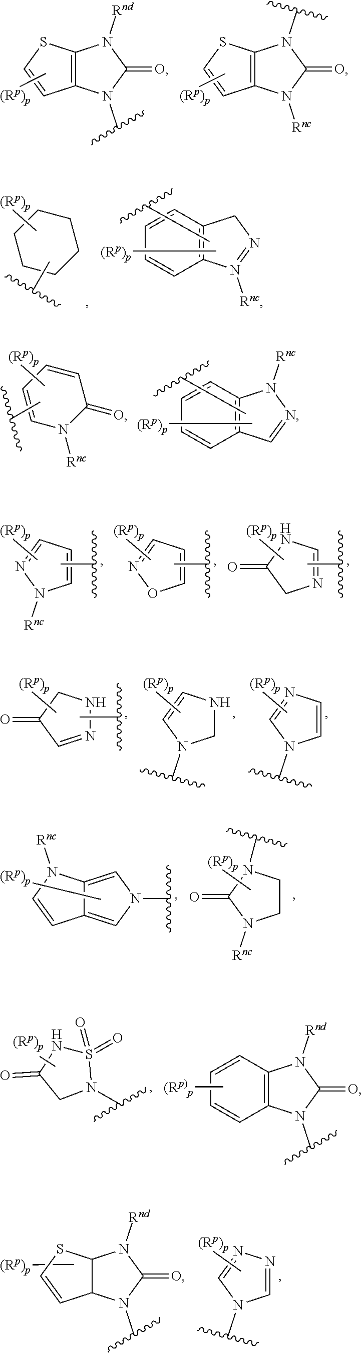

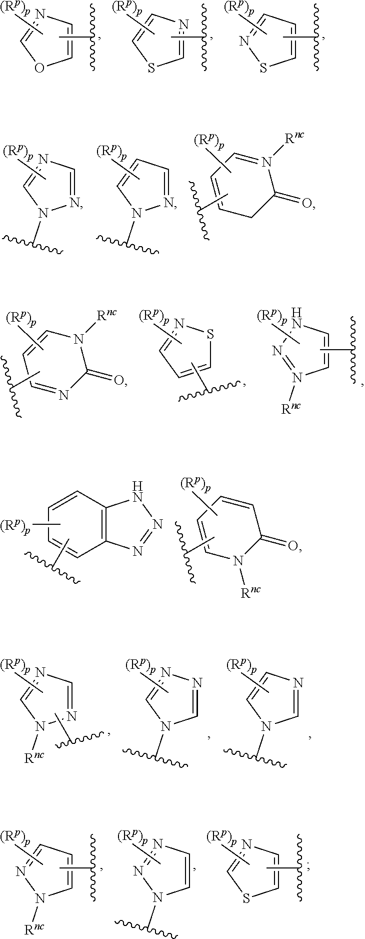

wherein p is 0, 1, or 2, as valency permits; R^{nd} is hydrogen, C₁-C₆ alkyl, or C₁-C₆ haloalkyl; and the remaining variables are as defined in the first, second, third, sixth, seventh, eighth, ninth, tenth, eleventh, twelfth, thirteenth, fourteenth, fifteenth, or sixteenth embodiment.

In an eighteenth embodiment, the invention provides a compound according to structural formula (I), or a structural formula recited in the fourth or fifth embodiment, or a pharmaceutically acceptable salt thereof, wherein R₂ is selected from one of the following structural formulae:

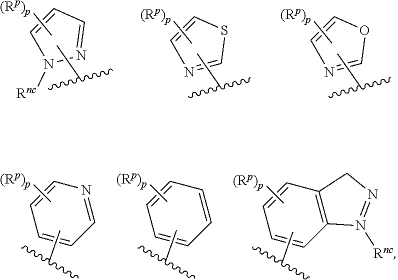

wherein p is 0 or 1; and the remaining variables are as defined in the first, second, third, sixth, seventh, eighth, ninth, tenth, eleventh, twelfth, thirteenth, fourteenth, fifteenth, or sixteenth embodiment.

In a nineteenth embodiment, the invention provides a compound according to structural formula (I), or a structural formula recited in the fourth or fifth embodiment, or a pharmaceutically acceptable salt thereof, wherein R^{nc} is hydrogen or CH₃; R^{p} is H, CH₃, CN, OCH₃, NH₂, or C(═O)NH₂; p is 0 or 1; and the remaining variables are as defined in the first, second, third, sixth, seventh, eighth, ninth, tenth, eleventh, twelfth, thirteenth, fourteenth, fifteenth, sixteenth, seventeenth, or eighteenth embodiment.

In one specific embodiment, the invention provides a compound of the seventeenth, eighteenth, or nineteenth embodiment, or a pharmaceutically acceptable salt thereof, wherein p is 0.

In a twentieth embodiment, the invention provides a compound according to structural formula (I), or a structural formula recited in the fourth or fifth embodiment, or a pharmaceutically acceptable salt thereof, wherein R₂ is C₁-C₂ alkyl; and the remaining variables are as defined in the first, second, third, sixth, seventh, eighth, ninth, tenth, eleventh, twelfth, thirteenth, fourteenth, or fifteenth embodiment.

In a twenty first embodiment, the invention provides a compound according to structural formula (I), or a structural formula recited in the fourth or fifth embodiment, or a pharmaceutically acceptable salt thereof, wherein -L₁-R₂ is —H, —CN, —CH₃, —OH, —Br, —CF₃, —CH═CH₂, or —CH₂OH; and the remaining variables are as defined in the first, second, third, sixth, seventh, eighth, eleventh, twelfth, thirteenth, fourteenth, or fifteenth embodiment.

In a twenty second embodiment, the invention provides a compound according to structural formula (I), or a structural formula recited in the fourth or fifth embodiment, or a pharmaceutically acceptable salt thereof, wherein R₂ is —CH₃; L₁ is —S(═O)₂—, —S(═O)—, —C(═O)O—*, —C(═O)NH—* or —NH—, wherein “*” designates the connection to R₂; and the remaining variables are as defined in the first, second, third, sixth, seventh, eighth, eleventh, twelfth, thirteenth, fourteenth, or fifteenth embodiment.

In a twenty third embodiment, the invention is any one the compounds from Tables 1-3 and in the Examples, or a pharmaceutical acceptable salt thereof.

Disclosed Compounds are useful as activators of PKR mutants having lower activities compared to the wild type, thus are useful for methods of the present invention. Such mutations in PKR can affect enzyme activity (catalytic efficiency), regulatory properties (modulation by fructose bisphosphate (FBP)/ATP), and/or thermostability of the enzyme. Examples of such mutations are described in Valentini et al, JBC 2002. Some examples of the mutants that are activated by the Disclosed Compounds include G332S, G364D, T384M, R479H, R479K, R486W, R532W, K410E, R510Q, and R490W. Without being bound by theory, Disclosed Compounds affect the activities of PKR mutants by activating FBP non-responsive PKR mutants, restoring thermostability to mutants with decreased stability, or restoring catalytic efficiency to impaired mutants. The activating activity of the present compounds against PKR mutants may be tested following a method described in Examples 24-26. Disclosed Compounds are also useful as activators of wild type PKR.

In an embodiment, to increase the lifetime of the red blood cells, a compound, composition or pharmaceutical composition described herein is added directly to whole blood or packed red blood cells extracorporeally or be provided to the patient directly (e.g., by i.p., i.v., i.m., oral, inhalation (aerosolized delivery), transdermal, sublingual and other delivery routes). Without being bound by theory, Disclosed Compounds increase the lifetime of the RBCs, thus counteract aging of stored blood, by impacting the level of 2,3-DPG and/or ATP from the blood. A decrease in the level of 2, 3-DPG concentration induces a leftward shift of the oxygen-hemoglobin dissociation curve and shifts the allosteric equilibrium to the R, or oxygenated state, thus producing a therapeutic inhibition of the intracellular polymerization that underlies sickling by increasing oxygen affinity due to the 2,3-DPG depletion, thereby stabilizing the more soluble oxy-hemoglobin. Accordingly, in one embodiment, compounds and pharmaceutical compositions described herein are useful as antisickling agents. In another embodiment, to regulate 2,3-diphosphoglycerate, a compound, composition or pharmaceutical composition described herein is added directly to whole blood or packed red blood cells extracorporeally or be provided to the patient directly (e.g., by i.p., i.v., i.m., oral, inhalation (aerosolized delivery), transdermal, sublingual and other delivery routes). In another embodiment, a compound, composition or pharmaceutical composition described herein can increase the level of ATP and help to protect the cells from reactive oxygen species (Mol Cell. 2012 Oct. 26; 48(2): 158-167).

In certain embodiments, Disclosed Compounds are useful as activators of PKM2 utilized in the methods and compositions described herein and operated by or has one or more of the following mechanisms or properties:

a. it is an allosteric activator of PKM2;

b. it modulates (e.g., stabilizes) the binding of FBP in a binding pocket of PKM2;

c. it modulates (e.g., promotes) the release of FBP from a binding pocket of PKM2;

d. it is a modulator (e.g., an agonist), e.g., an analog, of FBP, e.g., an agonist which binds PKM2 with a lower, about the same, or higher affinity than does FBP;

e. it modulates (e.g., promotes) the dissolution of tetrameric PKM2;

f. it modulates (e.g., promotes) the assembly of tetrameric PKM2;

g. it modulates (e.g., stabilizes) the tetrameric conformation of PKM2;

h. it modulates (e.g., promotes) the binding of a phosphotyrosine containing polypeptide to PKM2;

i. it modulates (e.g., promotes) the ability of a phosphotyrosine containing polypeptide to induce release of FBP from PKM2, e.g., by inducing a change in the conformation of PKM2, e.g., in the position of Lys 433, thereby hindering the release of FBP;

j. modulates the propensity of PKM2 to undergo post-translational modifications (e.g. oxidation at Cys358 or acetylation on Lys305) that affect activity of the enzyme.

k. it binds to or changes the position of Lys 433 relative to the FBP binding pocket;

l. it selectively modulates (e.g., activates) PKM2 over at least one other isoform of PK, e.g., it is selective for PKM2 over one or more of PKR, PKM1, or PKL;

m. it has an affinity for PKM2 which is greater than its affinity for at least one other isoform of PK, e.g., PKR, PKM1, or PKL.

In Tables 1 and 2, a compound described herein may have an AC50 of wild type PKR, PKR K410E or PKR 510Q. “A” refers to an AC50 less than 0.300 μM; “B” refers to an AC50 from 0.301 μM to 0.800 μM, and “C” refers to an AC50 greater than 0.800 μM. The AC50 of wild type PKR for certain compounds was additionally determined in a cell-based ATP assay. “AA” refers to an AC50 less than or equal to 1 μM and “BB” refers to an AC50 more than 1 μM. NA means not available.

TABLE 1
Activation of wild type and mutant PKR by exemplary compounds

| Cpd | |
|---|---|
| Nr | Compound |
| 1 | 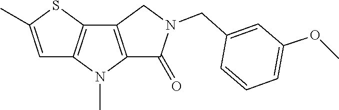 |
| 2 | 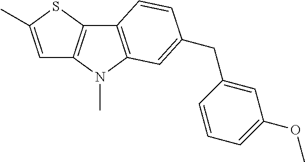 |
| 3 | 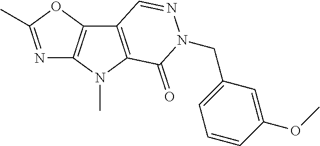 |
| 4 | 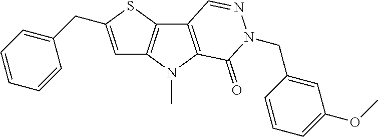 [SMILES: COc1cccc(Cn2ncc3c4sc(Cc5ccccc5)cc4n(C)c3c2=O)c1] |
| 5 | 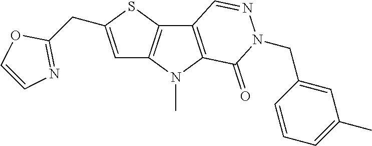 [SMILES: Cc1cccc(Cn2ncc3c4sc(Cc5ncco5)cc4n(C)c3c2=O)c1] |
| 6 | 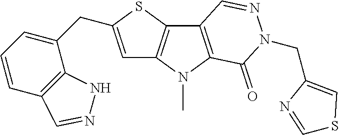 [SMILES: Cn1c2cc(Cc3cccc4cn[nH]c34)sc2c2cnn(Cc3cscn3)c(=O)c21] |
| 7 | 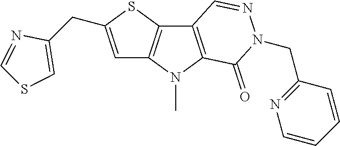 [SMILES: Cn1c2cc(Cc3cscn3)sc2c2cnn(Cc3ccccn3)c(=O)c21] |
| 8 | 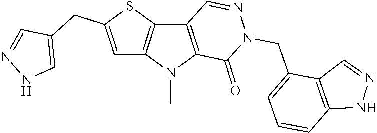 [SMILES: Cn1c2cc(Cc3cn[nH]c3)sc2c2cnn(Cc3cccc4[nH]ncc34)c(=O)c21] |
| 9 | 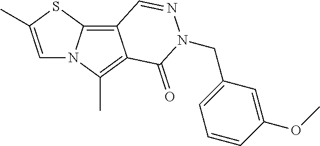 |
| 10 | 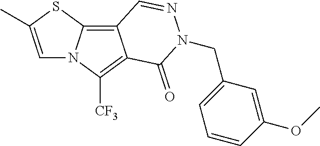 |
| 11 | 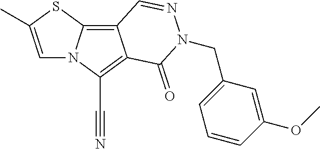 |
| 12 | 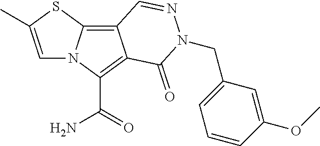 |
| 13 | 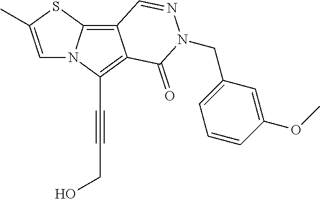 |
| 14 | 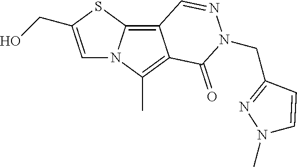 [SMILES: Cc1c2c(=O)n(Cc3ccn(C)n3)ncc2c2sc(CO)cn12] |
| 15 | 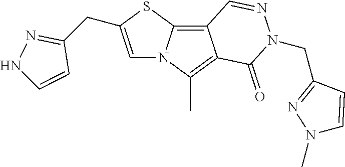 [SMILES: Cc1c2c(=O)n(Cc3ccn(C)n3)ncc2c2sc(Cc3cc[nH]n3)cn12] |
| 16 | 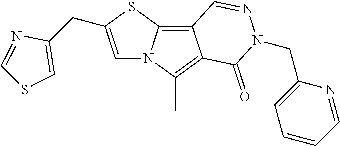 [SMILES: Cc1c2c(=O)n(Cc3ccccn3)ncc2c2sc(Cc3cscn3)cn12] |
| 17 | 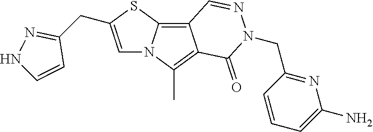 [SMILES: Cc1c2c(=O)n(Cc3cccc(N)n3)ncc2c2sc(Cc3cc[nH]n3)cn12] |
| 18 | 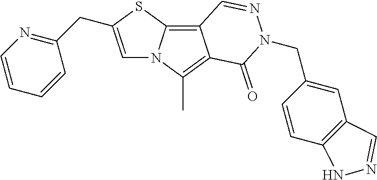 [SMILES: Cc1c2c(=O)n(Cc3ccc4[nH]ncc4c3)ncc2c2sc(Cc3ccccn3)cn12] |
| 19 | 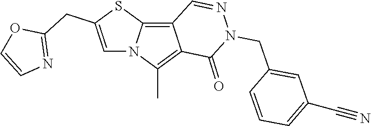 [SMILES: Cc1c2c(=O)n(Cc3cccc(C#N)c3)ncc2c2sc(Cc3ncco3)cn12] |
| 20 | 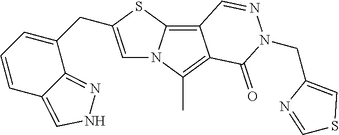 [SMILES: Cc1c2c(=O)n(Cc3cscn3)ncc2c2sc(Cc3cccc4c[nH]nc34)cn12] |
| 21 | 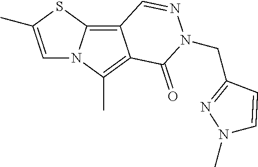 [SMILES: Cc1cn2c(C)c3c(=O)n(Cc4ccn(C)n4)ncc3c2s1] |
| 22 | 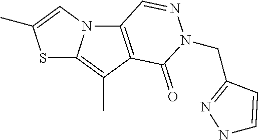 [SMILES: Cc1cn2c(s1)c(C)c1c(=O)n(Cc3cc[nH]n3)ncc12] |
| 23 | 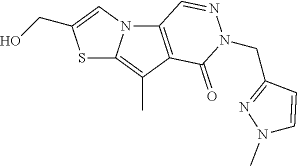 [SMILES: Cc1c2c(=O)n(Cc3ccn(C)n3)ncc2n2cc(CO)sc12] |
| 24 | 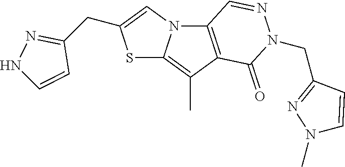 [SMILES: Cc1c2c(=O)n(Cc3ccn(C)n3)ncc2n2cc(Cc3cc[nH]n3)sc12] |
| 25 | 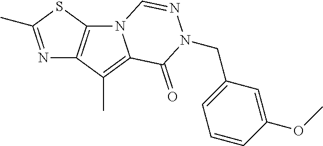 |
| 26 | 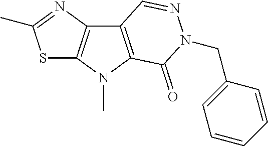 [SMILES: Cc1nc2c3cnn(Cc4ccccc4)c(=O)c3n(C)c2s1] |
| 27 | 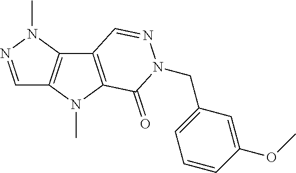 [SMILES: COc1cccc(Cn2ncc3c4c(cnn4C)n(C)c3c2=O)c1] |
| 28 | 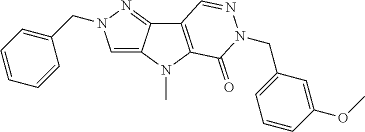 [SMILES: COc1cccc(Cn2ncc3c4nn(Cc5ccccc5)cc4n(C)c3c2=O)c1] |
| 29 | 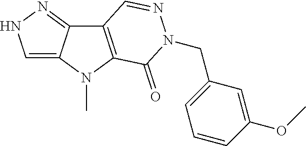 [SMILES: COc1cccc(Cn2ncc3c4n[nH]cc4n(C)c3c2=O)c1] |
| 30 | 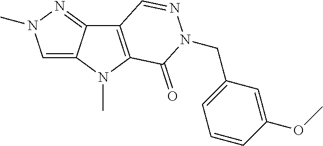 |
| 31 | 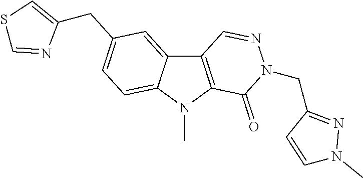 [SMILES: Cn1ccc(Cn2ncc3c4cc(Cc5cscn5)ccc4n(C)c3c2=O)n1] |
| 32 | 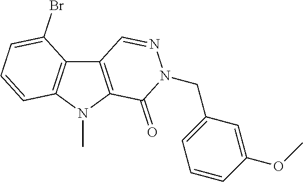 [SMILES: COc1cccc(Cn2ncc3c4c(Br)cccc4n(C)c3c2=O)c1] |
| 33 | 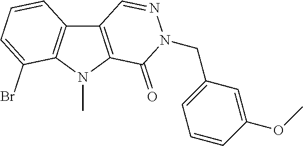 [SMILES: COc1cccc(Cn2ncc3c4cccc(Br)c4n(C)c3c2=O)c1] |
| 34 | 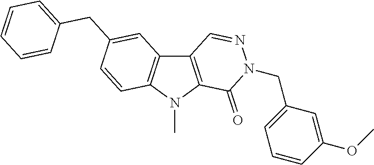 [SMILES: COc1cccc(Cn2ncc3c4cc(Cc5ccccc5)ccc4n(C)c3c2=O)c1] |
| 35 | 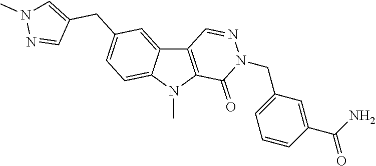 [SMILES: Cn1cc(Cc2ccc3c(c2)c2cnn(Cc4cccc(C(N)=O)c4)c(=O)c2n3C)cn1] |
| 36 | 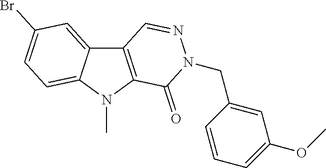 [SMILES: COc1cccc(Cn2ncc3c4cc(Br)ccc4n(C)c3c2=O)c1] |
| 37 | 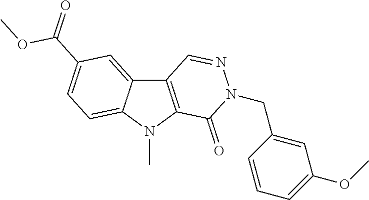 |
| 38 | 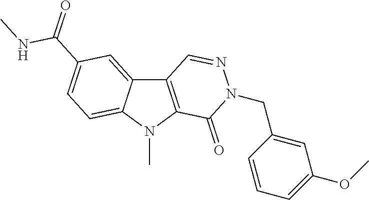 |
| 39 | 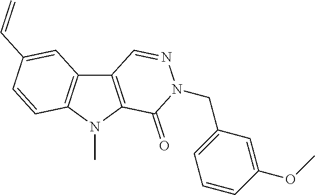 [SMILES: C=Cc1ccc2c(c1)c1cnn(Cc3cccc(OC)c3)c(=O)c1n2C] |
| 40 | 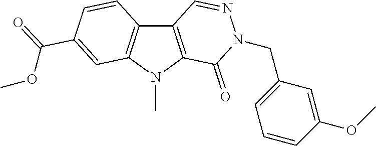 |
| 41 | 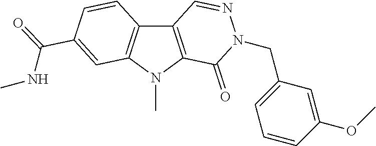 |
| 42 | 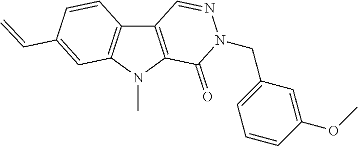 [SMILES: C=Cc1ccc2c3cnn(Cc4cccc(OC)c4)c(=O)c3n(C)c2c1] |
| 43 | 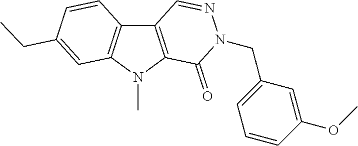 [SMILES: CCc1ccc2c3cnn(Cc4cccc(OC)c4)c(=O)c3n(C)c2c1] |
| 44 | 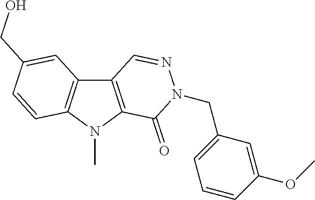 [SMILES: COc1cccc(Cn2ncc3c4cc(CO)ccc4n(C)c3c2=O)c1] |
| 45 | 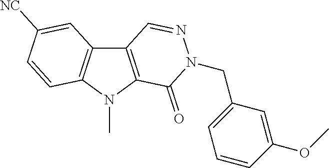 [SMILES: COc1cccc(Cn2ncc3c4cc(C#N)ccc4n(C)c3c2=O)c1] |
| 46 | 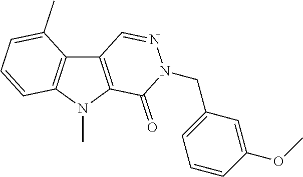 [SMILES: COc1cccc(Cn2ncc3c4c(C)cccc4n(C)c3c2=O)c1] |
| 47 | 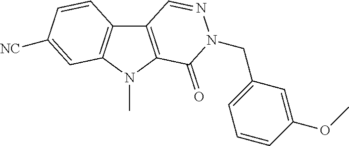 [SMILES: COc1cccc(Cn2ncc3c4ccc(C#N)cc4n(C)c3c2=O)c1] |
| 48 | 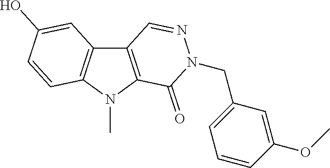 [SMILES: COc1cccc(Cn2ncc3c4cc(O)ccc4n(C)c3c2=O)c1] |
| 49 | 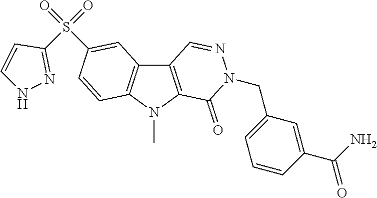 [SMILES: Cn1c2ccc(S(=O)(=O)c3cc[nH]n3)cc2c2cnn(Cc3cccc(C(N)=O)c3)c(=O)c21] |
| 50 | 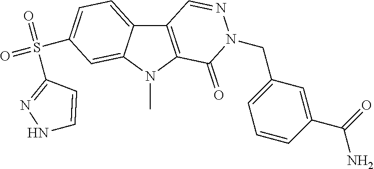 [SMILES: Cn1c2cc(S(=O)(=O)c3cc[nH]n3)ccc2c2cnn(Cc3cccc(C(N)=O)c3)c(=O)c21] |
| 51 | 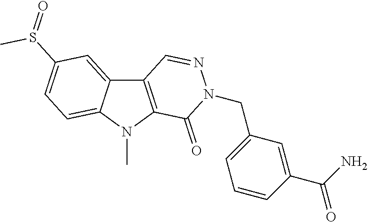 [SMILES: Cn1c2ccc(S(C)=O)cc2c2cnn(Cc3cccc(C(N)=O)c3)c(=O)c21] |
| 52 | 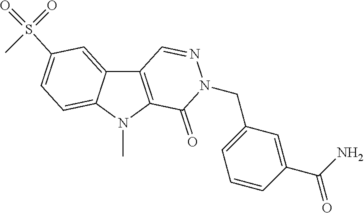 [SMILES: Cn1c2ccc(S(C)(=O)=O)cc2c2cnn(Cc3cccc(C(N)=O)c3)c(=O)c21] |
| 53 | 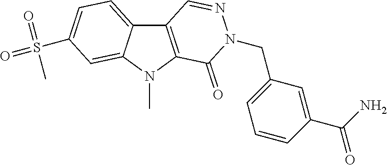 [SMILES: Cn1c2cc(S(C)(=O)=O)ccc2c2cnn(Cc3cccc(C(N)=O)c3)c(=O)c21] |
| 54 | 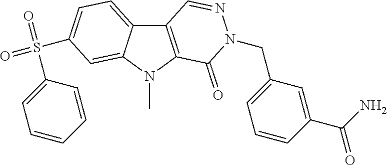 [SMILES: Cn1c2cc(S(=O)(=O)c3ccccc3)ccc2c2cnn(Cc3cccc(C(N)=O)c3)c(=O)c21] |
| 55 | 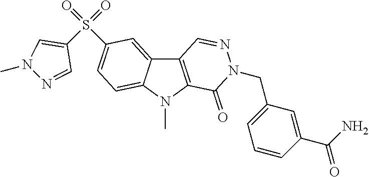 [SMILES: Cn1cc(S(=O)(=O)c2ccc3c(c2)c2cnn(Cc4cccc(C(N)=O)c4)c(=O)c2n3C)cn1] |
| 56 | 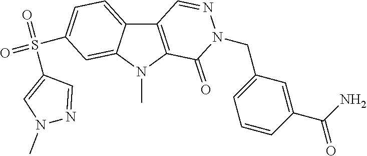 [SMILES: Cn1cc(S(=O)(=O)c2ccc3c4cnn(Cc5cccc(C(N)=O)c5)c(=O)c4n(C)c3c2)cn1] |
| 57 | 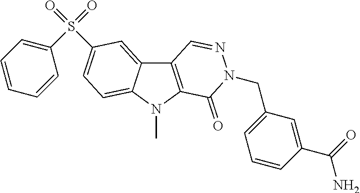 [SMILES: Cn1c2ccc(S(=O)(=O)c3ccccc3)cc2c2cnn(Cc3cccc(C(N)=O)c3)c(=O)c21] |
| 58 | 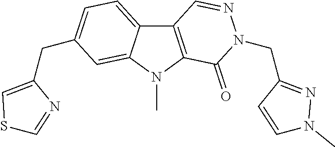 [SMILES: Cn1ccc(Cn2ncc3c4ccc(Cc5cscn5)cc4n(C)c3c2=O)n1] |
| 59 | 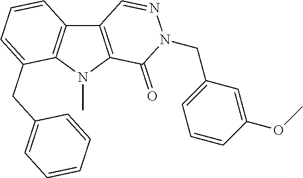 [SMILES: COc1cccc(Cn2ncc3c4cccc(Cc5ccccc5)c4n(C)c3c2=O)c1] |
| 60 | 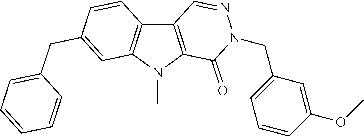 [SMILES: COc1cccc(Cn2ncc3c4ccc(Cc5ccccc5)cc4n(C)c3c2=O)c1] |
| 61 | 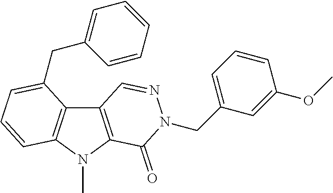 [SMILES: COc1cccc(Cn2ncc3c4c(Cc5ccccc5)cccc4n(C)c3c2=O)c1] |
| 62 | 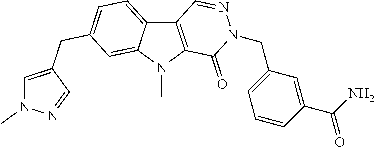 [SMILES: Cn1cc(Cc2ccc3c4cnn(Cc5cccc(C(N)=O)c5)c(=O)c4n(C)c3c2)cn1] |
| 63 | 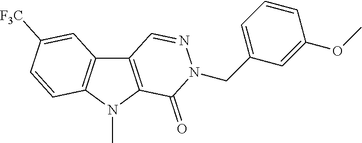 [SMILES: COc1cccc(Cn2ncc3c4cc(C(F)(F)F)ccc4n(C)c3c2=O)c1] |
| 64 | 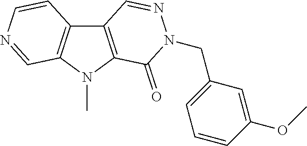 [SMILES: COc1cccc(Cn2ncc3c4ccncc4n(C)c3c2=O)c1] |
| 65 | 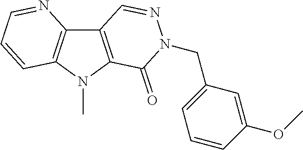 [SMILES: COc1cccc(Cn2ncc3c4ncccc4n(C)c3c2=O)c1] |
| 66 | 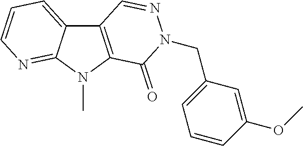 [SMILES: COc1cccc(Cn2ncc3c4cccnc4n(C)c3c2=O)c1] |
| 67 | 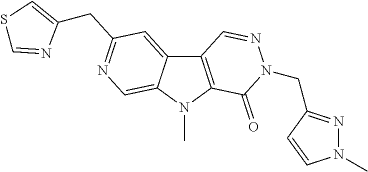 [SMILES: Cn1ccc(Cn2ncc3c4cc(Cc5cscn5)ncc4n(C)c3c2=O)n1] |
| 68 | 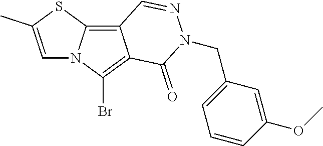 |

TABLE 2
AC₅₀ of Exemplary compounds for Wild Type and Mutant PKR

| Cpd | PKR WT | PKR K410E | PK R510Q | Cell based ATP |
|---|---|---|---|---|
| Nr | AC50 | AC50 | AC50 | assay AC50 |
| 1 | C | C | C | NA |
| 2 | No Fit | No Fit | No Fit | NA |
| 3 | C | C | C | NA |
| 4 | A | A | A | NA |
| 5 | A | A | A | AA |
| 6 | A | A | A | AA |
| 7 | A | A | A | AA |
| 8 | A | No Fit | A | AA |
| 9 | A | A | A | BB |
| 10 | A | No Fit | No Fit | No Fit |
| 11 | A | A | A | No Fit |
| 12 | A | A | A | BB |
| 13 | B | B | B | No Fit |
| 14 | A | A | A | AA |
| 15 | A | A | A | AA |
| 16 | A | A | A | AA |
| 17 | A | A | A | AA |
| 18 | A | A | A | AA |
| 19 | B | A | C | BB |
| 20 | A | A | A | AA |
| 21 | A | A | A | AA |
| 22 | C | C | C | No Fit |
| 23 | C | C | C | BB |
| 24 | A | A | A | AA |
| 25 | C | C | C | No Fit |
| 26 | C | C | C | No Fit |
| 27 | No Fit | No Fit | No Fit | NA |
| 28 | B | B | A | NA |
| 29 | C | C | C | NA |
| 30 | C | C | C | NA |
| 31 | C | C | C | BB |
| 32 | C | C | C | NA |
| 33 | No Fit | Inhibition | No Fit | NA |
| 34 | C | C | C | NA |
| 35 | No Fit | No Fit | No Fit | No Fit |
| 36 | C | C | C | NA |
| 37 | C | C | C | NA |
| 38 | No Fit | No Fit | No Fit | NA |
| 39 | C | C | C | NA |
| 40 | No Fit | A | No Fit | NA |
| 41 | C | No Fit | No Fit | NA |
| 42 | C | C | C | NA |
| 43 | C | C | C | NA |
| 44 | C | C | C | NA |
| 45 | C | C | C | NA |
| 46 | No Fit | C | No Fit | NA |
| 47 | B | A | A | NA |
| 48 | C | C | C | NA |
| 49 | A | A | A | AA |
| 50 | B | C | A | BB |
| 51 | C | C | C | No Fit |
| 52 | C | C | C | No Fit |
| 53 | B | B | A | BB |
| 54 | B | A | A | BB |
| 55 | B | A | A | BB |
| 56 | B | A | A | BB |
| 57 | B | C | A | BB |
| 58 | A | A | A | AA |
| 59 | C | C | No Fit | NA |
| 60 | C | C | B | NA |
| 61 | No Fit | No Fit | No Fit | NA |
| 62 | C | C | C | No Fit |
| 63 | C | C | C | NA |
| 64 | B | B | A | NA |
| 65 | C | C | C | NA |
| 66 | C | C | B | NA |
| 67 | A | A | A | AA |
| 68 | A | A | A | BB |

A Disclosed Compound may also be tested for its ability to activate PKM2. For simplicity, the activation activity of these compounds is represented as an AC₅₀ in Table 3. In Table 3, a Disclosed Compound of Table 1 may have an AC50 of wild type PKM2. “A” refers to an AC50 less than 0.300 μM; “B” refers to an AC50 from 0.301 μM to 0.800 μM, and “C” refers to an AC50 greater than 0.800 μM.

TABLE 3
AC₅₀ of Exemplary compounds
for Wild Type PKM2

| Cpd | PKM2 WT | Cpd | PKM2 WT |
|---|---|---|---|
| No. | AC50 | No. | AC50 |
| 1 | C | 35 | B |
| 2 | C | 36 | B |
| 3 | B | 37 | C |
| 4 | A | 38 | C |
| 25 | C | 39 | C |
| 26 | C | 41 | A |
| 29 | C | 42 | C |
| 30 | C | 43 | C |
| 31 | C | 44 | B |
| 32 | C | 45 | A |
| 34 | C | 46 | C |
| 48 | B | 51 | C |
| 52 | B | 59 | C |
| 60 | B | 61 | C |
| 62 | B | 63 | C |
| 65 | C | 66 | B |

Certain activator compounds useful as PKR wild type and/or mutant activators are those that demonstrate specificity and activation of PKR enzyme (wild type and/or a mutant enzyme) in the absence of FBP to a level greater than that of 10, 15, 20, 25, 30, 35, 40, 45, 50, 55, 60, 65, 70, 75, 80, 85, 90, 95, 99, or 100% in the presence of FBP.

The Disclosed Compounds can be made using a variety of synthetic techniques as set forth in the Examples. Synthetic chemistry transformations and protecting group methodologies (protection and deprotection) useful in synthesizing the Disclosed Compounds are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

In some embodiments, the Disclosed Compounds can be prepared using methods illustrated in Schemes 1-10.

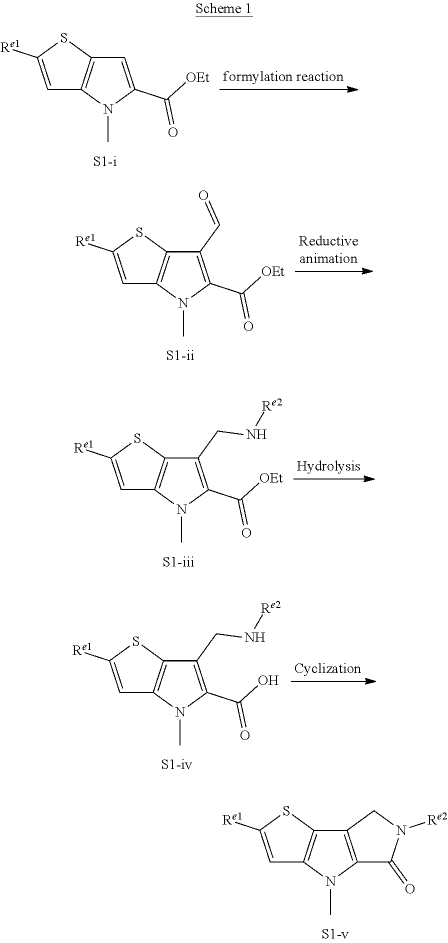

wherein R^{e1} is L₁-R₂; and R^{e2} is -L₂-Q. In certain embodiments, R^{e1} is independently C₁-C₆ alkyl, C₃-C₁₂ cycloalkyl-C₁₋₄ alkyl, 3- to 8-membered heterocyclyl-C₁₋₄ alkyl, 6- to 14-membered aryl-C₁₋₄ alkyl, or 5- to 14-membered heteroaryl-C₁₋₄ alkyl, wherein each cycloalkyl, heterocyclyl, aryl, and heteroaryl is optionally substituted at each substitutable ring carbon atom with R^{p} and optionally substituted at each substitutable ring nitrogen atom by R^{nc}; and R^{e2} is C₁-C₆ alkyl, C₃-C₁₂ cycloalkyl-C₁₋₄ alkyl, 3- to 8-membered heterocyclyl-C₁₋₄ alkyl, 6- to 14-membered aryl-C₁₋₄ alkyl, or 5- to 14-membered heteroaryl-C₁₋₄ alkyl, wherein each cycloalkyl, heterocyclyl, aryl, and heteroaryl is optionally substituted at each substitutable ring carbon atom with Rⁿ and optionally substituted at each substitutable ring nitrogen atom by R^{na}, wherein R^{p}, R^{nc}, Rⁿ, R^{na}, L₁, R₂, L₂, and Q are as defined. Compound S1-i undergoes a formylation reaction (e.g. POCl₃ in DMF) to give compound 51-ii. Reductive animation of compound S1-ii with a primary or secondary amine generates compound S1-iii, which is subsequently hydrolyzed (e.g. NaOH in methanol) to give S1-iv. Cyclization of compound S1-iv in the presence of coupling reagent (e.g. EDCI and DMAP) provides tricyclic compound Si-v.

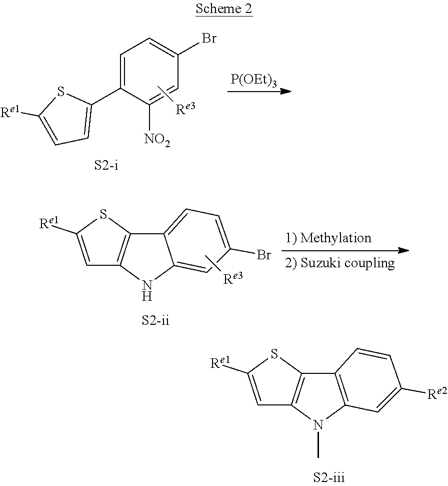

Wherein R^{e1} and R^{e2} are as defined in Scheme 1; and R^{e3} is hydrogen or C₁₋₄ alkyl. Compound S2-i undergoes a reductive cyclization reaction in the presence of P(OEt)₃ to give tricyclic compound S2-ii. Methylation and subsequent metal coupling (e.g. Suzuki coupling) gives compound S2-iii.

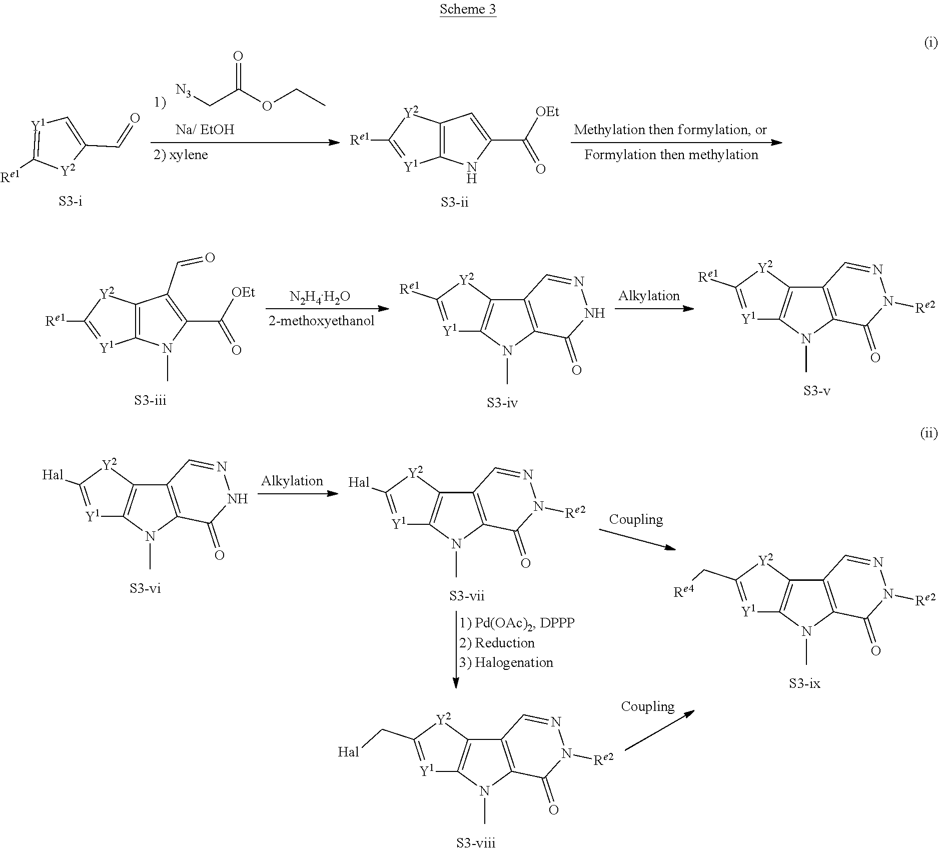

Wherein R^{e1} and R^{e2} are as defined in Scheme 1; R^{e4} is 6- to 14-membered aryl or 5- to 14-membered heteroaryl; each of which is optionally substituted at each substitutable ring carbon atom by R^{p} and optionally substituted at each substitutable ring nitrogen atom by R^{nc}; Hal is halogen (e.g., Br or I); Y¹ is C, N, or S; and Y² is S, O, or N. In certain embodiments, Y¹ is S and Y² is N. In certain embodiments, Y¹ is C and Y² is S. In certain embodiments, Y¹ is N and Y² is O.

In route (i), compound S3-i reacts with ethyl azidoacetate under nucleophilic addition conditions (e.g. a base) in an appropriate solvent (e.g. ethanol), followed by cyclization in xylene gives bicyclic compound S3-ii. Methylation and subsequent formylation (e.g. N-methyl-N-phenylformamide or DMF, POCl3 i), or formylation and subsequent methylation provide compound S3-iii. Cyclization of compound S3-iii in the presence of hydrazine provides tricyclic compound S3-iv. Subsequent alkylation of compound S3-iv gives compound S3-v. In certain embodiments when R^{e1} is halogen (e.g. Br), compound S3-iv is shown as compound S3-vi in route (ii) to undergo alkylation to give compound S3-vii. Compound S3-vii can undergo an organometallic coupling reaction (e.g. Suzuki reaction) to give compound S3-ix. Alternatively, compound S3-vii can be converted to compound S3-viii by palladium catalysed carbonylation reaction followed by reduction and halogen substitution. Coupling of compound S3-viii with organometal (such as aryl stannanes) in presence of a catalyst (e.g. Pd(Ph₃P)₄) gives compound S3-ix.

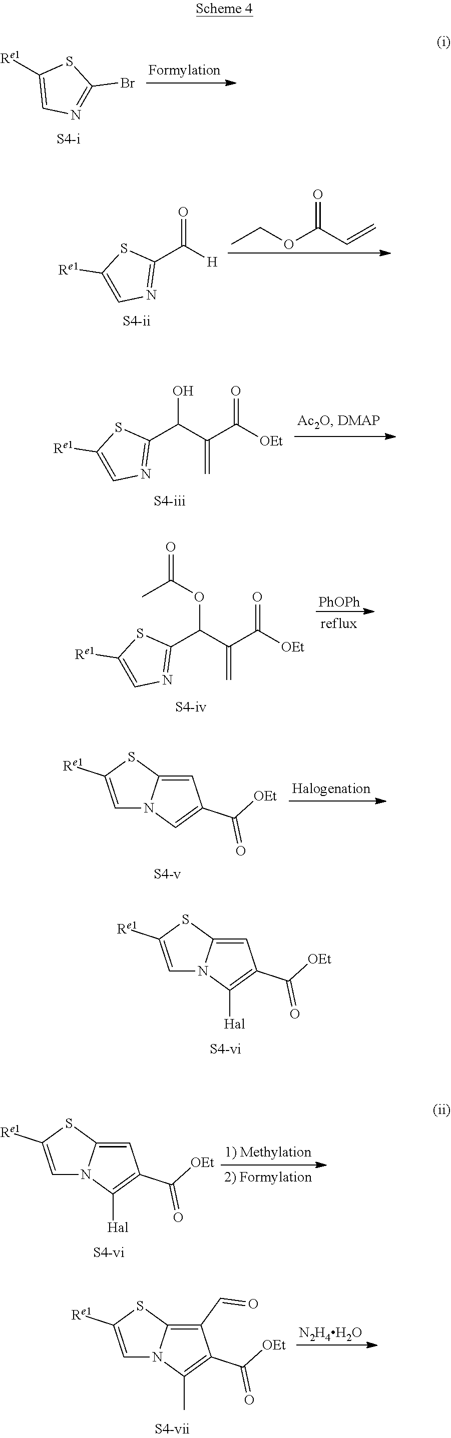

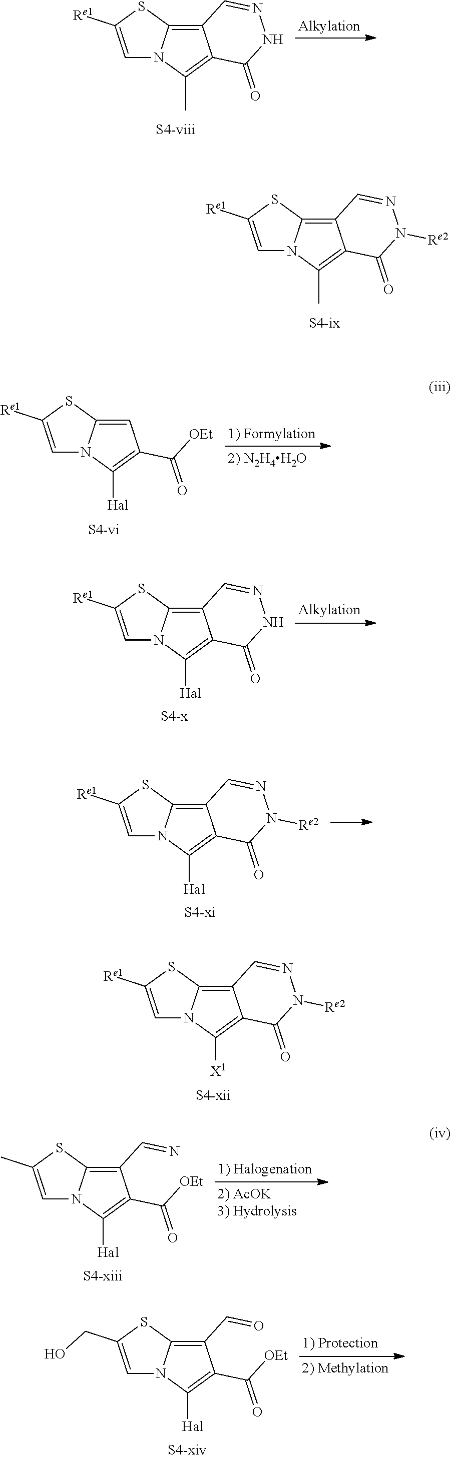

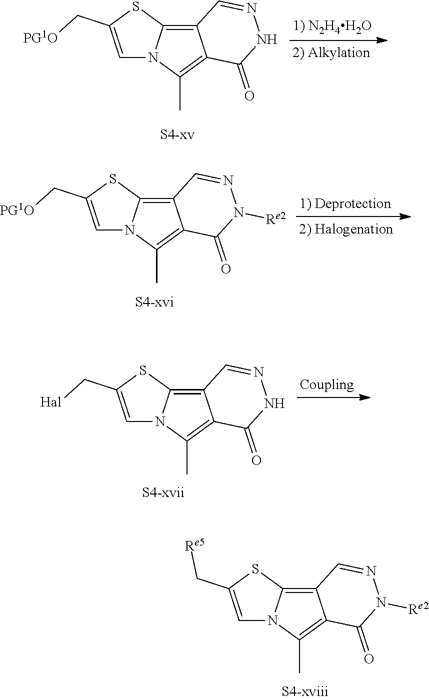

R^{e1}, R^{e2}, and Hal are as defined in Scheme 2. R^{e5} has the same definition as R₂. PG1 is an oxygen protecting group (e.g. methoxylmethyl (MOM), tetrahydropyranyl (THP), trimethylsilyl (TMS), triethylsilyl (TES), triisopropylsilyl (TIPS)). X¹ is C₁₋₄ alkyl or C₁₋₄ haloalkyl, cyano, amide, or C₁₋₄ alkynyl, wherein each of C₁₋₄ alkyl, C₁₋₄ haloalkyl, and C₂₋₄ alkynyl is independently optionally substituted with 1-3 instances of C₁₋₄ alkyl or halogen. Compound S4-i reacts with a formylation reagent (e.g. DMF) in the presence of a base (e.g. n-BuLi) to generate compound S4-ii, which was converted to S4-iii through Baylis-Hillman reaction (reaction with ethyl acrylate in the presence of DABCO). Esterification and subsequent cyclization gives compound S4-v. Halogenation (e.g. NBS in DCM, Hal is Br) of compound S4-v gives compound S4-vi. Compound S4-vi undergoes methylation (e.g. MeB(OH)2 and Pd(PPh3)4) and formylation gives compound S4-vii, which can provide compound S4-ix using the similar strategy as in Scheme 3(i). Alternatively, Compound S4-vi undergoes formylation at low temperature (e.g. −10° C.) first, followed with the cyclization using similar strategy as in Scheme 3(i) to give compound S4-x, which undergoes alkylation to give compound S4-xi. The halogen group (e.g. Hal is Br) in compound S4-xi can be functionalized with organometallic coupling reaction to generate compound S4-xii. In route (iv), compound S4-xiii is halogenated (e.g. NBS in BPO) and then reacts with AcOK followed by hydrolysis to give compound S4-xiv. Protection of the hydroxyl group followed by methylation gives compound S4-xv. Cyclization of S4-xv with N₂H₄ and subsequent alkylation gives compound S4-xvi. Deprotection and subsequent halogenation gives compound S4-xvii. Organometallic coupling (e.g. Stille reaction) of compound S4-xvii gives compound S4-xviii.

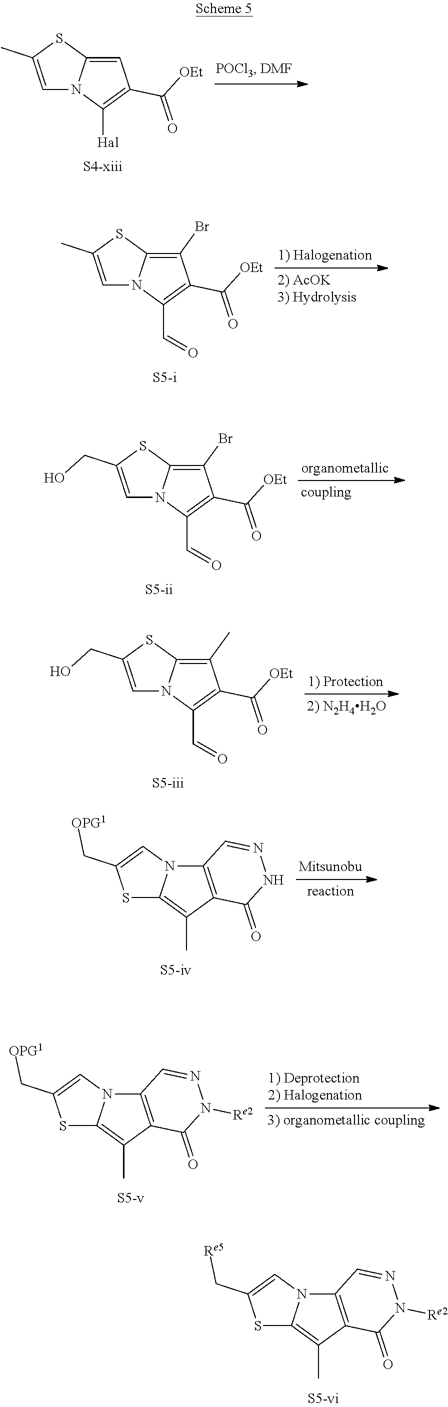

Hal is a halogen (e.g. Br), PG1 is as defined in Scheme 4. R^{e5} has the same definition as R² Formylation of compound S4-xiii-i at high temperature (POCl₃, DMF, 100° C.) generate compound S5-i with the bromide migration. Halogenation of the methyl group in compound S5-i, followed by treatment of AcOK and hydrolysis gives compound S5-ii. Organometallic coupling reaction (Me₄Sn Stille coupling) provides compound S5-iii. Protection and subsequent cyclization of compound S5-iii gives compound S5-iv. Compound S5-iv is subject to Mitsunobu reaction (cyanomethylenetributylphosphorane) to give compound S5-v. Deprotection, halogenation, and subsequent organometallic coupling reaction (Stille reaction) gives compound S5-vi.

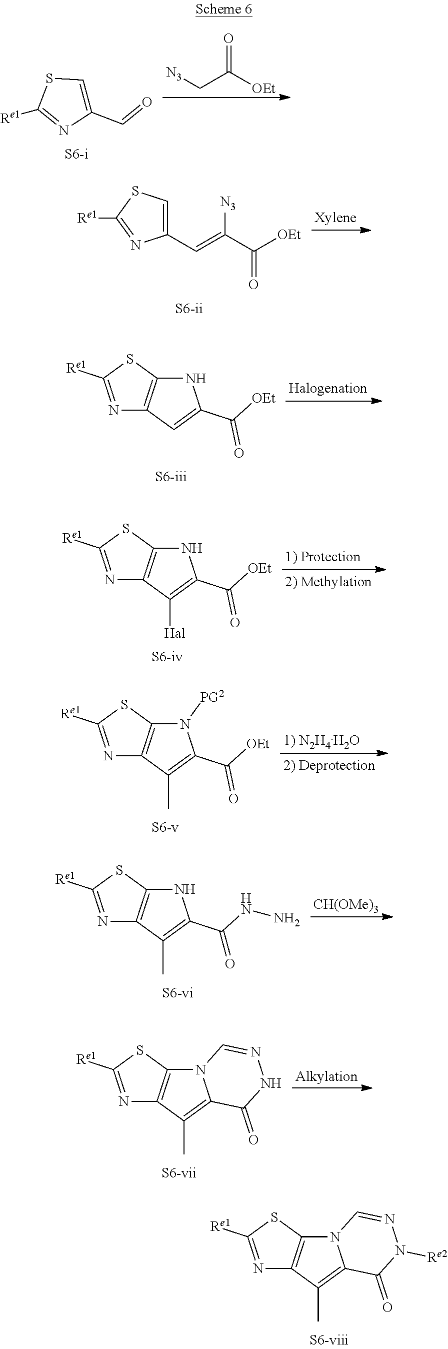

R^{e1} and R^{e2} are as defined in Scheme 1.

Compound S6-i reacts with ethyl azidoacetate under nucleophilic addition conditions (e.g., a base) in an appropriate solvent (e.g. ethanol), followed by reductive cyclization in xylene gives bicyclic compound S6-iii. Halogenation (e.g. NBS in DMF) of compound S6-iii provides compound S6-iv. Protection of the amino group in compound S6-iv followed by methylation gives compound S6-v. Compound S6-v reacts with hydrazine followed by deprotection and cyclization in trimethyloxymethane provides tricyclic compound S6-vii. Alkylation of S6-vii (alkyl halide and base) provides compound S6-viii.

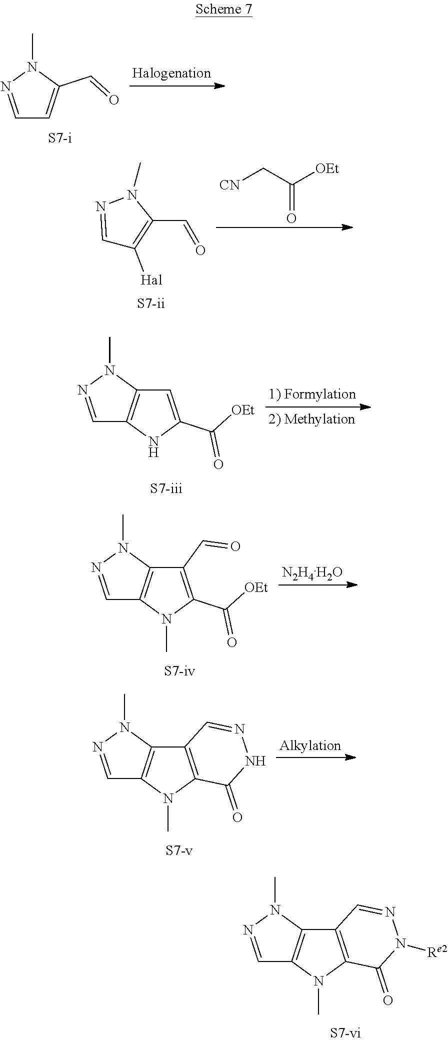

R^{e2} is as defined in Scheme 1.

Halogenation of compound S7-i gives compound S7-ii, which reacts with ethyl 2-isocyanoacetate to provide compound S7-iii. Formylation and subsequent methylation provides compound S7-iv. Compound S7-iv reacts with hydrazine followed by alkylation provides tricyclic compound S7-vi.

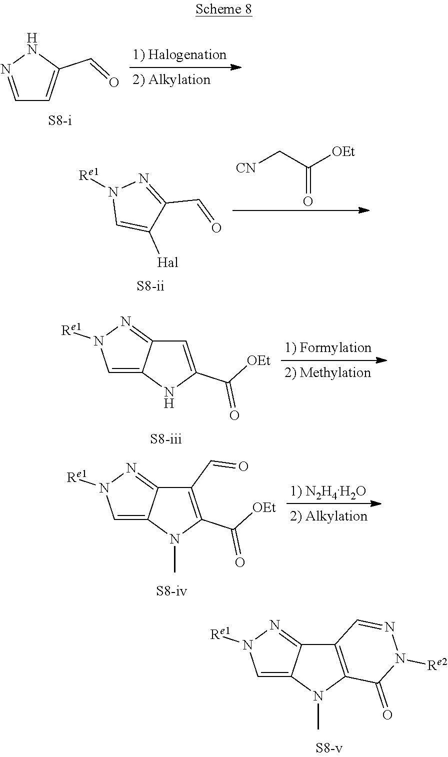

Hal is a halogen, and R^{e1} and R^{e2} are as defined in Scheme 1.

Halogenation of compound S8-i gives compound S8-ii, which can be subsequently alkylated to give compound S8-ii. Compound S8-ii reacts with ethyl 2-isocyanoacetate to provide compound S8-iii. Formylation and subsequent methylation provides compound S8-iv. Compound S8-iv reacts with hydrazine followed by alkylation provides tricyclic compound S8-v.

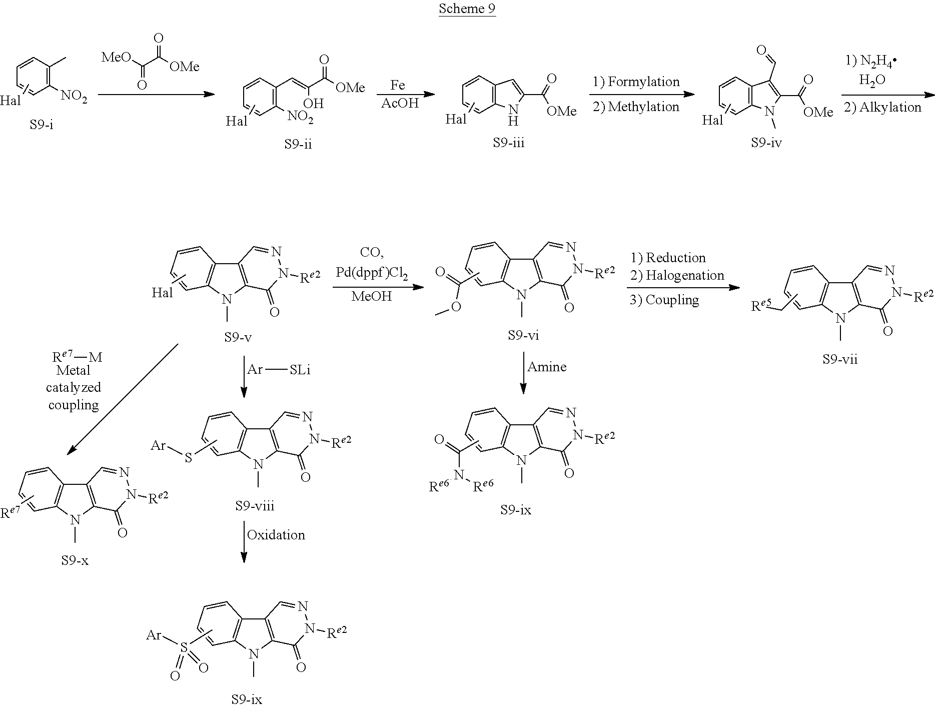

R^{e7} is C₁₋₆ alkyl; each R^{e6} is independently hydrogen or C₁₋₄ alkyl; Ar is an optionally substituted aryl, or optionally substituted heteroaryl; R^{e1} and R^{e2} are as defined in Scheme 1; R^{e5} is as defined in Scheme 5. M is metal. Ar is an optionally substituted aryl, or optionally substituted heteroaryl.

Compound S9-i reacts with dimethyl oxalate in the presence of base (e.g. NaH) to give compound S9-ii. Reduction and cyclization of compound S9-ii provides compound S9-iii. Formylation and methylation of compound S9-iii provides compound S9-iv, which reacts with hydrazine and undergoes alkylation to give tricyclic S9-v. Metal catalyzed coupling reactions of S9-v with different organometallic reagents give compound S9-x. Palladium catalyzed carbonylation of compound S9-v provided the ester S9-vi, which can react with primary or secondary amine to give amide S9-vi. Alternatively, the ester of compound S9-v can be reduced and halogenated to undergo a further organometallic coupling reaction to give compound S9-vii. In addition, compound S9-v can undergo a palladium catalyzed organometallic coupling reaction, for example with Ar-SLi (wherein Ar is an optionally substituted aryl, or optionally substituted heteroaryl) to give S9-viii, which undergo oxidation reaction to give compound S9-ix.

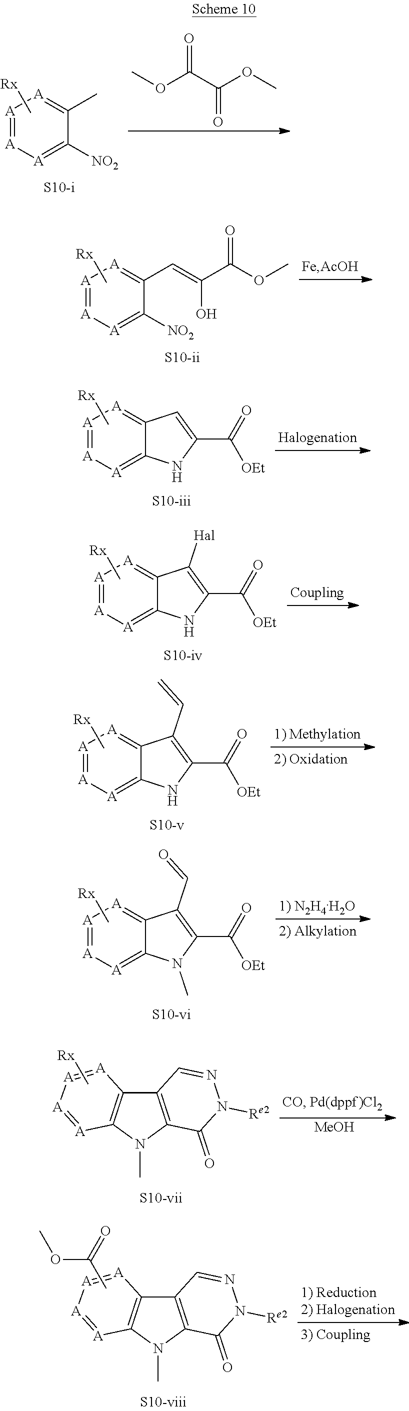

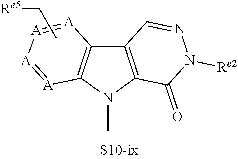

Each instance of A is independently CR₁ or N, provided only one A is N and the rest are CR₁, wherein R₁ is as defined; Rx is hydrogen or halogen; Hal is halogen; R² is as defined in Scheme 4. Similar to Scheme 9, compound S10-vii can be synthesized from nitro S10-i. When Rx is halogen (e.g., Br), S10-vii can undergo Palladium catalyzed carbonylation to give s10-viii, which can be reduced and halogenated to undergo a further organometallic coupling reaction to give compound S10-ix.

### Methods of Treatment

In one embodiment, provided is a method for treating a disease, condition or disorder as described herein (e.g., treating) comprising administering a compound, a pharmaceutically acceptable salt of a compound or pharmaceutical composition comprising a Disclosed Compound.

The compounds and compositions described herein can be administered to cells in culture, e.g. in vitro or ex vivo, or to a subject, e.g., in vivo, to treat, and/or diagnose a variety of disorders, including those described herein below.

In one embodiment of the invention provided is a method for increasing the lifetime of red blood cells (RBCs) comprising contacting red blood cells with an effective amount of (1) a Disclosed Compound or a pharmaceutically acceptable salt thereof; (2) a pharmaceutically acceptable composition comprising a Disclosed Compound or a pharmaceutically acceptable salt thereof, and a pharmaceutically acceptable carrier.

In a further embodiment the compound or pharmaceutical composition is added directly to whole blood comprising the red blood cells or packed red blood cells comprising the red blood cells (e.g. extracorporeally). In another embodiment, the compound or pharmaceutical composition is administered to a subject in need thereof comprising the red blood cells.

In one embodiment of the invention provided is a method for regulating 2,3-diphosphoglycerate levels in blood in need thereof comprising contacting blood with an effective amount of (1) a Disclosed Compound or a pharmaceutically acceptable salt thereof; (2) a pharmaceutically acceptable composition comprising a Disclosed Compound or a pharmaceutically acceptable salt thereof, and a pharmaceutically acceptable carrier.

In one embodiment of the invention provided is a method for treating sickle cell disease comprising administering to a subject in need thereof with an effective amount of (1) a Disclosed Compound or a pharmaceutically acceptable salt thereof; (2) a pharmaceutically acceptable composition comprising a Disclosed Compound or a pharmaceutically acceptable salt thereof, and a pharmaceutically acceptable carrier.

As used here, sickle cell disease (SCD), Hemoglobin SS disease, and sickle cell anemia are used interchangeably. Sickle cell disease (SCD) describes a group of inherited red blood cell disorders. In certain embodiments, subjects with SCD have abnormal hemoglobin, called hemoglobin S or sickle hemoglobin, in their red blood cells. In certain embodiments, a subject having SCD has at least one abnormal genes causing the body to make hemoglobin S. In certain embodiments, a subject having SCD has two hemoglobin S genes, Hemoglobin SS.

In one embodiment of the invention provided is a method of treating pyruvate kinase deficiency (PKD) in a subject comprising administering to the subject an effective amount of (1) a Disclosed Compound or a pharmaceutically acceptable salt thereof; (2) a pharmaceutically acceptable composition comprising a Disclosed Compound or a pharmaceutically acceptable salt thereof, and a pharmaceutically acceptable carrier.

As described herein, PKD is a deficiency of PKR. In certain embodiments, the deficiency of PKR is associated with a PKR mutation. In certain embodiments, PKD refers to presence of at least 2 mutant alleles in the PKLR gene. In certain embodiments, at least 1 of the at least 2 mutant allels in the PKLR gene is a missense mutation. In certain embodiments, a PKD patient has an Hb concentration less than or equal to 10.0 g/dL. In certain embodiments, the patient is not under regular transfusion (e.g. having had no more than 4 transfusion episodes in the 12-month period). In certain embodiments, the patient is under regular transfusion (e.g. having had ate least 4 transfusion episodes in the 12-month period). In certain embodiments, the patient is under a regular transfusion having at least 6 transfusion episodes in the 12-month period. In certain embodiments, the patient under regular transfusion has hemoglobin (Hb)≤12.0 g/dL (if male) or ≤11.0 g/dL (if female). In certain embodiments, the patient has undergone splenectomy.

In an embodiment, the mutant PKR is selected from the group consisting of A31V, A36G, G37Q, R40W, R40Q, L73P, S80P, P82H, R86P, I90N, T93I, G95R, M107T, G111R, A115P, S120F, H121Q, S130P, S130Y, V134D, R135D, A137T, G143S, I153T, A154T, L155P, G159V, R163C, R163L, T164N, G165V, L167M, G169G, E172Q, W201R, I219T, A221Y, D221N, G222A, I224T, G232C, N253D, G263R, G263W, E266K, V269F, L272V, L272P, G275R, G275R, E277K, V280G, D281N, F287V, F287L, V288L, D293N, D293V, A295I, A295V, I310N, I314T, E315K, N316K, V320L, V320M, S330R, D331N, D331G, D331E, G332S, V335M, A336S, R337W, R337P, R337Q, D339N, D339Q, G341A, G341D, I342F, K348N, A352D, I357T, G358R, G358E, R359C, R359H, C360Y, N361D, G364D, K365M, V368F, T371I, L374P, S376I, T384M, R385W, R385K, E387G, D390N, A392T, N393D, N393S, N393K, A394S, A394D, A394V, V395L, D397V, G398A, M403I, G406R, E407K, E407G, T408P, T408A, T408I, K410E, G411S, G411A, Q421K, A423A, A423A, R426W, R426Q, E427A, E427N, A431T, R449C, I457V, G458D, A459V, V460M, A468V, A468G, A470D, T477A, R479C, R479H, S485F, R486W, R486L, R488Q, R490W, I494T, A495T, A495V, R498C, R498H, A503V, R504L, Q505E, V506I, R510Q, G511R, G511E, R518S, R531C, R532W, R532Q, E538D, G540R, D550V, V552M, G557A, R559G, R559P, N566K, M568V, R569Q, R569L, Q58X, E174X, W201X, E241X, R270X, E440X, R486X, Q501X, L508X, R510X, E538X, R559X. These mutations are described in Canu et. al., Blood Cells, Molecules and Diseases 2016, 57, pp. 100-109. In an embodiment, the mutant PKR is selected from G332S, G364D, T384M, K410E, R479H, R479K, R486W, R532W, R510Q, and R490W. In certain embodiments, the mutant PKR is selected from A468V, A495V, I90N, T408I, and Q421K, and R498H. In certain embodiments, the mutant PKR is R532W, K410E, or R510Q.

In one embodiment of the invention provided is a method of treating anemia in a subject comprising administering to the subject an effective amount of (1) a Disclosed Compound or a pharmaceutically acceptable salt thereof; (2) a pharmaceutically acceptable composition comprising a Disclosed Compound or a pharmaceutically acceptable salt thereof, and a pharmaceutically acceptable carrier. In certain embodiments, the anemia is a dyserythropoietic anemia such as congenital dyserythropoietic anemia type I, II, III, or IV. In certain embodiments, the anemia is hemolytic anemia.

In certain embodiments, the hemolytic anemia is a congenital and/or hereditary form of hemolytic anemia such as PKD, sickle cell disease, thalassemias (e.g. alpha or beta), hereditary spherocytosis, hereditary elliptocytosis), paroxysmal nocturnal hemoglobinuria, abeta-liproteinemia (Bassen-Kornzweig syndrome). In certain embodiments, the hemolytic anemia is acquired hemolytic anemia such as autoimmune hemolytic anemia, drug-induced hemolytic anemia. In certain embodiments, the hemolytic anemia is chronic hemolytic anemia caused by phosphoglycerate kinase deficiency. In certain embodiments, the hemolytic anemia is anemia of chronic diseases, non-spherocytic hemolytic anemia, or hereditary spherocytosis. In certain embodiments, the hemolytic anemia is anemia as part of a multi-system disease, such as the anemia of Congenital Erythropoietic Purpura, Fanconi, Diamond-Blackfan.

As used herein, the term “anemia” refers to a deficiency of red blood cells (RBCs) and/or hemoglobin. As used herein, anemia includes all types of clinical anemia, for example (but not limited to): microcytic anemia, iron deficiency anemia, hemoglobinopathies, heme synthesis defect, globin synthesis defect, sideroblastic defect, normocytic anemia, anemia of chronic disease, aplastic anemia, hemolytic anemia, macrocytic anemia, megaloblastic anemia, pernicious anemia, dimorphic anemia, anemia of prematurity, Fanconi anemia, hereditary spherocytosis, sickle cell disease, warm autoimmune hemolytic anemia, cold agglutinin hemolytic anemia, osteopetrosis, thalassemia, and myelodysplastic syndrome.

In certain embodiments, anemia can be diagnosed on a complete blood count. In certain embodiments, anemia can be diagnosed based on the measurement of one or more markers of hemolysis (e.g. RBC count, hemoglobin, reticulocytes, schistocytes, lactate Dehydrogenase (LDH), haptoglobin, bilirubin, and ferritin) and/or hemosiderinuria mean corpuscular volume (MCV) and/or red cell distribution width (RDW). In the context of the present invention, anemia is present if an individual has a hemoglobin (Hb) less than the desired level, for example, the Hb concentration of less than 14 g/dL, more preferably of less than 13 g/dL, more preferably of less than 12 g/dL, more preferably of less than 11 g/dL, or most preferably of less than 10 g/dL.

In certain embodiments, provided herein is a method of increasing amount of hemoglobin in a subject in need thereof by administering an effective amount of a compound as described herein, or a pharmaceutically acceptable salt thereof, or a pharmaceutically acceptable composition thereof. In certain embodiments, the provided method increases hemoglobin concentration in the subject. In certain embodiments, the provided method increases increases Hb concentration to a desired level, for example, above 10 g/dL, more preferably above 11 g/dL, more preferably above 12 g/dL, more preferably above 13 g/dL, or most preferably above 14 g/dL. In certain embodiments, the provided method increases Hb concentration by at least about 0.5 g/dL. In certain embodiments, the provided method increases Hb concentration by at least about 1.0 g/dL. In certain embodiments, the provided method increases Hb concentration by at least about 1.5 g/dL. In certain embodiments, the provided method increases Hb concentration by at least about 2.0 g/dL. In certain embodiments, the provided method increases Hb concentration by at least about 2.5 g/dL. In certain embodiments, the provided method increases Hb concentration by at least about 3.0 g/dL. In certain embodiments, the provided method increases Hb concentration by at least about 3.5 g/dL. In certain embodiments, the provided method increases Hb concentration by at least about 4.0 g/dL. In certain embodiments, the provided method increases Hb concentration by at least about 4.5 g/dL. In certain embodiments, the provided method increases Hb concentration by at least about 5.0 g/dL. In certain embodiments, the provided method increases Hb concentration by at least about 5.5 g/dL. In certain embodiments, the provided method increases Hb concentration by at least about 6.0 g/dL.

In one embodiment of the invention provided is a method for treating hemolytic anemia comprising administering to a subject an effective amount of (1) a Disclosed Compound or a pharmaceutically acceptable salt thereof; (2) a pharmaceutically acceptable composition comprising a Disclosed Compound or a pharmaceutically acceptable salt thereof, and a pharmaceutically acceptable carrier.

In a further embodiment, the hemolytic anemia is hereditary and/or congenital hemolytic anemia, acquired hemolytic anemia, or anemia as part of a multisystem disease. In certain embodiments, the hemolytic anemia is congenital anemia. In certain embodiments, the hemolytic anemia is hereditary (e.g. non-spherocytic hemolytic anemia or hereditary spherocytosis).

In one embodiment of the invention provided is a method of treating thalassemia; hereditary spherocytosis; hereditary elliptocytosis; abetalipoproteinemia or Bassen-Kornzweig syndrome; paroxysmal nocturnal hemoglobinuria; acquired hemolytic anemia (e.g., congenital anemias (e.g., enzymopathies)); sickle cell disease; or anemia of chronic diseases comprising administering to a subject a therapeutically effective amount of (1) a Disclosed Compound or a pharmaceutically acceptable salt thereof; (2) a pharmaceutically acceptable composition comprising a Disclosed Compound or a pharmaceutically acceptable salt thereof, and a pharmaceutically acceptable carrier. In one embodiment, the acquired hemolytic anemia comprises congenital anemias. In certain embodiments, the provided method is to treat thalassemia. In certain embodiments, the provided method is to treat beta thalassemia.

As used herein, thalassemia is an inherited blood disorder in which the body makes an abnormal form of hemoglobin. In certain embodiments, the disorder results in large numbers of red blood cells being destroyed, which leads to anemia. In certain embodiments, the thalassemia is alpha thalassemia. In certain embodiments, the thalassemia is beta thalassemia.

In one embodiment of the invention provided is a method for activating mutant PKR in red blood cells comprising administering to a subject in need thereof an effective amount of (1) a Disclosed Compound or a pharmaceutically acceptable salt thereof; (2) a pharmaceutically acceptable composition comprising a Disclosed Compound or a pharmaceutically acceptable salt thereof, and a pharmaceutically acceptable carrier. In one embodiment, the method is an ex vivo method. In another embodiment, the method is an in vitro method. In some embodiments, the blood or the red blood cells are derived or obtained from a subject suffering from or susceptible to a disease or disorder selected from the group consisting of pyruvate kinase deficiency (PKD), thalassemia (e.g., beta thalassemia), hereditary spherocytosis, hereditary elliptocytosis, abetalipoproteinemia or Bassen-Kornzweig syndrome, sickle cell disease, paroxysmal nocturnal hemoglobinuria, anemia (e.g., dyserythropoetic anemia), hemolytic anemia, and anemia of chronic diseases. In some embodiments, the hemolytic anemia is hereditary and/or congenital hemolytic anemia, acquired hemolytic anemia, or anemia as part of a multi-system disease.

In one embodiment of the invention provided is a method for activating wild-type PKR in red blood cells comprising administering to a subject in need thereof an effective amount of (1) a Disclosed Compound or a pharmaceutically acceptable salt thereof; (2) a pharmaceutically acceptable composition comprising a Disclosed Compound or a pharmaceutically acceptable salt thereof, and a pharmaceutically acceptable carrier. In one embodiment, the method is an ex vivo method. In another embodiment, the method is an in vitro method. In some embodiments, the blood or the red blood cells are derived or obtained from a subject suffering from or susceptible to a disease or disorder selected from the group consisting of pyruvate kinase deficiency (PKD), thalassemia (e.g., beta thalassemia), hereditary spherocytosis, hereditary elliptocytosis, abetalipoproteinemia or Bassen-Kornzweig syndrome, sickle cell disease, paroxysmal nocturnal hemoglobinuria, anemia (e.g., dyserythropoetic anemia), hemolytic anemia, and anemia of chronic diseases. In some embodiments, the hemolytic anemia is hereditary and/or congenital hemolytic anemia, acquired hemolytic anemia, or anemia as part of a multi-system disease.

In one embodiment of the invention provided is a use of (1) a Disclosed Compound or a pharmaceutically acceptable salt thereof; (2) a pharmaceutically acceptable composition comprising a Disclosed Compound or a pharmaceutically acceptable salt thereof, and a pharmaceutically acceptable carrier for the preparation of a medicament for increasing the lifetime of red blood cells (RBCs) in need thereof.

In a further embodiment the compound or pharmaceutical composition is formulated to be added directly to whole blood or packed red blood cells extracorporeally. In another embodiment, the compound or pharmaceutical composition is formulated to be administered to a subject in need thereof.

In one embodiment of the invention provided is a use of (1) a Disclosed Compound or a pharmaceutically acceptable salt thereof; (2) a pharmaceutically acceptable composition comprising a Disclosed Compound or a pharmaceutically acceptable salt thereof, and a pharmaceutically acceptable carrier for the preparation of a medicament for regulating 2,3-diphosphoglycerate levels in blood in need thereof.

In one embodiment of the invention provided is a use of (1) a Disclosed Compound or a pharmaceutically acceptable salt thereof; (2) a pharmaceutically acceptable composition comprising a Disclosed Compound or a pharmaceutically acceptable salt thereof, and a pharmaceutically acceptable carrier for the preparation of a medicament for treating anemia. In certain embodiments, the anemia is a dyserythropoietic anemia such as congenital dyserythropoietic anemia type I, II, III, or IV. In certain embodiments, the anemia is hemolytic anemia. In certain embodiments, the hemolytic anemia is a congenital and/or hereditary form of hemolytic anemia such as PKD, sickle cell disease, thalassemias (e.g. alpha or beta), hereditary spherocytosis, hereditary elliptocytosis), paroxysmal nocturnal hemoglobinuria, abeta-liproteinemia (Bassen-Kornzweig syndrome). In certain embodiments, the hemolytic anemia is acquired hemolytic anemia such as autoimmune hemolytic anemia, drug-induced hemolytic anemia. In certain embodiments, the hemolytic anemia is anemia as part of a multi-system disease, such as the anemia of Congenital Erythropoietic Purpura, Fanconi, Diamond-Blackfan.

In one embodiment of the invention provided is a use of (1) a Disclosed Compound or a pharmaceutically acceptable salt thereof; (2) a pharmaceutically acceptable composition comprising a Disclosed Compound or a pharmaceutically acceptable salt thereof, and a pharmaceutically acceptable carrier for the preparation of a medicament for treating hemolytic anemia.

In one embodiment of the invention provided is a use of (1) a Disclosed Compound or a pharmaceutically acceptable salt thereof; (2) a pharmaceutically acceptable composition comprising a Disclosed Compound or a pharmaceutically acceptable salt thereof, and a pharmaceutically acceptable carrier for the preparation of a medicament for treating sickle cell disease.

In one embodiment of the invention provided is a use of (1) a Disclosed Compound or a pharmaceutically acceptable salt thereof; (2) a pharmaceutically acceptable composition comprising a Disclosed Compound or a pharmaceutically acceptable salt thereof, and a pharmaceutically acceptable carrier for the preparation of a medicament for treating pyruvate kinase deficiency (PKD) in a subject.

As described herein, PKD is a deficiency of PKR. In certain embodiments, the deficiency of PKR is associated with a PKR mutation.

In one embodiment of the invention provided is a use of (1) a Disclosed Compound or a pharmaceutically acceptable salt thereof; (2) a pharmaceutically acceptable composition comprising a Disclosed Compound or a pharmaceutically acceptable salt thereof, and a pharmaceutically acceptable carrier for the preparation of a medicament for treating thalassemia; hereditary spherocytosis; hereditary elliptocytosis; abetalipoproteinemia or Bassen-Kornzweig syndrome; paroxysmal nocturnal hemoglobinuria; acquired hemolytic anemia; or anemia of chronic diseases.

In one embodiment of the invention provided is a use of (1) a Disclosed Compound or a pharmaceutically acceptable salt thereof; (2) a pharmaceutically acceptable composition comprising a Disclosed Compound or a pharmaceutically acceptable salt thereof, and a pharmaceutically acceptable carrier for the preparation of a medicament for activating mutant PKR in red blood cells.

In one embodiment of the invention provided is a use of (1) a Disclosed Compound or a pharmaceutically acceptable salt thereof; (2) a pharmaceutically acceptable composition comprising a Disclosed Compound or a pharmaceutically acceptable salt thereof, and a pharmaceutically acceptable carrier for the preparation of a medicament for activating wild-type PKR in red blood cells.

In one embodiment of the invention, provided is a method of activating pyruvate kinase R (PKR), comprising contacting the PKR with an effective amount of of (1) a Disclosed Compound, or a pharmaceutically acceptable salt thereof; or (2) a pharmaceutically acceptable composition comprising a Disclosed Compound or a pharmaceutically acceptable salt thereof, and a pharmaceutically acceptable carrier. In one embodiment, the PKR is a wild-type PKR. In another embodiment, the PKR is a mutant PKR. In some embodiments, the PKR is expressed in red blood cells. In one embodiment, the method is an ex vivo method. In another embodiment, the method is an in vitro method. In some embodiments, the blood or the red blood cells are derived or obtained from a subject suffering from or susceptible to a disease or disorder selected from the group consisting of thalassemia (e.g., beta thalassemia), hereditary spherocytosis, hereditary elliptocytosis, abetalipoproteinemia or Bassen-Kornzweig syndrome, sickle cell disease, paroxysmal nocturnal hemoglobinuria, anemia (e.g., dyserythropoetic anemia), hemolytic anemia, and anemia of chronic diseases. In some embodiments, the hemolytic anemia is hereditary and/or congenital hemolytic anemia, acquired hemolytic anemia, or anemia as part of a multi-system disease.

Since the compounds and compositions described herein act on the same biological pathway and have the similar mode of action as the compounds described in WO2012/151451, the compounds and compositions presented herein can activate the PKR mutants as described in WO2012/151451.

### Proliferative Disease

In some embodiments, provided is a method of treating a proliferative disease comprising administering to a subject a compound, a pharmaceutically acceptable salt thereof, or pharmaceutical composition thereof, as described herein. As used here, “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases. In certain embodiments, the proliferative diease is cancer. In certain embodiments, the proliferative diease is an autoimmune disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include solid tumors, soft tissue tumors, and metastases thereof. The disclosed methods are also useful in treating non-solid cancers. Exemplary solid tumors include malignancies (e.g., sarcomas, adenocarcinomas, and carcinomas) of the various organ systems, such as those of lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine. Other exemplary cancers include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primaiy; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-CeIl Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular MeI anoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular MeI anoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-CeIl; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; MeI anoma; MeI anoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (MeI anoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor. Metastases of the aforementioned cancers can also be treated or prevented in accordance with the methods described herein.

### Cancer Combination Therapies

In some embodiments, the provided method further comprises administering one or more additional cancer treatments. Exemplary cancer treatments include, for example: chemotherapy, targeted therapies such as antibody therapies, immunotherapy, and hormonal therapy. Examples of each of these treatments are provided below.

In some embodiments, a Disclosed Compound is administered with one or morechemotherapies. Chemotherapy is the treatment of cancer with drugs that can destroy cancer cells. “Chemotherapy” usually refers to cytotoxic drugs which affect rapidly dividing cells in general, in contrast with targeted therapy. Chemotherapy drugs interfere with cell division in various possible ways, e.g., with the duplication of DNA or the separation of newly formed chromosomes. Most forms of chemotherapy target all rapidly dividing cells and are not specific for cancer cells, although some degree of specificity may come from the inability of many cancer cells to repair DNA damage, while normal cells generally can.

Examples of chemotherapeutic agents used in cancer therapy include, for example, antimetabolites (e.g., folic acid, purine, and pyrimidine derivatives) and alkylating agents (e.g., nitrogen mustards, nitrosoureas, platinum, alkyl sulfonates, hydrazines, triazenes, aziridines, spindle poison, cytotoxic agents, toposimerase inhibitors and others). Exemplary agents include Aclarubicin, Actinomycin, Alitretinon, Altretamine, Aminopterin, Aminolevulinic acid, Amrubicin, Amsacrine, Anagrelide, Arsenic trioxide, Asparaginase, Atrasentan, Belotecan, Bexarotene, endamustine, Bleomycin, Bortezomib, Busulfan, Camptothecin, Capecitabine, Carboplatin, Carboquone, Carmofur, Carmustine, Celecoxib, Chlorambucil, Chlormethine, Cisplatin, Cladribine, Clofarabine, Crisantaspase, Cyclophosphamide, Cytarabine, Dacarbazine, Dactinomycin, Daunorubicin, Decitabine, Demecolcine, Docetaxel, Doxorubicin, Efaproxiral, Elesclomol, Elsamitrucin, Enocitabine, Epirubicin, Estramustine, Etoglucid, Etoposide, Floxuridine, Fludarabine, Fluorouracil (5FU), Fotemustine, Gemcitabine, Gliadel implants, Hydroxycarbamide, Hydroxyurea, Idarubicin, Ifosfamide, Irinotecan, Irofulven, Ixabepilone, Larotaxel, Leucovorin, Liposomal doxorubicin, Liposomal daunorubicin, Lonidamine, Lomustine, Lucanthone, Mannosulfan, Masoprocol, MeI phalan, Mercaptopurine, Mesna, Methotrexate, Methyl aminolevulinate, Mitobronitol, Mitoguazone, Mitotane, Mitomycin, Mitoxantrone, Nedaplatin, Nimustine, Oblimersen, Omacetaxine, Ortataxel, Oxaliplatin, Paclitaxel, Pegaspargase, Pemetrexed, Pentostatin, Pirarubicin, Pixantrone, Plicamycin, Porfimer sodium, Prednimustine, Procarbazine, Raltitrexed, Ranimustine, Rubitecan, Sapacitabine, Semustine, Sitimagene ceradenovec, Satraplatin, Streptozocin, Talaporfin, Tegafur-uracil, Temoporfin, Temozolomide, Teniposide, Tesetaxel, Testolactone, Tetranitrate, Thiotepa, Tiazofurin, Tioguanine, Tipifarnib, Topotecan, Trabectedin, Triaziquone, Triethylenemelamine, Triplatin, Tretinoin, Treosulfan, Trofosfamide, Uramustine, Valrubicin, Verteporfin, Vinblastine, Vincristine, Vindesine, Vinflunine, Vinorelbine, Vorinostat, Zorubicin, and other cytostatic or cytotoxic agents described herein.

In some embodiments, a Disclosed Compound is administered with one or more targeted therapies. Targeted therapy constitutes the use of agents specific for the deregulated proteins of cancer cells. Small molecule targeted therapy drugs are generally inhibitors of enzymatic domains on mutated, overexpressed, or otherwise critical proteins within the cancer cell. Prominent examples are the tyrosine kinase inhibitors such as Axitinib, Bosutinib, Cediranib, dasatinib, erlotinib, imatinib, gefitinib, lapatinib, Lestaurtinib, Nilotinib, Semaxanib, Sorafenib, Sunitinib, and Vandetanib, and also cyclin-dependent kinase inhibitors such as Alvocidib and Seliciclib. Monoclonal antibody therapy is another strategy in which the therapeutic agent is an antibody which specifically binds to a protein on the surface of the cancer cells. Examples include the anti-HER2/neu antibody trastuzumab (HERCEPTIN®) typically used in breast cancer, and the anti-CD20 antibody rituximab and Tositumomab typically used in a variety of B-cell malignancies. Other exemplary anbibodies include Cetuximab, Panitumumab, Trastuzumab, Alemtuzumab, Bevacizumab, Edrecolomab, and Gemtuzumab. Exemplary fusion proteins include Aflibercept and Denileukin diftitox. In some embodiments, the targeted therapy can be used in combination with a Disclosed Compound.

Targeted therapy can also involve small peptides as “homing devices” which can bind to cell surface receptors or affected extracellular matrix surrounding the tumor. Radionuclides which are attached to these peptides (e.g., RGDs) eventually kill the cancer cell if the nuclide decays in the vicinity of the cell. An example of such therapy includes BEXXAR®.

In some embodiments, a Disclosed Compound is administered with one or more immunotherapies. Cancer immunotherapy refers to a diverse set of therapeutic strategies designed to induce the patient's own immune system to fight the tumor. Contemporary methods for generating an immune response against tumors include intravesicular BCG immunotherapy for superficial bladder cancer, and use of interferons and other cytokines to induce an immune response in renal cell carcinoma and melanoma patients.

Allogeneic hematopoietic stem cell transplantation can be considered a form of immunotherapy, since the donor's immune cells will often attack the tumor in a graft-versus-tumor effect. In some embodiments, the immunotherapy agents can be used in combination with a Disclosed Compound.

In some embodiments, a Disclosed Compound is administered with one or more hormonal therapies. The growth of some cancers can be inhibited by providing or blocking certain hormones. Common examples of hormone-sensitive tumors include certain types of breast and prostate cancers. Removing or blocking estrogen or testosterone is often an important additional treatment. In certain cancers, administration of hormone agonists, such as progestogens may be therapeutically beneficial. In some embodiments, the hormonal therapy agents can be used in combination with a Disclosed Compound.

### Obesity and Fat Disorders

In some embodiments, provided is a method of treating or preventing obesity in a human subject (e.g. a child or adult) by administering to the human subject an effective amount of the compound, pharmaceutically acceptable salt, or pharmaceutical composition thereof as described herein. “Obesity” refers to a condition in which a subject has a body mass index of greater than or equal to 30. Many Disclosed Compounds can be used to treat or prevent an over-weight condition. “Over-weight” refers to a condition in which a subject has a body mass index of greater or equal to 25.0. The body mass index (BMI) and other definitions are according to the “NIH Clinical Guidelines on the Identification and Evaluation, and Treatment of Overweight and Obesity in Adults” (1998). Treatment with the compound may be in an amount effective to alter the weight of the subject, e.g., by at least 2, 5, 7, 10, 12, 15, 20, 25, 30, 25, 40, 45, 50, or 55%. Treatment with a compound may be in an amount effective to reduce the body mass index of the subject, e.g., to less than 30, 28, 27, 25, 22, 20, or 18. The compounds can be used to treat or prevent aberrant or inappropriate weight gain, metabolic rate, or fat deposition, e.g., anorexia, bulimia, obesity, diabetes, or hyperlipidemia (e.g., elevated triglycerides and/or elevated cholesterol), as well as disorders of fat or lipid metabolism.

A compound or composition described herein can be administered to treat obesity associated with Prader-Willi Syndrome (PWS). PWS is a genetic disorder associated with obesity (e.g., morbid obesity).

A compound or composition described herein can be used to reduce body fat, prevent increased body fat, reduce cholesterol (e.g., total cholesterol and/or ratios of total cholesterol to HDL cholesterol), and/or reduce appetite in individuals having PWS associated obesity, and/or reduce comorbidities such as diabetes, cardiovascular disease, and stroke.

### Hyperglycemia

High glucose levels induce metabolic abnormalities in glucose metabolic pathways and induce mitochondrial dysfunction. This also overproduces reactive oxygen species (ROS). Elevated intracellular glucose leads to accumulation of the toxic glucose metabolites sorbitol, methylglyoxal (MG) and diacylglycerol (DAG), which have been proposed to contribute to microvascular complication, e.g., DN. Small-molecule PKM2 activators were found to reverse hyperglycemia-induced elevation in toxic glucose metabolites and mitochondrial dysfunction (Nat Med. 2017, 23(6): 753-762; U.S. Pat. No. 9,921,221).

In certain embodiments, provided herein is a method of treating hyperglycemia in a subject comprising comprising administering an effective amount of the compound, pharmaceutically acceptable salt, or pharmaceutical composition thereof.

In certain embodiments, provided herein is a method of treating a diabetic disease in a subject comprising administering an effective amount of the compound, pharmaceutically acceptable salt, or pharmaceutical composition thereof. A “diabetic disease” as used herein refers to diabetes and pre-diabetes as well as diabetic implications. Diabetes refers to a group of metabolic diseases in which a person has high blood sugar, either because the body does not produce enough insulin, or because cells do not respond to the insulin that is produced. This high blood sugar produces the classical symptoms of polyuria (frequent urination), polydipsia (increased thirst) and polyphagia (increased hunger). There are several types of diabetes. Type I diabetes results from the body's failure to produce insulin, and presently requires the person to inject insulin or wear an insulin pump. Type II diabetes results from insulin resistance a condition in which cells fail to use insulin properly, sometimes combined with an absolute insulin deficiency. Gestational diabetes occurs when pregnant women without a previous diagnosis of diabetes develop a high blood glucose level. Other forms of diabetes include congenital diabetes, which is due to genetic defects of insulin secretion, cystic fibrosis-related diabetes, steroid diabetes induced by high doses of glucocorticoids, and several forms of monogenic diabetes, e.g., mature onset diabetes of the young (e.g., MODY 1, 2, 3, 4, 5, 6, 7, 8, 9, or 10). Pre-diabetes indicates a condition that occurs when a person's blood glucose levels are higher than normal but not high enough for a diagnosis of diabetes. All forms of diabetes increase the risk of long-term complications. These typically develop after many years, but may be the first symptom in those who have otherwise not received a diagnosis before that time. The major long-term complications relate to damage to blood vessels. Exemplary diabetic implications include cardiovascular disease, macrovascular diseases such as ischemic heart disease (angina, myocardial infarction), stroke, and peripheral vascular disease, microvascular complications (e.g., damage to the small blood vessels), diabetic retinopathy (i.e. the impact of diabetes on blood vessel formation in the retina of the eye), diabetic nephropathy (i.e. the impact of diabetes on the kidneys), diabetic neuropathy (e.g. the impact of diabetes on the nervous system, most commonly causing numbness, tingling and pain in the feet and also increasing the risk of skin damage due to altered sensation), diabetic foot ulcers, and syndrome X. In certain embodiments, a “diabetic disease” includes one or more selected from hyperglycemia, hyperinsulinaemia, diabetes, insulin resistance, impaired glucose metabolism, conditions of impaired glucose tolerance (IGT), conditions of impaired fasting plasma glucose, diabetic retinopathy, diabetic nephropathy (“DN”), glomerulosclerosis, diabetic neuropathy and syndrome X.

In certain embodiments, the compound or composition described herein can be used to lower the reactive oxygen species (ROS) and/or at least one of the glucose metabolites (e.g. sorbitol, methylglyoxal (MG) and diacylglycerol (DAG)) in a subject.

In certain embodiments, the compound or composition described herein can be used to treat a microvascular complication.

In certain embodiments, the compound or composition described herein can be used to treat DN. In certain embodiments, the treatment of DN can include lessening of any symptom associated with DN, including, but not limited to, changes in appetite, change in sleep, protein in serum, weakness, and/or nausea.

In certain embodiments, the method further comprises administering to the subject an effective amount of one or more secondary agents that increase the level or activity of one or more of the DN protective factors. Exemplary DN protective factors include, but are not limited to SOD1-Superoxide dismutase; TPI1-Triosephosphate isomerase isoform 2; SORD-Sorbitol dehydrogenase; ALDOA-Aldolase A, fructose-bisphosphate; GAPDH Glyceraldehyde-3-phosphate dehydrogenase; PKM-Pyruvate kinase isozymes M1/M2; ENO1-Alpha-enolase; FGB-Fibrinogen beta chain; SELENBP1-Selenium binding protein 1; PEBP1-Phosphatidylethanolamine-binding protein 1; CRYL1-Lambda-crystallin homolog (U.S. Pat. No. 9,921,221, which is incorporated by reference on its entirety). A secondary agent may increase the level or activity of a protective factor or decrease the level or activity of a risk factor by at least 50%, 100% (1-fold), 1½-fold, 2-fold, 3-fold, 4-fold, 5-fold, 10-fold, 15-fold, 20-fold or more. In certain embodiments, the provided method comprises bringing the level or activity of a protective factor essentially to its level or activity in a subject that is protected from the development of a microvascular complication. “Essentially within its level,” refers to within less than 5%, 10%, 20%, 30%, 40%, 50%, 60%, 70%, 80%, 90% or 100% of the control value. The secondary agent may be a small molecule, a protein comprising the protective factor or a biologically active variant (e.g., fragment) thereof, or a nucleic acid encoding a protein comprising the protective factor or a biologically active variant (e.g., fragment) thereof. Biologically active variants of the proteins of protective factors also include full length immature and mature forms or fragments thereof that comprise an amino acid sequence that differs from the naturally occurring sequence or fragment thereof in at most 1, 2, 3, 4, 5, 10, 15, 20, 25, 30, 35, 40, 45, 50 or 100 amino acid deletions, additions or substitutions, such as conservative amino acid substitutions. Biologically active variants of the proteins of the DN protective factors may also include variants that are at least 70%, 80%, 85%, 90%, 95%, 97%, 98% or 99% identical to the full length mature or precursor human PEBP1 protein (or other biomarker identified in this specification) or a fragment thereof.

In some embodiments, the method provided further comprises selecting a subject for treatment. For example, a subject can be selected if the subject has or is at risk for developing DN, e.g., a subject having diabetes, e.g., type 1 or type 2 diabetes, or a subject who is prediabetic, e.g., having metabolic syndrome, insulin resistance, hyperglycemia, hyperlipidemia or a subject who is overweight or obese, e.g., having a BMI≥25. In some instances, a subject can be selected if the subject has or is at risk for developing type 1 and/or type 2 diabetes. In some instances, a subject can be selected if the subject is taking or will take insulin, e.g., to treat diabetes.

Cardiovascular disease is a chronic inflammatory condition. Increased glucose uptake and glycolytic flux promotes reactive oxygen species in mitochondria. ROS promotes dimerization of PKM2 and enable its nuclear translocation. Nuclear PKM2 functions as protein kinase and boosts IL-6 and IL-1β production. This results in systemic and tissue inflammation. Reducing glycolysis and enforcing PKM2 tetramerization was found to correct proinflammatory phenotype of coronary artery disease (CAD) macrophages (J. Exp. Med. 2016, 213(3): 337-354).

In certain embodiments, provided herein is a method of treating a cardiovascular disease in a subject comprising administering a therapeutic effective amount of the compound, pharmaceutically acceptable salt, or pharmaceutical composition thereof. The compounds or composition described herein can lower the plasma glucose level in a subject. A “cardiovascular disease” as defined in this application comprises, but is not limited to hypertension, congestive heart failure, diabetes, glomerulosclerosis, chronic renal failure, coronary heart disease, angina pectoris, myocardial infarction, stroke, vascular restenosis endothelial dysfunction, impaired vascular compliance and congestive heart failure. In certain embodiments, the cardiovascular disease is coronary artery disease (CAD). In certain embodiments, the compound or composition described herein can be used to lower the reactive oxygen species (ROS) in mitochondria in a subject.

In certain embodiments, provided herein is a method of treating an autoimmune disease in a subject comprising comprising administering a therapeutic effective amount of the compound, pharmaceutically acceptable salt, or pharmaceutical composition thereof. It was found that activation of PKM2 attenuated an LPS-induced pro-inflammatory M1 macrophage phenotype while promoting traits typical of an M2 macrophage. Additionally, it was found activation of PKM2 by TEPP-46 in vivo inhibited LPS and IL-1β production, whilst boosting production of IL-10. (Cell Metab. 2015, 21(1): 65-80) Accordingly, PKM2 activators can be useful to treat an autimmune disease by promoting IL-1β and/or IL-10 production.

An “autoimmune disease” refers to a disease arising from an inappropriate immune response of the body of a subject against substances and tissues normally present in the body. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy.

### Compositions and Routes of Administration

The compositions delineated herein include the compounds delineated herein (e.g., a Disclosed Compound), as well as additional therapeutic agents if present, in amounts effective for achieving a modulation of disease or disease symptoms, including those described herein.

The term “pharmaceutically acceptable carrier or adjuvant” refers to a carrier or adjuvant that may be administered to a patient, together with a compound provided herewith, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions provided herewith include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as β-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The pharmaceutical compositions provided herewith may be administered orally, parenterally, by inhalation spray, topically, rectally, nasally, buccally, vaginally or via an implanted reservoir, preferably by oral administration or administration by injection. The pharmaceutical compositions provided herewith may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The pharmaceutical compositions provided herewith may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

When the compositions provided herewith comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds provided herewith. Alternatively, those agents may be part of a single dosage form, mixed together with the compounds provided herewith in a single composition.

The Disclosed Compounds can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions provided herewith will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

### EXPERIMENTAL

Abbreviations list

| abbrv. | Full Name | abbrv. | Full Name |
|---|---|---|---|
| anhy. | anhydrous | aq. | aqueous |
| min | minute(s) | satd. | saturated |
| mL | milliliter | hrs | hours |
| mmol | millimole(s) | mol | mole(s) |
| MS | mass spectrometry | NMR | nuclear magnetic resonance |
| TLC | thin layer | HPLC | high-performance liquid |
| | chromatography | | chromatography |
| LCMS | Liquid chromato- | DCM | dichloromethane |
| | graphy—mass | | |
| | spectrometry | | |
| PE | petroleum ether | DMF | dimethylformamide |
| Et2O | diethyl ether | EtOH | ethyl alcohol |
| EA, | ethyl acetate | MeOH | methyl alcohol |
| EtOAc | | | |
| MeCN | acetonitrile | THF | tetrahydrofuran |
| DCE | 1,2-dichloroethane | DMSO | dimethyl sulfoxide |
| CHCl₃ | chloroform | Py or Pyr | pyridine |
| Et₃N or | triethylamine | TFA | trifluoroacetic acid |
| TEA | | | |
| DMAP | 4-(dimethylamino) | DIPEA | N,N-diisopropylethylamine |
| | pyridine | | |
| CMBP | cyanomethylene- | BPO | benzoyl peroxide |
| | tributylphosphorane | | |
| NBS | N-bromosuccinimide | NIS | N-iodosuccinimide |
| dppf | 1,1′-bis(diphenyl- | | |
| | phosphino) ferrocene | | |

### General Experimental

In the following examples, the chemical reagents were purchased from commercial sources (such as Alfa, Acros, Sigma Aldrich, TCI and Shanghai Chemical Reagent Company), and used without further purification. Flash chromatography was performed on an Isolera One (Biotage) via column with silica gel particles of 200-300 esh. Analytical and preparative thin layer chromatography plates (TLC) were HSGF 254 (0.15-0.2 mm thickness, Shanghai Anbang Company, China). Nuclear magnetic resonance (NMR) spectra were recorded using Brucker NMR Avance Neo 400 (Brucker, Switzerland). Chemical shifts were reported in parts per million (ppm, δ). etero(ESI) from a Shimadzu LCMS 2000 Mass Spectrometer. HPLC chromatographs were recorded on Shimadzu LC-2010AHT. Microwave reactions were run on a Microwave Synthesizer (CEM Discover SP)

HPLC conditions used in the experiments described herein are as follows:

### Method 1:

### Instrument: Shimadzu LC-2010AHT

### Column: YMC-Triart C18, 50×4.6 mm, 5 μm

Mobile phase: Solvent A:H₂O/CH₃OH/TFA=90/10/0.1,
Solvent B: H₂O/CH₃OH/TFA=10/90/0.1
   Flow rate: 2.5 mL/min; Column temperature: 35° C.; Wavelength: 220 nm/254 nm

### Method 2:

### Instrument: Shimadzu LC-2010AHT

### Column: YMC-Triart C18, 50×4.6 mm, 5 μm

Mobile phase: Solvent A:H₂O/CH₃CN/TFA=90/10/0.1,
Solvent B: H₂O/CH₃CN/TFA=10/90/0.1
   Flow rate: 2.5 mL/min; Column temperature: 35° C.; Wavelength: 220 nm/254 nm

Prep-HPLC conditions used in the experiments described herein are as follows:

### Instrument: Waters 2545B/2767

### Column: YMC-Triart C18, 250×20 mm, 5 μm

Mobile phase: Solvent A:H₂O (0.1% FA),
Solvent B: CH₃OH or CH₃CN
   Flow rate: 20 mL/min; Column temperature: 35° C.; Wavelength: 220 nm/254 nm

### Example 1: Synthesis of 6-(3-methoxybenzyl)-2,4-dimethyl-6,7-dihydropyrrolo[3,4-b]thieno[2,3-d]pyrrol-5 (4H)-one

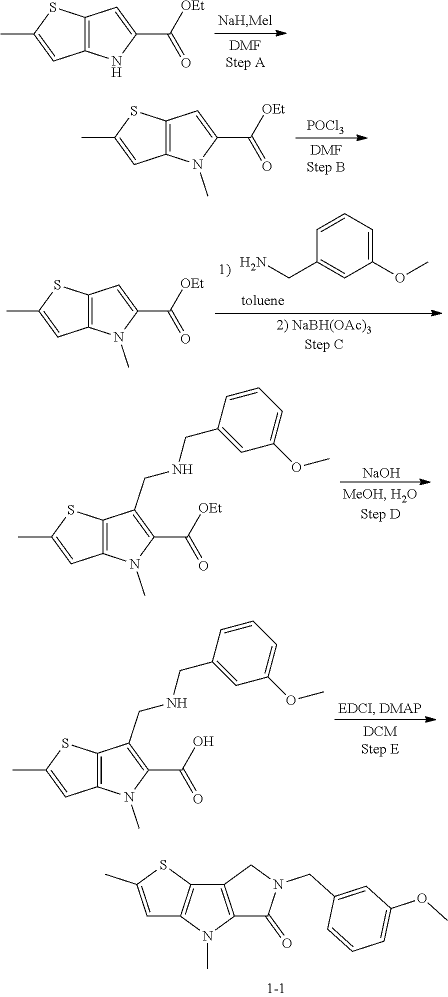

Step A. Synthesis of ethyl 2,4-dimethyl-4H-thieno[3,2-b]pyrrole-5-carboxylate To a mixture of ethyl 2-methyl-4H-thieno[3,2-b]pyrrole-5-carboxylate (500 mg, 2.4 mmol) in DMF (30 mL) was added NaH (114 mg, 4.8 mmol) at 0° C. The mixture was stirred at r.t. for 30 min, followed by the addition of MeI (678 mg, 4.78 mmol) at 0° C. After stirred at r.t. for 2 hr, the mixture was poured into satd. NH₄Cl, extracted with EtOAc. The combined organic layer was dried over anhy. Na₂SO₄ and concentrated. The residue was purified by silica gel chromatography to give ethyl 2,4-dimethyl-4H-thieno[3,2-b]pyrrole-5-carboxylate (420 mg). LC-MS (ESI): m/z 224 (M+H)⁺.

Step B. Synthesis of ethyl 6-formyl-2,4-dimethyl-4H-thieno[3,2-b]pyrrole-5-carboxylate To a mixture of ethyl 2,4-dimethyl-4H-thieno[3,2-b]pyrrole-5-carboxylate (300 mg, 1.3 mmol) in anhy. DMF (15 mL) was added POCl₃ (618 mg, 4.0 mmol) at 0° C. The mixture was stirred at 90° C. overnight. The mixture was cooled to r.t. and poured into ice water and neutralized with ammonia, extracted with EtOAc. The combined organic layer was dried over anhy. Na₂SO₄ and concentrated. The residue was purified by silica gel chromatography to give ethyl 6-formyl-2,4-dimethyl-4H-thieno[3,2-b]pyrrole-5-carboxylate (250 mg, 74% yield) as a yellow solid. LC-MS (ESI): m/z 252 (M+H)⁺.

Step C. Synthesis of ethyl 6-(((3-methoxybenzyl)amino)methyl)-2,4-dimethyl-4H-thieno[3,2-b]pyrrole-5-carboxylate A mixture of ethyl 6-formyl-2,4-dimethyl-4H-thieno[3,2-b]pyrrole-5-carboxylate (150 mg, 0.6 mmol) and (3-methoxyphenyl)methanamine (98 mg, 0.7 mmol) in toluene (20 mL) was stirred at 60° C. for 2 hr. Then NaBH(OAc)₃ (380 mg, 1.8 mmol) was added at 0° C., the mixture was stirred at r.t. overnight. The mixture was poured into water, extracted with EtOAc. The organic layer was dried over anhy. Na₂SO₄ and concentrated. The residue was purified by silica gel chromatography to give ethyl 6-(((3-methoxybenzyl)amino)methyl)-2,4-dimethyl-4H-thieno[3,2-b]pyrrole-5-carboxylate (80 mg). LC-MS (ESI): m/z 373 (M+H)⁺.

Step D. Synthesis of 6-(((3-methoxybenzyl)amino)methyl)-2,4-dimethyl-4H-thieno[3,2-b]pyrrole-5-carboxylic acid To a mixture of 6-(((3-methoxybenzyl)amino)methyl)-2,4-dimethyl-4H-thieno[3,2-b]pyrrole-5-carboxylate (50 mg, 0.13 mmol) in MeOH (5 mL) and H₂O (5 mL) was added NaOH (16 mg, 0.4 mmol). The mixture was stirred at 30° C. overnight, and acidified to pH=3 with aq. HCl, extracted with DCM. The organic layer was dried over anhy. Na₂SO₄ and concentrated to give 6-(((3-methoxybenzyl)amino)methyl)-2,4-dimethyl-4H-thieno[3,2-b]pyrrole-5-carboxylic acid (50 mg), which was used directly in the next step. LC-MS (ESI): m/z 345 (M+H)⁺.

Step E. Synthesis of 6-(3-methoxybenzyl)-2,4-dimethyl-6,7-dihydropyrrolo[3,4-b]thieno[2,3-d]pyrrol-5 (4H)-one To a mixture of 6-(((3-methoxybenzyl)amino)methyl)-2,4-dimethyl-4H-thieno[3,2-b]pyrrole-5-carboxylic acid (50 mg, 0.15 mmol) in DCM (10 mL) was added DMAP (35 mg, 0.3 mmol) and EDCI (55 mg, 0.3 mmol). After stirred at 30° C. overnight, the reaction mixture was poured into water and extracted with EtOAc. The organic layer was dried over anhy. Na₂SO₄ and concentrated. The residue was purified by prep-TLC (PE:EtOAc=10:1) to give 6-(3-methoxybenzyl)-2,4-dimethyl-6,7-dihydropyrrolo[3,4-b]thieno[2,3-d]pyrrol-5 (4H)-one (16 mg). LC-MS (ESI): m/z 327 (M+H)⁺. ¹H NMR (400 MHz, DMSO-d₆) δ 7.25 (t, 1H), 6.98 (s, 1H), 6.79-6.86 (m, 3H), 4.59 (s, 2H), 4.18 (s, 2H), 3.86 (s, 3H), 3.73 (s, 3H), 2.51 (s, 3H).

### Example 2: Synthesis of 6-(3-methoxybenzyl)-2,4-dimethyl-4H-thieno[3,2-b]indole

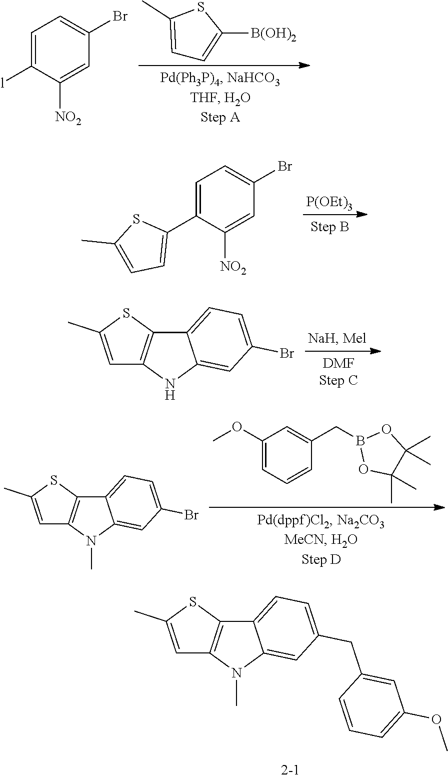

Step A. Synthesis of 2-(4-bromo-2-nitrophenyl)-5-methylthiophene To a mixture of 4-bromo-1-iodo-2-nitrobenzene (400 mg, 1.2 mmol) and 5-methylthiophen-2-ylboronic acid (278 mg, 1.9 mmol) in THF (8 mL) and water (2 mL) was added NaHCO₃ (257 mg, 3.0 mmol) and Pd(Ph₃P)₄ (140 mg, 0.12 mmol). The reaction mixture was stirred at 90° C. for 1 hr under nitrogen atmosphere. The mixture was cooled to r.t., diluted with water, extracted with EtOAc. The organic layer was dried over anhy. Na₂SO₄ and concentrated. The residue was purified by silica gel chromatography (eluted with PE:EtOAc=100:1) to give 2-(4-bromo-2-nitrophenyl)-5-methylthiophene (300 mg).

Step B. Synthesis of 6-bromo-2-methyl-4H-thieno[3,2-b]indole A mixture of 2-(4-bromo-2-nitrophenyl)-5-methylthiophene (300 mg, 1 mmol) in triethyl phosphate (2 mL) was stirred at 170° C. for 2 hr. The solvent was removed under reduce pressure and the residue was purified by silica gel chromatography (eluted with PE:EtOAc=10:1) to give 6-bromo-2-methyl-4H-thieno[3,2-b]indole (260 mg). LC-MS (ESI): m/z 266 (M+H)⁺.

Step C. Synthesis of 6-bromo-2,4-dimethyl-4H-thieno[3,2-b]indole To a solution of 6-bromo-2-methyl-4H-thieno[3,2-b]indole (260 mg, 1.0 mmol) in DMF (5 mL) was added NaH (80 mg, 2.0 mmol) at 0° C. The mixture was stirred at 0° C. for 15 min and then MeI (180 mg, 1.3 mmol) was added. The mixture was stirred at r.t. for another 2 hr. The mixture was poured into satd. NH₄Cl, extracted with EtOAc. The combined organic layer was dried over anhy. Na₂SO₄ and concentrated. The residue was purified by silica gel chromatography (eluted with PE:EtOAc=10:1) to give 6-bromo-2,4-dimethyl-4H-thieno[3,2-b]indole (160 mg). LC-MS (ESI): m/z 280 (M+H)⁺.

Step D. Synthesis of 6-(3-methoxybenzyl)-2,4-dimethyl-4H-thieno[3,2-b]indole To a mixture of 6-bromo-2,4-dimethyl-4H-thieno[3,2-b]indole (40 mg, 0.14 mmol) and 2-(3-methoxybenzyl)-4,4,5,5-tetramethyl-1,3,2-dioxaborolane (70 mg, 0.28 mmol) in MeCN (8 mL) and water (4 mL) was added Na₂CO₃ (45 mg, 0.42 mmol) and Pd(dppf)₂Cl₂ (11 mg, 0.014 mmol). The reaction mixture was stirred at 90° C. for 1 hr under nitrogen atmosphere. The mixture was cooled to r.t., diluted with water, extracted with EtOAc. The organic layer was dried over anhy. Na₂SO₄ and concentrated. The residue was purified by silica gel chromatography (eluted with PE:EtOAc=10:1) to give 6-(3-methoxybenzyl)-2,4-dimethyl-4H-thieno[3,2-b]indole (25 mg). LC-MS (ESI): m/z 322 (M+H)⁺. ¹H NMR (400 MHz, CDCl₃) δ 7.48 (d, 1H), 7.12 (t, 1H), 7.07 (s, 1H), 6.93 (dd, 1H), 6.76 (d, 1H), 6.72-6.64 (m, 3H), 4.04 (s, 2H), 3.70 (s, 3H), 3.69 (s, 3H), 2.56 (d, 3H).

### Example 3: Synthesis of 6-(3-methoxybenzyl)-2,4-dimethyl-4,6-dihydro-5H-oxazolo[5′,4′:4,5]pyrrolo[2,3-d]pyridazin-5-one

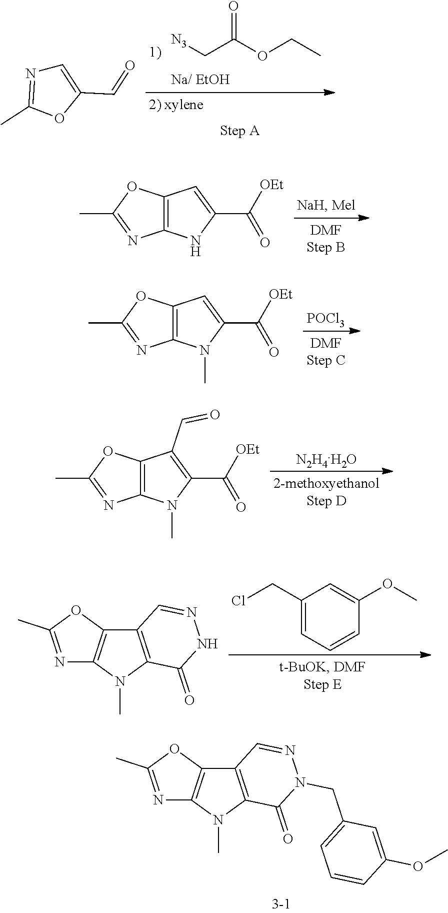

Step A. Synthesis of ethyl 2-methyl-4H-pyrrolo[2,3-d]oxazole-5-carboxylate To a solution of Na (0.65 g, 27 mmol) in dry EtOH (10 mL) was added a mixture of 2-methyloxazole-5-carbaldehyde (1.0 g, 9.0 mmol) and ethyl 2-azidoacetate (3.4 g, 27 mmol) at −10° C. over 1 hr. The reaction mixture was stirred at 5° C. for another 1 hr and quenched with satd. NH₄Cl, extracted with EtOAc. The combined organic layer was dried over anhy. Na₂SO₄ and concentrated to give ethyl 2-azido-3-(2-methyloxazol-5-yl)acrylate (1.1 g crude). A solution of ethyl 2-azido-3-(2-methyloxazol-5-yl)acrylate (1.1 g) in xylene (30 mL) was stirred at 160° C. for 30 min and concentrated under reduced pressure. The residue was purified by silica gel chromatography to give ethyl 2-methyl-4H-pyrrolo[2,3-d]oxazole-5-carboxylate (0.25 g). LC-MS (ESI): m/z 195 (M+H)⁺.

Step B. Synthesis of ethyl 2,4-dimethyl-4H-pyrrolo[2,3-d]oxazole-5-carboxylate To a solution of ethyl ethyl 2-methyl-4H-pyrrolo[2,3-d]oxazole-5-carboxylate (0.25 g, 1.3 mmol) in DMF (10 mL) was added NaH (104 mg, 2.6 mmol) at 0° C. The mixture was stirred at 0° C. for 15 min, MeI (0.23 g, 1.7 mmol) was added, and stirred at r.t. for 2 hr. The reaction mixture was poured into satd. NH₄Cl, extracted with EtOAc. The combined organic layer was dried over anhy. Na₂SO₄ and concentrated. The residue was purified by silica gel chromatography to give ethyl 2,4-dimethyl-4H-pyrrolo[2,3-d]oxazole-5-carboxylate (0.2 g). LC-MS (ESI): m/z 209 (M+H)⁺.

Step C. Synthesis of ethyl 6-formyl-2,4-dimethyl-4H-pyrrolo[2,3-d]oxazole-5-carboxylate A solution of ethyl 2,4-dimethyl-4H-pyrrolo[2,3-d]oxazole-5-carboxylate (0.2 g, 1.0 mmol) and POCl₃ (0.3 g, 2.0 mmol) in DMF (10 mL) was stirred at 100° C. overnight. The reaction mixture was poured into satd. NaHCO₃, extracted with EtOAc. The combined organic layer was dried over anhy. Na₂SO₄ and concentrated. The residue was purified by silica gel chromatography to give ethyl 6-formyl-2,4-dimethyl-4H-pyrrolo[2,3-d]oxazole-5-carboxylate (100 mg). LC-MS (ESI): m/z 237 (M+H)⁺.

Step D. Synthesis of 2,4-Dimethyl-4H-oxazole[5′,4′:4,5]pyrrolo[2,3-d]pyridazin-5 (6H)-one To a solution of ethyl 6-formyl-2,4-dimethyl-4H-pyrrolo[2,3-d]oxazole-5-carboxylate (100 mg, 0.42 mmol) in 2-methoxyethanol (15 mL) was added N₂H4′H₂O (100 mg, 2.0 mmol). The solution was stirred at 100° C. overnight and concentrated under reduced pressure. The residue was purified by prep-TLC to give 2,4-Dimethyl-4H-oxazole[5′,4′:4,5]pyrrolo[2,3-d]pyridazin-5 (6H)-one (50 mg). LC-MS (ESI): m/z 205 (M+H)⁺.

Step E. Synthesis of 6-(3-Methoxybenzyl)-2,4-dimethyl-4H-oxazole[5′,4′:4,5]pyrrolo[2,3-d]pyridazin-5 (6H)-one To a solution of 2,4-dimethyl-4H-oxazole[5′,4′:4,5]pyrrolo[2,3-d]pyridazin-5 (6H)-one (50 mg, 0.23 mmol) in DMF (5 mL) was added t-BuOK (40 mg, 0.34 mmol) under N₂ at 0° C. The mixture was stirred at 0° C. for 20 min, followed by the addition of 1-(chloromethyl)-3-methoxybenzene (40 mg, 0.3 mmol), and stirred at r.t. for another 2 hr. The mixture was poured into satd. NH₄Cl, extracted with EtOAc. The combined organic layer was dried over anhy. Na₂SO₄ and concentrated. The residue was purified by prep-HPLC to give 6-(3-Methoxybenzyl)-2,4-dimethyl-4H-oxazole[5′,4′:4,5]pyrrolo[2,3-d]pyridazin-5 (6H)-one (2 mg). LC-MS (ESI): m/z 325 (M+H)⁺. ¹H NMR (400 MHz, CDCl₃) δ 8.14 (s, 1H), 7.14-7.19 (m, 1H), 6.91-6.95 (m, 1H), 6.89 (s, 1H), 6.73 (dd, 1H), 5.33 (s, 2H), 4.20 (s, 3H), 3.71 (s, 3H), 2.61 (s, 3H).

### Example 4: Synthesis of 2-benzyl-6-(3-methoxybenzyl)-4-methyl-4H-thieno[2′,3′:4,5]pyrrolo[2,3-d]pyridazin-5 (6H)-one

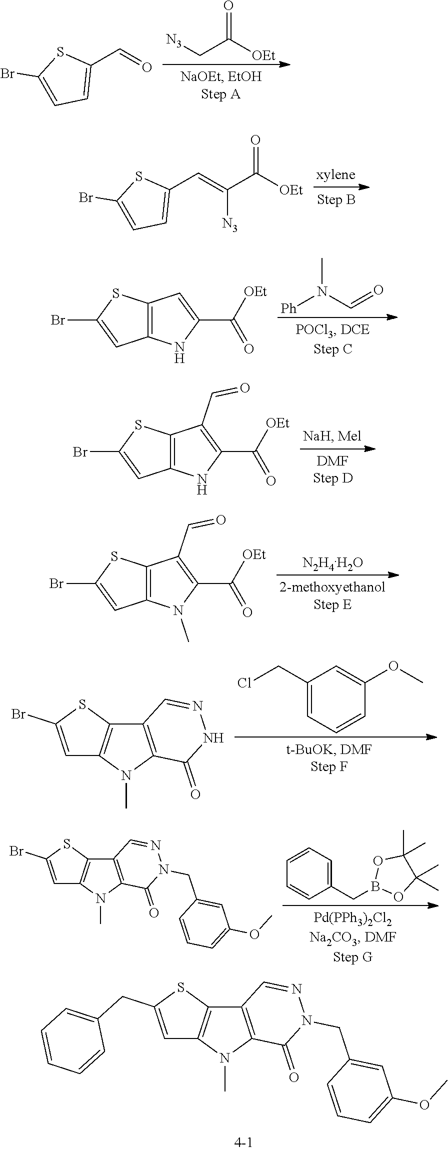

Step A. Synthesis of (Z)-ethyl 2-azido-3-(5-bromothiophen-2-yl)acrylate To a solution of NaOEt (4.3 g, 63.2 mmol) in EtOH (50 mL) was added a mixture of 5-bromothiophene-2-carbaldehyde (4 g, 10.8 mmol) and ethyl azidoacetate (5 g, 32.3 mmol) by dropwise at −10° C. After stirred at 0° C. for 1.5 hr, the mixture was poured into satd. NH₄Cl, extracted with EtOAc. The combined organic layer was dried over anhy. Na₂SO₄ and concentrated to give (Z)-ethyl 2-azido-3-(5-bromothiophen-2-yl)acrylate (4 g, crude). LC-MS (ESI): m/z 302 (M+H)⁺.

Step B. Synthesis of ethyl 2-bromo-4H-thieno[3,2-b]pyrrole-5-carboxylate A mixture of (Z)-ethyl 2-azido-3-(5-bromothiophen-2-yl)acrylate (4 g, crude) in xylene (20 mL) was stirred at 160° C. for 10 mins. The mixture was concentrated under reduced pressure and the residue was purified by silica gel chromatography to give 720 mg of ethyl 2-bromo-4H-thieno[3,2-b]pyrrole-5-carboxylate. LC-MS (ESI): m/z 274 (M+H)⁺.

Step C. Synthesis of ethyl 2-bromo-6-formyl-4H-thieno[3,2-b]pyrrole-5-carboxylate To a mixture of ethyl 2-bromo-4H-thieno[3,2-b]pyrrole-5-carboxylate (720 mg, 2.6 mmol) in 1,2-dichloroethane (DCE) (20 mL) was added N-methyl-N-phenylformamide (530 mg, 3.9 mmol) and POCl₃ (600 mg, 3.9 mmol) at 0° C. The mixture was stirred at 85° C. overnight. The reaction mixture was poured into water, extracted with EtOAc. The combined organic layer was dried over anhy. Na₂SO₄ and concentrated. The residue was purified by silica gel chromatography to give ethyl 2-bromo-6-formyl-4H-thieno[3,2-b]pyrrole-5-carboxylate (730 mg). LC-MS (ESI): m/z 302 (M+H)⁺.

Step D. Synthesis of ethyl 2-bromo-6-formyl-4-methyl-4H-thieno[3,2-b]pyrrole-5-carboxylate To a mixture of ethyl 2-bromo-6-formyl-4H-thieno[3,2-b]pyrrole-5-carboxylate (730 mg, 2.4 mmol) in DMF (20 mL) was added NaH (192 mg, 4.8 mmol) at 0° C. After stirred at r.t. for 30 min, MeI (567 mg, 4 mmol) was added at 0° C. The mixture was stirred at r.t. for 2 hr. The mixture was poured into satd. NH₄Cl, extracted with EtOAc. The combined organic layer was dried over anhy. Na₂SO₄ and concentrated. The residue was purified by silica gel chromatography to give 550 mg of ethyl 2-bromo-6-formyl-4-methyl-4H-thieno[3,2-b]pyrrole-5-carboxylate. LC-MS (ESI): m/z 316 (M+H)⁺.

Step E. Synthesis of 2-bromo-4-methyl-4H-thieno[2′,3′:4,5]pyrrolo[2,3-d]pyridazin-5 (6H)-one To a mixture of ethyl 2-bromo-6-formyl-4-methyl-4H-thieno[3,2-b]pyrrole-5-carboxylate (550 mg, 1.7 mmol) in 2-methoxyethanol (20 mL) was added hydrazine hydrate (2 mL, 98% w/w). The mixture was stirred at 110° C. for 2 hr and cooled down. The precipitate was collected by filtration and washed with water to give 400 mg of 2-bromo-4-methyl-4H-thieno[2′,3′:4,5]pyrrolo[2,3-d]pyridazin-5 (6H)-one. LC-MS (ESI): m/z 284 (M+H)⁺.

Step F. Synthesis of 2-bromo-6-(3-methoxybenzyl)-4-methyl-4H-thieno[2′,3′: 4,5]pyrrolo[2,3-d]pyridazin-5 (6H)-one To a mixture of 2-bromo-4-methyl-4H-thieno[2′,3′:4,5]pyrrolo[2,3-d]pyridazin-5 (6H)-one (200 mg, 0.7 mmol) in DMF (15 mL) was added t-BuOK (235 mg, 2.1 mmol) at 0° C., followed by 1-(chloromethyl)-3-methoxybenzene (219 mg, 1.4 mmol). The mixture was stirred at r.t. for 2 hr. The mixture was poured into satd. NH₄Cl, extracted with EtOAc. The organic layer was dried over anhy. Na₂SO₄ and concentrated. The residue was purified by silica gel chromatography to give 95 mg of 2-bromo-6-(3-methoxybenzyl)-4-methyl-4H-thieno[2′,3′:4,5]pyrrolo[2,3-d]pyridazin-5 (6H)-one. LC-MS (ESI): m/z 404 (M+H)⁺.

Step G. Synthesis of 2-benzyl-6-(3-methoxybenzyl)-4-methyl-4H-thieno[2′,3′: 4,5]pyrrolo[2,3-d]pyridazin-5 (6H)-one To a mixture of 2-bromo-6-(3-methoxybenzyl)-4-methyl-4H-thieno[2′,3′:4,5]pyrrolo[2,3-d]pyridazin-5 (6H)-one (95 mg, 0.23 mmol) and 2-benzyl-4,4,5,5-tetramethyl-1,3,2-dioxaborolane (103 mg, 0.47 mmol) in DMF (5 mL) was added Na₂CO₃ (75 mg, 0.7 mmol) and Pd(PPh₃)₂Cl₂ (51 mg, 0.07 mmol). The mixture was stirred under N₂ at 80° C. overnight. The mixture was diluted with water, extracted with EtOAc. The organic layer was dried over anhy. Na₂SO₄ and concentrated. The residue was purified by prep-HPLC to give 20 mg of 2-benzyl-6-(3-methoxybenzyl)-4-methyl-4H-thieno[2′,3′:4,5]pyrrolo[2,3-d]pyridazin-5 (6H)-one. LC-MS (ESI): m/z 416 (M+H)⁺. ¹H NMR (400 MHz, CDCl₃) δ 8.10 (s, 1H), 7.21-7.32 (m, 5H), 7.13-7.19 (m, 1H), 6.89-6.94 (m, 1H), 6.87 (s, 1H), 6.70-6.75 (m, 2H), 5.34 (s, 2H), 4.19 (s, 5H), 3.70 (s, 3H).

### Example 5: Synthesis of 4-methyl-6-(3-methylbenzyl)-2-(oxazol-2-ylmethyl)-4H-thieno[2′,3′:4,5]pyrrolo[2,3-d]pyridazin-5 (6H)-one

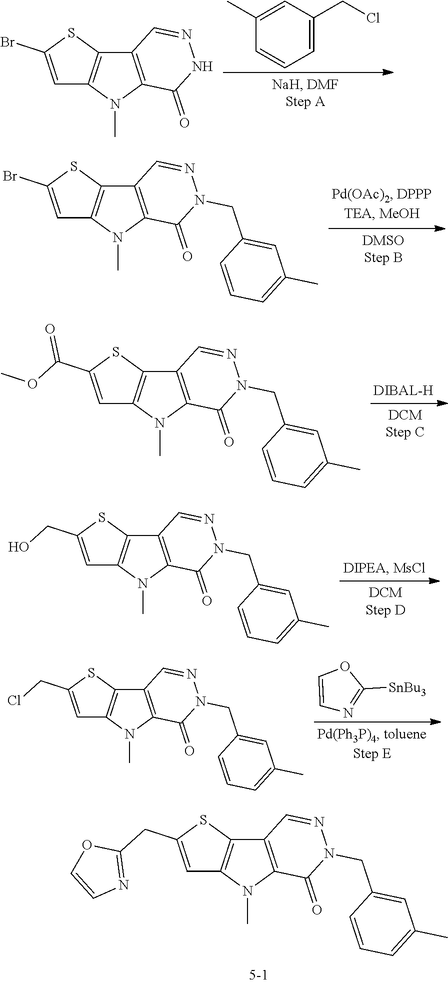

Step A. Synthesis of 2-bromo-4-methyl-6-(3-methylbenzyl)-4H-thieno[2′,3′:4,5]pyrrolo[2,3-d]pyridazin-5 (6H)-one To a mixture of 2-bromo-4-methyl-4H-thieno[2′,3′:4,5]pyrrolo[2,3-d]pyridazin-5 (6H)-one (1 g, 3.5 mmol) in DMF (15 mL) was added NaH (0.28 g, 7.0 mmol) in portions at 0° C. under N₂. After stirred for 30 min, 1-(chloromethyl)-3-methylbenzene (0.7 mL, 5.3 mmol) was added. The mixture was stirred at r.t. for 1 hr. The reaction mixture was quenched with satd. NH₄Cl, and the precipitate was collected by filtration to give 870 mg of 2-bromo-4-methyl-6-(3-methylbenzyl)-4H-thieno[2′,3′:4,5]pyrrolo[2,3-d]pyridazin-5 (6H)-one. LC-MS (ESI): m/z 388 (M+H)⁺.

Step B. Synthesis of methyl 4-methyl-6-(3-methylbenzyl)-5-oxo-5,6-dihydro-4H-thieno[2′,3′:4,5]pyrrolo[2,3-d]pyridazine-2-carboxylate A mixture of 2-bromo-4-methyl-6-(3-methylbenzyl)-4H-thieno[2′,3′:4,5]pyrrolo[2,3-d]pyridazin-5 (6H)-one (870 mg, 2.2 mmol), Pd(OAc)₂ (151 mg, 0.67 mmol), DPPP (1,3-bis(diphenylphosphino)propane 277 mg, 0.67 mmol) and TEA (453 mg, 4.5 mmol) in MeOH (10 mL) and DMSO (10 mL) was stirred at 65° C. under CO for 5 hrs. The reaction mixture was quenched with water, extracted with DCM. The organic layer was washed with brine, dried over anhy. Na₂SO₄ and concentrated. The residue was purified by Flash chromatography (silica gel, 0-100% EtOAc in PE) to give 330 mg of methyl 4-methyl-6-(3-methylbenzyl)-5-oxo-5,6-dihydro-4H-thieno[2′,3′:4,5]pyrrolo[2,3-d]pyridazine-2-carboxylate. LC-MS (ESI): m/z 368 (M+H)⁺.

Step C. Synthesis of 2-(hydroxymethyl)-4-methyl-6-(3-methylbenzyl)-4H-thieno[2′,3′:4,5]pyrrolo[2,3-d]pyridazin-5 (6H)-one A mixture of methyl 4-methyl-6-(3-methylbenzyl)-5-oxo-5,6-dihydro-4H-thieno[2′,3′:4,5]pyrrolo[2,3-d]pyridazine-2-carboxylate (330 mg, 0.9 mmol) in DCM (5 mL) was added DIBAL-H (1.2 mL, 1.8 mmol, 1.5 M in toluene) at 0° C. under N₂. The mixture was stirred at 0° C. for 1 hr. The reaction mixture was quenched with Na₂SO₄.10H₂O, and filtered through a Celite pad. The filtrate was concentrated and the residue was purified by Flash chromatography (silica gel, 0-10% MeOH in DCM) to give 194 mg of 2-(hydroxymethyl)-4-methyl-6-(3-methylbenzyl)-4H-thieno[2′,3′:4,5]pyrrolo[2,3-d]pyridazin-5 (6H)-one. LC-MS (ESI): m/z 340 (M+H)⁺.

Step D. Synthesis of 2-(chloromethyl)-4-methyl-6-(3-methylbenzyl)-4H-thieno[2′,3′: 4,5]pyrrolo[2,3-d]pyridazin-5 (6H)-one To a mixture of 2-(hydroxymethyl)-4-methyl-6-(3-methylbenzyl)-4H-thieno[2′,3′:4,5]pyrrolo[2,3-d]pyridazin-5 (6H)-one (100 mg, 0.3 mmol) and DIPEA (190 mg, 1.5 mmol) in DCM (5 mL) was added MsCl (50 mg, 0.45 mmol) at 0° C. under N₂. The mixture was stirred at r.t. overnight. The reaction mixture was quenched with water, extracted with DCM. The organic layer was washed with brine, dried over anhy. Na₂SO₄ and concentrated. The residue was purified by pre-TLC (PE:EA=1:1) to give 20 mg of 2-(chloromethyl)-4-methyl-6-(3-methylbenzyl)-4H-thieno[2′,3′:4,5]pyrrolo[2,3-d]pyridazin-5 (6H)-one. LC-MS (ESI): m/z 358 (M+H)⁺.

Step E. Synthesis of 4-methyl-6-(3-methylbenzyl)-2-(oxazol-2-ylmethyl)-4H-thieno[2′,3′:4,5]pyrrolo[2,3-d]pyridazin-5 (6H)-one A mixture of 2-(chloromethyl)-4-methyl-6-(3-methylbenzyl)-4H-thieno[2′,3′:4,5]pyrrolo[2,3-d]pyridazin-5 (6H)-one (20 mg, 0.06 mmol), 2-(tributylstannyl)-1,3-oxazole (30 mg, 0.08 mmol) and Pd(Ph₃P)₄ (19 mg, 0.02 mmol) in toluene (3 mL) was stirred at 120° C. under N₂ in microwave for 40 min. The reaction mixture was concentrated and the residue was purified by prep-HPLC to give 2.2 mg of 4-methyl-6-(3-methylbenzyl)-2-(oxazol-2-ylmethyl)-4H-thieno[2′,3′:4,5]pyrrolo[2,3-d]pyridazin-5 (6H)-one. LC-MS (ESI): m/z 391 (M+H)⁺. ¹H NMR (400 MHz, DMSO-d₆) δ 8.51 (s, 1H), 8.08 (s, 1H), 7.36 (s, 1H), 7.23-7.18 (m, 2H), 7.11-7.05 (m, 3H), 5.31 (s, 2H), 4.54 (s, 2H), 4.23 (s, 3H), 2.27 (s, 3H).

| Cpd No. | Structure and chemical name | Characterization |
|---|---|---|
| 5-2 | 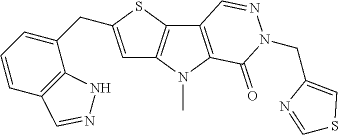 [SMILES: Cn1c2cc(Cc3cccc4cn[nH]c34)sc2c2cnn(Cc3cscn3)c(=O)c21] | LC-MS (ESI): m/z 433 (M + H)⁺. ¹H NMR (400 MHz, DMSO- d₆) δ 13.28 (s, 1H), 9.02 (d, 1H), 8.44 (s, 1H), 8.11 (s, 1H), 7.67 (d, 1H), 7.37 (s, 2H), 7.26 (d, 1H), 7.09 (d, 1H), 5.46 (s, 2H), 4.60 (s, 2H), 4.21 (s, 3H). |
| | 2-((1H-indazol-7-yl)methyl)-4-methyl-6- | |
| | (thiazol-4-ylmethyl)-4H-thieno[2′,3′:4,5] | |
| | pyrrolo[2,3-d]pyridazin-5(6H)-one | |
| 5-3 | 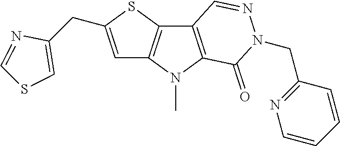 [SMILES: Cn1c2cc(Cc3cscn3)sc2c2cnn(Cc3ccccn3)c(=O)c21] | LC-MS: m/z 394 (M + H)⁺. ¹H NMR (400 MHz, DMSO- d₆) δ 9.09 (d, 1H), 8.48 (d, 2H), 7.72 (td, 1H), 7.56 (d, 1H), 7.29-7.23 (m, 2H), 7.10 (d, 1H), 5.44 (s, 2H), 4.47 (s, 2H), 4.21 (s, 3H) |
| | 4-methyl-6-(pyridin-2-ylmethyl)-2- | |
| | (thiazol-4-ylmethyl)-4H-thieno[2′,3′:4,5] | |
| | pyrrolo[2,3-d]pyridazin-5(6H)-one | |
| 5-4 | 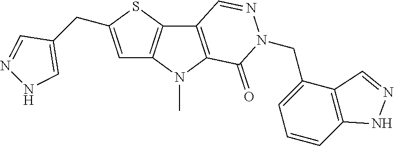 [SMILES: Cn1c2cc(Cc3cn[nH]c3)sc2c2cnn(Cc3cccc4[nH]ncc34)c(=O)c21] | LC-MS: m/z 416 (M + H)⁺. ¹H NMR (400 MHz, DMSO- d₆) δ 13.10 (s, 1H), 12.70 (s, 1H), 8.48 (s, 1H), 8.12 (s, 1H), 7.67 (s, 1H), 7.44 (d, 2H), 7.29-7.24 (m, 1H), 7.21 (s, 1H), 6.94 (d, 1H), 5.64 (s, 2H), 4.21 (s, 3H), 4.14 (s, 2H) |
| | 6-((1H-indazol-4-yl)methyl)-2-((1H- | |
| | pyrazol-4-yl)methyl)-4-methyl-4H- | |
| | thieno[2′,3′:4,5] | |
| | pyrrolo[2,3-d]pyridazin-5(6H)-one | |

### Example 6. Synthesis of 7-(3-methoxybenzyl)-2,5-dimethylthiazolo[3′,2′:1,2]pyrrolo[3,4-d]pyridazin-6 (7H)-one

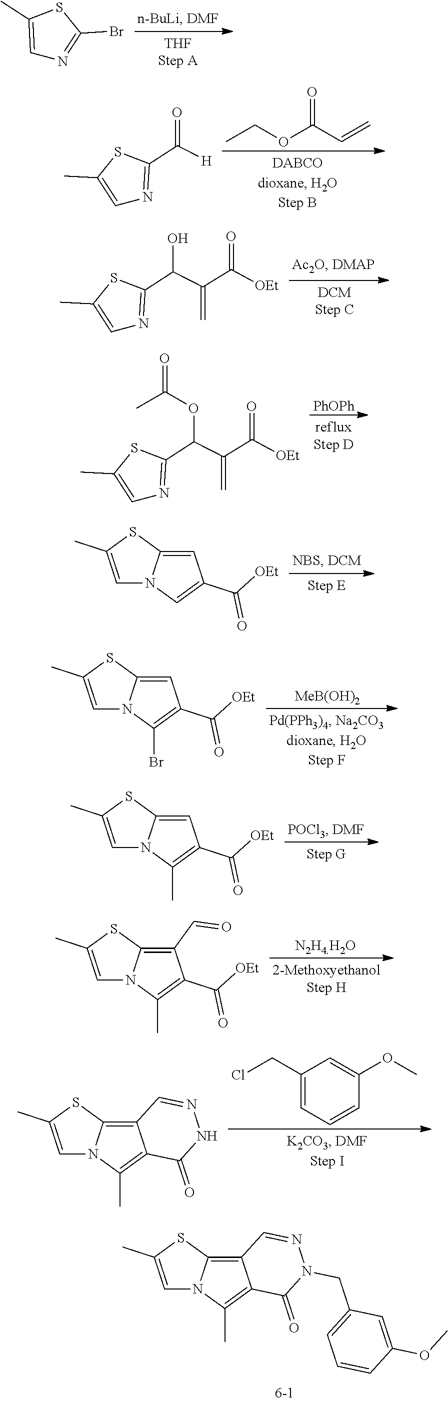

Step A. 5-Methylthiazole-2-carbaldehyde. To a solution of n-BuLi (2.7 mL, 6.74 mmol) in THF (15 mL) under N₂ at −70° C. was added dropwise 2-bromo-5-methylthiazole (1.0 g, 5.56 mmol). The mixture was stirred at that temperature for 1.5 hr, followed dropwise addition of DMF (0.65 mL, 8.42 mmol). The resulting mixture was stirred at that temperature for 1 hr then quenched with aqueous saturated NH₄Cl and extracted with EtOAc The combined organic layers were washed with brine, dried over anhydrous Na₂SO₄ and concentrated under reduced pressure to give the desired product (600 mg) which was directly used in the next step without any purification. LC-MS: m/z 128.0 (M+H)⁺.

Step B. Ethyl 2-(hydroxy(5-methylthiazol-2-yl)methyl)acrylate. To a stirred mixture of 5-methylthiazole-2-carbaldehyde (600 mg, 4.72 mmol) in dioxane and H₂O (V/V=1:1, 20 mL) was added ethyl acrylate (1.89 g, 18.9 mmol) and DABCO (1,4-diazabicyclo[2.2.2]octane 529 mg, 4.72 mmol). The mixture was stirred at r.t. for 30 then quenched with water and extracted with EtOAc. The combined organic layers were washed with brine, dried over anhydrous Na₂SO₄ and concentrated under reduced pressure. The residue was purified by column chromatography on silica gel (eluent: PE/EtOAc=2/1) to give the desired product (900 mg). LC-MS: m/z 228 (M+H)⁺.

Step C. Ethyl 2-(acetoxy(5-methylthiazol-2-yl)methyl)acrylate. To a stirred mixture of ethyl 2-(hydroxy(5-methylthiazol-2-yl)methyl)acrylate (900 mg, 3.96 mmol) in DCM (20 mL) were added Ac₂O (606 mg, 5.94 mmol) and DMAP (96 mg, 0.792 mmol). The mixture was stirred at r.t. for 2 hr then concentrated under reduced pressure. The residue was purified by column chromatography on silica gel (eluent: PE/EtOAc=3/1) to give the desired product (850 mg). LC-MS: m/z 270 (M+H)⁺.

Step D. Ethyl 2-methylpyrrolo[2,1-b]thiazole-6-carboxylate. Ethyl 2-(acetoxy(5-methylthiazol-2-yl)methyl)acrylate (750 mg, 2.79 mmol) was added to a nearly boiled diphenyl oxide (5 mL). The mixture was refluxed for 30 min then cooled down and directly purified by column chromatography on silica gel (eluent: PE/EtOAc=2/1) to give the desired product (420 mg). LC-MS: m/z 210 (M+H)⁺.

Step E. Ethyl 5-bromo-2-methylpyrrolo[2,1-b]thiazole-6-carboxylate. To a stirred mixture of ethyl 2-methylpyrrolo[2,1-b]thiazole-6-carboxylate (320 mg, 1.53 mmol) in DCM (20 mL) at 0° C. was added NBS (269 mg, 1.53 mmol). The mixture was stirred at 0° C. for 30 min then quenched with water and extracted with DCM. The combined organic layers were washed with brine, dried over anhydrous Na₂SO₄ and concentrated under reduced pressure. The residue was purified by column chromatography on silica gel (eluent: PE/EtOAc=5/1) to give the desired product (300 mg). LC-MS: m/z 288 (M+H)⁺.

Step F. Ethyl 2,5-dimethylpyrrolo[2,1-b]thiazole-6-carboxylate. To a stirred mixture of ethyl 5-bromo-2-methylpyrrolo[2,1-b]thiazole-6-carboxylate (300 mg, 1.04 mmol) and methylboronic acid (125 mg, 2.08 mmol) in dioxane (15 mL) were added Pd(PPh₃)₄ (120 mg, 0.1 mmol) and Na₂CO₃ (333 mg, 3.14 mmol). The resulting mixture was stirred at 80° C. under N₂ overnight then filtered through Celite. The filtrate was concentrated under reduced pressure and the residue was purified by column chromatography on silica gel (eluent: PE/EtOAc=3/1) to give the desired product (100 mg). LC-MS: m/z 224 (M+H)⁺.

Step G Ethyl 7-formyl-2,5-dimethylpyrrolo[2,1-b]thiazole-6-carboxylate. To a stirred mixture of ethyl 2,5-dimethylpyrrolo[2,1-b]thiazole-6-carboxylate (65 mg, 0.29 mmol) in DMF (5 mL) was added POCl₃ (221 mg, 1.4 mmol). The resulting mixture was stirred at 100° C. for 3 hr then quenched with water and extracted with EtOAc. The combined organic layers were washed with brine, dried over anhydrous Na₂SO₄ and concentrated under reduced pressure. The residue was purified by column chromatography on silica gel (eluent: PE/EtOAc=3/1) to give the desired product (70 mg). LC-MS: m/z 252 (M+H)⁺.

Step H. 2,5-dimethylthiazolo[3′,2′:1,2]pyrrolo[3,4-d]pyridazin-6 (7H)-one. To a stirred mixture of ethyl 7-formyl-2,5-dimethylpyrrolo[2,1-b]thiazole-6-carboxylate (50 mg, 0.2 mmol) in 2-methoxyethanol (10 mL) was added N₂H4-H₂O (50 mg, 1 mmol). The mixture was stirred at 100° C. under N₂ for 16 hr then quenched with ice-water and extracted with DCM. The combined organic layers were washed with brine, dried over anhydrous Na₂SO₄ and concentrated under reduced pressure. The residue was purified by column chromatography on silica gel (eluent: PE/EtOAc=1/1) to give the desired product (30 mg). LC-MS: m/z 220 (M+H)⁺.

Step I. 7-(3-Methoxybenzyl)-2,5-dimethylthiazolo[3′,2′:1,2]pyrrolo[3,4-d]pyridazin-6 (7H)-one. To a stirred mixture of 2,5-dimethylthiazolo[3′,2′:1,2]pyrrolo[3,4-d]pyridazin-6 (7H)-one (30 mg, 0.137 mmol) in anhydrous DMF (1 mL) were added K₂CO₃ (38 mg, 0.273 mmol), 1-(chloromethyl)-3-methoxybenzene (43 mg, 0.273 mmol) and TBAB (tetra-n-butylammonium bromide, 2 mg). The reaction mixture was stirred at 100° C. for 40 min under microwave. The resulting mixture was quenched with water and extracted with EtOAc. The organic layer was separated, dried over anhydrous Na₂SO₄ and concentrated under reduced pressure. The residue was purified by prep-HPLC to give the desired product (3.2 mg). LC-MS: m/z 340 (M+H)⁺. ¹H NMR (400 MHz, CDCl₃): δ 7.97 (s, 1H), 7.12-7.16 (m, 2H), 6.87-6.92 (m, 2H), 6.71-6.69 (d, 1H), 5.24 (s, 2H), 3.69 (s, 3H), 2.76 (s, 3H), 2.44 (s, 3H).

### Example 7. Synthesis of 7-(3-methoxybenzyl)-2-methyl-5-(trifluoromethyl)thiazolo[3′,2′:1,2]pyrrolo[3,4-d]pyridazin-6 (7H)-one

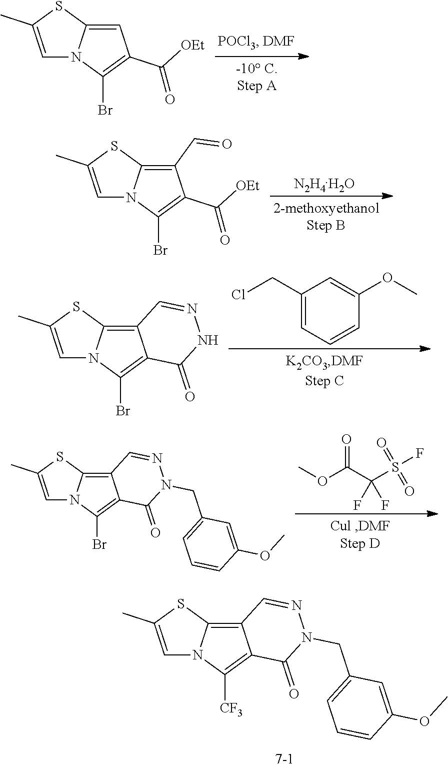

Step A. Synthesis of ethyl 5-bromo-7-formyl-2-methylpyrrolo[2,1-b]thiazole-6-carboxylate To a mixture of ethyl 5-bromo-2-methylpyrrolo[2,1-b]thiazole-6-carboxylate (1.3 g, 4.5 mmol) in DMF (15 mL) was added POCl₃ (2.1 mL, 22.5 mmol) at −10° C. The reaction mixture was stirred at −10° C. for 0.5 hr. The mixture was poured into cooled satd. NaHCO₃, extracted with EtOAc. The organic layer was washed with brine, dried over anhy. Na₂SO₄ and concentrated. The residue was purified by flash chromatography (silica gel, 0-20% EtOAc in PE) to afford ethyl 5-bromo-7-formyl-2-methylpyrrolo[2,1-b]thiazole-6-carboxylate (950 mg). LC-MS (ESI): m/z 316 (M+H)⁺.

Step B. Synthesis of 5-bromo-2-methylthiazolo[3′,2′:1,2]pyrrolo[3,4-d]pyridazin-6 (7H)-one To a stirred mixture of ethyl 5-bromo-7-formyl-2-methylpyrrolo[2,1-b]thiazole-6-carboxylate (600 mg, 1.9 mmol) in 2-methoxyethanol (20 mL) was added hydrazine hydrate (0.45 mL, 9.5 mmol). The reaction mixture was stirred at 100° C. for 18 hr. The reaction mixture was cooled to r.t. and filtered. The filtered cake was washed with EtOH and MBTE, dried under vacuum to afford 5-bromo-2-methylthiazolo[3′,2′:1,2]pyrrolo[3,4-d]pyridazin-6 (7H)-one (390 mg). LC-MS (ESI): m/z 284 (M+H)⁺.

Step C. Synthesis of 5-bromo-7-(3-methoxybenzyl)-2-methylthiazolo[3′,2′:1,2]pyrrolo[3,4-d]pyridazin-6 (7H)-one To a stirred mixture of 5-bromo-2-methylthiazolo[3′,2′:1,2]pyrrolo[3,4-d]pyridazin-6 (7H)-one (370 mg, 1.3 mmol) in anhy. DMF (5 mL) was added K₂CO₃ (359.4 mg, 2.6 mmol) and 1-(chloromethyl)-3-methoxybenzene (0.38 mL, 2.6 mmol). The resulting mixture was stirred at 60° C. for 1 hr. The reaction mixture was quenched with water and extracted with EtOAc. The organic layer was dried over anhy. Na₂SO₄ and concentrated. The residue was purified by flash chromatography (silica gel, 10-30% EtOAc in PE) to afford 500 mg of 5-bromo-7-(3-methoxybenzyl)-2-methylthiazolo[3′,2′:1,2]pyrrolo[3,4-d]pyridazin-6 (7H)-one (LC-MS (ESI): m/z 404 (M+H)⁺. ¹H NMR (400 MHz, DMSO-d₆) δ 8.46 (s, 1H), 7.98 (s, 1H), 7.22 (t, 1H), 6.85-6.80 (m, 3H), 5.17 (s, 2H), 3.71 (s, 3H), 2.52 (d, 3H).

Step D. Synthesis of 7-(3-methoxybenzyl)-2-methyl-5-(trifluoromethyl)thiazolo[3′,2′: 1,2]pyrrolo[3,4-d]pyridazin-6 (7H)-one To a stirred mixture of 5-bromo-7-(3-methoxybenzyl)-2-methylthiazolo[3′,2′:1,2]pyrrolo[3,4-d]pyridazin-6 (7H)-one (50 mg, 0.12 mmol) in anhy. DMF (3 mL) was added methyl 2,2-difluoro-2-(fluorosulfonyl)acetate (238 mg, 1.2 mmol) and Cu (71 mg, 0.37 mmol). The resulting mixture was stirred at 100° C. for 18 hr. The reaction mixture was filtered and concentrated. The residue was purified by prep-HPLC to afford 3 mg of 7-(3-methoxybenzyl)-2-methyl-5-(trifluoromethyl)thiazolo[3′,2′: 1,2]pyrrolo[3,4-d]pyridazin-6 (7H)-one. LC-MS (ESI): m/z 394 (M+H)⁺. ¹H NMR (400 MHz, DMSO-d₆) δ 8.59 (s, 1H), 8.19 (d, 1H), 7.23 (t, 1H), 6.86-6.81 (m, 3H), 5.24 (s, 2H), 3.72 (s, 3H), 2.56 (d, 3H).

| Cpd No. | Structure and chemical name | Characterization |
|---|---|---|
| 7-2 | 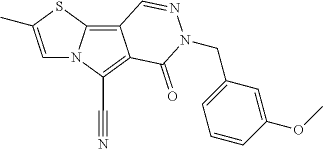 | LC-MS: m/z 351 (M + H)⁺. ¹H NMR (400 MHz, DMSO- d₆) δ 8.65 (s, 1H), 8.34 (d, 1H), 7.23 (t, 1H), 6.87-6.82 (m, 3H), 5.25 (s, 2H), 3.72 (s, 3H), 2.57 (s, 3H) |
| | 7-(3-methoxybenzyl)-2-methyl-6-oxo-6,7- | |
| | dihydrothiazolo[3′,2′:1,2]pyrrolo[3,4- | |
| | d]pyridazine-5-carbonitrile | |
| 7-3 | 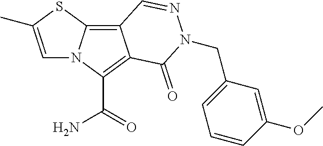 | LC-MS: m/z 369 (M + H)⁺. ¹H NMR (400 MHz, DMSO- d₆) δ 10.03 (s, 1H), 8.96 (s, 1H), 8.71 (s, 1H), 7.59 (s, 1H), 7.24 (t, 1H), 6.89-6.83 (m, 3H), 5.30 (s, 2H), 3.72 (s, 3H), 2.59 (s, 3H) |
| | 7-(3-methoxybenzyl)-2-methyl-6-oxo-6,7- | |
| | dihydrothiazolo[3′,2′:1,2]pylrolo[3,4- | |
| | d]pyridazine-5-carboxamide | |
| 7-4 | 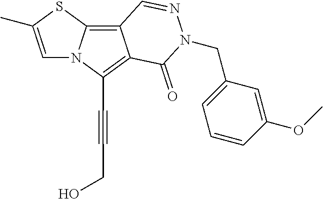 | LC-MS: m/z 380 (M + H)⁺. ¹H NMR (400 MHz, DMSO- d₆) δ 8.47 (s, 1H), 8.04 (s, 1H), 7.22 (t, 1H), 6.83 (d, 3H), 5.44 (s, 1H), 5.19 (s, 2H), 4.44 (d, 2H), 3.71 (s, 3H), 2.54 (s, 3H) |
| | 5-(3-hydroxyprop-1-yn-1-yl)-7-(3- | |
| | methoxybenzyl)-2-methylthiazolo[3′,2′:1,2] | |
| | pyrrolo[3,4-d]pyridazin-6(7H)-one | |

### Example 8. Synthesis of 2-((1H-pyrazol-3-yl)methyl)-5-methyl-7-((1-methyl-1H-pyrazol-3-yl)methyl)thiazolo[3′,2′:1,2]pyrrolo[3,4-d]pyridazin-6 (7H)-one

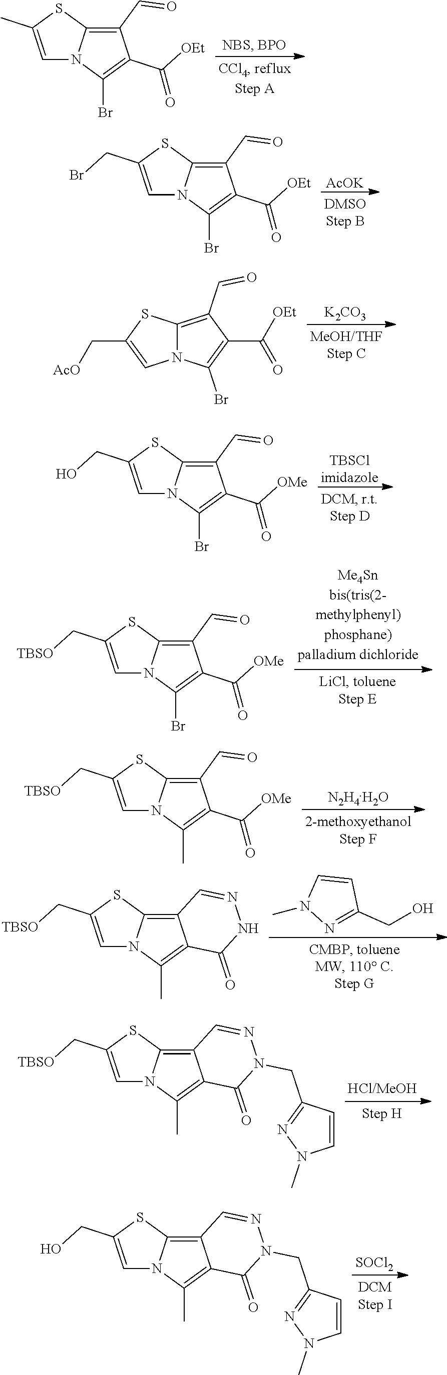

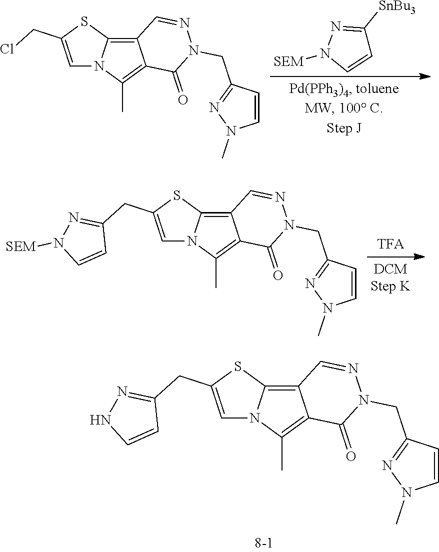

Step A. Synthesis of ethyl 5-bromo-2-(bromomethyl)-7-formylpyrrolo[2,1-b]thiazole-6-carboxylate To a stirred mixture of ethyl 5-bromo-7-formyl-2-methylpyrrolo[2,1-b]thiazole-6-carboxylate (2.7 g, 8.5 mmol) in CCl₄ (60 mL) was added NBS (2.28 g, 12.8 mmol) and BPO (benzoyl peroxide 0.2 g, 0.83 mmol). The reaction mixture was refluxed under N₂ for 2 hr and then cooled down. The reaction mixture was concentrated and the residue was used in the next step without further purification.

Step B. Synthesis of ethyl 2-(acetoxymethyl)-5-bromo-7-formylpyrrolo[2,1-b]thiazole-6-carboxylate To a stirred mixture of ethyl 5-bromo-2-(bromomethyl)-7-formylpyrrolo[2,1-b]thiazole-6-carboxylate in DMSO (50 mL) was added AcOK (2.5 g, 25.6 mmol). The reaction mixture was stirred at 50° C. for 0.5 hr. The reaction mixture was diluted with water, extracted with EtOAc. The organic phase was washed with brine, dried over anhy. Na₂SO₄ and concentrated. The residue was purified by flash chromatography (silica gel, 17% EtOAc and 12% DCM in PE to 25% EtOAc and 12% DCM in PE) to afford ethyl 2-(acetoxymethyl)-5-bromo-7-formylpyrrolo[2,1-b]thiazole-6-carboxylate (1.2 g). LC-MS (ESI): m/z 374 (M+H)⁺.

Step C. Synthesis of methyl 5-bromo-7-formyl-2-(hydroxymethyl)pyrrolo[2,1-b]thiazole-6-carboxylate To a stirred mixture of ethyl 2-[(acetyloxy)methyl]-5-bromo-7-formylpyrrolo[2,1-b]thiazole-6-carboxylate (1 g, 2.7 mmol) in MeOH (10 mL) and THF (10 mL) was added K₂CO₃ (1.1 g, 8.0 mmol). The reaction mixture was stirred at r.t. for 30 min. The reaction mixture was diluted with DCM and filtered through a pad of celite. The filtrate was concentrated to give methyl 5-bromo-7-formyl-2-(hydroxymethyl)pyrrolo[2,1-b]thiazole-6-carboxylate (800 mg). LC-MS (ESI): m/z 318 (M+H)⁺.

Step D. Synthesis of methyl 5-bromo-2-(((tert-butyldimethylsilyl)oxy)methyl)-7-formylpyrrolo[2,1-b]thiazole-6-carboxylate To a stirred mixture of methyl 5-bromo-7-formyl-2-(hydroxymethyl)pyrrolo[2,1-b]thiazole-6-carboxylate (800 mg, 2.4 mmol) in DCM (20 mL) was added imidazole (491 mg, 7.2 mmol) and TBSCl (544 mg, 3.6 mmol). The reaction mixture was stirred at r.t. for 1 hr, diluted with water, extracted with DCM. The organic phase was washed with brine, dried over anhy. Na₂SO₄ and concentrated. The residue was purified by flash chromatography (silica gel, 5% EtOAc and 2.5% DCM in PE to 7% EtOAc and 2.5% DCM in PE) to give 220 mg of methyl 5-bromo-2-(((tert-butyldimethylsilyl)oxy)methyl)-7-formylpyrrolo[2,1-b]thiazole-6-carboxylate. LC-MS (ESI): m/z 432 (M+H)⁺.

Step E. Synthesis of methyl 2-(((tert-butyldimethylsilyl)oxy)methyl)-7-formyl-5-methylpyrrolo[2,1-b]thiazole-6-carboxylate To a stirred mixture of methyl 5-bromo-2-(((tert-butyldimethylsilyl)oxy)methyl)-7-formylpyrrolo[2,1-b]thiazole-6-carboxylate (280 mg, 0.63 mmol) in toluene (5 mL) was added LiCl (53 mg, 1.25 mmol), bis(tris(2-methylphenyl)phosphane) palladium dichloride (49 mg, 0.06 mmol) and Me₄Sn (224 mg, 1.25 mmol). The reaction mixture was stirred at 105° C. for 2 hr. The reaction mixture was concentrated. The residue was purified by flash chromatography (silica gel, 10% EtOAc and 5% DCM in PE to 20% EtOAc and 5% DCM in PE) to give methyl 2-(((tert-butyldimethylsilyl)oxy)methyl)-7-formyl-5-methylpyrrolo[2,1-b]thiazole-6-carboxylate (210 mg). LC-MS (ESI): m/z 368 (M+H)⁺.

Step F. Synthesis of 2-(((tert-butyldimethylsilyl)oxy)methyl)-5-methylthiazolo[3′,2′:1,2]pyrrolo[3,4-d]pyridazin-6 (7H)-one To a stirred mixture of methyl 2-(((tert-butyldimethylsilyl)oxy)methyl)-7-formyl-5-methylpyrrolo[2,1-b]thiazole-6-carboxylate (210 mg, 0.55 mmol) in 2-methoxyethanol (10 mL) was added hydrazine hydrate (270 mg, 5.5 mmol). The reaction mixture was stirred at 100° C. overnight. The reaction mixture was diluted with water and filtered. The filter cake was washed with water and dried in vacuum to give 2-(((tert-butyldimethylsilyl)oxy)methyl)-5-methylthiazolo[3′,2′:1,2]pyrrolo[3,4-d]pyridazin-6 (7H)-one (180 mg). LC-MS (ESI): m/z 350 (M+H)⁺.

Step G. Synthesis of 2-(((tert-butyldimethylsilyl)oxy)methyl)-5-methyl-7-((1-methyl-1H-pyrazol-3-yl)methyl)thiazolo[3′,2′:1,2]pyrrolo[3,4-d]pyridazin-6 (7H)-one To a stirred solution of 2-(((tert-butyldimethylsilyl)oxy)methyl)-5-methylthiazolo[3′,2′:1,2]pyrrolo[3,4-d]pyridazin-6 (7H)-one (150 mg, 0.43 mmol) and (1-methyl-1H-pyrazol-3-yl)methanol (48 mg, 0.43 mmol) in toluene (5 mL) was added CMBP (cyanomethylenetributylphosphorane 0.45 mL, 1.3 mmol). The mixture was stirred under N₂ at 120° C. for 40 min on microwave. The mixture was concentrated and purified by flash chromatography (silica gel, 50% EtOAc in PE to 2% MeOH in DCM) and washed with PE to give 2-(((tert-butyldimethylsilyl)oxy)methyl)-5-methyl-7-((1-methyl-1H-pyrazol-3-yl)methyl)thiazolo[3′,2′:1,2]pyrrolo[3,4-d]pyridazin-6 (7H)-one (100 mg). LC-MS (ESI): m/z 444 (M+H)⁺.

Step H. Synthesis of 2-(hydroxymethyl)-5-methyl-7-((1-methyl-1H-pyrazol-3-yl)methyl)thiazolo[3′,2′:1,2]pyrrolo[3,4-d]pyridazin-6 (7H)-one To a stirred solution of 2-(((tert-butyldimethylsilyl)oxy)methyl)-5-methyl-7-((1-methyl-1H-pyrazol-3-yl)methyl)thiazolo[3′,2′:1,2]pyrrolo[3,4-d]pyridazin-6 (7H)-one (100 mg, 0.23 mmol) in MeOH (3 mL) was added HCl/dioxane (3 mL, 4 M). The mixture was stirred at r.t. for 1 hr. The mixture was concentrated and washed with MTBE to afford 2-(hydroxymethyl)-5-methyl-7-((1-methyl-1H-pyrazol-3-yl)methyl)thiazolo[3′,2′:1,2]pyrrolo[3,4-d]pyridazin-6 (7H)-one (74 mg). LC-MS (ESI): m/z 330 (M+H)⁺. ¹H NMR (400 MHz, DMSO-d₆) δ 8.31 (s, 1H), 8.09 (s, 1H), 7.54 (d, 1H), 6.02 (d, 1H), 5.12 (s, 2H), 4.68 (d, 2H), 3.76 (s, 3H), 2.77 (s, 3H).

Step I. Synthesis of 2-(chloromethyl)-5-methyl-7-((1-methyl-1H-pyrazol-3-yl)methyl)thiazolo[3′,2′:1,2]pyrrolo[3,4-d]pyridazin-6 (7H)-one To a stirred solution of 2-(hydroxymethyl)-5-methyl-7-((1-methyl-1H-pyrazol-3-yl)methyl)thiazolo[3′,2′:1,2]pyrrolo[3,4-d]pyridazin-6 (7H)-one (74 mg, 0.33 mmol) in DCM (5 mL) was added thionyl chloride (0.05 mL, 0.67 mmol). The mixture was stirred at r.t. for 1 hr under N₂. The mixture was concentrated in vacuum and the residue was adjusted to pH=7˜8 with satd. NaHCO₃, extracted with DCM/i-PrOH. The organic phase was washed with brine, dried over anhy. Na₂SO₄ and concentrated. The residue was purified by flash chromatography (silica gel, 50% EtOAc in PE) to give 2-(chloromethyl)-5-methyl-7-((1-methyl-1H-pyrazol-3-yl)methyl)thiazolo[3′,2′:1,2]pyrrolo[3,4-d]pyridazin-6 (7H)-one (49 mg). LC-MS (ESI): m/z 348 (M+H)⁺.

Step J. Synthesis of 5-methyl-7-((1-methyl-1H-pyrazol-3-yl)methyl)-2-((1-((2-(trimethylsilyl)ethoxy)methyl)-1H-pyrazol-3-yl)methyl)thiazolo[3′,2′:1,2]pyrrolo[3,4-d]pyridazin-6 (7H)-one To a stirred solution of 2-(chloromethyl)-5-methyl-7-((1-methyl-1H-pyrazol-3-yl)methyl)thiazolo[3′,2′:1,2]pyrrolo[3,4-d]pyridazin-6 (7H)-one (30 mg, 0.09 mmol) and 3-(tributylstannyl)-1-((2-(trimethylsilyl)ethoxy)methyl)-1H-pyrazole (63 mg, 0.13 mmol) in toluene (2 mL) was added Pd(PPh₃)₄ (10 mg, 0.01 mmol). The mixture was stirred at 120° C. for 0.5 hr on microwave. The mixture was concentrated in vacuum and purified by prep-TLC (60% EtOAc in PE) to give 5-methyl-7-((1-methyl-1H-pyrazol-3-yl)methyl)-2-((1-((2-(trimethylsilyl)ethoxy)methyl)-1H-pyrazol-3-yl)methyl)thiazolo[3′,2′:1,2]pyrrolo[3,4-d]pyridazin-6 (7H)-one (15 mg). LC-MS (ESI): m/z 510 (M+H)3.

Step K. Synthesis of 2-((1H-pyrazol-3-yl)methyl)-5-methyl-7-((1-methyl-1H-pyrazol-3-yl)methyl)thiazolo[3′,2′:1,2]pyrrolo[3,4-d]pyridazin-6 (7H)-one A mixture of 5-methyl-7-((1-methyl-1H-pyrazol-3-yl)methyl)-2-((1-((2-(trimethylsilyl)ethoxy)methyl)-1H-pyrazol-3-yl)methyl)thiazolo[3′,2′:1,2]pyrrolo[3,4-d]pyridazin-6 (7H)-one (15 mg, 0.03 mmol) in DCM/TFA (2 mL/2 mL) was stirred at r.t. for 1 hr. The reaction mixture was concentrated. The residue was purified by prep-HPLC to give 4.8 mg of 2-((1H-pyrazol-3-yl)methyl)-5-methyl-7-((1-methyl-1H-pyrazol-3-yl)methyl)thiazolo[3′,2′:1,2]pyrrolo[3,4-d]pyridazin-6 (7H)-one. LC-MS (ESI): m/z 380 (M+H)⁺. ¹H NMR (400 MHz, DMSO-d₆) δ 12.68 (s, 1H), 8.26 (s, 1H), 8.03 (s, 1H), 7.63 (s, 1H), 7.53 (d, 1H), 6.20 (d, 1H), 6.01 (d, 1H), 5.11 (s, 2H), 4.20 (s, 2H), 3.76 (s, 3H), 2.77 (s, 3H).

| Cpd No. | Structure and chemical name | Characterization |
|---|---|---|
| 8-2 | 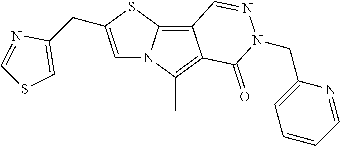 [SMILES: Cc1c2c(=O)n(Cc3ccccn3)ncc2c2sc(Cc3cscn3)cn12] | LC-MS (ESI): m/z 394 (M + H)⁺. ¹H NMR (400 MHz, DMSO- d₆) δ 9.12 (d, 1H), 8.48 (d, 1H), 8.34 (s, 1H), 8.11 (s, 1H), 7.70 (td, 1H), 7.62 (d, 1H), 7.25 (dd, 1H), 7.04 (d, 1H), 5.30 (s, 2H), 4.41 (s, 2H), 2.78 (s, 3H). |
| | 5-methyl-7-(pyridin-2-ylmethyl)-2- | |
| | (thiazol-4-ylmethyl)thiazolo[3′,2′:1,2] | |
| | pyrrolo[3,4-d]pyridazin-6(7H)-one | |
| 8-3 | 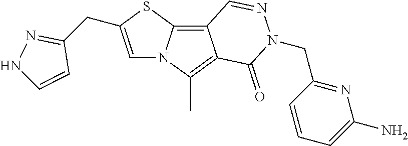 [SMILES: Cc1c2c(=O)n(Cc3cccc(N)n3)ncc2c2sc(Cc3cc[nH]n3)cn12] | LC-MS: m/z 392 (M + H)⁺. ¹H NMR (400 MHz, DMSO- d₆) δ 8.31 (s, 1H), 8.06 (s, 1H), 7.63 (s, 1H), 7.25-7.21 (m, 1H), 6.27 (d, 1H), 6.20 (d, 1H), 6.03 (d, 1H), 5.87 (s, 2H), 5.04 (s, 2H), 4.21 (s, 2H), 2.77 (s, 3H). |
| | 2-((1H-pyrazol-3-yl)methyl)-7-((6-aminopyridin-2- | |
| | yl)methyl)-5-methylthiazolo[3′,2′:1,2]pyrrolo[3,4- | |
| | d]pyridazin-6(7H)-one | |
| 8-4 | 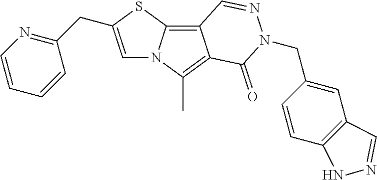 [SMILES: Cc1c2c(=O)n(Cc3ccc4[nH]ncc4c3)ncc2c2sc(Cc3ccccn3)cn12] | LC-MS: m/z 427 M + H)⁺. ¹H NMR (400 MHz, DMSO- d₆) δ 12.99 (s, 1H), 8.55 (d, 1H), 8.31 (s, 1H), 8.11 (s, 1H), 8.01 (s, 1H), 7.79 (td, 1H), 7.62 (s, 1H), 7.45 (t, 2H), 7.36-7.32 (m, 1H), 7.30 (dd, 2H), 5.27 (s, 2H), 4.35 (s, 2H), 2.78 (s, 3H). |
| | 7-((1H-indazol-5-yl)methyl)-5-methyl-2- | |
| | (pyridin-2-ylmethyl)thiazolo[3′,2′:1,2] | |
| | pyrrolo[3,4-d]pyridazin-6(7H)-one | |
| 8-5 | 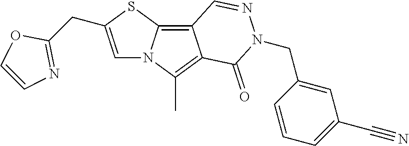 [SMILES: Cc1c2c(=O)n(Cc3cccc(C#N)c3)ncc2c2sc(Cc3ncco3)cn12] | LC-MS: m/z 402 (M + H)⁺. ¹H NMR (400 MHz, DMSO- d₆) δ 8.40 (s, 1H), 8.20 (s, 1H), 8.11 (s, 1H), 7.74 (d, 1H), 7.70 (s, 1H), 7.60 (d, 1H), 7.54 (t, 1H), 7.22 (s, 1H), 5.26 (s, 2H), 4.51 (s, 2H), 2.79 (s, 3H). |
| | 3-((5-methyl-2-(oxazol-2-ylmethyl)-6- | |
| | oxothiazolo[3′,2′:1,2]pyrrolo[3,4- | |
| | d]pyridazin-7(6H)-yl)methyl)benzonitrile | |
| 8-6 | 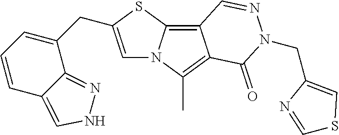 [SMILES: Cc1c2c(=O)n(Cc3cscn3)ncc2c2sc(Cc3cccc4c[nH]nc34)cn12] | LC-MS (ESI): m/z 433 (M + H)⁺. ¹H NMR (400 MHz, DMSO-d₆) δ 13.26 (s, 1H), 9.01 (d, 1H), 8.28 (s, 1H), 8.18 (s, 1H), 8.13 (s, 1H), 7.70 (d, 1H), 7.33 (d, 1H), 7.29 (s, 1H), 7.16-7.07 (m, 1H), 5.32 (s, 2H), 4.53 (s, 2H), 2.80 (s, 3H). |
| | 2-((2H-indazol-7-yl)methyl)-5-methyl-7- | |
| | (thiazol-4-ylmethyl)thiazolo[3′,2′:1,2] | |
| | pyrrolo[3,4-d]pyridazin-6(7H)-one | |

### Example 9. Synthesis of 2,5-dimethyl-7-((1-methyl-1H-pyrazol-3-yl)methyl) thiazolo[3′,2′:1,2]pyrrolo[3,4-d]pyridazin-6 (7H)-one

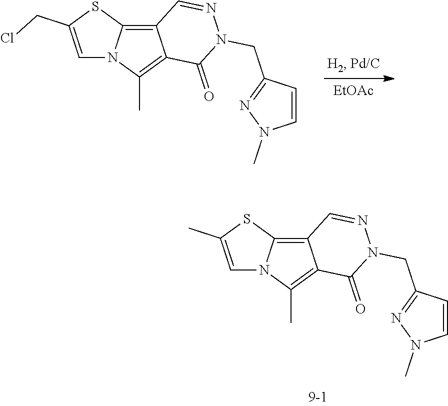 [SMILES: "EtOAc" ; Cc1c2c(=O)n(Cc3ccn(C)n3)ncc2c2sc(CCl)cn12 ; Cc1cn2c(C)c3c(=O)n(Cc4ccn(C)n4)ncc3c2s1]

### Synthesis of 2,5-dimethyl-7-((1-methyl-1H-pyrazol-3-yl)methyl)thiazolo[3′,2′: 1,2]pyrrolo[3,4-d]pyridazin-6 (7H)-one

To a mixture of 2-(chloromethyl)-5-methyl-7-((1-methyl-1H-pyrazol-3-yl)methyl)thiazolo[3′,2′:1,2]pyrrolo[3,4-d]pyridazin-6 (7H)-one (30 mg, 0.09 mmol) in EtOAc (5 mL) was added Pd/C (30 mg, 10% wt). The reaction mixture was stirred under H₂ at r.t. for 1 hr. The mixture was filtered through a celite pad and the filtrate was concentrated under reduced pressure. The residue was purified by prep-HPLC to give 2,5-dimethyl-7-((1-methyl-1H-pyrazol-3-yl)methyl)thiazolo[3′,2′:1,2]pyrrolo[3,4-d]pyridazin-6 (7H)-one (1.4 mg). LC-MS: m/z 314 (M+H)⁺. ¹H NMR (400 MHz, DMSO-d₆) δ 8.29 (s, 1H), 7.94 (s, 1H), 7.54 (d, 1H), 6.02 (d, 1H), 5.12 (s, 2H), 3.77 (s, 3H), 2.76 (s, 3H), 2.49 (s, 3H)

| Cpd No. | Structure and chemical name | Characterization |
|---|---|---|
| 9-2 | 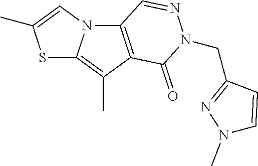 [SMILES: Cc1cn2c(s1)c(C)c1c(=O)n(Cc3ccn(C)n3)ncc12] | LC-MS: m/z 314 (M + H)⁺. ¹H NMR (400 MHz, DMSO- d₆) δ 8.54 (s, 1H), 8.13 (d, 1H), 7.54 (d, 1H), 6.03 (d, 1H), 5.22 (s, 2H), 3.76 (s, 3H), 2.44 (s, 2H), 2.43 (d, 3H). |
| | 2,9-dimethyl-7-((1-methyl-1H-pyrazol-3- | |
| | yl)methyl)thiazolo[3′,2′:1,5]pyrrolo[2,3- | |
| | d]pyridazin-8(7H)-one | |

### Example 10. Synthesis of 2-((1H-pyrazol-3-yl)methyl)-9-methyl-7-((1-methyl-1H-pyrazol-3-yl)methyl)thiazolo[3′,2′:1,5]pyrrolo[2,3-d]pyridazin-8 (7H)-one

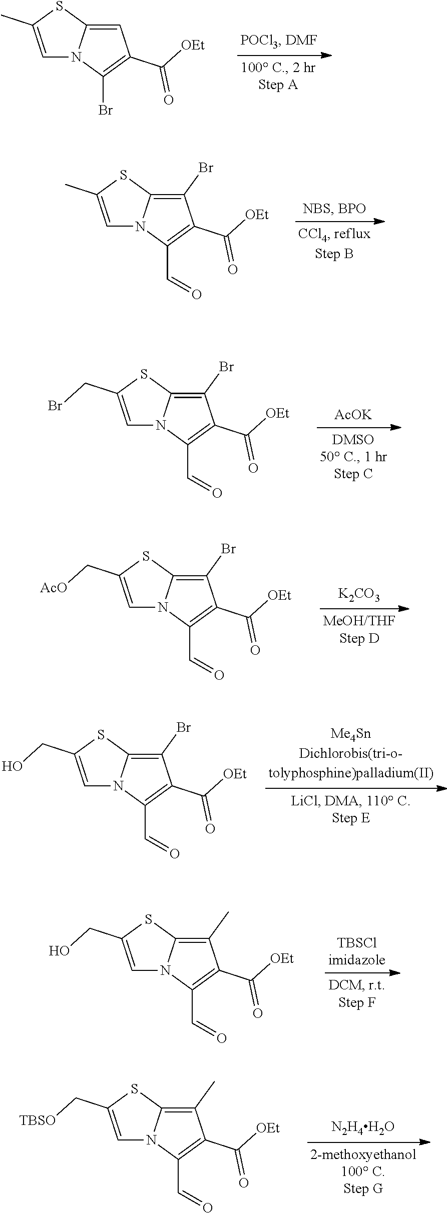

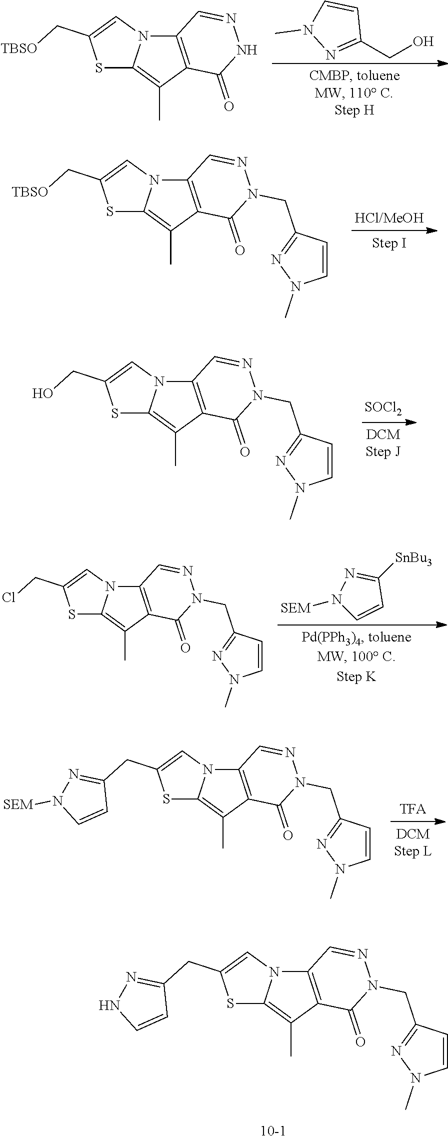

Step A. Synthesis of ethyl 7-bromo-5-formyl-2-methylpyrrolo[2,1-b]thiazole-6-carboxylate To a stirred mixture of ethyl 5-bromo-2-methylpyrrolo[2,1-b][1,3]thiazole-6-carboxylate (5.0 g, 17 mmol) in DMF (50 mL) was added POCl₃ (8.1 mL, 86 mmol) at r.t. The reaction mixture was stirred at 100° C. for 2 hr and then cooled to r.t. The reaction mixture was poured into ice-water, extracted with EtOAc. The organic phase was washed with brine, dried over anhy. Na₂SO₄ and concentrated. The residue was purified by flash chromatography (silica gel, 10% EtOAc and 10% DCM in PE) to give 1.8 g of ethyl 7-bromo-5-formyl-2-methylpyrrolo[2,1-b]thiazole-6-carboxylate. LC-MS (ESI): m/z 316 (M+H)⁺.

Step B. Synthesis of ethyl 7-bromo-2-(bromomethyl)-5-formylpyrrolo[2,1-b]thiazole-6-carboxylate To a stirred mixture of ethyl 7-bromo-5-formyl-2-methylpyrrolo[2,1-b]thiazole-6-carboxylate (400 mg, 1.3 mmol) in CCl₄ (40 mL) was added NBS (270 mg, 1.5 mmol) and BPO (30 mg, 0.13 mmol). The reaction mixture was refluxed under N₂ for 2 hr and then cooled down. The reaction mixture was concentrated and the residue was used in the next step without further purification.

Step C. Synthesis of ethyl 2-(acetoxymethyl)-7-bromo-5-formylpyrrolo[2,1-b]thiazole-6-carboxylate To a stirred mixture of ethyl 7-bromo-2-(bromomethyl)-5-formylpyrrolo[2,1-b]thiazole-6-carboxylate in DMSO (15 mL) was added AcOK (621 mg, 6.3 mmol). The reaction mixture was stirred at 50° C. for 1 hr. The reaction mixture was diluted with water, extracted with EtOAc. The organic phase was washed with brine, dried over anhy. Na₂SO₄ and concentrated. The residue was purified by flash chromatography (silica gel, 8˜ 16% EtOAc in PE) to give 120 mg of ethyl 2-(acetoxymethyl)-7-bromo-5-formylpyrrolo[2,1-b]thiazole-6-carboxylate. LC-MS (ESI): m/z 374 (M+H)⁺.

Step D. Synthesis of ethyl 7-bromo-5-formyl-2-(hydroxymethyl)pyrrolo[2,1-b]thiazole-6-carboxylate To a stirred mixture of ethyl 2-[(acetyloxy)methyl]-5-bromo-7-formylpyrrolo[2,1-b][1,3]thiazole-6-carboxylate (650 mg, 1.7 mmol) in MeOH (10 mL) and THF (20 mL) was added K₂CO₃ (719 mg, 5.2 mmol). The reaction mixture was stirred at r.t. for 30 min. The reaction mixture was diluted with water, extracted with DCM. The organic phase was washed with brine, dried over anhy. Na₂SO₄ and concentrated. The residue was purified by flash chromatography (silica gel, 33% EtOAc and 17% DCM in PE) to give 470 mg of ethyl 7-bromo-5-formyl-2-(hydroxymethyl)pyrrolo[2,1-b][1,3]thiazole-6-carboxylate. LC-MS (ESI): m/z 332 (M+H)⁺.

Step E. Synthesis of ethyl 5-formyl-2-(hydroxymethyl)-7-methylpyrrolo[2,1-b]thiazole-6-carboxylate To a stirred mixture of ethyl 7-bromo-5-formyl-2-(hydroxymethyl)pyrrolo[2,1-b][1,3]thiazole-6-carboxylate (470 mg, 1.4 mmol) and LiCl (119 mg, 2.8 mmol) in DMA (10 mL) was added dichlorobis(tri-o-tolylphosphine)palladium(II) (111 mg, 0.14 mmol) and (CH₃)₄Sn (506 mg, 2.8 mmol). The reaction mixture was purged with N₂ and stirred at 110° C. for 2 hr. The cooled reaction mixture was diluted with water, extracted with EtOAc. The organic phase was washed with brine, dried over anhy. Na₂SO₄ and concentrated. The residue was purified by flash chromatography (silica gel, 33% EtOAc and 17% DCM in PE) to give 300 mg of ethyl 5-formyl-2-(hydroxymethyl)-7-methylpyrrolo[2,1-b]thiazole-6-carboxylate. LC-MS (ESI): m/z 268 (M+H)⁺.

Step F. Synthesis of ethyl 2-(((tert-butyldimethylsilyl)oxy)methyl)-5-formyl-7-methylpyrrolo[2,1-b]thiazole-6-carboxylate To a stirred mixture of ethyl 5-formyl-2-(hydroxymethyl)-7-methylpyrrolo[2,1-b]thiazole-6-carboxylate (300 mg, 1.3 mmol) in DCM (10 mL) was added imidazole (260 mg, 3.8 mmol) and TBSCl (288 mg, 1.9 mmol). The reaction mixture was stirred at r.t. for 1 hr. The reaction mixture was diluted with water, extracted with DCM. The organic phase was washed with brine, dried over anhy. Na₂SO₄ and concentrated. The residue was purified by flash chromatography (silica gel, 10% EtOAc in PE) to give 170 mg of ethyl 2-(((tert-butyldimethylsilyl)oxy)methyl)-5-formyl-7-methylpyrrolo[2,1-b]thiazole-6-carboxylate. LC-MS (ESI): m/z 382 (M+H)⁺.

Step G. Synthesis of 2-(((tert-butyldimethylsilyl)oxy)methyl)-9-methylthiazolo[3′,2′:1,5]pyrrolo[2,3-d]pyridazin-8 (7H)-one To a stirred mixture of ethyl 2-(((tert-butyldimethylsilyl)oxy)methyl)-5-formyl-7-methylpyrrolo[2,1-b]thiazole-6-carboxylate (500 mg, 1.3 mmol) in 2-methoxyethanol (15 mL) was added hydrazine hydrate (655 mg, 13 mmol, 98% w/w). The reaction mixture was stirred at 100° C. overnight. The reaction mixture was diluted with water, extracted with DCM. The organic phase was washed with brine, dried over anhy. Na₂SO₄ and concentrated. The residue was purified by flash chromatography (silica gel, 50% EtOAc in PE, 3% MeOH in DCM) to give 310 mg of 2-(((tert-butyldimethylsilyl)oxy)methyl)-9-methylthiazolo[3′,2′:1,5]pyrrolo[2,3-d]pyridazin-8 (7H)-one. LC-MS (ESI): m/z 350 (M+H)⁺.

Step H. Synthesis of 2-(((tert-butyldimethylsilyl)oxy)methyl)-9-methyl-7-((1-methyl-1H-pyrazol-3-yl)methyl)thiazolo[3′,2′:1,5]pyrrolo[2,3-d]pyridazin-8 (7H)-one To a stirred mixture of 2-(((tert-butyldimethylsilyl)oxy)methyl)-9-methylthiazolo[3′,2′:1,5]pyrrolo[2,3-d]pyridazin-8 (7H)-one (310 mg, 0.89 mmol) and (1-methyl-1H-pyrazol-3-yl)methanol (99 mg, 0.89 mmol) in toluene (30 mL) was added CMBP (0.7 mL, 2.66 mmol). The mixture was stirred under N₂ at 110° C. for 2 hr in microwave. The mixture was concentrated and purified by flash chromatography (silica gel, 50% EtOAc in PE) and washed with PE to give 250 mg of 2-(((tert-butyldimethylsilyl)oxy)methyl)-9-methyl-7-((1-methyl-1H-pyrazol-3-yl)methyl)thiazolo[3′,2′:1,5]pyrrolo[2,3-d]pyridazin-8 (7H)-one. LC-MS (ESI): m/z 444 (M+H)⁺.

Step I. Synthesis of 2-(hydroxymethyl)-9-methyl-7-((1-methyl-1H-pyrazol-3-yl)methyl)thiazolo[3′,2′:1,5]pyrrolo[2,3-d]pyridazin-8 (7H)-one To a stirred solution of 2-(((tert-butyldimethylsilyl)oxy)methyl)-9-methyl-7-((1-methyl-1H-pyrazol-3-yl)methyl)thiazolo[3′,2′:1,5]pyrrolo[2,3-d]pyridazin-8 (7H)-one (250 mg, 0.56 mmol) in MeOH (5 mL) was added HCl/dioxane (5 mL, 4 M). The mixture was stirred at r.t. for 1 hr. The mixture was concentrated to give 200 mg of 2-(hydroxymethyl)-9-methyl-7-((1-methyl-1H-pyrazol-3-yl)methyl)thiazolo[3′,2′:1,5]pyrrolo[2,3-d]pyridazin-8 (7H)-one. LC-MS (ESI): m/z 330 (M+H)⁺.

¹H NMR (400 MHz, DMSO-d₆) δ 8.59 (s, 1H), 8.31 (s, 1H), 7.55 (d, 1H), 6.04 (d, 1H), 5.82 (s, 1H), 5.22 (s, 2H), 4.62 (d, 2H), 3.76 (s, 3H), 2.46 (s, 3H).

Step J. Synthesis of 2-(chloromethyl)-9-methyl-7-((1-methyl-1H-pyrazol-3-yl)methyl)thiazolo[3′,2′:1,5]pyrrolo[2,3-d]pyridazin-8 (7H)-one To a stirred solution of 2-(hydroxymethyl)-9-methyl-7-((1-methyl-1H-pyrazol-3-yl)methyl)thiazolo[3′,2′:1,5]pyrrolo[2,3-d]pyridazin-8 (7H)-one (210 mg, 0.64 mmol) in DCM (5 mL) was added thionyl chloride (0.14 mL, 1.9 mmol). The mixture was stirred at r.t. for 1 hr under N₂. The mixture was concentrated in vacuum and the residue was adjusted to pH=7˜8 with satd. NaHCO₃, extracted with DCM/i-PrOH. The organic phase was washed with brine, dried over anhy. Na₂SO₄ and concentrated. The residue was purified by flash chromatography (silica gel, 50% EtOAc in PE) to give 170 mg of 2-(chloromethyl)-9-methyl-7-((1-methyl-1H-pyrazol-3-yl)methyl)thiazolo[3′,2′:1,5]pyrrolo[2,3-d]pyridazin-8 (7H)-one. LC-MS (ESI): m/z 348 (M+H)⁺.

Step K. Synthesis of 9-methyl-7-((1-methyl-1H-pyrazol-3-yl)methyl)-2-((1-((2-(trimethylsilyl)ethoxy)methyl)-1H-pyrazol-3-yl)methyl)thiazolo[3′,2′:1,5]pyrrolo[2,3-d]pyridazin-8 (7H)-one To a stirred solution of 2-(chloromethyl)-9-methyl-7-((1-methyl-1H-pyrazol-3-yl)methyl)thiazolo[3′,2′:1,5]pyrrolo[2,3-d]pyridazin-8 (7H)-one (30 mg, 0.09 mmol) and 3-(tributylstannyl)-1-((2-(trimethylsilyl)ethoxy)methyl)-1H-pyrazole (63 mg, 0.13 mmol) in toluene (2 mL) was added Pd(PPh₃)₄ (10 mg, 0.01 mmol). The mixture was stirred at 120° C. for 0.5 hr in microwave. The mixture was concentrated in vacuum and purified by prep-TLC (60% EtOAc in PE) to give 18 mg of 9-methyl-7-((1-methyl-1H-pyrazol-3-yl)methyl)-2-((1-((2-(trimethylsilyl)ethoxy)methyl)-1H-pyrazol-3-yl)methyl)thiazolo[3′,2′:1,5]pyrrolo[2,3-d]pyridazin-8 (7H)-one. LC-MS (ESI): m/z 510 (M+H)⁺.

Step L. Synthesis of 2-((1H-pyrazol-3-yl)methyl)-9-methyl-7-((1-methyl-1H-pyrazol-3-yl)methyl)thiazolo[3′,2′:1,5]pyrrolo[2,3-d]pyridazin-8 (7H)-one A mixture of 9-methyl-7-((1-methyl-1H-pyrazol-3-yl)methyl)-2-((1-((2-(trimethylsilyl)ethoxy)methyl)-1H-pyrazol-3-yl)methyl)thiazolo[3′,2′:1,5]pyrrolo[2,3-d]pyridazin-8 (7H)-one (18 mg, 0.04 mmol) in DCM/TFA (2 mL/2 mL) was stirred at r.t. for 1 hr. The reaction mixture was concentrated. The residue was purified by prep-HPLC to give 1.1 mg of 2-((1H-pyrazol-3-yl)methyl)-9-methyl-7-((1-methyl-1H-pyrazol-3-yl)methyl)thiazolo[3′,2′:1,5]pyrrolo[2,3-d]pyridazin-8 (7H)-one. LC-MS (ESI): m/z 380 (M+H)⁺. ¹H NMR (400 MHz, DMSO-d₆) δ 12.71 (s, 1H), 8.57 (s, 1H), 8.21 (s, 1H), 7.63 (s, 1H), 7.54 (d, 1H), 6.20 (d, 1H), 6.03 (d, 1H), 5.21 (s, 2H), 4.13 (s, 2H), 3.76 (s, 3H), 2.42 (s, 3H).

### Example 11. Synthesis of 7-(3-methoxybenzyl)-2,9-dimethylthiazolo[4′,5′:4,5]pyrrolo[1,2-d][1,2,4]triazin-8 (7H)-one

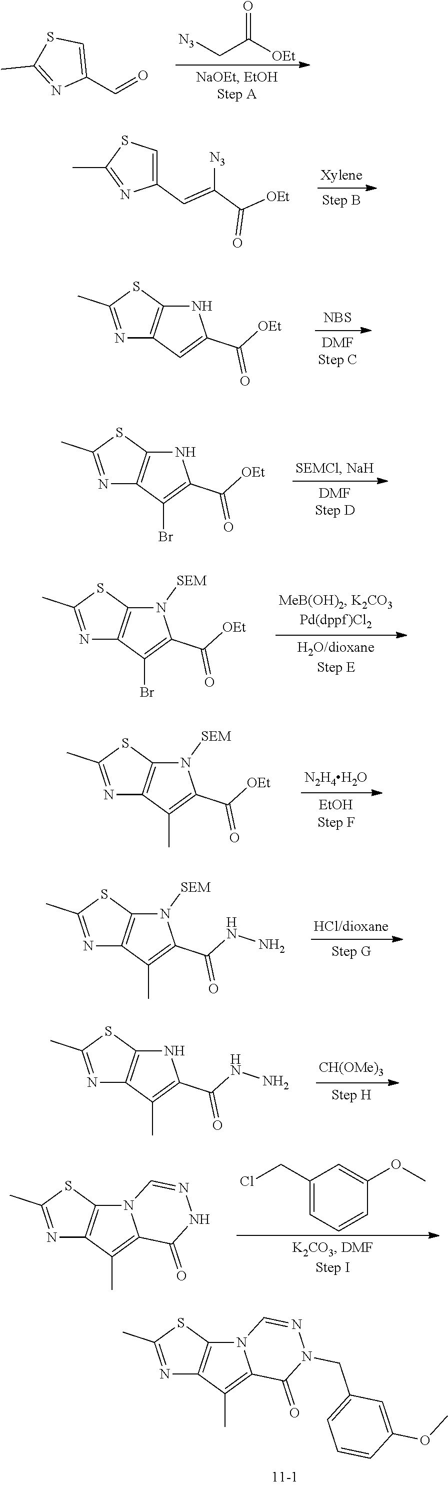

Step A. Synthesis of ethyl 2-azido-3-(2-methylthiazol-4-yl)acrylate To a solution of NaOEt (4.8 g, 70.7 mmol) in EtOH (60 mL) was added a solution of 2-methylthiazole-4-carbaldehyde (3 g, 23.6 mmol) and ethyl 2-azidoacetate (9.2 g, 70.7 mmol) in anhydrous EtOH (18 mL) by dropwise at −5° C. The reaction mixture was stirred below 0° C. for 1 hr and warmed to r.t. for another 2 hr. The resulting mixture was poured into satd. NH₄Cl at 0° C. and extracted with EtOAc. The organic layer was washed with brine, dried over anhy. Na₂SO₄ and concentrated to give 3 g of ethyl 2-azido-3-(2-methylthiazol-4-yl)acrylate. LC-MS (ESI): m/z 239 (M+H)⁺.

Step B. Synthesis of ethyl 2-methyl-4H-pyrrolo[3,2-d]thiazole-5-carboxylate A mixture of ethyl (Z)-2-azido-3-(2-methylthiazol-4-yl)acrylate (3 g, 12.6 mmol) in o-xylene (30 mL) was stirred at 140° C. for 2 hr and concentrated. The residue was purified by silica gel chromatography (eluent with PE/EtOAc=6/1) to give 1.2 g of ethyl 2-methyl-4H-pyrrolo[3,2-d]thiazole-5-carboxylate. LC-MS (ESI): m/z 211 (M+H)⁺.

Step C. Synthesis of ethyl 6-bromo-2-methyl-4H-pyrrolo[3,2-d]thiazole-5-carboxylate To a solution of ethyl ethyl 2-methyl-4H-pyrrolo[3,2-d]thiazole-5-carboxylate (1.2 g, 5.7 mmol) in DMF (60 mL) was added NBS (1 g, 5.7 mmol) by portions. The resulting mixture was stirred at r.t. for 2 hr, poured into satd. NaHCO₃ and extracted with EtOAc. The organic layer was washed with brine, dried over anhy. Na₂SO₄ and concentrated. The residue was purified by silica gel chromatography (eluent with PE/EtOAc=6/1) to give 800 mg of ethyl 6-bromo-2-methyl-4H-pyrrolo[3,2-d]thiazole-5-carboxylate. LC-MS (ESI): m/z 289 (M+H)⁺.

Step D. Synthesis of ethyl 6-bromo-2-methyl-4-((2-(trimethylsilyl)ethoxy)methyl)-4H-pyrrolo[3,2-d]thiazole-5-carboxylate To a mixture of ethyl 6-bromo-2-methyl-4H-pyrrolo[3,2-d]thiazole-5-carboxylate (800 mg, 2.8 mmol) in DMF (30 mL) was added NaH (167 mg, 4.2 mmol, 60% wt) at 0° C. The reaction mixture was stirred at r.t. for 0.5 hr, followed by the addition of SEMCl (695 mg, 4.2 mmol). The resulting mixture was stirred at r.t. for 1.5 hr, poured into satd. NH₄Cl and extracted with EtOAc. The organic layer was washed with brine, dried over anhy. Na₂SO₄ and concentrated. The residue was purified by silica gel chromatography (eluent with PE/EtOAc=10/1) to give 500 mg of ethyl 6-bromo-2-methyl-4-((2-(trimethylsilyl)ethoxy)methyl)-4H-pyrrolo[3,2-d]thiazole-5-carboxylate. LC-MS (ESI): m/z 419 (M+H)⁺.

Step E. Synthesis of ethyl 2,6-dimethyl-4-((2-(trimethylsilyl)ethoxy)methyl)-4H-pyrrolo[3,2-d]thiazole-5-carboxylate To a mixture of ethyl 6-bromo-2-methyl-4-((2-(trimethylsilyl)ethoxy)methyl)-4H-pyrrolo[3,2-d]thiazole-5-carboxylate (500 mg, 1.2 mmol) in H₂O (2 mL) and 1,4-dioxane (10 mL) was added methylboronic acid (107 mg, 1.8 mmol), K₂CO₃ (494 mg, 3.6 mmol) and Pd(dppf)Cl₂ (87 mg, 0.12 mmol). The reaction mixture was stirred under N₂ at 90° C. for 16 hr. The mixture was diluted with water and extracted with EtOAc. The organic layer was washed with brine, dried over anhy. Na₂SO₄ and concentrated. The residue was purified by silica gel chromatography (eluent with PE/EtOAc=10/1) to give 300 mg of ethyl 2,6-dimethyl-4-((2-(trimethylsilyl)ethoxy)methyl)-4H-pyrrolo[3,2-d]thiazole-5-carboxylate. LC-MS (ESI): m/z 355 (M+H)⁺.

Step F. Synthesis of 2,6-Dimethyl-4-((2-(trimethylsilyl)ethoxy)methyl)-4H-pyrrolo[3,2-d]thiazole-5-carbohydrazide To a mixture of ethyl 2,6-dimethyl-4-((2-(trimethylsilyl)ethoxy)methyl)-4H-pyrrolo[3,2-d]thiazole-5-carboxylate (300 mg, 0.85 mmol) in EtOH (9 mL) was added hydrazine hydrate (1 mL). The reaction mixture was stirred at 90° C. for 16 hr and concentrated. The residue was purified by silica gel chromatography (eluent with DCM/MeOH=20/1) to give 270 mg of 2,6-dimethyl-4-((2-(trimethylsilyl)ethoxy)methyl)-4H-pyrrolo[3,2-d]thiazole-5-carbohydrazide. LC-MS (ESI): m/z 341 (M+H)⁺.

Step G. Synthesis of 2,6-Dimethyl-4H-pyrrolo[3,2-d]thiazole-5-carbohydrazide A mixture of 2,6-dimethyl-4-((2-(trimethylsilyl)ethoxy)methyl)-4H-pyrrolo[3,2-d]thiazole-5-carbohydrazide (80 mg, 0.24 mmol) in HCl/dioxane (30 mL, 4 M) was stirred at r.t. for 2 days. The mixture was concentrated under reduced pressure, diluted with MeOH (10 mL) and NH₃.H₂O (10 mL). The resulting mixture was stirred at r.t. for 5 min and concentrated to give 50 mg of 2,6-dimethyl-4H-pyrrolo[3,2-d]thiazole-5-carbohydrazide. LC-MS (ESI): m/z 211 (M+H)⁺.

Step H. Synthesis of 2,9-Dimethylthiazolo[4′,5′:4,5]pyrrolo[1,2-d][1,2,4]triazin-8 (7H)-one A mixture of 2,6-dimethyl-4H-pyrrolo[3,2-d]thiazole-5-carbohydrazide (50 mg, 0.24 mmol) in trimethyloxymethane (10 mL) was stirred at 100° C. for 2 hr and concentrated. The residue was purified by perp-TLC (eluent: DCM/MeOH=50/1) to give 15 mg of 2,9-dimethylthiazolo[4′,5′:4,5]pyrrolo[1,2-d][1,2,4]triazin-8 (7H)-one. LC-MS (ESI): m/z 221 (M+H)⁺.

Step I. Synthesis of 7-(3-Methoxybenzyl)-2,9-dimethylthiazolo[4′,5′:4,5]pyrrolo[1,2-d][1,2,4]triazin-8 (7H)-one To a mixture of 2,9-dimethylthiazolo[4′,5′:4,5]pyrrolo[1,2-d][1,2,4]triazin-8 (7H)-one (15 mg, 0.07 mmol) in anhydrous DMF (5 mL) was added K₂CO₃ (28 mg, 0.2 mmol) and 1-(chloromethyl)-3-methoxybenzene (16 mg, 0.1 mmol). The mixture was stirred at r.t. for 16 hr. The mixture was diluted with water and extracted with EtOAc. The organic layer was washed with brine, dried over anhy. Na₂SO₄ and concentrated. The residue was purified by prep-HPLC to give 9 mg of 7-(3-Methoxybenzyl)-2,9-dimethylthiazolo[4′,5′:4,5]pyrrolo[1,2-d][1,2,4]triazin-8 (7H)-one. LC-MS (ESI): m/z 341 (M+H)⁺. ¹H NMR (400 MHz, CD₃OD) δ 8.98 (s, 1H), 7.22 (dd, 1H), 6.88-6.83 (m, 1H), 6.80-6.70 (m, 2H), 5.68 (s, 2H), 3.72 (s, 3H), 2.68 (s, 3H), 2.60 (s, 3H).

### Example 12. Synthesis of 6-benzyl-2,4-dimethyl-4H-thiazolo[4′,5′:4,5]pyrrolo[2,3-d]pyridazin-5 (6H)-one

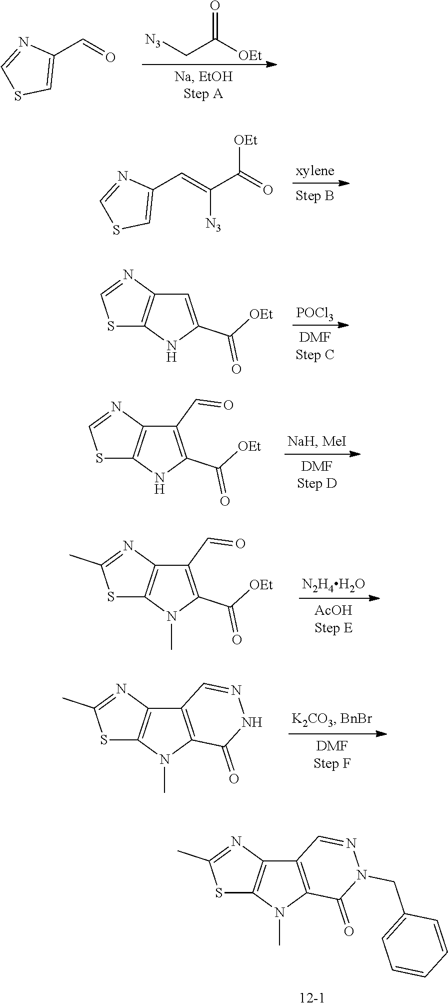

Step A. Synthesis of ethyl 2-azido-3-(thiazol-4-yl)acrylate A solution of 1,3-thiazole-4-carbaldehyde (5 g, 44 mmol) and ethyl 2-azidoacetate (17 g, 132 mmol) in anhy. EtOH (50 mL) was added dropwise to a solution of Na (3 g, 132 mmol) in anhy. EtOH (150 mL) between −10˜−5° C. The reaction mixture was stirred for 1 hr below 0° C., and warmed to r.t. for 1 hr. The mixture was quenched with satd. NH₄Cl, extracted with EtOAc. The combined organic phase was washed with brine, dried over anhy. Na₂SO₄ and concentrated to give 4 g of ethyl 2-azido-3-(thiazol-4-yl)acrylate. LC-MS (ESI): m/z 225 (M+H)⁺.

Step B. Synthesis of ethyl 4H-pyrrolo[3,2-d]thiazole-5-carboxylate A mixture of ethyl 2-azido-3-(thiazol-4-yl)acrylate (4 g, 17.8 mmol) in xylene (20 mL) was refluxed for 15 min. The mixture was concentrated and the residue was purified by flash chromatography (silica gel, 0˜ 50% EtOAc in PE) to give 1.5 g of ethyl 4H-pyrrolo[3,2-d]thiazole-5-carboxylate. LC-MS (ESI): m/z 197 (M+H)⁺.

Step C. Synthesis of ethyl 6-formyl-4H-pyrrolo[3,2-d]thiazole-5-carboxylate To a solution of ethyl 4H-pyrrolo[3,2-d]thiazole-5-carboxylate (1.4 g, 7.2 mmol) in DMF (10 mL) was added POCl₃ (10 mL). The mixture was stirred at 100° C. overnight. The mixture was quenched with satd. NaHCO₃ at 0° C. and extracted with DCM. The combined organic layer was washed with brine, dried over anhy. Na₂SO₄ and concentrated. The residue was purified by flash chromatography (silica gel, 0˜ 100% EtOAc in PE) to give 400 mg of ethyl 6-formyl-4H-pyrrolo[3,2-d]thiazole-5-carboxylate. LC-MS (ESI): m/z 225 (M+H)⁺.

Step D. Synthesis of ethyl 6-formyl-2,4-dimethyl-4H-pyrrolo[3,2-d]thiazole-5-carboxylate To a stirred mixture of ethyl 6-formyl-4H-pyrrolo[3,2-d]thiazole-5-carboxylate (300 mg, 1.3 mmol) in DMF (5 mL) was added NaH (107 mg, 60% wt, 2.7 mmol) under N₂. The reaction mixture was stirred at 0° C. for 0.5 hr followed by the addition of MeI (0.17 mL, 2.7 mmol). The reaction mixture was poured into satd. NH₄Cl, extracted with EtOAc. The combined organic layer was washed with brine, dried over anhy. Na₂SO₄ and concentrated. The residue was purified by flash chromatography (silica gel, 0˜ 80% EtOAc in PE) to give 80 mg of ethyl 6-formyl-2,4-dimethyl-4H-pyrrolo[3,2-d]thiazole-5-carboxylatet. LC-MS (ESI): m/z 253 (M+H)⁺.

Step E. Synthesis of 2,4-dimethyl-4H-thiazolo[4′,5′:4,5]pyrrolo[2,3-d]pyridazin-5 (6H)-one To a mixture of ethyl 6-formyl-2,4-dimethyl-4H-pyrrolo[3,2-d]thiazole-5-carboxylate (50 mg, 0.2 mmol) in AcOH (3 mL) was added hydrazine hydrate (22 mg, 0.6 mmol). The reaction mixture was stirred at 100° C. for 2 hr. The precipitate was collected by filtration to give 30 mg of 2,4-dimethyl-4H-thiazolo[4′,5′:4,5]pyrrolo[2,3-d]pyridazin-5 (6H)-one. LC-MS (ESI): m/z 221 (M+H)⁺.

Step F. Synthesis of 6-benzyl-2,4-dimethyl-4H-thiazolo[4′,5′:4,5]pyrrolo[2,3-d]pyridazin-5 (6H)-one To a stirred mixture of 2,4-dimethyl-4H-thiazolo[4′,5′:4,5]pyrrolo[2,3-d]pyridazin-5 (6H)-one (30 mg, 0.14 mmol) and K₂CO₃ (58 mg, 0.42 mmol) in DMF (5 mL) was added BnBr (36 mg, 0.21 mmol). The reaction mixture was stirred at 60° C. for 2 hr. The reaction mixture was poured into satd. NH₄Cl, extracted with EtOAc. The combined organic layer was washed with brine, dried over anhy. Na₂SO₄ and concentrated. The residue was purified by prep-TLC (EtOAc/PE=1/100) to give 5 mg of 6-benzyl-2,4-dimethyl-4H-thiazolo[4′,5′:4,5]pyrrolo[2,3-d]pyridazin-5 (6H)-one. LC-MS (ESI): m/z 311 (M+H)⁺. ¹H NMR (400 MHz, DMSO-d₆) δ 8.39 (s, 1H), 7.34-7.26 (m, 5H), 5.32 (s, 2H), 4.14 (s, 3H), 2.49 (s, 3H).

### Example 13. Synthesis of 6-(3-methoxybenzyl)-1,4-dimethyl-4,6-dihydropyrazolo[3′,4′:4,5]pyrrolo[2,3-d]pyridazin-5 (1H)-one

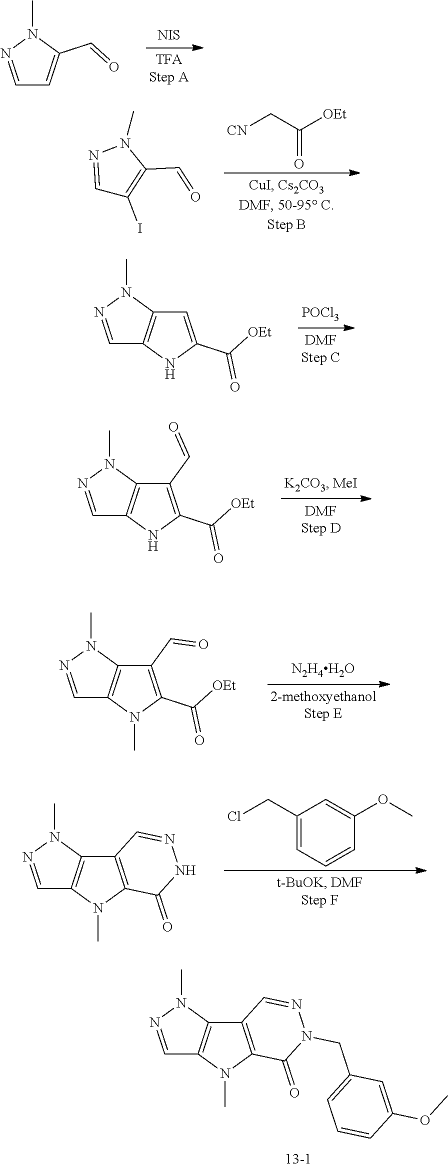

Step A. Synthesis of 4-iodo-1-methyl-1H-pyrazole-5-carbaldehyde to a stirred mixture of 1-methyl-1H-pyrazole-5-carbaldehyde (1.1 g, 10 mmol) in TFA (10 mL) was added NIS (3.4 g, 15 mmol) at 0° C. After stirred at r.t. for 16 hr, the reaction mixture was poured into satd. NaHCO₃, extracted with DCM. The organic layer was dried over anhy. Na₂SO₄ and concentrated. The residue was purified by silica gel chromatography (eluted with PE:EtOAc=35:1) to afford 1.8 g of 4-iodo-1-methyl-1H-pyrazole-5-carbaldehyde. LC-MS (ESI): m/z 237 (M+H)⁺.

Step B. Synthesis of ethyl 1-methyl-1,4-dihydropyrrolo[3,2-c]pyrazole-5-carboxylate To a stirred mixture of 4-iodo-1-methyl-1H-pyrazole-5-carbaldehyde (100 mg, 0.42 mmol) in DMF (10 mL) was added Cs₂CO₃ (274 mg, 0.84 mmol), ethyl 2-isocyanoacetate (53 mg, 0.47 mmol) and CuI (15 mg, 0.08 mmol). The reaction mixture was stirred under N₂ at 50° C. for 1 hr and 95° C. for 16 hr. The reaction mixture was poured into water, extracted with EtOAc. The organic layer was dried over anhy. Na₂SO₄ and concentrated. The residue was purified by silica gel chromatography (eluted with DCM:MeOH=35:1) to afford 40 mg of ethyl 1-methyl-1,4-dihydropyrrolo[3,2-c]pyrazole-5-carboxylate. LC-MS (ESI): m/z 194 (M+H)⁺.

Step C. Synthesis of ethyl 6-formyl-1-methyl-1,4-dihydropyrrolo[3,2-c]pyrazole-5-carboxylate To a stirred mixture of ethyl 1-methyl-1,4-dihydropyrrolo[3,2-c]pyrazole-5-carboxylate (193 mg, 1 mmol) in dry DMF (5 mL) was added POCl₃ (230 mg, 1.5 mmol) by dropwise at 0° C. The reaction mixture was stirred at 100° C. under N₂ for 3 hr and then cooled down. The reaction mixture was poured into water, extracted with DCM. The organic layer was dried over anhy. Na₂SO₄ and concentrated to afford 180 mg of ethyl 6-formyl-1-methyl-1,4-dihydropyrrolo[3,2-c]pyrazole-5-carboxylate. LC-MS (ESI): m/z 222 (M+H)⁺.

Step D. Synthesis of ethyl 6-formyl-1,4-dimethyl-1,4-dihydropyrrolo[3,2-c]pyrazole-5-carboxylate To a stirred mixture of ethyl 6-formyl-1-methyl-1,4-dihydropyrrolo[3,2-c]pyrazole-5-carboxylate (220 mg, 1 mmol) in dry DMF (5 mL) was added K₂CO₃ (276 mg, 2 mmol) and MeI (280 mg, 2 mmol). After stirred at r.t. overnight, the reaction mixture was poured into satd. NH₄Cl, extracted with EtOAc. The organic layer was dried over anhy. Na₂SO₄ and concentrated. The residue was purified by silica gel chromatography (eluted with PE:EtOAc=15:1) to afford 200 mg of ethyl 6-formyl-1,4-dimethyl-1,4-dihydropyrrolo[3,2-c]pyrazole-5-carboxylate. LC-MS (ESI): m/z 236 (M+H)⁺.

Step E. Synthesis of 1,4-dimethyl-4,6-dihydropyrazolo[3′,4′:4,5]pyrrolo[2,3-d]pyridazin-5 (1H)-one To a stirred mixture of ethyl 6-formyl-1,4-dimethyl-1,4-dihydropyrrolo[3,2-c]pyrazole-5-carboxylate (470 mg, 2 mmol) in 2-methoxyethanol (5 mL) was added N₂H4-H₂O (200 mg, 4 mmol, 98% w/w). The reaction mixture was stirred at 105° C. for 3 hr. The reaction mixture was diluted with water, extracted with DCM. The organic phase was with brine, dried over anhy. Na₂SO₄ and concentrated to afford 400 mg of 1,4-dimethyl-4,6-dihydropyrazolo[3′,4′:4,5]pyrrolo[2,3-d]pyridazin-5 (1H)-one. LC-MS (ESI): m/z 204 (M+H)⁺.

Step F. Synthesis of 6-(3-methoxybenzyl)-1,4-dimethyl-4,6-dihydropyrazolo[3′,4′:4,5]pyrrolo[2,3-d]pyridazin-5 (1H)-one To a stirred mixture of 1,4-dimethyl-4,6-dihydropyrazolo[3′,4′:4,5]pyrrolo[2,3-d]pyridazin-5 (1H)-one (203 mg, 1.0 mmol) in DMF (4 mL) was added t-BuOK (224 mg, 2.0 mmol) and 1-(chloromethyl)-3-methoxybenzene (312 mg, 2 mmol). The reaction mixture was stirred at r.t. for 2 hr. The reaction mixture was poured into satd. NH₄Cl, extracted with DCM. The organic layer was dried over anhy. Na₂SO₄ and concentrated. The residue was purified by silica gel chromatography (eluted with DCM:MeOH=30:1) to afford 30 mg of 6-(3-methoxybenzyl)-1,4-dimethyl-4,6-dihydropyrazolo[3′,4′:4,5]pyrrolo[2,3-d]pyridazin-5 (1H)-one. LC-MS (ESI): m/z 324 (M+H)⁺. ¹H NMR (400 MHz, DMSO-d₆) δ 8.64 (s, 1H), 7.74 (s, 1H), 724 (t, 1H), 6.88-6.80 (m, 3H), 5.33 (s, 2H), 4.16 (s, 3H). 4.13 (s, 3H) 3.72 (s, 3H).

### Example 14. Synthesis of 2-benzyl-6-(3-methoxybenzyl)-4-methyl-4,6-dihydropyrazolo[3′,4′:4,5]pyrrolo[2,3-d]pyridazin-5 (2H)-one

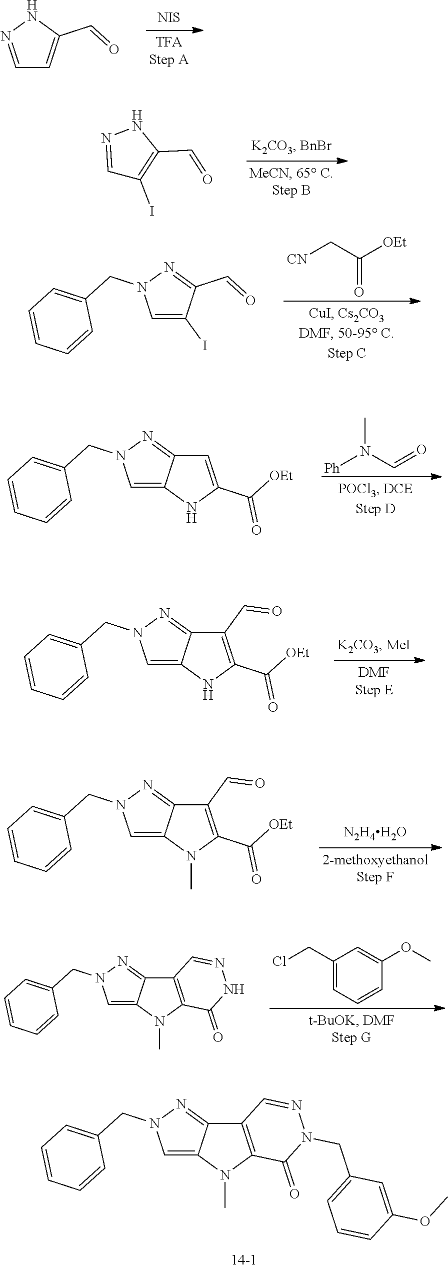

Step A. Synthesis of 4-iodo-1H-pyrazole-3-carbaldehyde To a mixture of 1H-pyrazole-3-carbaldehyde (5 g, 52 mmol) in TFA (20 mL) was added NIS (11.7 g, 52 mmol) in portions. The mixture was stirred at r.t. for 3 hrs. The reaction was quenched with satd. NaHCO₃. The precipitate was collected by filtration to give 10 g of 4-iodo-1H-pyrazole-3-carbaldehyde. LC-MS (ESI): m/z 223 (M+H)⁺.

Step B. Synthesis of 1-benzyl-4-iodo-1H-pyrazole-3-carbaldehyde To a mixture of 4-iodo-1H-pyrazole-3-carbaldehyde (5 g, 22 mmol) in MeCN (20 mL) was added K₂CO₃ (9.1 g, 66 mmol) and benzyl bromide (5.8 g, 33 mmol). The reaction mixture was stirred at r.t. overnight. The mixture was diluted with EtOAc, washed with water and brine. The organic layer was dried over anhy. Na₂SO₄ and concentrated. The residue was purified by silica gel chromatography (eluted with PE:EtOAc=20:1) to give 5 g of 1-benzyl-4-iodo-1H-pyrazole-3-carbaldehyde. LC-MS (ESI): m/z 313 (M+H)⁺.

Step C. Synthesis of ethyl 2-benzyl-2,4-dihydropyrrolo[3,2-c]pyrazole-5-carboxylate To a mixture of 1-benzyl-4-iodo-1H-pyrazole-3-carbaldehyde (5 g, 16 mmol), CuI (611 mg, 3.2 mmol) and Cs₂CO₃ (10.4 g, 32 mmol) in dry DMF (20 mL) was added ethyl 2-isocyanoacetate (2.1 g, 19 mmol). The mixture was stirred at 50° C. for 1 hr, then stirred at 95° C. overnight. The reaction mixture was poured into water, extracted with EtOAc. The organic layer was dried over anhy. Na₂SO₄ and concentrated. The residue was purified by silica gel chromatography (eluted with PE:EtOAc=10:1) to give 400 mg of ethyl 2-benzyl-2,4-dihydropyrrolo[3,2-c]pyrazole-5-carboxylate. LC-MS (ESI): m/z 270 (M+H)⁺.

Step D. Synthesis of ethyl 2-benzyl-6-formyl-2,4-dihydropyrrolo[3,2-c]pyrazole-5-carboxylate To a mixture of ethyl 2-benzyl-2,4-dihydropyrrolo[3,2-c]pyrazole-5-carboxylate (400 mg, 1.5 mmol) in DCE (8 mL) was added N-methyl-N-phenylformamide (303 mg, 2.25 mmol) and POCl₃ (0.26 mL, 2.25 mmol) under N₂. The mixture was stirred at 85° C. overnight. The reaction mixture was poured into water, extracted with DCM. The organic layer was dried over anhy. Na₂SO₄ and concentrated. The residue was purified by silica gel chromatography (eluted with PE:EtOAc=1:2) to give 450 mg of ethyl 2-benzyl-6-formyl-2,4-dihydropyrrolo[3,2-c]pyrazole-5-carboxylate. LC-MS (ESI): m/z 298 (M+H)⁺.

Step E. Synthesis of ethyl 2-benzyl-6-formyl-4-methyl-2,4-dihydropyrrolo[3,2-c]pyrazole-5-carboxylate To a solution of ethyl 2-benzyl-6-formyl-2,4-dihydropyrrolo[3,2-c]pyrazole-5-carboxylate (900 mg, 3.0 mmol) in DMF (6 mL) was added K₂CO₃ (836 mg, 9.0 mmol) and iodomethane (640 mg, 4.5 mmol). The reaction mixture was stirred at r.t. for 2.5 hrs. The reaction mixture was poured into satd. NH₄Cl, extracted with EtOAc. The organic layer was dried over anhy. Na₂SO₄ and concentrated. The residue was purified by silica gel chromatography (eluted with PE:EtOAc=1:2) to give 100 mg of ethyl 2-benzyl-6-formyl-4-methyl-2,4-dihydropyrrolo[3,2-c]pyrazole-5-carboxylate. LC-MS (ESI): m/z 312 (M+H)⁺.

Step F. Synthesis of 2-benzyl-4-methyl-4,6-dihydropyrazolo[3′,4′:4,5]pyrrolo[2,3-d]pyridazin-5 (2H)-one A mixture of ethyl 2-benzyl-6-formyl-4-methyl-2,4-dihydropyrrolo[3,2-c]pyrazole-5-carboxylate (100 mg, 0.32 mmol) and hydrazine hydrate (3 mL, 98% w/w) in 2-methoxyethanol (2 mL) was stirred at 100° C. for 1.5 hrs. The mixture was cooled down. The precipitate was collected by filtration, washed with PE to give 70 mg of 2-benzyl-4-methyl-4,6-dihydropyrazolo[3′,4′:4,5]pyrrolo[2,3-d]pyridazin-5 (2H)-one. LC-MS (ESI): m/z 280 (M+H)⁺.

Step G. Synthesis of 2-benzyl-6-(3-methoxybenzyl)-4-methyl-4,6-dihydropyrazolo[3′,4′:4,5]pyrrolo[2,3-d]pyridazin-5 (2H)-one To a mixture of 2-benzyl-4-methyl-4,6-dihydropyrazolo[3′,4′:4,5]pyrrolo[2,3-d]pyridazin-5 (2H)-one (70 mg, 0.25 mmol) in DMF (4 mL) was added t-BuOK (59 mg, 0.60 mmol) and 1-(chloromethyl)-3-methoxybenzene (42 mg, 0.30 mmol). The reaction mixture was stirred at r.t. for 2.5 hrs. The reaction mixture was poured into satd. NH₄Cl. The precipitate was collected by filtration, washed with PE to give 65 mg of 2-benzyl-6-(3-methoxybenzyl)-4-methyl-4,6-dihydropyrazolo[3′,4′:4,5]pyrrolo[2,3-d]pyridazin-5 (2H)-one. LC-MS (ESI): m/z 400 (M+H)⁺. ¹H NMR (400 MHz, DMSO-d₆) δ 8.54 (s, 1H), 8.23 (s, 1H), 7.24-7.43 (m, 6H), 6.83-6.95 (m, 3H), 5.63 (s, 2H), 5.39 (s, 2H), 4.18 (s, 3H), 3.79 (s, 3H).

### Example 15. Synthesis of 6-(3-methoxybenzyl)-4-methyl-4,6-dihydropyrazolo [3′,4′: 4,5]pyrrolo[2,3-d]pyridazin-5 (2H)-one

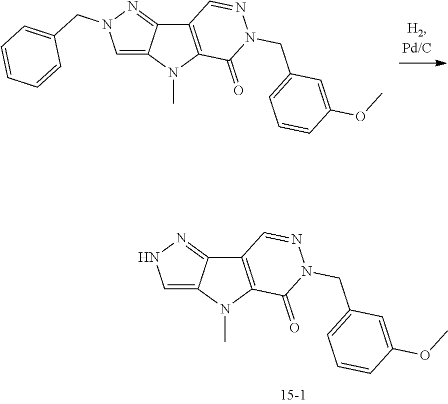 [SMILES: COc1cccc(Cn2ncc3c4nn(Cc5ccccc5)cc4n(C)c3c2=O)c1 ; COc1cccc(Cn2ncc3c4n[nH]cc4n(C)c3c2=O)c1]

To a mixture of 2-benzyl-6-(3-methoxybenzyl)-4-methyl-4,6-dihydropyrazolo [3′,4′:4,5]pyrrolo[2,3-d]pyridazin-5 (2H)-one (50 mg, 0.13 mmol) in MeOH (3 mL) under N₂ was added Pd/C. The mixture was stirred at r.t. under H₂ for 2 hr then concentrated under reduced pressure. The residue was purified by prep-TLC (eluent: PE/EtOAc=1/1) to give 7 mg of the desired product. LCMS: 310 (M+H)⁺. ¹H NMR (400 MHz, DMSO-d₆) δ 13.23 (brs, 1H), 8.41 (s, 1H), 7.87 (s, 1H), 7.23 (t, 1H), 6.89-6.73 (m, 3H), 5.33 (s, 2H), 4.17 (s, 3H), 3.72 (s, 3H).

### Example 16. Synthesis of 6-(3-methoxybenzyl)-2,4-dimethyl-4,6-dihydropyrazolo [3′,4′:4,5]pyrrolo[2,3-d]pyridazin-5 (2H)-one

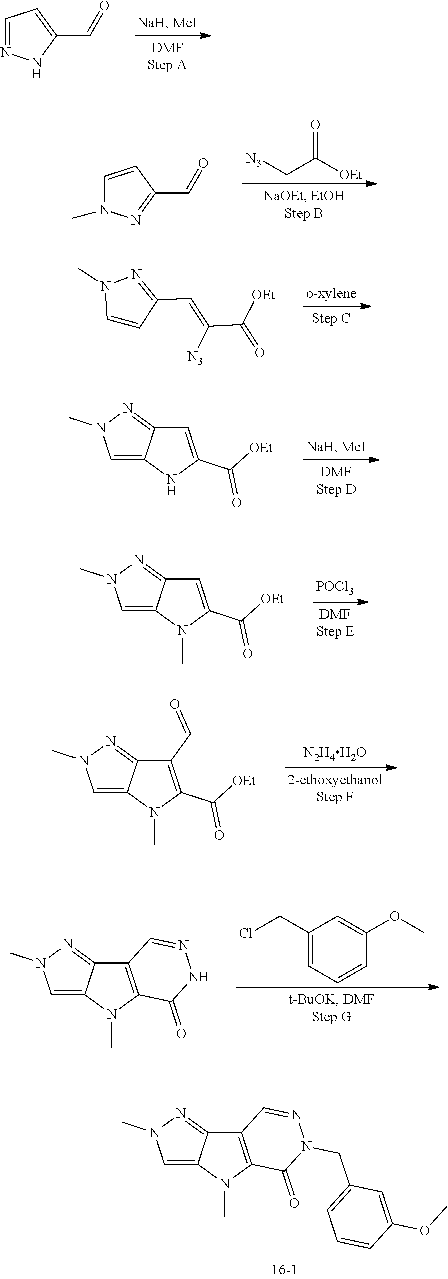

Step A. Synthesis of 1-methyl-1H-pyrazole-3-carbaldehyde To a solution of 1H-pyrazole-3-carbaldehyde (5.2 g, 54 mmol) in DMF (30 mL) was added NaH (4.3 g, 108 mmol) and iodomethane (1.15 g, 81 mmol). The mixture was stirred at r.t. for 2 hr. The reaction mixture was poured into satd. NH₄Cl, extracted with EtOAc. The organic phase was washed with brine, dried over anhy. Na₂SO₄, filtered and concentrated. The residue was purified by silica gel chromatography (eluted with PE:EtOAc=20:1) to afford 4.18 g of 1-methyl-1H-pyrazole-3-carbaldehyde. LC-MS (ESI): m/z 111 (M+H)⁺.

Step B. Synthesis of ethyl 2-azido-3-(1-methyl-1H-pyrazol-3-yl)acrylate To a solution of EtONa (1.8 g, 18.4 mmol) in EtOH (20 mL) was added 1-methyl-1H-pyrazole-3-carbaldehyde (1.0 g, 9.2 mmol) and azido-acetic acid ethyl ester (1.3 g, 10.1 mmol) at −10° C. After stirred for 3 hr, the reaction mixture was poured into satd. NH₄Cl, extracted with EtOAc. The organic phase was washed with brine, dried over anhy. Na₂SO₄, filtered and concentrated. The residue was purified by silica gel chromatography (eluted with PE:EtOAc=10:1) to afford 0.77 g of ethyl 2-azido-3-(1-methyl-1H-pyrazol-3-yl)acrylate. LC-MS (ESI): m/z 222 (M+H)⁺.

Step C. Synthesis of ethyl 2-methyl-2,4-dihydropyrrolo[3,2-c]pyrazole-5-carboxylate A mixture of (Z)-ethyl 2-azido-3-(1-methyl-1H-pyrazol-3-yl)acrylate (0.77 g, 3.5 mmol) in o-xylene (15 mL) was heated to reflux for 2 hr. The reaction mixture was concentrated and the residue was purified by silica gel chromatography (eluted with PE:EtOAc=5:1) to afford ethyl 2-methyl-2,4-dihydropyrrolo[3,2-c]pyrazole-5-carboxylate (500 mg, 82% yield) as a white solid. LC-MS (ESI): m/z 194 (M+H)⁺.

Step D. Synthesis of ethyl 2,4-dimethyl-2,4-dihydropyrrolo[3,2-c]pyrazole-5-carboxylate To a solution of ethyl 2-methyl-2,4-dihydropyrrolo[3,2-c]pyrazole-5-carboxylate (500 mg, 2.6 mmol) in DMF (10 mL) was added NaH (207 mg, 5.2 mmol) and iodomethane (552 mg, 3.9 mmol). The mixture was stirred at r.t. for 2 hr. The reaction mixture was poured into satd. NH₄Cl, extracted with EtOAc. The organic phase was washed with brine, dried over anhy. Na₂SO₄, filtered and concentrated. The residue was purified by silica gel chromatography (eluted with PE:EtOAc=15:1) to afford 500 mg of ethyl 2,4-dimethyl-2,4-dihydropyrrolo[3,2-c]pyrazole-5-carboxylate. LC-MS (ESI): m/z 208 (M+H)⁺.

Step E. Synthesis of ethyl 6-formyl-2,4-dimethyl-2,4-dihydropyrrolo[3,2-c]pyrazole-5-carboxylate To a mixture of ethyl 2,4-dimethyl-2,4-dihydropyrrolo[3,2-c]pyrazole-5-carboxylate (500 mg, 2.4 mmol) in DMF (10 mL) was added POCl₃ (1.85 g, 12.1 mmol). The reaction mixture was stirred at 90° C. for 3 hr. The reaction mixture was poured into water, extracted with EtOAc. The organic phase was washed with brine, dried over anhy. Na₂SO₄, filtered and concentrated. The residue was purified by silica gel chromatography (eluted with PE:EtOAc=15:1) to afford 150 mg of ethyl 6-formyl-2,4-dimethyl-2,4-dihydropyrrolo[3,2-c]pyrazole-5-carboxylate. LC-MS (ESI): m/z 236 (M+H)⁺.

Step F. Synthesis of 2,4-dimethyl-4,6-dihydropyrazolo[3′,4′:4,5]pyrrolo[2,3-d]pyridazin-5 (2H)-one To a solution of ethyl 6-formyl-2,4-dimethyl-2,4-dihydropyrrolo[3,2-c]pyrazole-5-carboxylate (150 mg, 0.64 mmol) in 2-ethoxyethanol (5 mL) was added N₂H4-H₂O (319 mg, 6.4 mmol). The reaction mixture was stirred at 100° C. for 2 hr. The reaction mixture was poured into water, extracted with EtOAc. The organic phase was washed with brine, dried over anhy. Na₂SO₄, filtered and concentrated. The residue was purified by silica gel chromatography (eluted with PE:EtOAc=3:1) to afford 120 mg of 2,4-dimethyl-4,6-dihydropyrazolo[3′,4′:4,5]pyrrolo[2,3-d]pyridazin-5 (2H)-one. LC-MS (ESI): m/z 204 (M+H)⁺.

Step G. Synthesis of 6-(3-methoxybenzyl)-2,4-dimethyl-4,6-dihydropyrazolo[3′,4′:4,5]pyrrolo[2,3-d]pyridazin-5 (2H)-one To a solution of 2,4-dimethyl-4,6-dihydropyrazolo[3′,4′:4,5]pyrrolo[2,3-d]pyridazin-5 (2H)-one (30 mg, 0.15 mmol) in DMF (3 mL) was added t-BuOK (33 mg, 0.3 mmol) and 1-chloromethyl-3-methoxy-benzene (46 mg, 0.3 mmol). The mixture was stirred at r.t. for 2 hr. The reaction mixture was poured into sadt. NH₄Cl, extracted with EtOAc. The organic phase was washed with brine, dried over anhy. Na₂SO₄, filtered and concentrated. The residue was purified by prep-TLC (EtOAc:PE=3:1) to afford 10 mg of 6-(3-methoxybenzyl)-2,4-dimethyl-4,6-dihydropyrazolo[3′,4′:4,5]pyrrolo[2,3-d]pyridazin-5 (2H)-one. LC-MS (ESI): m/z 324 (M+H)⁺. ¹H NMR (400 MHz, DMSO-d₆) δ 8.47 (s, 1H), 8.02 (s, 1H), 7.24 (t, 1H), 6.82-6.89 (m, 3H), 5.33 (s, 2H), 4.12 (s, 3H), 4.11 (s, 3H), 3.72 (s, 3H).

### Example 17. Synthesis of 5-methyl-3-((1-methyl-1H-pyrazol-3-yl)methyl)-8-(thiazol-4-ylmethyl)-3H-pyridazino[4,5-b]indol-4 (5H)-one

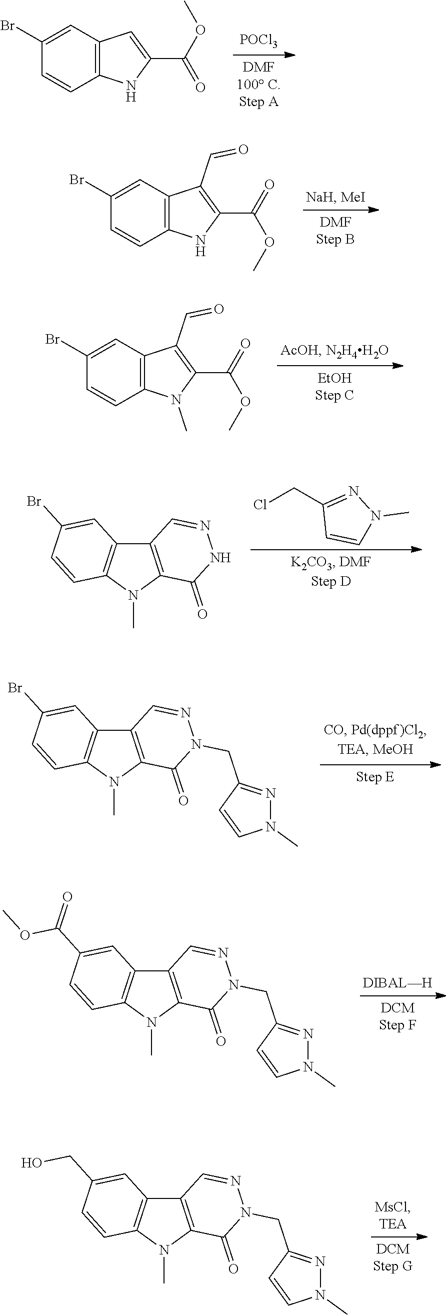

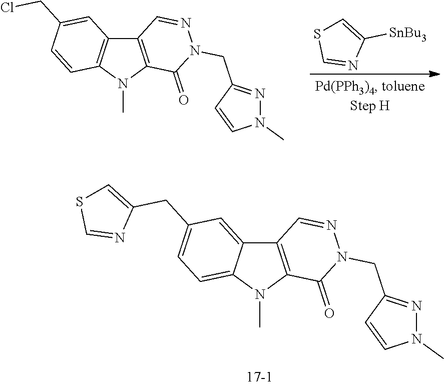

Step A: Synthesis of methyl 5-bromo-3-formyl-1H-indole-2-carboxylate. To a mixture of ethyl 5-bromo-1H-indole-2-carboxylate (5 g, 18.65 mmol) in DMF (30 mL) was added phosphoroyl trichloride (18 mL, 186.65 mmol) at 0° C. The reaction mixture was stirred at 100° C. overnight. The reaction mixture was diluted with ice-H₂O and extracted with EtOAc. The combined organic phase was evaporated under reduced pressure. The residue was purified by flash chromatography (silica gel, 0-10% EtOAc in PE) to give 4 g of ethyl 5-bromo-3-formyl-1H-indole-2-carboxylate. LC-MS(ESI): m/z 282 (M+H)⁺.

Step B: Synthesis of methyl 5-bromo-3-formyl-1-methyl-1H-indole-2-carboxylate to a mixture of methyl 5-bromo-3-formyl-1H-indole-2-carboxylate (4 g, 14.18 mmol) in DMF (30 mL) was added sodium hydride (0.68 g, 28.35 mmol). After stirred at r.t. for 0.5 hr, iodomethane (1.3 mL, 21.27 mmol) was added. The mixture was stirred at r.t. for 3 hr. The reaction mixture was diluted with satd. NH₄Cl and extracted with EtOAc. The combined organic phase was evaporated under reduced pressure. The residue was purified by flash chromatography (silica gel, 0-30% EtOAc in PE) to give 3.6 g of methyl 5-bromo-3-formyl-1-methyl-1H-indole-2-carboxylate. LC-MS (ESI): m/z 296 (M+H)⁺.

Step C: Synthesis of 8-bromo-5-methyl-3H-pyridazino[4,5-b]indol-4 (5H)-one To a mixture of methyl 5-bromo-3-formyl-1-methyl-1H-indole-2-carboxylate (4 g, 13.5 mmol) in EtOH (40 mL) was added hydrazine hydrate (0.67 g, 13.5 mmol) and acetic acid (0.77 mL, 13.5 mmol). The mixture was stirred at 70° C. for 1.5 hr. The reaction mixture was diluted with H₂O and extracted with EtOAc. The combined organic phase was evaporated under reduced pressure. The residue was purified by flash chromatography (silica gel, 0-30% EtOAc in PE) to give 3.5 g of 8-bromo-5-methyl-3H-pyridazino[4,5-b]indol-4 (5H)-one. LC-MS (ESI): m/z 278 (M+H)⁺.

Step D: Synthesis of 8-bromo-5-methyl-3-((1-methyl-1H-pyrazol-3-yl)methyl)-3H-pyridazino[4,5-b]indol-4 (5H)-one To a mixture of 8-bromo-5-methyl-3H-pyridazino[4,5-b]indol-4 (5H)-one (200 mg, 0.72 mmol) in DMF (5 mL) was added K₂CO₃ (200 mg, 1.44 mmol). After stirred at 70° C. for 1.5 hr, 3-(chloromethyl)-1-methyl-1H-pyrazole (187 mg, 1.44 mmol) was added. The mixture was stirred at 70° C. for 1 hr. The reaction mixture was diluted with water and extracted with EtOAc. The combined organic phase was evaporated under reduced pressure. The residue was purified by flash chromatography (silica gel, 0-40% EtOAc in PE) to give 180 mg of 8-bromo-5-methyl-3-((1-methyl-1H-pyrazol-3-yl)methyl)-3H-pyridazino[4,5-b]indol-4 (5H)-one. LC-MS (ESI): m/z 372 (M+H)⁺.

Step E: Synthesis of methyl 5-methyl-3-((1-methyl-1H-pyrazol-3-yl)methyl)-4-oxo-4,5-dihydro-3H-pyridazino[4,5-b]indole-8-carboxylate To a mixture of 8-bromo-5-methyl-3-((1-methyl-1H-pyrazol-3-yl)methyl)-3H-pyridazino[4,5-b]indol-4 (5H)-one (120 mg, 0.32 mmol) in MeOH (1 mL) and DMF (1 mL) was added TEA (1 mL) and Pd(dppf)Cl₂ (23 mg, 0.03 mmol). The mixture was stirred under CO at 100° C. overnight. The reaction mixture was diluted with H₂O and extracted with EtOAc. The combined organic phase was evaporated under reduced pressure. The residue was purified by flash chromatography (silica gel, 0-50% EtOAc in PE) to give 60 mg of methyl 5-methyl-3-((1-methyl-1H-pyrazol-3-yl)methyl)-4-oxo-4,5-dihydro-3H-pyridazino[4,5-b]indole-8-carboxylate. LC-MS (ESI): m/z 352 (M+H)⁺.

Step F: Synthesis of 8-(hydroxymethyl)-5-methyl-3-((1-methyl-1H-pyrazol-3-yl)methyl)-3H-pyridazino[4,5-b]indol-4 (5H)-one To a mixture of methyl 5-methyl-3-((1-methyl-1H-pyrazol-3-yl)methyl)-4-oxo-4,5-dihydro-3H-pyridazino[4,5-b]indole-8-carboxylate (60 mg, 0.17 mmol) in DCM (5 mL) was added DIBAL-H (0.4 mL, 1.3 M in toluene, 0.52 mmol) at 0° C. After stirred for 1.5 hr, the reaction mixture was quenched with satd. NH₄Cl, extracted with DCM. The organic phase was washed with brine and dried over anhy. Na₂SO₄, evaporated under reduced pressure. The residue was purified by pre-TLC (15% MeOH in DCM) to give 30 mg of 8-(hydroxymethyl)-5-methyl-3-((1-methyl-1H-pyrazol-3-yl)methyl)-3H-pyridazino[4,5-b]indol-4 (5H)-one. LC-MS (ESI): m/z 324 (M+H)⁺.

Step G: Synthesis of 8-(chloromethyl)-5-methyl-3-((1-methyl-1H-pyrazol-3-yl)methyl)-3H-pyridazino[4,5-b]indol-4 (5H)-one To a mixture of 8-(hydroxymethyl)-5-methyl-3-((1-methyl-1H-pyrazol-3-yl)methyl)-3H-pyridazino[4,5-b]indol-4 (5H)-one (40 mg, 0.12 mmol) in DCM (3 mL) was added TEA (63 mg, 0.62 mmol) and methanesulfonyl chloride (43 mg, 0.37 mmol) at 0° C. After stirred for 1.5 hr, the reaction mixture was diluted with DCM, washed with water and brine. The organic phase was dried over anhy. Na₂SO₄ and evaporated under reduced pressure. The residue was purified by pre-TLC (15% MeOH in DCM) to give 30 mg of 8-(chloromethyl)-5-methyl-3-((1-methyl-1H-pyrazol-3-yl)methyl)-3H-pyridazino[4,5-b]indol-4 (5H)-one. LC-MS (ESI): m/z 341 (M+H)⁺.

Step H: Synthesis of 5-methyl-3-((1-methyl-1H-pyrazol-3-yl)methyl)-8-(thiazol-4-ylmethyl)-3H-pyridazino[4,5-b]indol-4 (5H)-one To a mixture of 8-(chloromethyl)-5-methyl-3-((1-methyl-1H-pyrazol-3-yl)methyl)-3H-pyridazino[4,5-b]indol-4 (5H)-one (30 mg, 0.09 mmol) in toluene (3 mL) was added Pd(PPh₃)₄ (11 mg, 0.01 mmol) and 4-(tributylstannyl)-1,3-thiazole (99 mg, 0.27 mmol) under N₂. The reaction mixture was stirred at 100° C. for 1.5 hr and concentrated. The residue was purified by pre-TLC (20% MeOH in DCM) to give 2 mg of 5-methyl-3-((1-methyl-1H-pyrazol-3-yl)methyl)-8-(thiazol-4-ylmethyl)-3H-pyridazino[4,5-b]indol-4 (5H)-one. LC-MS (ESI): m/z 391 (M+H)⁺. ¹HNMR (400 MHz, DMSO-d₆) δ 9.03 (d, 1H), 8.74 (s, 1H), 8.09 (s, 1H), 7.69 (d, 1H), 7.57 (d, 1H), 7.53 (dd, 1H), 7.35 (d, 1H), 6.09 (d, 1H), 5.35 (s, 2H), 4.29 (s, 2H), 4.27 (s, 3H), 3.77 (s, 3H).

| Cpd No. | Structure and chemical name | Characterization |
|---|---|---|
| 17-2 | 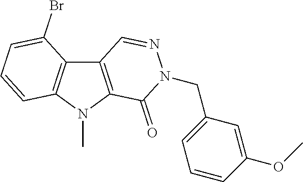 [SMILES: COc1cccc(Cn2ncc3c4c(Br)cccc4n(C)c3c2=O)c1] | LC-MS: m/z 398 (M + 1)⁺. ¹NMR (400 MHz, DMSO- d₆) δ 9.02 (s, 1H), 7.83 (d, 1H), 7.69 (d, 1H), 7.50 (t, 1H), 7.24 (t, 1H), 6.99-6.80 (m, 3H), 5.39 (s, 2H), 4.31 (s, 3H), 3.72 (s, 3H) |
| | 9-bromo-3-(3-methoxybenzyl)-5-methyl- | |
| | 3H-pyridazino[4,5-b]indol-4(5H)-one | |
| 17-3 | 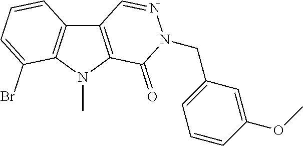 [SMILES: COc1cccc(Cn2ncc3c4cccc(Br)c4n(C)c3c2=O)c1] | LC-MS: m/z 398/400 (M + 2)⁺. ¹NMR (400 MHz, DMSO- d₆) δ 8.86 (s, 1H), 8.25-8.27 (m, 1H), 7.79-7.81 (m, 1H), 7.24-7.30 (m, 2H), 6.86-6.88 (m, 3H), 5.38 (s, 2H), 4.68 (s, 3H), 3.72 (s, 3H). |
| | 6-bromo-3-(3-methoxybenzyl)-5-methyl- | |
| | 3H-pyridazino[4,5-b]indol-4(5H)-one | |
| 17-4 | 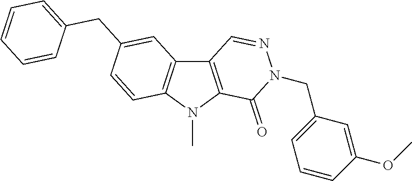 [SMILES: COc1cccc(Cn2ncc3c4cc(Cc5ccccc5)ccc4n(C)c3c2=O)c1] | LC-MS: m/z 410 (M + H)⁺. ¹H NMR (400 MHz, DMSO- d₆) δ1H NMR (400 MHz, DMSO) δ 8.76 (s, 1H), 8.07 (s, 1H), 7.66 (d, 1H), 7.47 (d, 1H), 7.34-7.12 (m, 6H), 6.91- 6.78 (m, 3H), 5.36 (s, 2H), 4.24 (s, 3H), 4.12 (s, 2H), 3.71 (s, 3H). |
| | 8-benzyl-3-(3-methoxybenzyl)-5-methyl- | |
| | 3H-pyridazino[4,5-b]indol-4(5H)-one | |
| 17-5 | 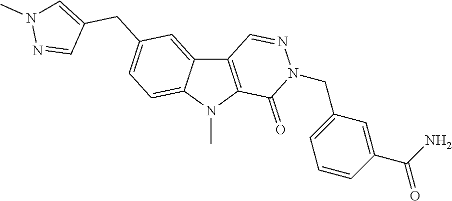 [SMILES: Cn1cc(Cc2ccc3c(c2)c2cnn(Cc4cccc(C(N)=O)c4)c(=O)c2n3C)cn1] | LC-MS: m/z 427 (M + H)⁺. ¹H NMR (400 MHz, DMSO- d₆) δ 8.79 (s, 1H), 8.03 (s, 1H), 7.97 (s, 1H), 7.81 (d, 1H), 7.77 (d, 1H), 7.67 (d, 1H), 7.47 (d, 1H), 7.46-7.44 (m, 2H), 7.40 (t, 1H), 7.34 (s, 1H), 7.26 (s, 1H), 5.44 (s, 2H), 4.26 (s, 3H), 3.92 (s, 2H), 3.75 (s, 3H) |
| | 3-((5-methyl-8-((1-methyl-1H-pyrazol-4- | |
| | yl)methyl)-4-oxo-4,5-dihydro-3H-pyridazino | |
| | [4,5-b]indol-3-yl)methyl)benzamide | |

### Example 18. Synthesis of 3-(3-methoxybenzyl)-N,5-dimethyl-4-oxo-4,5-dihydro-3H-pyridazino[4,5-b]indole-8-carboxamide

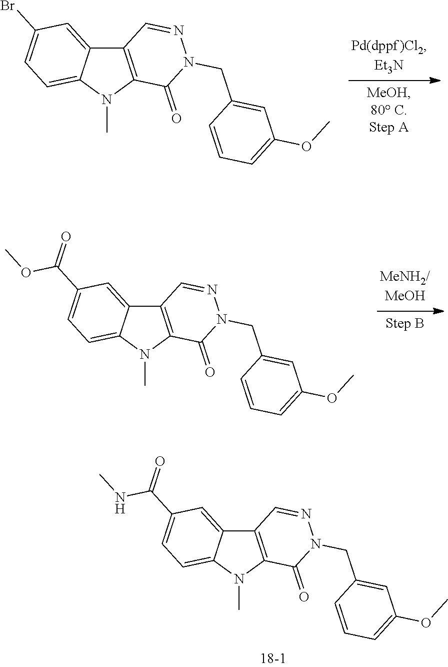

Step A. Synthesis of methyl 3-(3-methoxybenzyl)-5-methyl-4-oxo-4,5-dihydro-3H-pyridazino[4,5-b]indole-8-carboxylate To a mixture of 8-bromo-3-(3-methoxybenzyl)-5-methyl-3H-pyridazino[4,5-b]indol-4 (5H)-one (300 mg, 0.76 mmol) in methanol (15 mL) was added Et₃N (230 mg, 2.3 mmol) and Pd(dppf)₂Cl₂ (62 mg, 0.1 mmol). The reaction mixture was stirred at 80° C. overnight under CO balloon. The mixture was concentrated and the residue was purified by silica gel chromatography (eluted with PE:EtOAc=5:1) to give 150 mg of methyl 3-(3-methoxybenzyl)-5-methyl-4-oxo-4,5-dihydro-3H-pyridazino[4,5-b]indole-8-carboxylate. LC-MS (ESI): m/z 378 (M+H)⁺. ¹H NMR (400 MHz, DMSO-d₆) δ 8.98 (s, 1H), 8.92 (d, 1H), 8.15 (dd, 1H), 7.85 (d, 1H), 7.26 (t, 1H), 6.96-6.81 (m, 3H), 5.38 (s, 2H), 4.30 (s, 3H), 3.92 (s, 3H), 3.72 (s, 3H).

Step B. Synthesis of 3-(3-methoxybenzyl)-N,5-dimethyl-4-oxo-4,5-dihydro-3H-pyridazino[4,5-b]indole-8-carboxamide A mixture of methyl 3-(3-methoxybenzyl)-5-methyl-4-oxo-4,5-dihydro-3H-pyridazino[4,5-b]indole-8-carboxylate (40 mg, 0.1 mmol) and methanamine (2 mL, 30% wt in MeH) in a seal-tube was stirred at 100° C. overnight. The reaction mixture was concentrated. The residue was purified by prep-HPLC to give 18 mg of 3-(3-methoxybenzyl)-N,5-dimethyl-4-oxo-4,5-dihydro-3H-pyridazino[4,5-b]indole-8-carboxamide. LC-MS (ESI): m/z 377 (M+H)⁺. ¹H NMR (400 MHz, DMSO-d₆) δ 8.85 (s, 1H), 8.74 (d, 1H), 8.54 (d, 1H), 8.07 (dd, 1H), 7.82 (d, 1H), 7.24 (t, 1H), 6.93-6.79 (m, 3H), 5.38 (s, 2H), 4.30 (s, 3H), 3.72 (s, 3H), 2.84 (d, 3H).

| Cpd No. | Structure and chemical name | Characterization |
|---|---|---|
| 18-2 | 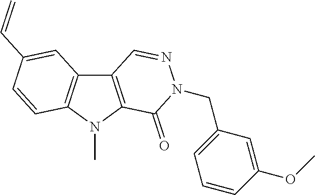 [SMILES: C=Cc1ccc2c(c1)c1cnn(Cc3cccc(OC)c3)c(=O)c1n2C] | LC-MS: m/z 346 (M + H)⁺. ¹H NMR (400 MHz, DMSO-d₆) δ 8.81 (s, 1H), 8.28 (s, 1H), 7.77 (dt, 2H), 7.24 (t, 1H), 6.96-6.81 (m, 4H), 5.91 (d, 1H), 5.37 (s, 2H), 5.29 (d, 1H), 4.28 (s, 3H), 3.72 (s, 3H). |
| | 3-(3-methoxybenzyl)-5-methyl-8-vinyl- | |
| | 3H-pyridazino[4,5-b]indol-4 (5H)-one | |
| 18-3 | 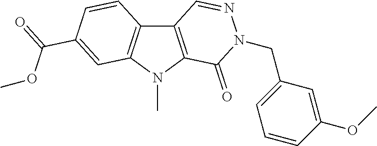 | LC-MS: m/z 378 (M + H)⁺. ¹H NMR (400 MHz, DMSO-d₆) δ 8.87 (s, 1H), 8.32 (d, 2H), 7.95 (d, 1H), 7.23 (d, 1H), 7.04-6.69 (m, 3H), 5.38 (s, 2H), 4.35 (s, 3H), 3.94 (s, 3H), 3.72 (s, 3H) |
| | methyl 3-(3-methoxybenzyl)-5-methyl-4- | |
| | oxo-4,5-dihydro-3H-pyridazino[4,5- | |
| | b]indole-7-carboxylate | |
| 18-4 | 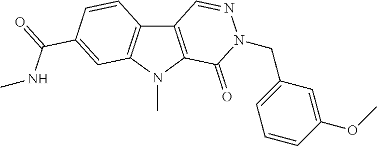 | LC-MS: m/z 377 (M + H)⁺. ¹H NMR (400 MHz, DMSO-d₆) δ 8.85 (s, 1H), 8.64 (d, 1H), 8.27 (d, 2H), 7.87 (dd, 1H), 7.25 (t, 1H), 6.86 (dd, 3H), 5.39 (s, 2H), 4.34 (s, 3H), 3.73 (s, 3H), 2.86 (d, 3H) |
| | 3-(3-methoxybenzyl)-N,5-dimethyl-4-oxo- | |
| | 4,5-dihydro-3H-pyridazino[4,5-b]indole-7-carboxamide | |
| 18-5 | 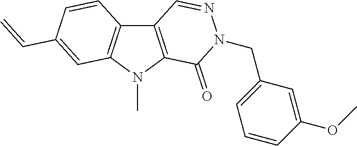 [SMILES: C=Cc1ccc2c3cnn(Cc4cccc(OC)c4)c(=O)c3n(C)c2c1] | LC-MS: m/z 346 (M + H)⁺. ¹H NMR (400 MHz, DMSO) δ 8.80 (s, 1H), 8.16 (d, 1H), 7.83 (s, 1H), 7.58 (d, 1H), 7.24 (t, 1H), 6.90 (ddd, 4H), 6.05 (d, 1H), 5.40-5.38 (m, 3H), 4.29 (s, 3H), 3.72 (s, 3H). |
| | 3-(3-methoxybenzyl)-5-methyl-7-vinyl- | |
| | 3H-pyridazino[4,5-b]indol-4(5H)-one | |
| 18-6 | 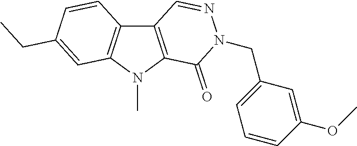 [SMILES: CCc1ccc2c3cnn(Cc4cccc(OC)c4)c(=O)c3n(C)c2c1] | LC-MS: m/z 348 (M + H)⁺. ¹H NMR (400 MHz, DMSO) δ 8.78 (s, 1H), 8.10 (d, 1H), 7.57 (s, 1H), 7.30-7.20 (m, 2H), 6.84 (m, 3H), 5.37 (s, 2H), 4.27 (s, 3H), 3.72 (s, 3H), 2.83 (q, 2H), 1.29 (t, 3H). |
| | 7-ethyl-3-(3-methoxybenzyl)-5-methyl- | |
| | 3H-pyridazino[4,5-b]indol-4(5H)-one | |
| 18-7 | 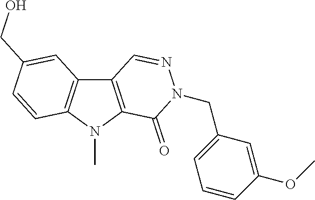 [SMILES: COc1cccc(Cn2ncc3c4cc(CO)ccc4n(C)c3c2=O)c1] | LC-MS: m/z 350 (M + H)⁺. ¹H NMR (400 MHz, DMSO) δ 8.83 (s, 1H), 8.15 (s, 1H), 7.72 (d, 1H), 7.57 (d, 1H), 7.25 (t, 1H), 6.87 (m, 3H), 5.38 (s, 2H), 5.30 (t, 1H), 4.68 (d, 2H), 4.29 (s, 3H), 3.72 (s, 3H). |
| | 8-(hydroxymethyl)-3-(3-methoxybenzyl)- | |
| | 5-methyl-3H-pyridazino[4,5-b]indol-4(5H)-one | |
| 18-8 | 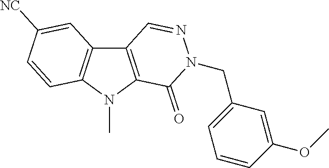 [SMILES: COc1cccc(Cn2ncc3c4cc(C#N)ccc4n(C)c3c2=O)c1] | LC-MS: m/z 345 (M + H)⁺. ¹H NMR (400 MHz, DMSO-d₆) δ 8.88 (m, 2H), 7.97 (m, 2H), 7.25 (t, 1H), 6.86 (m, 3H), 5.39 (s, 2H), 4.32 (s, 3H), 3.73 (s, 3H). |
| | 3-(3-methoxybenzyl)-5-methyl-4-oxo-4,5- | |
| | dihydro-3H-pyridazino[4,5-b]indole-8-carbonitrile | |
| 18-9 | 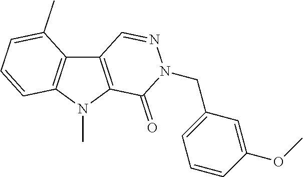 [SMILES: COc1cccc(Cn2ncc3c4c(C)cccc4n(C)c3c2=O)c1] | LC-MS: m/z 334 (M + H)⁺. ¹H NMR (400 MHz, DMSO-d₆) δ 8.75 (s, 1H), 7.59 (d, 1H), 7.54- 7.46 (m, 1H), 7.24 (t, 1H), 7.18 (d, 1H), 6.85 (dd, 3H), 5.39 (s, 2H), 4.30 (s, 3H), 3.72 (s, 3H), 2.78 (s, 3H). |
| | 3-(3-methoxybenzyl)-5,9-dimethyl-3H- | |
| | pyridazino[4,5-b]indol-4(5H)-one | |
| 18-10 | 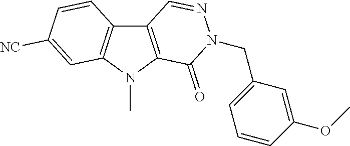 [SMILES: COc1cccc(Cn2ncc3c4ccc(C#N)cc4n(C)c3c2=O)c1] | LC-MS: m/z 345 (M + H)⁺. ¹H NMR (400 MHz, DMSO-d₆) δ 8.90 (s, 1H), 8.46 (s, 1H), 8.41 (d, 1H), 7.76 (dd, 1H), 7.23 (t, 1H), 6.88 (m, 3H), 5.38 (s, 2H), 4.32 (s, 3H), 3.72 (s, 3H) |
| | 3-(3-methoxybenzyl)-5-methyl-4-oxo-4,5- | |
| | dihydro-3H-pyridazino[4,5-b]indole-7-carbonitrile | |
| 18-11 | 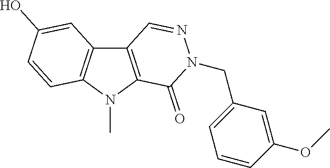 [SMILES: COc1cccc(Cn2ncc3c4cc(O)ccc4n(C)c3c2=O)c1] | LC-MS: m/z 336 (M + H)⁺. ¹H NMR (400 MHz, DMSO) δ 9.46 (s, 1H), 8.72 (s, 1H), 7.58 (d, 1H), 7.45 (d, 1H), 7.23 (t, 1H), 7.11 (dd, 1H), 6.85 (m, 3H), 5.35 (s, 2H), 4.23 (s, 3H), 3.71 (s, 3H). |
| | 8-hydroxy-3-(3-methoxybenzyl)-5-methyl- | |
| | 3H-pyridazino[4,5-b]indol-4(5H)-one | |

### Example 19. Synthesis of 3-((8-((1H-pyrazol-3-yl)sulfonyl)-5-methyl-4-oxo-4,5-dihydro-3H-pyridazino[4,5-b]indol-3-yl)methyl)benzamide

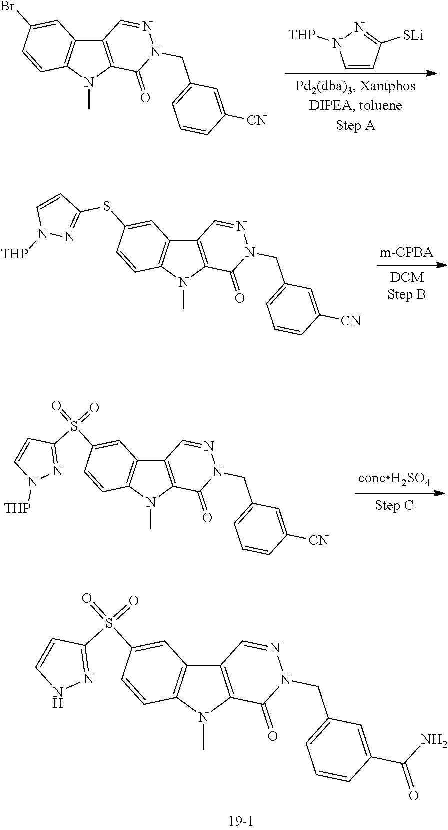

Step A. Synthesis of 3-((5-methyl-4-oxo-8-((1-(tetrahydro-2H-pyran-2-yl)-1H-pyrazol-3-yl)thio)-4,5-dihydro-3H-pyridazino[4,5-b]indol-3-yl)methyl)benzonitrile To a mixture of 3-((8-bromo-5-methyl-4-oxo-4,5-dihydro-3H-pyridazino[4,5-b]indol-3-yl)methyl)benzonitrile (200 mg, 0.51 mmol) and lithium 1-(tetrahydro-2H-pyran-2-yl)-1H-pyrazole-3-thiolate (97 mg, 0.51 mmol) in toluene (5 mL) was added DIPEA (197 mg, 1.5 mmol), Pd₂(dba)₃ (42 mg, 0.05 mmol) and Xantphos (26 mg, 0.05 mmol). The mixture was stirred at 110° C. for 2 hr. The mixture was diluted with EtOAc, washed with water and brine. The organic layer was dried over anhy. Na₂SO₄, filtered and concentrated. The residue was purified by flash chromatography (silica gel, 0-50% EtOAc in PE) to afford 200 mg of 3-((8-((1H-pyrazol-3-yl)thio)-5-methyl-4-oxo-4,5-dihydro-3H-pyridazino[4,5-b]indol-3-yl)methyl)benzonitrile. LC-MS (ESI): m/z 497 (M+H)⁺.

Step B. Synthesis of 3-((5-methyl-4-oxo-8-((1-(tetrahydro-2H-pyran-2-yl)-1H-pyrazol-3-yl)sulfonyl)-4,5-dihydro-3H-pyridazino[4,5-b]indol-3-yl)methyl)benzonitrile At 0° C., to a solution of 3-((8-((1H-pyrazol-3-yl)thio)-5-methyl-4-oxo-4,5-dihydro-3H-pyridazino[4,5-b]indol-3-yl)methyl)benzonitrile (100 mg, 0.24 mmol) in DCM (5 mL) was added m-CPBA (148 mg, 0.72 mmol, 85% wt). The suspension was stirred at r.t. for 3 hr. The reation mixture was poured into satd. Na₂S₂O₃, extracted with DCM. The organic layer was washed with brine, dried over anhy. Na₂SO₄ and concentrated. The residue was purified by prep-TLC (PE/EtOAc=1:1) to afford 60 mg of 3-((8-((1H-pyrazol-3-yl)sulfonyl)-5-methyl-4-oxo-4,5-dihydro-3H-pyridazino[4,5-b]indol-3-yl)methyl)benzonitrile. LC-MS (ESI): m/z 529 (M+H)⁺.

Step C. Synthesis of 3-((8-((1H-pyrazol-3-yl)sulfonyl)-5-methyl-4-oxo-4,5-dihydro-3H-pyridazino[4,5-b]indol-3-yl)methyl)benzamide A solution of 3-((8-((1H-pyrazol-3-yl)sulfonyl)-5-methyl-4-oxo-4,5-dihydro-3H-pyridazino[4,5-b]indol-3-yl)methyl)benzonitrile (60 mg, 0.13 mmol) in conc. H₂SO₄ (1 mL) was stirred at r.t. for overnight. The solution was poured into ice water slowly, neutralized with satd. NaHC₃, extracted with EtOAc. The combined organic layer was dried over anhy. Na₂SO₄ and concentrated. The residue was purified by prep-TLC (DCM/MeOH=10:1) to afford 10 mg of 3-((8-((1H-pyrazol-3-yl)sulfonyl)-5-methyl-4-oxo-4,5-dihydro-3H-pyridazino[4,5-b]indol-3-yl)methyl)benzamide. LC-MS (ESI): m/z 463 (M+H)⁺. ¹H NMR (400 MHz, DMSO-d₆) δ 13.76 (s, 1H), 9.08 (s, 1H), 8.99 (d, 1H), 8.06 (dd, 1H), 8.00-7.92 (m, 3H), 7.82 (s, 1H), 7.77 (d, 1H), 7.48 (d, 1H), 7.41 (t, 1H), 7.35 (s, 1H), 6.86 (d, 1H), 5.45 (s, 2H), 4.31 (s, 3H).

| Cpd No. | Structure and chemical name | Characterization |
|---|---|---|
| 19-2 | 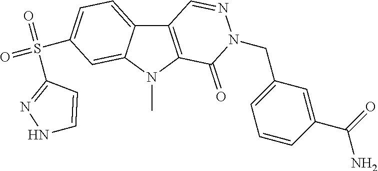 [SMILES: Cn1c2cc(S(=O)(=O)c3cc[nH]n3)ccc2c2cnn(Cc3cccc(C(N)=O)c3)c(=O)c21] | LC-MS (ESI): m/z 463 (M + H)⁺. ¹H NMR (400 MHz, DMSO-d₆) δ 13.83 (s, 1H), 8.91 (s, 1H), 8.45 (d, 1H), 8.36 (s, 1H), 7.97 (d, 2H), 7.89 (dd, 1H), 7.82 (s, 1H), 7.77 (d, 1H), 7.48 (d, 1H), 7.41 (t, 1H), 7.35 (s, 1H), 6.94 (d, 1H), 5.46 (s, 2H), 4.38 (s, 3H). |
| | 3-((7-(1H-pyrazol-3-yl)sulfonyl)-5- | |
| | methyl-4-oxo-4,5-dihydro-3H- | |
| | pyridazino[4,5-b]indol-3-yl)methyl)benzamide | |
| 19-3 | 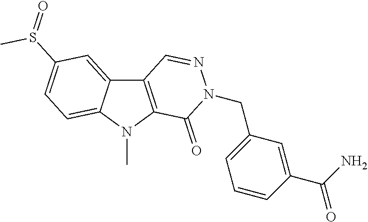 [SMILES: Cn1c2ccc(S(C)=O)cc2c2cnn(Cc3cccc(C(N)=O)c3)c(=O)c21] | LC-MS: m/z 395 (M + H)⁺. ¹H NMR (400 MHz, DMSO-d₆) δ 9.02 (s, 1H), 8.69 (dd, 1H), 8.06- 8.01 (m, 2H), 7.93 (dd, 1H), 7.88 (d, 1H), 7.83 (dt, 1H), 7.54 (dt, 1H), 7.47 (t, 1H), 7.41 (s, 1H), 5.52 (s, 2H), 4.39 (s, 3H), 2.86 (s, 3H) |
| | 3-((5-methyl-8-(methylsulfinyl)-4- | |
| | oxo-4,5-dihydro-3H-pyridazino[4,5- | |
| | b]indol-3-yl)methyl)benzamide | |
| 19-4 | 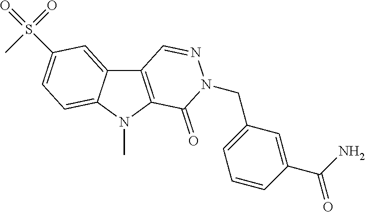 [SMILES: Cn1c2ccc(S(C)(=O)=O)cc2c2cnn(Cc3cccc(C(N)=O)c3)c(=O)c21] | LC-MS: m/z 411 (M + H)⁺. ¹H NMR (400 MHz, DMSO-d₆) δ 9.08 (s, 1H), 8.98 (d, 1H), 8.15 (dd, 1H), 8.07 (d, 1H), 8.03 (s, 1H), 7.88 (d, 1H), 7.84 (dd, 1H), 7.55 (dt, 1H), 7.47 (t, 1H), 7.41 (s, 1H), 5.52 (s, 2H), 4.39 (s, 3H), 3.32 (s, 3H) |
| | 3-((5-methyl-8-(methylsulfonyl)-4- | |
| | oxo-4,5-dihydro-3H-pyridazino[4,5- | |
| | b]indol-3-yl)methyl)benzamide | |
| 19-5 | 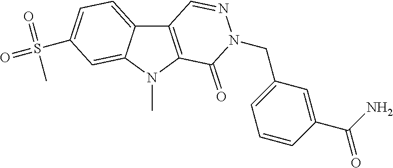 [SMILES: Cn1c2cc(S(C)(=O)=O)ccc2c2cnn(Cc3cccc(C(N)=O)c3)c(=O)c21] | LC-MS: m/z 411 (M + H)⁺. ¹H NMR (400 MHz, DMSO-d₆) δ 8.94 (s, 1H), 8.49 (d, 1H), 8.37 (s, 1H), 7.98 (s, 1H), 7.94-7.90 (m, 1H), 7.84 (s, 1H), 7.78 (d, 1H), 7.50 (d, 1H), 7.42 (t, 1H), 7.36 (s, 1H), 5.47 (s, 2H), 4.38 (s, 3H), 3.33 (s, 3H) |
| | 3-((5-methyl-7-(methylsulfonyl)-4- | |
| | oxo-4,5-dihydro-3H-pyridazino[4,5- | |
| | b]indol-3-yl)methyl)benzamide | |
| 19-6 | 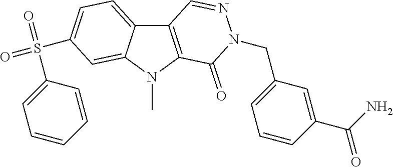 [SMILES: Cn1c2cc(S(=O)(=O)c3ccccc3)ccc2c2cnn(Cc3cccc(C(N)=O)c3)c(=O)c21] | LC-MS: m/z 473 (M + H)⁺. ¹H NMR (400 MHz, DMSO-d₆) δ 8.90 (s, 1H), 8.45 (d, 2H), 8.10- 8.04 (m, 2H), 7.96 (s, 1H), 7.90 (d, 1H), 7.83-7.75 (m, 2H), 7.66 (m, 3H), 7.42 (m, 3H), 5.45 (s, 2H), 4.39 (s, 3H) |
| | 3-((5-methyl-4-oxo-7-(phenylsulfonyl)- | |
| | 4,5-dihydro-3H-pyridazino[4,5-b] | |
| | indol-3-yl)methyl)benzamide | |
| 19-7 | 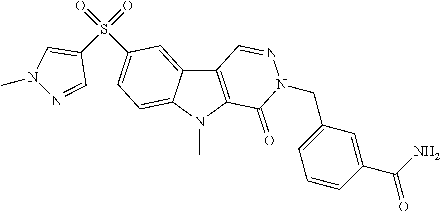 [SMILES: Cn1cc(S(=O)(=O)c2ccc3c(c2)c2cnn(Cc4cccc(C(N)=O)c4)c(=O)c2n3C)cn1] | LC-MS: m/z 477 (M + H)⁺. ¹H NMR (400 MHz, DMSO-d₆) δ 8.99 (d, 2H), 8.43 (s, 1H), 8.13- 7.88 (m, 4H), 7.80 (d, 2H), 7.57- 7.23 (m, 3H), 5.46 (s, 2H), 4.31 (s, 3H), 3.84 (s, 3H) |
| | 3-((5-methyl-8-((1-methyl-1H-pyrazol-4-yl) | |
| | sulfonyl)-4-oxo-4,5-dihydro-3H-pyridazino | |
| | [4,5-b]indol-3-yl)methyl)benzamide | |
| 19-8 | 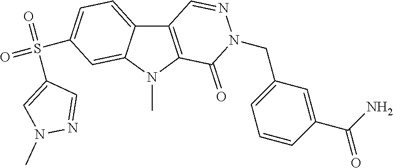 [SMILES: Cn1cc(S(=O)(=O)c2ccc3c4cnn(Cc5cccc(C(N)=O)c5)c(=O)c4n(C)c3c2)cn1] | LC-MS: m/z 477 (M + H)⁺. ¹H NMR (400 MHz, DMSO-d₆) δ 8.90 (s, 1H), 8.51 (s, 1H), 8.43 (d, 1H), 8.35 (d, 1H), 8.01 (s, 1H), 7.98 (s, 1H), 7.89 (dd, 1H), 7.82 (s, 1H), 7.77 (d, 1H), 7.48 (d, 1H), 7.41 (t, 1H), 7.35 (s, 1H), 5.45 (s, 2H), 4.37 (s, 3H), 3.86 (s, 3H) |
| | 3-((5-methyl-7-((1-methyl-1H- | |
| | pyrazol-4-yl)sulfonyl)-4-oxo-4,5- | |
| | dihydro-3H-pyridazino[4,5-b]indol-3- | |
| | yl)methyl)benzamide | |
| 19-9 | 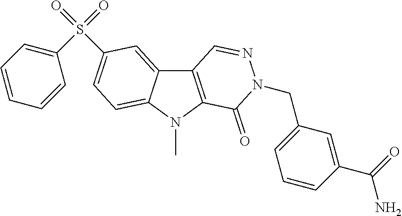 [SMILES: Cn1c2ccc(S(=O)(=O)c3ccccc3)cc2c2cnn(Cc3cccc(C(N)=O)c3)c(=O)c21] | LC-MS: m/z 473 (M + H)⁺. ¹H NMR (400 MHz, DMSO-d₆) δ 9.14-9.86 (m, 2H), 8.12-7.90 (m, 5H), 7.82 (s, 1H), 7.78 (d, 1H), 7.70-7.58 (m, 3H), 7.52-7.30 (m, 3H), 5.44 (s, 2H), 4.28 (s, 3H) |
| | 3-((5-methyl-4-oxo-8-(phenylsulfonyl)- | |
| | 4,5-dihydro-3H-pyridazino[4,5-b] | |
| | indol-3-yl)methyl)benzamide | |

### Example 20. Synthesis of 5-methyl-3-((1-methyl-1H-pyrazol-3-yl)methyl)-7-(thiazol-4-ylmethyl)-3H-pyridazino[4,5-b]indol-4 (5H)-one

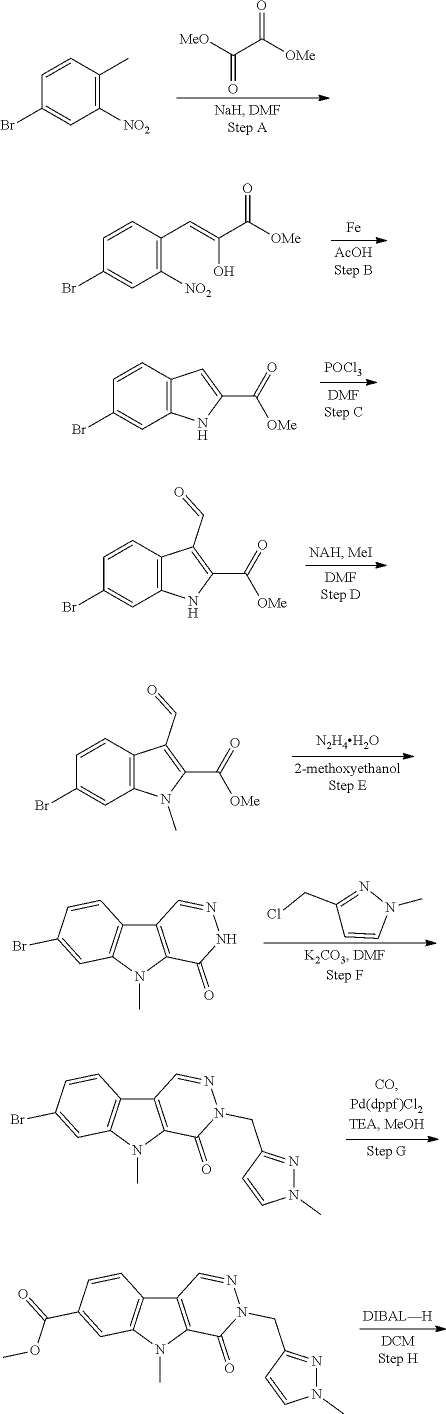

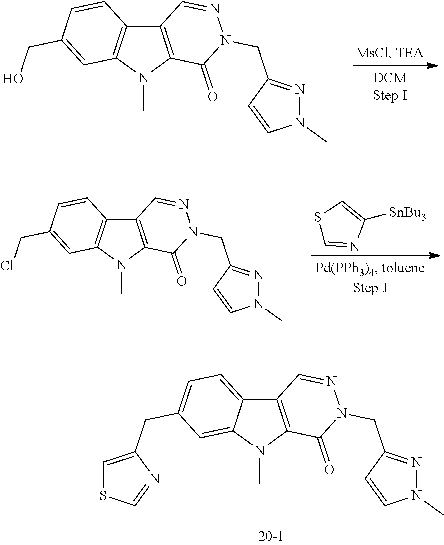

Step A. Synthesis of methyl 3-(4-bromo-2-nitrophenyl)-2-hydroxyacrylate To a suspension of NaH (7.4 g, 60% in oil, 184 mmol) in dry DMF (100 mL) was added a solution of 4-bromo-1-methyl-2-nitrobenzene (10 g, 46 mmol) and dimethyl oxalate (21.6 g, 184 mmol) in dry DMF (60 mL) at 0° C. The mixture was stirred at 40° C. for 1 hr. The reaction mixture was quenched with satd. NH₄Cl, extracted with EtOAc. The combined organic layer was dried over anhy. Na₂SO₄ and concentrated to give 15 g of crude methyl 3-(4-bromo-2-nitrophenyl)-2-hydroxyacrylate which was used in next step without further purification. LC-MS (ESI): m/z 302 (M+H)⁺.

Step B. Synthesis of methyl 6-bromo-1H-indole-2-carboxylate To a solution of methyl 3-(4-bromo-2-nitrophenyl)-2-hydroxyacrylate (15 g, crude) in AcOH (150 mL) was added Fe (7.7 g, 138 mmol) by portions at 90° C. After addition, the mixture was stirred at 90° C. for 0.5 hr. The reaction mixture was poured into water. The precipitate was collected by filtration and purified by silica gel chromatography (eluted with PE/EtOAc=5/1) to give 2 g of methyl 6-bromo-1H-indole-2-carboxylate. LC-MS (ESI): m/z 254 (M+H)⁺.

Step C. Synthesis of methyl 6-bromo-3-formyl-1H-indole-2-carboxylate To a solution of methyl 6-bromo-1H-indole-2-carboxylate (2 g, 8 mmol) in anhy. DMF (20 mL) was added phosphorus oxychloride (2.4 g, 16 mmol). The reaction mixture was stirred at 100° C. for 2 hr. The reaction mixture was poured into ice-water. The precipitate was collected by filtration to give 1.5 g of methyl 6-bromo-3-formyl-1H-indole-2-carboxylate. LC-MS (ESI): m/z 282 (M+H)⁺.

Step D. Synthesis of methyl 6-bromo-3-formyl-1-methyl-1H-indole-2-carboxylate To a solution of methyl 6-bromo-3-formyl-1H-indole-2-carboxylate (1.5 g, 5.1 mmol) in anhy. DMF (20 mL) was added NaH (400 mg, 60% in oil, 10 mmol). After stirred at r.t. for 15 min, MeI (1 g, 7.7 mmol) was added, the reaction mixture was stirred for another 2 hr. The mixture was quenched with satd. NH₄Cl. The precipitate was collected by filtration to give 700 mg of methyl 6-bromo-3-formyl-1-methyl-1H-indole-2-carboxylate. LC-MS (ESI): m/z 296 (M+H)⁺.

Step E. Synthesis of 7-bromo-5-methyl-3H-pyridazino[4,5-b]indol-4 (5H)-one To a solution of methyl 6-bromo-3-formyl-1-methyl-1H-indole-2-carboxylate (700 mg, 2.5 mmol) in 2-methoxyethanol (10 mL) was added hydrazine hydrate (2 mL, 98%). The reaction mixture was stirred at 110° C. for 1 hr and cooled down. The precipitate was collected by filtration, washed with MeOH to give 500 mg of 7-bromo-5-methyl-3H-pyridazino[4,5-b]indol-4 (5H)-one. LC-MS (ESI): m/z 278 (M+H)⁺.

Step F. Synthesis of 7-bromo-5-methyl-3-((1-methyl-1H-pyrazol-3-yl)methyl)-3H-pyridazino[4,5-b]indol-4 (5H)-one To a mixture of ethyl 7-bromo-5-methyl-3H-pyridazino[4,5-b]indol-4 (5H)-one (500 mg, 1.8 mmol) in DMF (10 mL) was added K₂CO₃ (406 mg, 3.6 mmol). After stirred at 70° C. for 1.5 h, 3-(chloromethyl)-1-methyl-1H-pyrazole (157 mg, 1.2 mmol) was added, the mixture was stirred for another 1 hr. The reaction mixture was poured into satd. NH₄Cl, extracted with EtOAc. The organic layer was dried over anhy. Na₂SO₄ and concentrated. The residue was purified by flash chromatography (silica gel, 0˜40% EtOAc in PE) to give 220 mg of 7-bromo-5-methyl-3-((1-methyl-1H-pyrazol-3-yl)methyl)-3H-pyridazino[4,5-b]indol-4 (5H)-one. LC-MS (ESI): m/z 372 (M+H)⁺.

Step G. Synthesis of methyl 5-methyl-3-((1-methyl-1H-pyrazol-3-yl)methyl)-4-oxo-4,5-dihydro-3H-pyridazino[4,5-b]indole-7-carboxylate To a mixture of 7-bromo-5-methyl-3-((1-methyl-1H-pyrazol-3-yl)methyl)-3H-pyridazino[4,5-b]indol-4 (5H)-one (220 mg, 0.6 mmol) in MeOH (2 mL) and DMF (2 mL) was added TEA (2 mL) and Pd(dppf)Cl₂ (45 mg, 0.06 mmol). The mixture was stirred under CO at 100° C. overnight. The reaction mixture was diluted with H₂O and extracted with EtOAc. The combined organic phase was evaporated under reduced pressure. The residue was purified by flash chromatography (silica gel, 0˜ 50% EtOAc in PE) to give 80 mg of methyl 5-methyl-3-((1-methyl-1H-pyrazol-3-yl)methyl)-4-oxo-4,5-dihydro-3H-pyridazino[4,5-b]indole-7-carboxylate. LC-MS (ESI): m/z 352 (M+H)⁺.

Step H. Synthesis of 7-(hydroxymethyl)-5-methyl-3-((1-methyl-1H-pyrazol-3-yl)methyl)-3H-pyridazino[4,5-b]indol-4 (5H)-one. To a mixture of methyl 5-methyl-3-((1-methyl-1H-pyrazol-3-yl)methyl)-4-oxo-4,5-dihydro-3H-pyridazino[4,5-b]indole-7-carboxylate (80 mg, 0.23 mmol) in DCM (5 mL) was added DIBAL-H (0.5 mL, 1.3 M in toluene, 0.69 mmol) at 0° C. After stirred for 1.5 hr, the reaction mixture was quenched with satd. NH₄Cl, extracted with DCM. The organic phase was washed with brine, dried over anhy. Na₂SO₄ and concentrated. The residue was purified by prep-TLC (15% MeOH in DCM) to give 7-(hydroxymethyl)-5-methyl-3-((1-methyl-1H-pyrazol-3-yl)methyl)-3H-pyridazino[4,5-b]indol-4 (5H)-one (20 mg, 27.2% yield) as a yellow solid. LC-MS (ESI): m/z 324 (M+H)⁺.

Step I. Synthesis of 7-(chloromethyl)-5-methyl-3-((1-methyl-1H-pyrazol-3-yl)methyl)-3H-pyridazino[4,5-b]indol-4 (5H)-one To a mixture of 7-(hydroxymethyl)-5-methyl-3-((1-methyl-1H-pyrazol-3-yl)methyl)-3H-pyridazino[4,5-b]indol-4 (5H)-one (20 mg, 0.06 mmol) in DCM (3 mL) was added TEA (20 mg, 0.2 mmol) and methane sulfonyl chloride (12 mg, 0.1 mmol) at 0° C. After stirred for 1.5 hr, the reaction mixture was diluted with DCM, washed with water and brine. The organic phase was dried over anhy. Na₂SO₄ and evaporated under reduced pressure. The residue was purified by pre-TLC (15% MeOH in DCM) to give 10 mg of 7-(chloromethyl)-5-methyl-3-((1-methyl-1H-pyrazol-3-yl)methyl)-3H-pyridazino[4,5-b]indol-4 (5H)-one. LC-MS (ESI): m/z 341 (M+H)⁺.

Step J. Synthesis of 5-methyl-3-((1-methyl-1H-pyrazol-3-yl)methyl)-7-(thiazol-4-ylmethyl)-3H-pyridazino[4,5-b]indol-4 (5H)-one. To a mixture of 7-(chloromethyl)-5-methyl-3-((1-methyl-1H-pyrazol-3-yl)methyl)-3H-pyridazino[4,5-b]indol-4 (5H)-one (10 mg, 0.03 mmol) in toluene (3 mL) was added Pd(PPh₃)₄ (6 mg) and 4-(tributylstannyl)-1,3-thiazole (33 mg, 0.09 mmol) under N₂. The reaction mixture was stirred at 100° C. for 1.5 hr and concentrated. The residue was purified by pre-TLC (20% MeOH in DCM) to give 4 mg of 5-methyl-3-((1-methyl-1H-pyrazol-3-yl)methyl)-7-(thiazol-4-ylmethyl)-3H-pyridazino[4,5-b]indol-4 (5H)-one. LC-MS (ESI): m/z 391 (M+H)⁺. ¹H NMR (400 MHz, DMSO-d₆) δ 9.04 (d, 1H), 8.73 (s, 1H), 8.10 (d, 1H), 7.66 (s, 1H), 7.56 (d, 1H), 7.40 (s, 1H), 7.31 (d, 1H), 6.09 (d, 1H), 5.32 (s, 2H), 4.33 (s, 2H), 4.25 (s, 3H), 3.77 (s, 3H).

| Cpd No. | Structure and chemical name | Characterization |
|---|---|---|
| 20-2 | 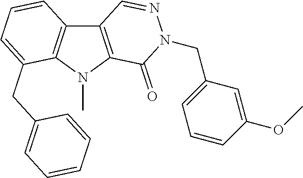 [SMILES: COc1cccc(Cn2ncc3c4cccc(Cc5ccccc5)c4n(C)c3c2=O)c1] | LC-MS: m/z 410 (M + H)⁺. ¹H NMR (400 MHz, CDCl3) δ 8.44 (s, 1H), 7.87 (dd, 1H), 7.26- 7.10 (m, 6H), 6.98-6.91 (m, 3H), 6.89 (s, 1H), 6.72 (dd, 1H), 5.37 (s, 2H), 4.52 (s, 2H), 4.38 (s, 3H), 3.70 (s, 3H). |
| | 6-benzyl-3-(3-methoxybenzyl)-5-methyl- | |
| | 3,5-dihydro-4H-pyridazino[4,5-b]indol-4-one | |
| 20-3 | 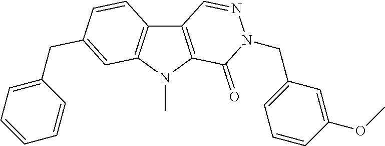 [SMILES: COc1cccc(Cn2ncc3c4ccc(Cc5ccccc5)cc4n(C)c3c2=O)c1] | LC-MS: m/z 410 (M + H)⁺. ¹H NMR (400 MHz, DMSO-d₆) δ8.77 (s, 1H), 8.10 (d, 1H), 7.67 (s, 1H), 7.31-7.17 (m, 7H), 6.88- 6.82 (m, 3H), 5.37 (s, 2H), 4.26 (s, 3H), 4.16 (s, 2H), 3.71 (s, 3H). |
| | 7-benzyl-3-(3-methoxybenzyl)-5-methyl- | |
| | 3H-pyridazino[4,5-b]indol-4(5H)-one | |
| 20-4 | 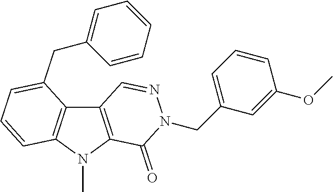 [SMILES: COc1cccc(Cn2ncc3c4c(Cc5ccccc5)cccc4n(C)c3c2=O)c1] | LC-MS: m/z 410 (M + H)⁺. ¹H NMR (400 MHz, DMSO-d₆) δ 8.54 (s, 1H), 7.77-7.53 (m, 2H), 7.38-7.12 (m, 7H), 6.94-6.76 (m, 3H), 5.33 (s, 2H), 4.55 (s, 2H), 4.31 (s, 3H), 3.70 (s, 3H) |
| | 9-benzyl-3-(3-methoxybenzyl)-5-methyl- | |
| | 3H-pyridazino[4,5-b]indol-4 (5H)-one | |
| 20-5 | 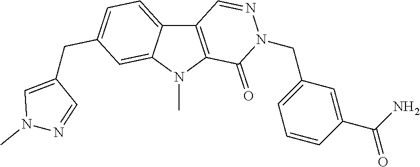 [SMILES: Cn1cc(Cc2ccc3c4cnn(Cc5cccc(C(N)=O)c5)c(=O)c4n(C)c3c2)cn1] | LC-MS: m/z 427 (M + H)⁺. ¹H NMR (400 MHz, DMSO-d₆) δ 8.79 (s, 1H), 8.10 (d, 1H), 7.97 (s, 1H), 7.81 (s, 1H), 7.76 (d, 1H), 7.59 (s, 1H), 7.49 (s, 1H), 7.46 (d, 1H), 7.40 (t, 1H), 7.34 (s, 1H), 7.29 (s, 1H), 7.26 (dd, 1H), 5.44 (s, 2H), 4.25 (s, 3H), 3.96 (s, 2H), 3.76 (s, 3H) |
| | 3-((5-methyl-7-((l-methyl-1H-pyrazol-4-yl)methyl)- | |
| | 4-oxo-4,5-dihydro-3H-pyridazino[4,5-b]indol-3-yl)methyl)benzamide | |

### Example 21. Synthesis of 3-(3-methoxybenzyl)-5-methyl-8-(trifluoromethyl)-3H-pyridazino[4,5-b]indol-4 (5H)-one

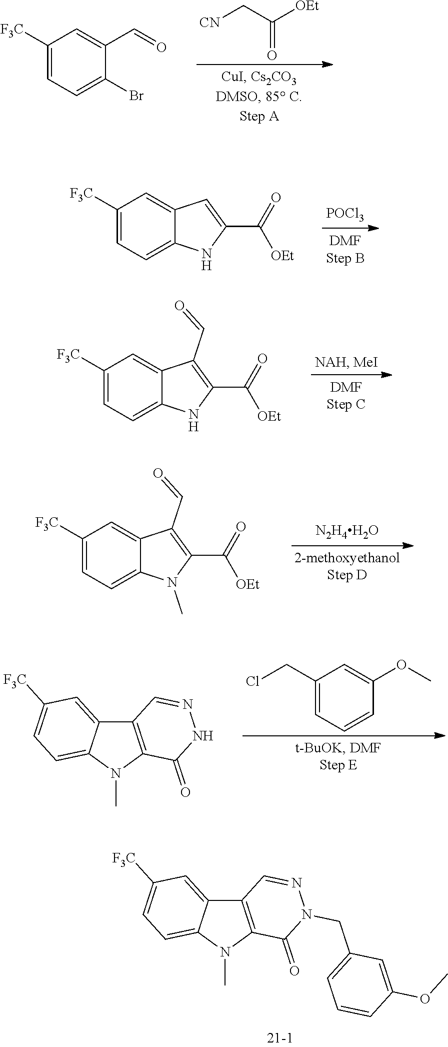

Step A. Synthesis of ethyl 5-(trifluoromethyl)-1H-indole-2-carboxylate To a stirred mixture of 2-bromo-5-(trifluoromethyl)benzaldehyde (1 g, 4 mmol) and ethyl 2-isocyanoacetate (494 mg, 4.4 mmol) in DMSO (30 mL) was added Cs₂CO₃ (2.6 g, 8 mmol) and CuI (76 mg, 0.4 mmol). The reaction mixture was stirred under N₂ at 85° C. overnight. The reaction mixture was poured into water, extracted with EtOAc. The organic layer was dried over anhy. Na₂SO₄ and concentrated. The residue was purified by silica gel chromatography to give 650 mg of ethyl 5-(trifluoromethyl)-1H-indole-2-carboxylate. LC-MS (ESI): m/z 258 (M+H)⁺.

Step B. Synthesis of ethyl 3-formyl-5-(trifluoromethyl)-1H-indole-2-carboxylate To a stirred mixture of ethyl 5-(trifluoromethyl)-1H-indole-2-carboxylate (650 mg, 2.5 mmol) in dry DMF (5 mL) was added POCl₃ (1.5 g, 10 mmol) by dropwise at 0° C. The reaction mixture was stirred at 100° C. under N₂ overnight. The reaction mixture was poured into satd. NaHCO₃, extracted with EtOAc. The organic layer was dried over anhy. Na₂SO₄ and concentrated. The residue was purified by silica gel chromatography to give 880 mg of ethyl 3-formyl-5-(trifluoromethyl)-1H-indole-2-carboxylate. LC-MS (ESI): m/z 286 (M+H)⁺.

Step C. Synthesis of ethyl 3-formyl-1-methyl-5-(trifluoromethyl)-1H-indole-2-carboxylate To a stirred mixture of ethyl 3-formyl-5-(trifluoromethyl)-1H-indole-2-carboxylate (480 mg, 1.7 mmol) in dry DMF (5 mL) was added NaH (136 mg, 3.4 mmol) at 0° C. After stirred for 15 min, MeI (480 mg, 2.5 mmol) was added. The reaction mixture was stirred at r.t. for 2 hr, poured into satd. NH₄Cl, extracted with EtOAc. The organic layer was dried over anhy. Na₂SO₄ and concentrated. The residue was purified by silica gel chromatography to give 350 mg of ethyl 3-formyl-1-methyl-5-(trifluoromethyl)-1H-indole-2-carboxylate. LC-MS (ESI): m/z 300 (M+H)⁺.

Step D. Synthesis of 5-methyl-8-(trifluoromethyl)-3H-pyridazino[4,5-b]indol-4 (5H)-one To a stirred mixture of ethyl 3-formyl-1-methyl-5-(trifluoromethyl)-1H-indole-2-carboxylate (350 mg, 1.2 mmol) in 2-methoxyethanol (5 mL) was added N₂H4′H₂O (344 mg, 6 mmol, 85% w/w). The reaction mixture was stirred at 100° C. overnight. The reaction mixture was concentrated and washed with water to give 5-methyl-8-(trifluoromethyl)-3H-pyridazino[4,5-b]indol-4 (5H)-one 260 mg. LC-MS (ESI): m/z 268 (M+H)⁺.

Step E. Synthesis of 3-(3-methoxybenzyl)-5-methyl-8-(trifluoromethyl)-3H-pyridazino[4,5-b]indol-4 (5H)-one To a stirred mixture of 5-methyl-8-(trifluoromethyl)-3H-pyridazino[4,5-b]indol-4 (5H)-one (100 mg, 0.37 mmol) in DMF (5 mL) was added t-BuOK (125 mg, 1.11 mmol) and 1-(chloromethyl)-3-methoxybenzene (115 mg, 0.74 mmol). The reaction mixture was stirred at r.t. for 2 hr. The reaction mixture was poured into satd. NH₄Cl, extracted with DCM. The organic layer was dried over anhy. Na₂SO₄ and concentrated. The residue was purified by prep-HPLC to give 3-(3-methoxybenzyl)-5-methyl-8-(trifluoromethyl)-3H-pyridazino[4,5-b]indol-4 (5H)-one (50 mg). LC-MS (ESI): m/z 388 (M+H)⁺. ¹H NMR (400 MHz, DMSO-d₆) δ 8.57 (s, 1H), 8.33 (s, 1H), 7.83 (dd, 1H), 7.64 (d, 1H), 7.27 (d, 1H), 7.07 (d, 1H), 7.03 (t, 1H), 6.85 (dd, 1H), 5.51 (s, 2H), 4.42 (s, 3H), 3.82 (s, 3H).

### Example 22. Synthesis of 3-(3-methoxybenzyl)-5-methyl-3H-pyrido[4′,3′:4,5]pyrrolo[2,3-d]pyridazin-4 (5H)-one

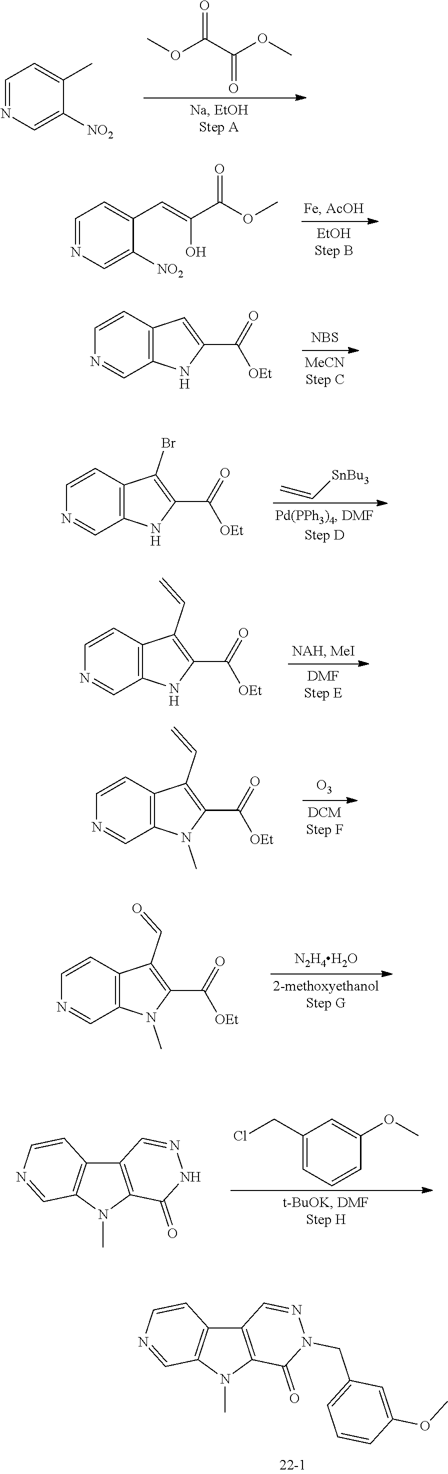

Step A. Synthesis of methyl 2-hydroxy-3-(3-nitropyridin-4-yl)acrylate To a cooled solution of sodium (1.9 g, 84.7 mmol) in absolute EtOH (40 mL) was added a mixture of 4-methyl-3-nitropyridine (4 g, 29 mmol) and dimethyl oxalate (10 g, 84.7 mmol) by dropwise. After stirred at 40° C. for 1 hr, the mixture was poured into satd. NH₄Cl, extracted with EtOAc. The combined organic layer was dried over anhy. Na₂SO₄ and concentrated. The residue was purified by silica gel chromatography (eluted with PE:EtOAc=10:1 to 2:1) to afford methyl 2-hydroxy-3-(3-nitropyridin-4-yl)acrylate 6 g. LC-MS (ESI): m/z 225 (M+H)⁺.

Step B. Synthesis of ethyl 1H-pyrrolo[2,3-c]pyridine-2-carboxylate. To a mixture of methyl 2-hydroxy-3-(3-nitropyridin-4-yl)acrylate (6 g, 26.7 mmol) in EtOH (50 mL) and HOAc (10 mL) was added Fe powder (7.56 g, 135 mmol). The reaction mixture was stirred at 70° C. for 2 hr, filtered through a pad of celite. The filtrate was poured into satd. NaHCO₃, extracted with DCM. The combined organic layer was dried over anhy. Na₂SO₄ and concentrated. The residue was purified by silica gel chromatography (eluted with PE:EtOAc=10:1 to 2:1) to afford ethyl 1H-pyrrolo[2,3-c]pyridine-2-carboxylate 2.4 g. LC-MS (ESI): m/z 191 (M+H)⁺.

Step C. Synthesis of ethyl 3-bromo-1H-pyrrolo[2,3-c]pyridine-2-carboxylate. A solution of ethyl 1H-pyrrolo[2,3-c]pyridine-2-carboxylate (2.4 g, 12.6 mmol) in MeCN (25 mL) was added NBS (2.7 g, 15.1 mmol). The reaction mixture was stirred at r.t. for 0.5 hr. The mixture was diluted with EtOAc, washed with water and brine. The organic layer was dried over anhy. Na₂SO₄ and concentrated. The residue was purified by silica gel chromatography (eluted with PE:EtOAc=10:1 to 2:1) to afford 1 g of ethyl 3-bromo-1H-pyrrolo[2,3-c]pyridine-2-carboxylate. LC-MS (ESI): m/z 269 (M+H)⁺.

Step D. Synthesis of ethyl 3-vinyl-1H-pyrrolo[2,3-c]pyridine-2-carboxylate. To a mixture of ethyl 3-bromo-1H-pyrrolo[2,3-c]pyridine-2-carboxylate (360 mg, 1.3 mmol) and Pd(PPh₃)₄ (154 mg, 0.13 mmol) in DMF (6 mL) was added tributyl(vinyl)stannane (1.2 mL, 4 mmol) via syringe under N₂. The reaction mixture was stirred under N₂ at 100° C. for 12 hr. The mixture was poured into water, extracted with EtOAc. The organic layer was dried over anhy. Na₂SO₄ and concentrated. The residue was purified by silica gel chromatography (eluted with PE:EtOAc=10:1 to 2:1) to afford 200 mg of ethyl 3-vinyl-1H-pyrrolo[2,3-c]pyridine-2-carboxylate. LC-MS (ESI): m/z 217 (M+H)⁺.

Step E. Synthesis of ethyl 1-methyl-3-vinyl-1H-pyrrolo[2,3-c]pyridine-2-carboxylate To a mixture of ethyl 3-vinyl-1H-pyrrolo[2,3-c]pyridine-2-carboxylate (200 mg, 0.93 mmol) in DMF (2 mL) was added NaH (150 mg, 3.75 mmol). After stirred at r.t. for 10 min, iodomethane (131 mg, 0.93 mmol) was added and stirred for another 30 min. The reaction mixture was poured into satd. NH₄Cl, extracted with EtOAc. The organic layer was dried over anhy. Na₂SO₄ and concentrated. The residue was purified by silica gel chromatography (eluted with PE:EtOAc=1:1) to afford ethyl 1-methyl-3-vinyl-1H-pyrrolo[2,3-c]pyridine-2-carboxylate (145 mg, 68.3% yield) as an oil. LC-MS (ESI): m/z 231 (M+H)⁺.

Step F. Synthesis of ethyl 3-formyl-1-methyl-1H-pyrrolo[2,3-c]pyridine-2-carboxylate An ozone-enriched stream of oxygen was bubbled through a cold solution of ethyl 1-methyl-3-vinyl-1H-pyrrolo[2,3-c]pyridine-2-carboxylate (145 mg, 0.63 mmol) in DCM (5 mL) at −78° C. until the colour turned light blue. The solution was quenched with dimethyl sulfide at −78° C. The mixture was concentrated under reduced pressure to afford 100 mg of ethyl 3-formyl-1-methyl-1H-pyrrolo[2,3-c]pyridine-2-carboxylate which was used in the next step without further purification. LC-MS (ESI): m/z 233 (M+H)⁺.

Step G. Synthesis of 5-methyl-3H-pyrido[4′,3′: 4,5]pyrrolo[2,3-d]pyridazin-4 (5H)-one A mixture of ethyl 3-formyl-1-methyl-1H-pyrrolo[2,3-c]pyridine-2-carboxylate (100 mg, 0.43 mmol) and hydrazine hydrate (0.5 mL, 98% w/w) in 2-methoxyethanol (0.5 mL) was stirred at 100° C. for 2 hr. The mixture was filtered and the filter cake was washed with PE, dried in vacuum to afford 5-methyl-3H-pyrido[4′,3′:4,5]pyrrolo[2,3-d]pyridazin-4 (5H)-one (80 mg, crude). LC-MS (ESI): m/z 201 (M+H)⁺.

Step H. Synthesis of 3-(3-methoxybenzyl)-5-methyl-3H-pyrido[4′,3′:4,5]pyrrolo[2,3-d]pyridazin-4 (5H)-one To a mixture of 5-methyl-3H-pyrido[4′,3′:4,5]pyrrolo[2,3-d]pyridazin-4 (5H)-one (80 mg, 0.4 mmol) in DMF (1 mL) was added t-BuOK (80 mg, 0.5 mmol) and 1-(chloromethyl)-3-methoxybenzene (56 mg, 0.5 mmol). After stirred at r.t. for 10 min, the mixture was poured into satd. NH₄Cl, extracted with EtOAc. The organic layer was dried over anhy. Na₂SO₄ and concentrated. The residue was purified by prep-TLC (DCM:MeOH=30:1) to afford 10 mg of 3-(3-methoxybenzyl)-5-methyl-3H-pyrido[4′,3′:4,5]pyrrolo[2,3-d]pyridazin-4 (5H)-one. LC-MS (ESI): m/z 321 (M+H)⁺. ¹H NMR (400 MHz, DMSO-d₆) δ 9.24 (s, 1H), 8.88 (s, 1H), 8.53 (d, 1H), 8.19 (d, 1H), 7.25 (t, 1H), 6.83-6.92 (m, 3H), 5.39 (s, 2H), 4.39 (s, 3H), 3.73 (s, 3H).

| Cpd No. | Structure and chemical name | Characterization |
|---|---|---|
| 22-2 | 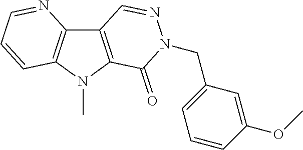 [SMILES: COc1cccc(Cn2ncc3c4ncccc4n(C)c3c2=O)c1] | LC-MS: m/z 321 (M + H)⁺. ¹H NMR (400 MHz, DMSO- d₆) δ 8.76 (s, 1H), 8.71 (dd, 1H), 8.28 (dd, 1H), 7.62 (dd, 1H), 7.25 (t,1H), 6.92-6.83 (m, 3H), 5.40 (s, 2H), 4.31 (s, 3H), 3.73 (s, 3H). |
| | 7-(3-methoxybenzyl)-5-methyl-5H- | |
| | pyrido[2′,3′:4,5]pyrrolo[2,3-d]pyridazin-6(7H)-one | |
| 22-3 | 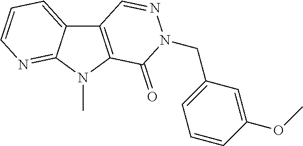 [SMILES: COc1cccc(Cn2ncc3c4cccnc4n(C)c3c2=O)c1] | LC-MS: m/z 321 (M + H)⁺. ¹H NMR (400 MHz, CDCl3) δ 8.64 (d, 1H), 8.44 (s, 1H), 8.38 (d, 1H), 7.38 (d, 1H), 7.17 (d, 1H), 7.00-6.92 (m, 2H), 6.75 (dd, 1H), 5.41 (s, 2H), 4.44 (s, 3H), 3.72 (s, 3H). |
| | 7-(3-methoxybenzyl)-9-methyl-7H- | |
| | pyrido[3′,2′:4,5]pyrrolo[2,3-d]pyridazin-8(9H)-one | |

### Example 23. Synthesis of 5-methyl-3-((1-methyl-1H-pyrazol-3-yl)methyl)-8-(thiazol-4-ylmethyl)-3H-pyrido[4′,3′:4,5]pyrrolo[2,3-d]pyridazin-4 (5H)-one

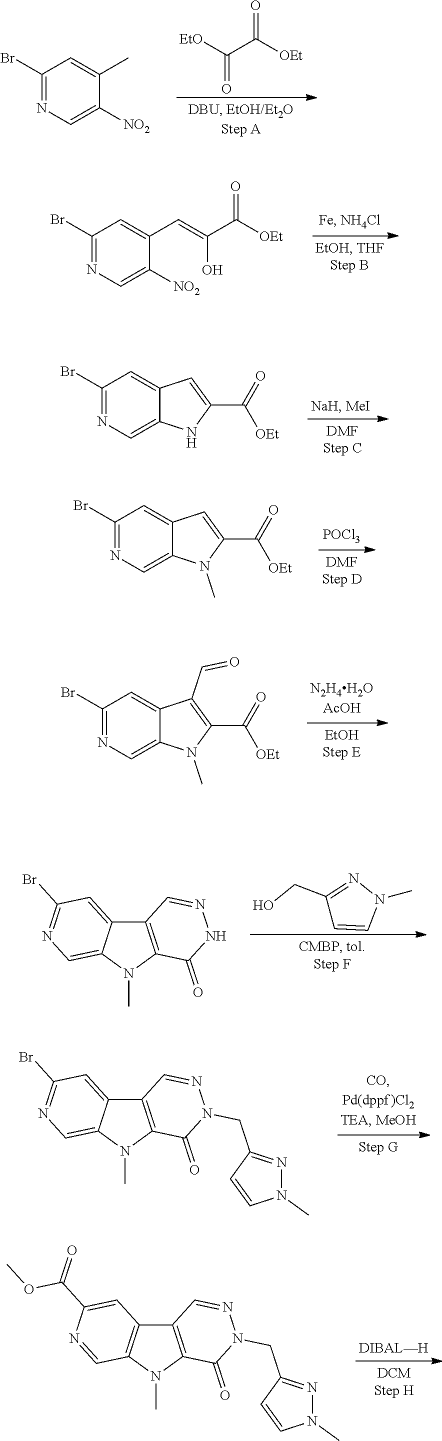

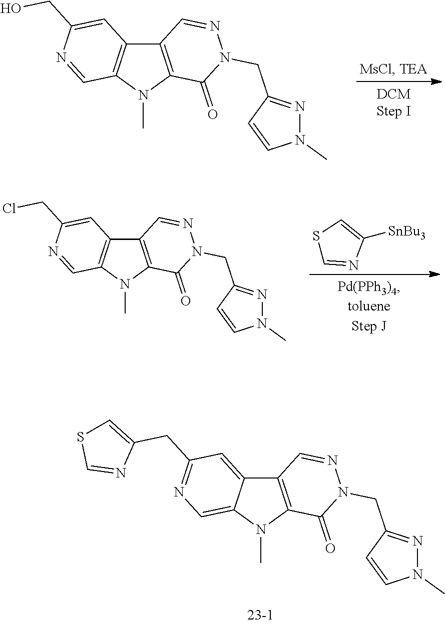

Step A. Synthesis of ethyl 3-(2-bromo-5-nitropyridin-4-yl)-2-hydroxyacrylate To a mixture of 2-bromo-4-methyl-5-nitropyridine (10 g, 46.5 mmol) in EtOH (100 mL) and Et₂O (100 mL) was added DBU (7.7 g, 51.2 mmol) and diethyl oxalate (33.7 g, 232.5 mmol). The reaction mixture was stirred at r.t. for 2 hr. The mixture was diluted with water and extracted with EtOAc. The combined organic layer was dried over anhy. Na₂SO₄ and concentrated. The residue was purified by flash chromatography (silicagel, 0˜ 30% EtOAc in PE) to give (Z)-ethyl 3-(2-bromo-5-nitropyridin-4-yl)-2-hydroxyacrylate (10 g). LC-MS (ESI): m/z 317 (M+H)⁺.

Step B. Synthesis of ethyl 5-bromo-1H-pyrrolo[2,3-c]pyridine-2-carboxylate To a mixture of (Z)-ethyl 3-(2-bromo-5-nitropyridin-4-yl)-2-hydroxyacrylate (10 g, 33.3 mmol) in EtOH (100 mL) and THF (100 mL) was added NH₄Cl (17.9 g, 332 mmol) and Fe (18.2 g, 332 mmol). The reaction mixture was stirred at 100° C. for 1.5 hr. The mixture was diluted with water and extracted with EtOAc. The combined organic layer was dried over anhy. Na₂SO₄ and concentrated. The residue was purified by flash chromatography (silica gel, 0˜ 10% EtOAc in PE) to give ethyl 5-bromo-1H-pyrrolo[2,3-c]pyridine-2-carboxylate (8 g). LC-MS (ESI): m/z 269 (M+H)⁺.

Step C. Synthesis of ethyl 5-bromo-1-methyl-1H-pyrrolo[2,3-c]pyridine-2-carboxylate To a mixture of ethyl 5-bromo-1H-pyrrolo[2,3-c]pyridine-2-carboxylate (800 mg, 2.9 mmol) in DMF (8 mL) was added NaH (236 mg, 5.9 mmol) at 0° C. After stirred for 0.5 hr, MeI (0.2 mL, 2.9 mmol) was added, the reaction mixture was stirred for another 1 hr. The reaction mixture was poured into satd. NH₄Cl, extracted with EtOAc. The combined organic layer was dried over anhy. Na₂SO₄ and concentrated. The residue was purified by flash chromatography (silicagel, 0˜ 50% EtOAc in PE) to give ethyl 5-bromo-1-methyl-1H-pyrrolo[2,3-c]pyridine-2-carboxylate (200 mg). LC-MS (ESI): m/z 283 (M+H)⁺.

Step D. Synthesis of ethyl 5-bromo-3-formyl-1-methyl-1H-pyrrolo[2,3-c]pyridine-2-carboxylate To a mixture of ethyl 5-bromo-1-methyl-1H-pyrrolo[2,3-c]pyridine-2-carboxylate (200 mg, 0.7 mmol) in DMF (5 mL) was added phosphoroyl trichloride (0.3 mL, 3.5 mmol). The mixture was stirred at 100° C. overnight. The reaction mixture was poured into satd. NaHCO₃ and extracted with EtOAc. The combined organic layer was dried over anhy. Na₂SO₄ and concentrated. The residue was purified by flash chromatography (silica gel, 0˜ 20% EtOAc in PE) to give ethyl 5-bromo-3-formyl-1-methyl-1H-pyrrolo[2,3-c]pyridine-2-carboxylate (180 mg). LC-MS (ESI): m/z 311 (M+H)⁺.

Step E. Synthesis of 8-bromo-5-methyl-3H-pyrido[4′,3′:4,5]pyrrolo[2,3-d]pyridazin-4 (5H)-one To a mixture of ethyl 5-bromo-3-formyl-1-methyl-1H-pyrrolo[2,3-c]pyridine-2-carboxylate (500 mg, 1.6 mmol) in EtOH (10 mL) was added acetic acid (0.1 mL, 1.6 mmol) and hydrazine hydrate (80 mg, 1.6 mmol). The mixture was stirred at 100° C. for 1 hr. The precipitate was collected by filtration and washed with EtOH to give 8-bromo-5-methyl-3H-pyrido[4′,3′:4,5]pyrrolo[2,3-d]pyridazin-4 (5H)-one (160 mg). LC-MS (ESI): m/z 279 (M+H)⁺.

Step F. Synthesis of 8-bromo-5-methyl-3-((1-methyl-1H-pyrazol-3-yl)methyl)-3H-pyrido[4′,3′:4,5]pyrrolo[2,3-d]pyridazin-4 (5H)-one A three-neck round bottom flask was charged with 8-bromo-5-methyl-3H-pyrido[4′,3′:4,5]pyrrolo[2,3-d]pyridazin-4 (5H)-one (160 mg, 0.58 mmol) and (1-methyl-1H-pyrazol-3-yl)methanol (98 mg, 0.87 mmol). The system was capped, followed by the addition of toluene (5 mL), and purged for 5 minutes with argon. CMBP (210 mg, 0.87 mmol) was added to the reaction mixture and the solution was stirred at 100° C. for 3 hrs. The reaction was cooled to r.t. and concentrated under reduced pressure. The residue was purified by flash chromatography (silica gel, 0˜ 80% EtOAc in PE) to give 8-bromo-5-methyl-3-((1-methyl-1H-pyrazol-3-yl)methyl)-3H-pyrido[4′,3′:4,5]pyrrolo[2,3-d]pyridazin-4 (5H)-one (200 mg). LC-MS (ESI): m/z 373 (M+H)⁺.

Step G. Synthesis of methyl 5-methyl-3-((1-methyl-1H-pyrazol-3-yl)methyl)-4-oxo-4,5-dihydro-3H-pyrido[4′,3′:4,5]pyrrolo[2,3-d]pyridazine-8-carboxylate To a mixture of 8-bromo-5-methyl-3-((1-methyl-1H-pyrazol-3-yl)methyl)-3H-pyrido[4′,3′:4,5]pyrrolo[2,3-d]pyridazin-4 (5H)-one (200 mg, 0.54 mmol) in MeOH (5 mL) were added Et₃N (0.2 mL, 1.6 mmol) and Pd(dppf)Cl₂ (38 mg, 0.05 mmol) under N₂. The reaction was refluxed for 3 hr under CO, then concentrated. The residue was purified by flash chromatography (silica gel, 0˜5% MeOH in DCM) to give methyl 5-methyl-3-((1-methyl-1H-pyrazol-3-yl)methyl)-4-oxo-4,5-dihydro-3H-pyrido[4′,3′:4,5]pyrrolo[2,3-d]pyridazine-8-carboxylate (170 mg). LC-MS (ESI): m/z 353 (M+H)⁺.

Step H. Synthesis of 8-(hydroxymethyl)-5-methyl-3-((1-methyl-1H-pyrazol-3-yl)methyl)-3H-pyrido[4′,3′:4,5]pyrrolo[2,3-d]pyridazin-4 (5H)-one To a solution of methyl 5-methyl-3-((1-methyl-1H-pyrazol-3-yl)methyl)-4-oxo-4,5-dihydro-3H-pyrido[4′,3′:4,5]pyrrolo[2,3-d]pyridazine-8-carboxylate (170 mg, 0.48 mmol) in anhy. THF (5 mL) was added LiAlH₄ (27 mg, 0.72 mmol) in portions at 0° C. The mixture was stirred for 1 hr, quenched with sodium sulfate decahydrate and filtered through a Celite pad. The filtrate was concentrated and the redsidue was purified by flash chromatography (silica gel, 0˜ 6% MeOH in DCM) to give 8-(hydroxymethyl)-5-methyl-3-((1-methyl-1H-pyrazol-3-yl)methyl)-3H-pyrido[4′,3′:4,5]pyrrolo[2,3-d]pyridazin-4 (5H)-one (30 mg). LC-MS (ESI): m/z 325 (M+H)⁺.

Step I. Synthesis of 8-(chloromethyl)-5-methyl-3-((1-methyl-1H-pyrazol-3-yl)methyl)-3H-pyrido[4′,3′:4,5]pyrrolo[2,3-d]pyridazin-4 (5H)-one To a mixture of 8-(hydroxymethyl)-5-methyl-3-((1-methyl-1H-pyrazol-3-yl)methyl)-3H-pyrido[4′,3′:4,5]pyrrolo[2,3-d]pyridazin-4 (5H)-one (30 mg, 0.09 mmol) in DCM (3 mL) was added TEA (0.05 mL, 0.36 mmol) and MsCl (0.10 mL, 0.14 mmol). The reaction mixture was stirred at r.t. for 4 hr. The reaction was poured into water, extracted with DCM. The organic layer was washed with brine, dried over anhy. Na₂SO₄ and concentrated. The residue was purified by prep-TLC (eluent DCM/MeOH=15/1) to give 8-(chloromethyl)-5-methyl-3-((1-methyl-1H-pyrazol-3-yl)methyl)-3H-pyrido[4′,3′:4,5]pyrrolo[2,3-d]pyridazin-4 (5H)-one (20 mg). LC-MS (ESI): m/z 343 (M+H)⁺.

Step J. Synthesis of 5-methyl-3-((1-methyl-1H-pyrazol-3-yl)methyl)-8-(thiazol-4-ylmethyl)-3H-pyrido[4′,3′:4,5]pyrrolo[2,3-d]pyridazin-4 (5H)-one A three-neck round bottom flask was charged with 8-(chloromethyl)-5-methyl-3-((1-methyl-1H-pyrazol-3-yl)methyl)-3H-pyrido[4′,3′:4,5]pyrrolo[2,3-d]pyridazin-4 (5H)-one (20 mg, 0.06 mmol) and 4-(tributylstannyl)thiazole (68 mg, 0.18 mmol). The system was capped, followed by the addition of toluene (5 mL), and purged for 2 min with N₂. Pd(PPh₃)₄ (8 mg, 0.006 mmol) was added and the mixture was stirred at 100° C. for 1 hr. The reaction was concentrated and the residue was purified by prep-HPLC to give 5-methyl-3-((1-methyl-1H-pyrazol-3-yl)methyl)-8-(thiazol-4-ylmethyl)-3H-pyrido[4′,3′:4,5]pyrrolo[2,3-d]pyridazin-4 (5H)-one (5 mg). LC-MS (ESI): m/z 392 (M+H)⁺. ¹H NMR (400 MHz, DMSO-d₆) δ 9.12 (s, 1H), 9.02 (d, 1H), 8.79 (s, 1H), 8.06 (s, 1H), 7.57 (d, 1H), 7.38 (d, 1H), 6.11 (d, 1H), 5.32 (s, 2H), 4.43 (s, 2H), 4.36 (s, 3H), 3.77 (s, 3H).

### Example 24. PKR Mutant Assay

### Procedure:

PKR (WT or mutant) enzyme stock solution was diluted to prepare a 1.25× Reaction Mix (without ADP). 1 μL of test compound was first added to the wells followed by 40 μL of 1.25× Reaction Mix (without ADP) and incubated at room temperature (25° C.) for 60 min. The reaction was initiated with 10 μL ADP, bringing the final Reaction Mix to 1x, and the reaction progress was measured as changes in absorbance at 340 nm wavelength at room temperature.

Test compound preparation: Test compounds were prepared at 50× final concentration in DMSO. 1 to 3 dilutions were made for 11 points (for example 50 μL of 5000 μM compound was added to 100 μL 100% DMSO to yield a 1667 μM, 50 μL of this added to 100 μL DMSO to yield 556 μM, and so forth). The compounds were added to the assay as a 1 to 50 dilution (1 μL in 50 μL) to yield a top concentration of 100 μM, decreasing 3-fold for 11 points.

Reaction Mix: PKR (1.25-1000 ng/well, 0.025-20 μg/ml depending on the PKR mutant), ADP (0.05-2.3 mM depending on the PKR mutant), PEP (0.031-2 mM depending on the PKR mutant), NADH (180 μM), LDH (0.005 U/μL, Sigma #L3888), 1 mM DTT, 0.03% BSA in 1× Reaction Buffer

Reaction Buffer: 100 mM KCl, 50 mM Tris pH 7.5, 5 mM MgCl₂.

### Example 25. Red Blood Cell (RBC) Purification

Fresh blood drawn from healthy volunteers into K₂EDTA tubes was collected. Whole blood was pelleted by spinning at 500 g for 10 minutes. The transfusion bag port was cut off Purecell leukocyte reduction neofilter (Fisher NC0267633) one (1) inch above filter. A 10 ml syringe barrel was attached to the remaining cut tubing attached to the neofilter. The plasma layer was removed from the pellet of the whole blood and the pellet was resuspended in 2× volume of phosphate buffered saline (PBS). 9 ml re-suspended blood cell pellet was transferred to a 10 ml syringe that was attached to the neofilter. The whole blood was allowed to gravity flow through filter until all fluid ran through upper tubing into a filter disc. The plunger was added to the syringe. The filter was inverted and air was plunged through the syringe filter system. A new 5 ml syringe was used to remove filtered RBCs from the bag by the syringe port. Purified RBCs were transferred to a 5 ml snap cap tube that had been incubated on ice. The 5 ml snap cap tube was spun at 500 g for 10 minutes at 15 C. Supernatant was aspirated and resuspended in AGAM (1×PBS, 1% glucose, 170 mg/L adenine, 5.25 g/L mannitol) at a density of 4×10⁹ cells/mL.

### Example 26. Cell Based ATP Assay

For cell based ATP assays, the compound as described herein was prepared in 100% DMSO as a 10 mM stock. Serial dilutions (1:4) were performed in 96-well V-bottom storage plate and then added 1:100 to 96-well V-bottom plates containing AGAM. 10 μL/well of compound diluted in AGAM was added to black clear bottom assay plates. RBCs were diluted in AGAM media to a density of 1×10⁷ cells/mL before added 90 μL/well to black clear bottom assay plates (final compound concentration at 0.1% DMSO concentration). Assay plates were sealed using aluminum foil seals and incubated overnight at 37° C. in a humidified chamber. ATP levels were read out using Cell-Titer-Glo (Promega).

Having thus described several aspects of several embodiments, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

### Example 27. PKM2 Assay

Procedure:

PKM2 enzyme stock solution was diluted to prepare a 1.25× Reaction Mix (without ADP). 1 μL of test compound was first added to the wells followed by 40 μL of 1.25× Reaction Mix (without ADP) and incubated at room temperature (25° C.) for 60 min. The reaction was initiated with 10 μL ADP (0.4 mM final concentration), bringing the final Reaction Mix to 1x, and the reaction progress was measured as changes in absorbance at 340 nm wavelength at room temperature.

Test compound preparation: Test compounds were prepared at 50× final concentration in DMSO. 1 to 3 dilutions were made for 11 points (for example 50 μL of 5000 μM compound was added to 100 μL 100% DMSO to yield a 1667 μM, 50 μL of this added to 100 μL DMSO to yield 556 μM, and so forth). The compounds were added to the assay as a 1 to 50 dilution (1 μL in 50 μL) to yield a top concentration of 100 μM, decreasing 3-fold for 11 points.

Reaction Mix: PKM2 (5 ng/well, 0.1 μg/ml), ADP (0.4 mM), PEP (0.11 mM), NADH (180 μM), LDH (0.005 U/μL, Sigma #L3888), 1 mM DTT, 0.03% BSA in 1×

### Reaction Buffer

Reaction Buffer: 100 mM KCl, 50 mM Tris pH 7.5, 5 mM MgCl₂.

Having thus described several aspects of several embodiments, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A compound represented by the following structural formula: 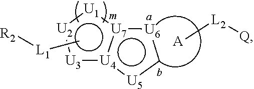
or a pharmaceutically acceptable salt thereof, wherein:
U₁, U₂, and U₃ are each independently N, O, S, C, or CR₁, as valency permits;
U₄, U₆, and U₇ are each independently N or C, as valency permits;
U₅ is N, NR₃, or CR₄, as valency permits;
m is 1 or 2;
Ring A is phenyl, 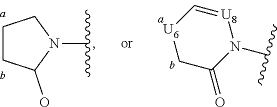
U₈ is N or CR₁;
each instance of R₁ is independently hydrogen or C₁-C₆ alkyl;
L₁ is —S—, —S—CH₂—, —CH₂—S—, —S(═O)₂—, —S(═O)—, —S(═O)₂O—, —OS(═O)₂—, —S(═O)O—, —OS(═O)—, —S(═O)CH₂—, —CH₂S(═O)—, —S(═O)₂CH₂—, —CH₂S(═O)₂—, —S(═O)₂NR₅—, —NR₅S(═O)₂—, —S(═O)NR₅—, —NR₅S(═O)—, —NR₅S(═O)₂O—, —OS(═O)₂NR₅—, —NR₅S(═O)O—, —OS(═O)NR₅—, —S(═O)(═NR₅)—, —C(═O)—, —C(═O)O—, —OC(═O)—, —C(═O)NR₅—, —N(R₅)C(═O)—, —NR₅C(═O)O—, —OC(═O)NR₅—, —NR₅C(═O)NR₅—, —NR₅—, —C(═S)NR₅—, —N(R₅)C(═S)—, or —(CRⱼRₖ)_{q}—;
R₂ is C₁-C₆ alkyl, C₃-C₁₂ cycloalkyl, 3- to 8-membered heterocyclyl, 6- to 14-membered aryl, or 5- to 14-membered heteroaryl, wherein the alkyl is optionally substituted with 0 to 3 groups each independently selected from halogen, OH, CN, and NR₅R₅, and wherein each cycloalkyl, heterocyclyl, aryl, and heteroaryl is optionally substituted at each substitutable ring carbon atom with R^{p} and optionally substituted at each substitutable ring nitrogen atom by R^{nc}; or
-L₁-R₂ is —H, —CN, —CH₃, —OH, Br, C₁-C₆ haloalkyl, C₂-C₆ alkenyl, C₁-C₆ alkyl, C₃-C₁₂ cycloalkyl, 3- to 8-membered heterocyclyl, 6- to 14-membered aryl, or 5- to 14-membered heteroaryl; wherein each alkyl and alkenyl is optionally substituted with 0 to 3 groups each independently selected from halogen, OH, CN, and NR₅R₅, and wherein each cycloalkyl, heterocyclyl, aryl, and heteroaryl is optionally substituted at each substitutable ring carbon atom with R^{p} and optionally substituted at each substitutable ring nitrogen atom by R^{nc};
each instance of R^{p} is independently hydrogen, halogen, —CN, —NO₂, —N₃, C₁-C₆ alkyl, C₂-C₆ alkenyl, C₂-C₆ alkynyl, —OR^{c3}, —SR^{c3}, —N(R^{c3})₂, —C(═O)N(R^{c3})₂, —N(R^{c3})C(═O)R^{c3}, —C(═O)R^{c3}, —C(═O)OR^{c3}, —OC(═O)R^{c3}, —S(═O)R^{c3}, —S(═O)₂R^{c3}, —S(═O)OR^{c3}, —OS(═O)R^{c3}, —S(═O)₂OR^{c3}, —OS(═O)₂R^{c3}, —S(═O)N(R^{c3})₂, —S(═O)₂N(R^{c3})₂, —N(R^{c3})S(═O)R^{c3}, —N(R^{c3})S(═O)₂R^{c3}, —N(R^{c3})C(═O)OR^{c3}, —OC(═O)N(R^{c3})₂, —N(R^{c3})C(═O)N(R^{c3})₂, —N(R^{c3})S(═O)N(R^{c3})₂, —N(R^{c3})S(═O)₂N(R^{c3})₂, —N(R^{c3})S(═O)OR^{c3}, —N(R^{c3})S(═O)₂OR^{c3}, —OS(═O)N(R^{c3})₂, —OS(═O)₂N(R^{c3})₂; or alternatively
two instances of R^{p} attached to the adjacent ring carbon atoms, can be taken together with the carbon atoms to which they are attached to form 3- to 8-membered cycloalkyl, 5- to 6-membered saturated or partially saturated monocyclic heterocyclyl, or 5- to 6-membered monocyclic heteroaryl; wherein:
each instance of R^{c3} is independently hydrogen or C₁-C₆ alkyl;
L₂ is —S—, —S—CH₂—, —CH₂—S—, —S(═O)₂—, —S(═O)—, —S(═O)₂O—, —OS(═O)₂—, —S(═O)O—, —OS(═O)—, —S(═O)CH₂—, —CH₂S(═O)—, —S(═O)₂CH₂—, —CH₂S(═O)₂—, —S(═O)₂NR₅—, —NR₅S(═O)₂—, —S(═O)NR₅—, —NR₅S(═O)—, —NR₅S(═O)₂O—, —OS(═O)₂NR₅—, —NR₅S(═O)O—, —OS(═O)NR₅—, —S(═O)(═NR₅)—, —C(═O)—, —C(═O)O—, —OC(═O)—, —C(═O)NR₅—, —N(R₅)C(═O)—, —NR₅C(═O)O—, —OC(═O)NR₅—, —NR₅C(═O)NR₅—, —NR₅—, —C(═S)NR₅—, —N(R₅)C(═S)—, or —(CRₐR_{b})ᵣ—;
each instance of R^{a} and R^{b} are independently hydrogen, halogen, CN, OH, NO₂, N₃, or C₁-C₆ alkyl; wherein the C₁-C₆ alkyl represented by R^{a} or R^{b} are each optionally substituted with 0 to 3 groups each independently selected from halogen, OH, CN, and NR₅R₅;
each instance of R^{j} and R^{k} are independently hydrogen, halogen, CN, OH, NO₂, N₃, or C₁-C₆ alkyl; wherein the C₁-C₆ alkyl represented by R^{a} or R^{b} are each optionally substituted with 0 to 3 groups each independently selected from halogen, OH, CN, and NR₅R₅;
q is 1 or 2;
r is 1 or 2;
Q is C₃-C₁₂ cycloalkyl, 3- to 8-membered heterocyclyl, 6- to 14-membered aryl, or 5- to 14-membered heteroaryl, each of which is optionally substituted at each substitutable ring carbon atom with Rⁿ and optionally substituted at each substitutable ring nitrogen atom by R^{na}; or
-L₂-Q is —H, —CN, —CH₃, —OH, Br, C₁-C₆ haloalkyl, C₂-C₆ alkenyl, C₁-C₆ alkyl, C₃-C₁₂ cycloalkyl, 3- to 8-membered heterocyclyl, 6- to 14-membered aryl, or 5- to 14-membered heteroaryl; wherein each alkyl and alkenyl is optionally substituted with 0 to 3 groups each independently selected from halogen, OH, CN, and NR₅R₅, and wherein each cycloalkyl, heterocyclyl, aryl, and heteroaryl is optionally substituted at each substitutable ring carbon atom with Rⁿ and optionally substituted at each substitutable ring nitrogen atom by R^{na};
each instance of Rⁿ is independently hydrogen, halogen, —CN, —NO₂, —N₃, C₁-C₆ alkyl, C₂-C₆ alkenyl, C₂-C₆ alkynyl, —OR^{c4}, —SR^{c4}, —N(R^{c4})₂, —C(═O)N(R^{c4})₂, —N(R^{c4})C(═O)R^{c4}, —C(═O)R^{c4}, —C(═O)OR^{c4}, —OC(═O)R^{c4}, —S(═O)R^{c4}, —S(═O)₂R^{c4}, —S(═O)OR^{c4}, —OS(═O)R^{c4}, —S(═O)₂OR^{c4}, —OS(═O)₂R^{c4}, —S(═O)N(R^{c4})₂, —S(═O)₂N(R^{c4})₂, —N(R^{c4})S(═O)R^{c4}, —N(R^{c4})S(═O)₂R₄, —N(R^{c4})C(═O)OR^{c4}, —OC(═O)N(R^{c4})₂, —N(R^{c4})C(═O)N(R^{c4})₂, —N(R^{c4})S(═O)N(R^{c4})₂, —N(R^{c4})S(═O)₂N(R^{c4})₂, —N(R^{c4})S(═O)OR^{c4}, —N(R^{c4})S(═O)₂OR^{c4}, —OS(═O)N(R^{c4})₂, or —OS(═O)₂N(R^{c4})₂; or alternatively
two instances of Rⁿ attached to the adjacent ring carbon atoms, can be taken together with the carbon atoms to which they are attached to form an optionally substituted 3- to 8-membered cycloalkyl, 5- to 6-membered saturated or partially saturated monocyclic heterocyclyl, or 5- to 6-membered monocyclic heteroaryl; wherein:
each instance of R^{c4} is independently hydrogen or C₁-C₆ alkyl;
R₃ is hydrogen or C₁-C₆ alkyl;
R₄ is hydrogen, C₁-C₆ alkyl, C₁-C₆ haloalkyl, C₂-C₆ alkynyl, halogen, CN, —C(═O)NR₅R₅, or C≡C(CH₂)_{w}OH, wherein w is 1, 2, 3, 4, 5, or 6, and wherein each alkyl, haloalkyl, and alkynyl is independently optionally substituted with 1-3 instances of C₁-C₄ alkyl or halogen;
each instance of R^{na} and R^{nc} is independently hydrogen, C₁-C₆ alkyl, or C₁-C₆ haloalkyl; and
each instance of R₅ is independently hydrogen or C₁-C₆ alkyl;
provided that 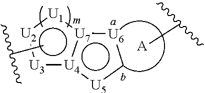
is other than 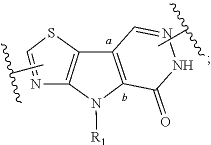
and provided that when 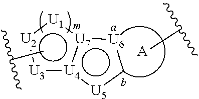
is 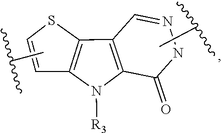
L₂ is —(CRₐR_{b})ᵣ— and Q is phenyl optionally substituted with Rⁿ and R^{na}, then L₁ is —(CRⱼRₖ)_{q}— and R₂ is cycloalkyl, heterocyclyl, aryl, or heteroaryl optionally substituted with R^{p} and R^{nc}.

2. The compound of claim 1, or a pharmaceutically acceptable salt thereof, wherein:
L₁ is —S—, —S—CH₂—, —CH₂—S—, —S(═O)₂—, —S(═O)—, —S(═O)₂O—, —OS(═O)₂—, —S(═O)O—, —OS(═O)—, —S(═O)CH₂—, —CH₂S(═O)—, —S(═O)₂CH₂—, —CH₂S(═O)₂—, —S(═O)₂NR₅—, —NR₅S(═O)₂—, —S(═O)NR₅—, —NR₅S(═O)—, —NR₅S(═O)₂O—, —OS(═O)₂NR₅—, —NR₅S(═O)O—, —OS(═O)NR₅—, —S(═O)(═NR₅)—, —C(═O)—, —C(═O)O—, —OC(═O)—, —C(═O)NR₅—, —N(R₅)C(═O)—, —NR₅C(═O)O—, —OC(═O)NR₅—, —NR₅C(═O)NR₅—, —NR₅—, —C(═S)NR₅—, —N(R₅)C(═S)—, or —(CRⱼRₖ)_{q}—;
R₂ is C₁-C₆ alkyl, C₃-C₁₂ cycloalkyl, 3- to 8-membered heterocyclyl, 6- to 14-membered aryl, or 5- to 14-membered heteroaryl, wherein the alkyl is optionally substituted with 0 to 3 groups each independently selected from halogen, OH, CN, and NR₅R₅, and wherein each cycloalkyl, heterocyclyl, aryl, and heteroaryl is optionally substituted at each substitutable ring carbon atom with R^{p} and optionally substituted at each substitutable ring nitrogen atom by R^{nc}; or
-L₁-R₂ is —H, —CN, —CH₃, —OH, Br, C₁-C₆ haloalkyl, or C₂-C₆ alkenyl wherein the alkenyl is optionally substituted with 0 to 3 groups each independently selected from halogen, OH, CN, and NR₅R₅; Q is C₃-C₁₂ cycloalkyl, 3- to 8-membered heterocyclyl, 6- to 14-membered aryl, or 5- to 14-membered heteroaryl, each of which is optionally substituted at each substitutable ring carbon atom with Rⁿ and optionally substituted at each substitutable ring nitrogen atom by R^{na}; and
R₄ is hydrogen, C₁-C₆ alkyl, C₁-C₆ haloalkyl, halogen, CN, —C(═O)NR₅R₅, or C≡C(CH₂)_{w}OH, wherein w is 1, 2, 3, 4, 5, or 6.

3. The compound of claim 1 or 2, or a pharmaceutically acceptable salt thereof, wherein:
each instance of R^{p} is independently hydrogen, halogen, CN, OH, NO₂, N₃, C₁-C₆ alkyl, C₁-C₆ alkoxy, —C(═O)NR₅R₅, or NR₅R₅; or alternatively two instances of R^{p} attached to the adjacent ring carbon atoms, can be taken together with the carbon atoms to which they are attached to form 3- to 8-membered cycloalkyl, 5- to 6-membered saturated or partially saturated monocyclic heterocyclyl, or 5- to 6-membered monocyclic heteroaryl; and
each instance of Rⁿ is independently hydrogen, halogen, CN, OH, NO₂, N₃, C₁-C₆ alkyl, C₁-C₆ alkoxy, —C(═O)NR₅R₅, or NR₅R₅; or alternatively two instances of Rⁿ attached to the adjacent ring carbon atoms, can be taken together with the carbon atoms to which they are attached to form 3- to 8-membered cycloalkyl, 5- to 6-membered saturated or partially saturated monocyclic heterocyclyl, or 5- to 6-membered monocyclic heteroaryl.

4. A compound represented by the following structural formula: 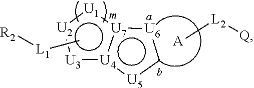
or a pharmaceutically acceptable salt thereof, wherein:
U₁, U₂, and U₃ are each independently N, O, S, C, or CR₁, as valency permits;
U₄, U₆, and U₇ are each independently N or C, as valency permits;
U₅ is N, NR₃, or CR₄, as valency permits;
m is 1 or 2;
Ring A is phenyl, 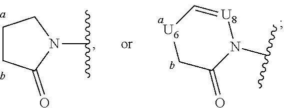
U₈ is N or CR₁;
each instance of R₁ is independently hydrogen or C₁-C₆ alkyl;
L₁ is —S(═O)₂—, —S(═O)—, —C(═O)—, —C(═O)O—, —OC(═O)—, —C(═O)NR₅—, —N(R₅)C(═O)—, —NR₅—, or —(CRⱼRₖ)_{q}—; and
R₂ is C₁-C₆ alkyl, phenyl or 5- to 14-membered heteroaryl, wherein each phenyl and heteroaryl is optionally substituted at each substitutable ring carbon atom with R^{p} and optionally substituted at each substitutable ring nitrogen atom by R^{nc}; or
-L₁-R₂ is —H, —CN, —CH₃, —OH, Br, C₁-C₂ haloalkyl, —CH═CH₂, or C₁-C₆ hydroxyalkyl; and
each instance of R^{p} is independently hydrogen, halogen, CN, OH, NO₂, N₃, C₁-C₆ alkyl, C₁-C₆ alkoxy, —C(═O)NR₅R₅, or NR₅R₅; or alternatively
two instances of R^{p} attached to the adjacent ring carbon atoms, can be taken together with the carbon atoms to which they are attached to form 5- to 6-membered monocyclic heteroaryl;
L₂ is —S(═O)₂—, —S(═O)—, —C(═O)—, —C(═O)O—, —OC(═O)—, —C(═O)NR₅—, —N(R₅)C(═O)—, —NR₅—, or —(CRₐR_{b})ᵣ—;
each instance of R^{a} and R^{b} are independently hydrogen, halogen, CN, OH, NO₂, N₃, or C₁-C₆ alkyl; wherein the C₁-C₆ alkyl represented by R^{a} or R^{b} are each optionally substituted with 0 to 3 groups each independently selected from halogen, OH, CN, and NR₅R₅;
each instance of R^{j} and R^{k} are independently hydrogen, halogen, CN, OH, NO₂, N₃, or C₁-C₆ alkyl; wherein the C₁-C₆ alkyl represented by R^{a} or R^{b} are each optionally substituted with 0 to 3 groups each independently selected from halogen, OH, CN, and NR₅R₅;
q is 1 or 2;
r is 1 or 2;
Q is phenyl or 5- to 14-membered heteroaryl, each of which is optionally substituted at each substitutable ring carbon atom with Rⁿ and optionally substituted at each substitutable ring nitrogen atom by R^{na};
each instance of Rⁿ is independently hydrogen, halogen, CN, OH, NO₂, N₃, C₁-C₆ alkyl, C₁-C₆ alkoxy, —C(═O)NR₅R₅, or NR₅R₅; or alternatively
two instances of Rⁿ attached to the adjacent ring carbon atoms, can be taken together with the carbon atoms to which they are attached to form 5- to 6-membered monocyclic heteroaryl;
R₃ is hydrogen or C₁-C₆ alkyl;
R₄ is hydrogen, C₁-C₆ alkyl, C₁-C₆ haloalkyl, halogen, CN, —C(═O)NR₅R₅, or C≡C(CH₂)_{w}OH, wherein w is 1, 2, 3, 4, 5, or 6;
each instance of R^{na} and R^{nc} is independently hydrogen, C₁-C₆ alkyl, or C₁-C₆ haloalkyl; and
each instance of R₅ is independently hydrogen or C₁-C₆ alkyl;
provided that 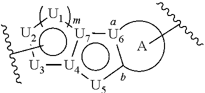
is other than 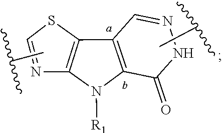
and provided that when 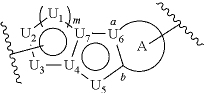
is 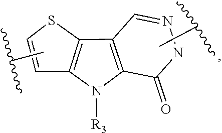
L₂ is —(CRₐR_{b})ᵣ— and Q is phenyl optionally substituted with Rⁿ and R^{na}, then L₁ is —(CRⱼRₖ)_{q}— and R₂ is phenyl or heteroaryl optionally substituted with R^{p} and R^{nc}.

5. The compound of any one of claims 1 to 4, represented by a structural formula selected from: 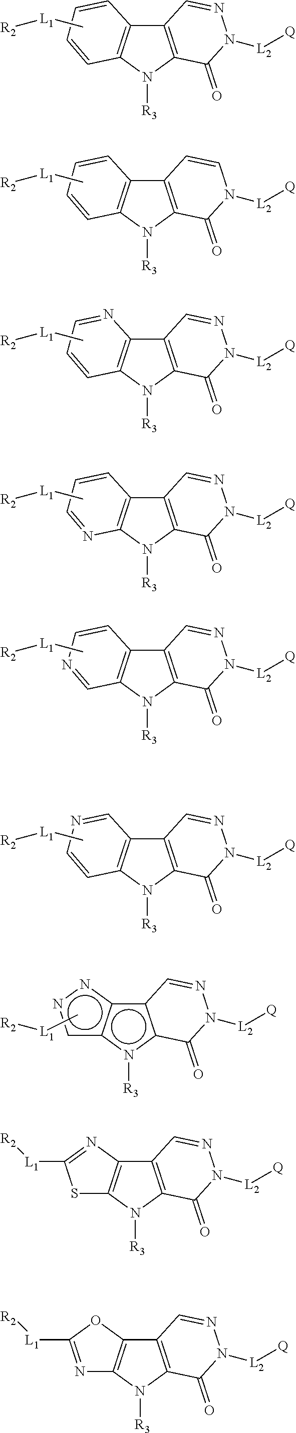 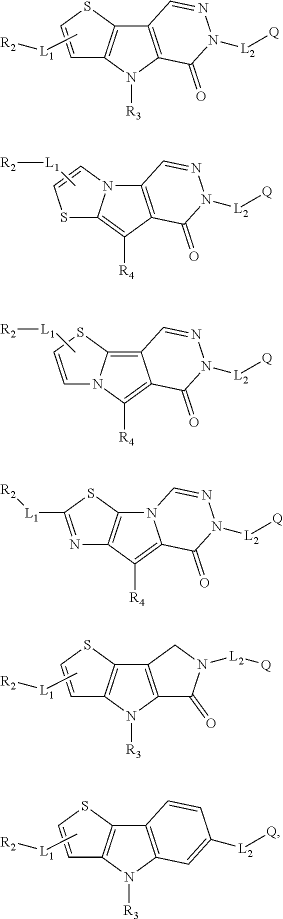
or a pharmaceutically acceptable salt thereof.

6. The compound of any one of claims 1 to 5, represented by a structural formula selected from: 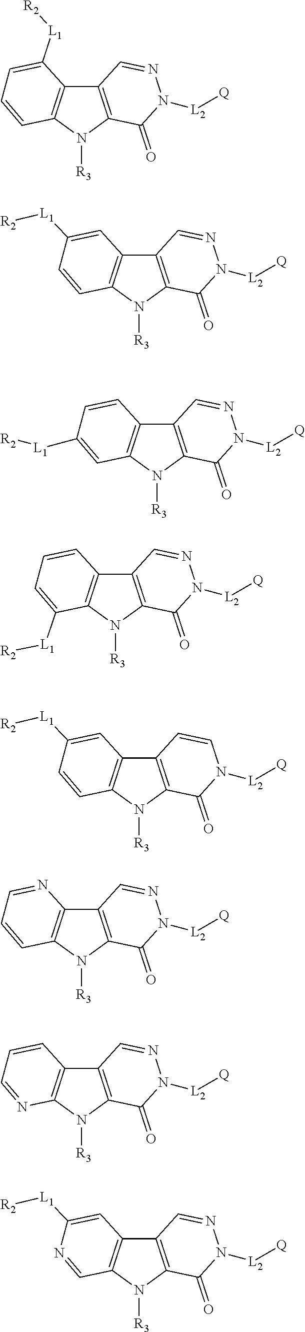 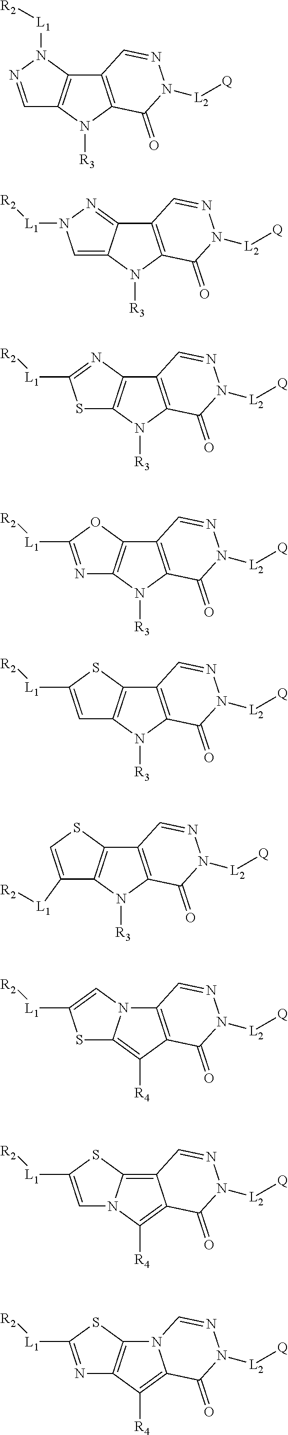 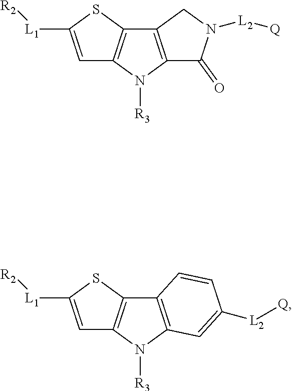
or a pharmaceutically acceptable salt thereof.

7. The compound of any one of claims 1 to 6, or a pharmaceutically acceptable salt thereof, wherein:
R₃ is C₁-C₂ alkyl; and
R₄ is C₁-C₂ alkyl, C₁-C₂ haloalkyl, halogen, CN, —C(═O)NR₅R₅, or C≡C(CH₂)_{w}OH, wherein w is 1 or 2.

8. The compound of any one of claims 1 to 7, or a pharmaceutically acceptable salt thereof, wherein:
R₃ is CH₃; and
R₄ is CH₃, CF₃, Br, CN, C(═O)NH₂, or C≡CCH₂OH.

9. The compound of any one of claims 1 to 8, or a pharmaceutically acceptable salt thereof, wherein R₁ is H or CH₃ and each instance of R₅ is H or CH₃.

10. The compound of any one of claims 1 to 9, or a pharmaceutically acceptable salt thereof, wherein:
L₁ is —S(═O)₂—, —S(═O)—, —C(═O)O—*, —C(═O)NRs-*, —NR₅—, or —(CRⱼRₖ)_{q}—, wherein “*” designates the connection to R₂;
L₂ is —(CRₐR_{b})ᵣ—; and
wherein R^{a}, R^{b}, R^{j} and R^{k} are each independently hydrogen or halogen.

11. The compound of any one of claims 1 to 10, or a pharmaceutically acceptable salt thereof, wherein L₁ is —S(═O)₂—, —S(═O)—, —C(═O)O—*, —C(═O)NH—*, —NH—, —CH₂—, or —CF₂—, wherein “*” designates the connection to R₂.

12. The compound of any one of claims 1 to 11, or a pharmaceutically acceptable salt thereof, wherein L₂ is —CH₂—.

13. The compound of any one of claims 1 to 12, or a pharmaceutically acceptable salt thereof, wherein:
each instance of R^{na} is independently hydrogen, C₁-C₂ alkyl, or C₁-C₂ haloalkyl; and
each instance of Rⁿ is independently hydrogen, CN, OH, C₁-C₄ alkyl, C₁-C₄ alkoxy, —C(═O)NR₅R₅, or NR₅R₅, or two Rⁿ attached to the adjacent carbon atoms of the phenyl ring of Q, can be taken together with the carbon atoms to which they are attached to form 5- to 6-membered monocyclic heteroaryl.

14. The compound of any one of claims 1 to 13, or a pharmaceutically acceptable salt thereof, wherein Q is selected from one of the following structural formulae: 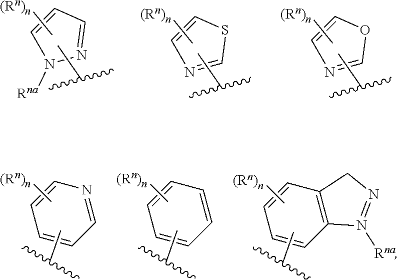
wherein n is 0 or 1.

15. The compound of any one of claims 1 to 14, or a pharmaceutically acceptable salt thereof, wherein R^{na} is hydrogen or CH₃; Rⁿ is H, CH₃, CN, OCH₃, NH₂, or C(═O)NH₂; and n is 0 or 1.

16. The compound of any one of claims 1 to 15, or a pharmaceutically acceptable salt thereof, wherein:
each instance of R^{nc} is independently hydrogen, C₁-C₂ alkyl, or C₁-C₂ haloalkyl; and
each instance of R^{p} is independently hydrogen, CN, OH, C₁-C₄ alkyl, C₁-C₄ alkoxy, —C(═O)NR₅R₅, or NR₅R₅, or two R^{p} attached to the adjacent carbon atoms of the phenyl ring of Q, can be taken together with the carbon atoms to which they are attached to form 5- to 6-membered monocyclic heteroaryl.

17. The compound of any one of claims 1 to 16, or a pharmaceutically acceptable salt thereof, wherein R₂ is selected from one of the following structural formulae: 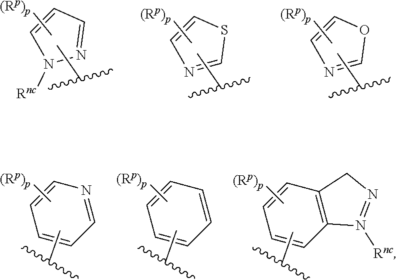
wherein p is 0 or 1.

18. The compound of any one of claims 1 to 17, or a pharmaceutically acceptable salt thereof, wherein R^{nc} is hydrogen or CH₃; R^{p} is H, CH₃, CN, OCH₃, NH₂, or C(═O)NH₂; and p is 0 or 1.

19. The compound of claim 17 or 18, or a pharmaceutically acceptable salt thereof, wherein p is 0.

20. The compound of any one of claims 1 to 15, or a pharmaceutically acceptable salt thereof, wherein R₂ is C₁-C₂ alkyl.

21. The compound of any one of claims 1 to 9 or 12 to 15, or a pharmaceutically acceptable salt thereof, wherein -L₁-R₂ is —H, —CN, —CH₃, —OH, —Br, —CF₃, —CH═CH₂, or —CH₂OH.

22. The compound of any one of claims 1 to 9 or 12 to 15, or a pharmaceutically acceptable salt thereof, wherein R₂ is —CH₃; and L₁ is —S(═O)₂—, —S(═O)—, —C(═O)O—*, —C(═O)NH—* or —NH—, wherein “*” designates the connection to R₂.

23. The compound of any one of claims 1 to 22, or a pharmaceutically acceptable salt thereof, wherein the compound is any one from Table 1.

24. A pharmaceutical composition comprising an effective amount of the compound according to any one of claims 1 to 23 or a pharmaceutically acceptable salt thereof, and a pharmaceutically acceptable carrier.

25. A method for increasing the lifetime of red blood cells (RBCs) comprising contacting the red blood cells with an effective amount of the compound according to any one of claims 1 to 23, or a pharmaceutically acceptable salt thereof; or a pharmaceutical composition thereof.

26. The method of claim 25, wherein the compound or the pharmaceutical composition is added directly to whole blood comprising the red blood cells or packed red blood cells comprising the red blood cells extracorporeally.

27. The method of claim 26, wherein the compound or the pharmaceutical composition is administered to a subject comprising the red blood cells.

28. A method for regulating 2,3-diphosphoglycerate levels in blood comprising contacting the blood with an effective amount of the compound according to any one of claims 1 to 23, or a pharmaceutically acceptable salt thereof; or a pharmaceutical composition thereof.

29. A method for treating anemia in a subject comprising administering to the subject an effective amount of the compound according to any one of claims 1 to 23, or a pharmaceutically acceptable salt thereof; or a pharmaceutical composition thereof.

30. The method of claim 29, wherein the anemia is dyserythropoietic anemia.

31. A method for treating hemolytic anemia in a subject comprising administering to the subject an effective amount of the compound according to any one of claims 1 to 23, or a pharmaceutically acceptable salt thereof; or a pharmaceutical composition thereof.

32. The method of claim 31, wherein the hemolytic anemia is hereditary and/or congenital hemolytic anemia, acquired hemolytic anemia, chronic hemolytic anemia caused by phosphoglycerate kinase deficiency, anemia of chronic diseases, non-spherocytic hemolytic anemia, or hereditary spherocytosis.

33. A method for treating sickle cell disease in a subject comprising administering to the subject an effective amount of the compound according to any one of claims 1 to 23, or a pharmaceutically acceptable salt thereof; or a pharmaceutical composition thereof.

34. A method of treating pyruvate kinase deficiency (PKD) in a subject comprising administering to the subject an effective amount of the compound according to any one of claims 1 to 23, or a pharmaceutically acceptable salt thereof; or a pharmaceutical composition thereof.

35. A method of treating thalassemia, hereditary spherocytosis, hereditary elliptocytosis, abetalipoproteinemia or Bassen-Kornzweig syndrome, sickle cell disease, paroxysmal nocturnal hemoglobinuria, acquired hemolytic anemia, or anemia of chronic diseases comprising administering to a subject an effective amount of the compound according to any one of claims 1 to 23, or a pharmaceutically acceptable salt thereof; or a pharmaceutical composition thereof.

36. A method of treating thalassemia comprising administering to a subject an effective amount of the compound according to any one of claims 1 to 23, or a pharmaceutically acceptable salt thereof; or a pharmaceutical composition thereof.

37. The method of claim 36, wherein the thalassemia is beta thalassemia.

38. A method for activating mutant pyruvate kinase R (PKR) in red blood cells in a subject in need thereof comprising administering to the subject an effective amount of the compound according to any one of claims 1 to 23, or a pharmaceutically acceptable salt thereof; or a pharmaceutical composition thereof.

39. A method for activating wild-type pyruvate kinase R (PKR) in red blood cells in a subject in need thereof comprising administering to the subject an effective amount of the compound according to any one of claims 1 to 23, or a pharmaceutically acceptable salt thereof; or a pharmaceutical composition thereof.

40. A method of increasing amount of hemoglobin in a subject in need thereofcomprising administering to the subject an effective amount of the compound according to any one of claims 1 to 23, or a pharmaceutically acceptable salt thereof; or a pharmaceutical composition thereof.

41. A method of modulating pyruvate kinase M2 (PKM2) activity in a subject in need thereof comprising administering an effective amount of the compound according to any one of claims 1 to 23, or a pharmaceutically acceptable salt thereof; or a pharmaceutical composition thereof.

42. A method of modulating the level of plasma glucose in a subject in need thereof comprising administering an effective amount of the compound according to any one of claims 1 to 23, or a pharmaceutically acceptable salt thereof; or a pharmaceutical composition thereof.

43. A method of inhibiting cell proliferation in a subject suffering from or susceptible to a disease or disorder associated with function of PKM2 comprising administering an effective amount of the compound according to any one of claims 1 to 23, or a pharmaceutically acceptable salt thereof; or a pharmaceutical composition thereof.

44. A method of treating a disease associated with the aberrant activity of PKM2 in a subject in need thereof comprising administering an effective amount of the compound according to any one of claims 1 to 23, or a pharmaceutically acceptable salt thereof; or a pharmaceutical composition thereof.

45. The method of claim 44, wherein the disease is a proliferative disease.

46. The method of claim 45, wherein the disease is cancer, obesity, a diabetic disease (e.g. diabetic nephropathy (DN)), atherosclerosis, restenosis, coronary artery disease (CAD), Bloom Syndrome (BS), benign prostatic hyperplasia (BPH), or an autoimmune disease.

47. A method of treating hyperglycemia in a subject in need thereof comprising administering an effective amount of the compound according to any one of claims 1 to 23, or a pharmaceutically acceptable salt thereof; or a pharmaceutical composition thereof.

48. A method of treating a diabetic disease in a subject in need thereof comprising administering an effective amount of the compound according to any one of claims 1 to 23, or a pharmaceutically acceptable salt thereof; or a pharmaceutical composition thereof.

49. The method of claim 48, wherein the diabetic disease is diabetic nephropathy.

50. The method of any one of claims 41 to 49, further comprising identifying a subject who would benefit from modulation of PKM2.

51. The method of claim 41, wherein the modulating is activating.
